(19) United States
(12) Patent Application Publication
(10) Pub. No.: US 2003/0198953 A1
(43) Pub. Date: Oct. 23, 2003

(54) Novel proteins and nucleic acids encoding same

(76) Inventors: Kimberly A. Spytek, New Haven, CT (US); Kumud Majumder, Stamford, CT (US); Velizar T. Tchernev, Branford, CT (US); Vishnu Mishra, Gainesville, FL (US); Muralidhara Padigaru, Bronx, NY (US); Steven K. Spaderna, Berlin, CT (US); Suresh G. Shenoy, Branford, CT (US); Luca Rastelli, Guilford, CT (US); Li Li, Branford, CT (US); Raymond J. Taupier, East Haven, CT (US); Esha Gangolli, Madison, CT (US)

Correspondence Address: Ivor R. Elrifi, Esq., MINTZ, LEVIN, COHN, FERRIS,, GLOVSKY & POPEO, P.C., One Financial Center, Boston, MA, 02111, US

(21) Appl. No.: 09/863,776

(22) Filed: May 23, 2001

Related U.S. Application Data
(63) Continuation-in-part of application No. 09/540,763, filed on Mar. 30, 2000.
(60) Provisional application No. 60/206,679, filed on May 24, 2000.
(60) Provisional application No. 60/206,688, filed on May 24, 2000.
(60) Provisional application No. 60/206,829, filed on May 24, 2000.
(60) Provisional application No. 60/207,748, filed on May 30, 2000.
(60) Provisional application No. 60/207,798, filed on May 30, 2000.
(60) Provisional application No. 60/208,263, filed on May 31, 2000.
(60) Provisional application No. 60/208,831, filed on Jun. 2, 2000.
(60) Provisional application No. 60/209,451, filed on Jun. 5, 2000.
(60) Provisional application No. 60/210,060, filed on Jun. 7, 2000.
(60) Provisional application No. 60/219,507, filed on Jul. 20, 2000.
(60) Provisional application No. 60/221,337, filed on Jul. 26, 2000.
(60) Provisional application No. 60/221,927, filed on Jul. 31, 2000.
(60) Provisional application No. 60/263,135, filed on Jan. 19, 2001.
(60) Provisional application No. 60/263,688, filed on Jan. 24, 2001.
(60) Provisional application No. 60/263,694, filed on Jan. 24, 2001.

(51) Int. Cl. C12Q 1/68; C07H 21/04; C12N 9/00; C12P 21/02; C12N 5/06

(52) U.S. Cl. 435/6; 435/69.1; 435/183; 435/320.1; 435/325; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application is a continuation in part of U.S. Ser. No. 09/540,763, filed Mar. 30, 2000, and claims priority from Applications U.S. Ser. No. 60/206,679, filed May 24, 2000; U.S. Ser. No. 60/206,688, filed May 24, 2000; U.S. Ser. No. 60/206,829, filed May 24, 2000; U.S. Ser. No. 60/207,748, filed May 30, 2000; U.S. Ser. No. 60/207,798, filed May 30, 2000; U.S. Ser. No. 60/208,263, filed May 31, 2000; U.S. Ser. No. 60/208,831, filed Jun. 2, 2000; U.S. Ser. No. 60/209,451, filed Jun. 5, 2000; U.S. Ser. No. 60/210,060, filed Jun. 7, 2000; U.S. Ser. No. 60/219,507 filed Jul. 20, 2000, U.S. Ser. No. 60/221,337, filed Jul. 26, 2000; U.S. Ser. No. 60/221,927, filed Jul. 31, 2000; U.S. Ser. No. 60/263,135, filed Jan. 19, 2001; U.S. Ser. No. 60/263,688, filed Jan. 24, 2001; and 60/263,694, filed Jan. 24, 2001, each of which is incorporated by reference in its entirety.

### FIELD OF THE INVENTION

The invention generally relates to nucleic acids and polypeptides encoded therefrom.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, and NOV9 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1, 3, 5, 7, 9, 11, 13, 15, 17, 19, 21, 23, 25, 27, 29, 31, and 33. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 26, 28, 30, 32, and 34. The nucleic acid can be, for example, a genomic DNA fragment or a cDNA molecule that includes the nucleic acid sequence of any of SEQ ID NOS: 1, 3, 5,7,9, 11, 13, 15, 17, 19,21,23,25,27,29,31, and 33.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, 25, 27, 29, 31, and 33) or a complement of said oligonucleotide.

Also included in the invention are substantially purified NOVX polypeptides (SEQ ID NOS:2, 4, 6, 8, 10, 12, 14, 16, 18, 20, 22, 24, 26, 28, 30, 32, and 34). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, or other disorders related to cell signal processing and metabolic pathway modulation. The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from: developmental diseases, MHCII and III diseases (immune diseases), taste and scent detectability Disorders, Burkitt's lymphoma, corticoneurogenic disease, signal transduction pathway disorders, Retinal diseases including those involving photoreception, Cell growth rate disorders; cell shape disorders, feeding disorders; control of feeding; potential obesity due to over-eating; potential disorders due to starvation (lack of appetite), noninsulin-dependent diabetes mellitus (NIDDM1), bacterial, fungal, protozoal and viral infections (particularly infections caused by HIV-1 or HIV-2), pain, cancer (including but not limited to neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation. Dentatorubro-pallidoluysian atrophy (DRPLA) Hypophosphatemic rickets, autosomal dominant (2) Acrocallosal syndrome and dyskinesias, such as Huntington's disease or Gilles de la Tourette syndrome and/or other pathologies and disorders of the like.

The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from bacterial, fungal, protozoal and viral infections (particularly infections caused by HIV-1 or HIV-2), pain, cancer (including but not limited to Neoplasm; adenocarcinoma; lymphoma; prostate cancer; uterus cancer), anorexia, bulimia, asthma, Parkinson's disease, acute heart failure, hypotension, hypertension, urinary retention, osteoporosis, Crohn's disease; multiple sclerosis; and Treatment of Albright Hereditary Ostoeodystrophy, angina pectoris, myocardial infarction, ulcers, asthma, allergies, benign prostatic hypertrophy, and psychotic and neurological disorders, including anxiety, schizophrenia, manic depression, delirium, dementia, severe mental retardation and dyskinesias, such as Huntington's disease or Gilles de la Tourette syndrome and/or other pathologies and disorders.

The invention further includes a method for screening for a modulator of disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders or other disorders related to cell signal processing and metabolic pathway modulation by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., diabetes, metabolic disturbances associated with obesity, the metabolic syndrome X, anorexia, wasting disorders associated with chronic diseases, metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 shows a TaqMan tissue profile result for NOV7.

FIG. 2 shows a replicate TaqMan profiles for NOV7 in a broader range of cancer cells that were derived from surgical specimens.

### DETAILED DESCRIPTION OF THE INVENTION

The present invention provides novel nucleotides and polypeptides encoded thereby. Included in the invention are the novel nucleic acid sequences and their polypeptides. The sequences are collectively referred to as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ ID | |
|---|---|---|---|---|
| NOVX | | NO | NO | |
| Assign- | Internal | (nucleic | (poly- | |
| ment | Identification | acid) | peptide) | Homology |
| 1 | 30235661_EXT1 | 1 | 2 | TSK-1-like |
| 2a | ba518k17A | 3 | 4 | beta Thymosin |
| 2b | 518k17_A1 | 5 | 6 | beta Thymosin |
| 2c | 518k17_A | 7 | 8 | beta Thymosin |
| 3a | GM_ba63k6_A | 9 | 10 | Connexin-like |
| 3b | CG54734-02 | 11 | 12 | Connexin-like |
| 4a | 85731808_EXT | 13 | 14 | Hepatoma |
| | | | | Derived |
| | | | | Growth Factor |
| 4b | 21143463.0.45 | 15 | 16 | Hepatoma |
| | | | | Derived |
| | | | | Growth Factor |
| 4c | 21143463_A.0.45_EXT | 17 | 18 | Hepatoma |
| | | | | Derived |
| | | | | Growth Factor |
| 4d | 117477333_EXT | 19 | 20 | Hepatoma |
| | | | | Derived |
| | | | | Growth Factor |
| 5a | 21647246_EXT | 21 | 22 | Cortexin-like |
| 5b | 21647246_da1 | 21 | 22 | Cortexin-like |
| 6 | 27926453_EXT1 | 23 | 24 | Sialoadhesin-like |
| 7 | 105180778 | 25 | 26 | Trio |
| | | | | Phosphoprotein |
| 8a | 3277789_EXT | 27 | 28 | Stra6-like |
| 8b | CG52276-03 | 29 | 30 | Stra6-like |
| 8c | CG52276-04 | 31 | 32 | Retinoic Acid |
| | | | | Responsive-like |
| 9 | SC_108341967_A | 33 | 34 | Thyroid |
| | | | | Regulated Gene |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

For example, NOV1 is homologous to a testis specific serine/threonine protein kinase (TSK-1) family of proteins that exhibits dual specific protein kinase activity on both serine/threonine and tyrosine and is expressed in testis. Thus, the NOV1 nucleic acids, polypeptides, antibodies and related compounds according to the invention will be useful in therapeutic and diagnostic applications implicated in, for example; Spermatogenesis, Male Reproductive Health, Fertility and/or other pathologies/disorders.

Also, NOV2a, 2b and 2c are homologous to the beta thymosin family of proteins. Thus NOV2 nucleic acids, polypeptides, antibodies and related compounds according to the invention will be useful in therapeutic and diagnostic applications implicated in, for example; prostate cancer, apoptosis, angiogenesis and wound healing, neurodegenerative and neuropsychiatric disease, immune and autoimmune disorders, age-related disorders and/or other pathologies/disorders.

Further, NOV3a and 3b are homologous to a family of connexin-like proteins which are important in forming specialized cell-cell contact sites. Thus, the NOV3 nucleic acids and polypeptides, antibodies and related compounds according to the invention will be useful in therapeutic and diagnostic applications implicated in, for example; Clouston syndrome and deafness, mutilating palmoplantar keratoderma (PPK), X-linked Charcot-Marie-Tooth neuropathy, hereditary peripheral neuropathy and/or other pathologies/disorders.

Also, NOV4a, 4b, 4c and 4d are homologous to the hepatoma derived growth factor family of proteins which are important endothelial cell mitogens. Thus, NOV4 nucleic acids, polypeptides, antibodies and related compounds according to the invention will be useful in therapeutic and diagnostic applications implicated in, for example; Adrenoleukodystrophy, Hemophilia, Hypercoagulation, Immunodeficiencies, Alzheimer's disease, Stroke, Parkinson's disease, Huntington's disease, Cerebral palsy, Epilepsy, Multiple sclerosis and/or other pathologies/disorders.

Additionally, NOV5a and NOV5b are homologous to the cortexin family of proteins. Thus NOV5 nucleic acids, polypeptides, antibodies and related compounds according to the invention will be useful in treating a variety of conditions, including, e.g., Von Hippel-Lindau (VHL) syndrome, Alzheimer's disease, stroke, tuberous sclerosis, hypercalceimia, etc.

Also, NOV6 is homologous to the sialoadhesin-like family of proteins. Thus NOV6 nucleic acids, polypeptides, antibodies and related compounds according to the invention will be useful in therapeutic and diagnostic applications in various disorders, including, for example, involving cell-cell interactions.

Further, NOV7 is homologous to members of the Trio Phosphoprotein family of proteins. Thus, the NOV7 nucleic acids, polypeptides, antibodies and related compounds according to the invention will be useful in therapeutic and diagnostic applications in disorders characterized by, e.g., impaired cell migration and anchorage-independent growth.

Still further, NOV8 is homologous to a family of Stra6-like or retinoic acid responsive-like proteins that are important in a variety of functions. Thus, NOV8 nucleic acids and polypeptides, antibodies and related compounds according to the invention will be useful in therapeutic and diagnostic applications in disorders including, for example, osteoporosis, hypercalceimia, arthritis, ankylosing spondylistis, scoliosis, muscular dystrophy, Lesch-Nyhan syndrome, myasthenia gravis, reproductive disorders, fertility disorders, developmental disorders, endocrine/growth disorders, disorders in pubertal development, surgery/wound healing, and/or endocrine/growth disorders.

Finally, NOV9 is homologous to the thyroid regulated gene family of proteins. Thus, NOV9 nucleic acids and polypeptides, antibodies and related compounds according to the invention will be useful in therapeutic and diagnostic applications in various disorders including, for example, hypo- and hyperthyroidism, disorders of the thyroid, and thyroid-related cancers.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

A NOV1 sequence (also referred to as 30235661_EXT1) according to the invention includes a nucleic acid sequence encoding a polypeptide related to the testis specific serine/threonine protein kinase (TSK-1) family of proteins. Tables 1A and 1B show a NOV1 nucleic acid and its encoded polypeptide sequence, respectively. A disclosed NOV1 nucleic acid of 1149 nucleotides is shown in Table 1A. The disclosed NOV1 open reading frame (“ORF”) was identified beginning with an ATG initiation codon at nucleotides 1-3 and ending with a TAA codon at nucleotides 1147-1149. As shown in Table 1A, the start and stop codons are in bold letters.

TABLE 1A
NOV1 nucleotide sequence.

(SEQ ID NO:1)

ATGTCGGGAGACAAACTTCTGAGCGAACTCGGTTATAAGCTGGGCCGCACAATTGGAGAGGGCAGCTACTCC
AAGGTGAAGGTGGCCACATCCAAGAAGTACAAGGGTACCGTGGCCATCAAGGTGGTGGACCGGCGGCGAGCG
CCCCCGGACTTCGTCAACAAGTTCCTGCCGCGAGAGCTGTCCATCCTGCGGGGCGTGCGACACCCGCACATC
GTGCACGTCTTCGAGTTCATCGAGGTGTGCAACGGGAAACTGTACATCGTGATGGAAGCGGCCGCCACCGAC
CTGCTGCAAGCCGTGCAGCGCAACGGGCGCATCCCCGGAGTTCAGGCGCGCGACCTCTTTGCGCAGATCGCC
GGCGCCGTGCGCTACCTGCACGATCATCACCTGGTGCACCGCGACCTCAAGTGCGAAAACGTGCTGCTGAGC
CCGGACGAGCGCCGCGTCAAGCTCACCGACTTCGGCTTCGGCCGCCAGGCCCATGGCTACCCAGACCTGAGC
ACCACCTACTGCGGCTCAGCCGCCTACGCGTCACCCGAGGTGCTCCTGGGCATCCCCTACGACCCCAAGAAG
TACGATGTGTGGAGCATGGGCGTCGTGCTCTACGTCATGGTCACCGGGTGCATGCCCTTCGACGACTCGGAC
ATCGCCGGCCTGCCCCGGCGCCAGAAACGCGGCGTGCTCTATCCCGAAGGCCTCGAGCTGTCCGAGCGCTGC
AAGGCCCTGATCGCCGAGCTGCTGCAGTTCAGCCCGTCCGCCAGGCCCTCCGCGGGCCAGGTAGCGCGCAAC
TGCTGGCTGCGCGCCGGGGACTCCGGCAGGAACGGAAGGAAGGGAAGGAAAGAAGGAAGGGAAGGAAGGGAA
GGAAGGGAAGGAAGGGAAGGAAAGGAAGGAAAGGAAGGAAAGGGAAGGAAGAAAAGGAAGGAAAGGGAAGGA
AGAAAAGGAAGGAAAGGGAAGGAAGGAAAGAGGGACAAAAGAAAAGTCCAGTTGACACATCATCCATTTATT
ATCCTTCAGAGTCTAAAACTTCCTCGTGATACAACGTATAGCCACCCATTCCAGCCTGCTTATTGGAACTTC
TATTCCCATCTGGTGGCAAACATTTCTTTTTACATTTGTTTTACTATCAAAGATTTAGAGTCACAATAA

In a search of public sequence databases, it was found, for example, that the NOV1 nucleic acid sequence disclosed in this invention has 219 of 314 bases (69%) identical to one region of a Homo Sapiens DGS-G mRNA, 3′ end, 1806 bp, with an E-value of 4.7e⁻³⁷ (GENBANK-ID: HUMDGSG|acc:L77564). It also had 326 of 527 bp (61%) identical to a second region in this same sequence. Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., Homo sapiens DGS-G mRNA, matched the Query NOV1 sequence purely by chance is 4.7×10⁻³⁷. The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996.

A disclosed encoded NOV1 protein has 382 amino acid residues, referred to as the NOV1 protein. The NOV1 protein was analyzed for signal peptide prediction and cellular localization. The SignalP and Psort results predict that NOV1 does not have a signal peptide and is likely to be localized to the nucleus, with a certainty of 0.9800. The disclosed NOV1 polypeptide sequence is presented in Table 1B using the one-letter amino acid code.

TABLE 1B
Encoded NOV1 protein sequence.

(SEQ ID NO:2)

MSGDKLLSELGYKLGRTIGEGSYSKVKVATSKKYKGTVAIKVVDRRRAPPDFVNKFLPRELSILRGVRHPHI
VHVFEFIEVCNGKLYIVMEAAATDLLQAVQRNGRIPGVQARDLFAQIAGAVRYLHDHHLVHRDLKCENVLLS
PDERRVKLTDFGFGRQAHGYPDLSTTYCGSAAYASPEVLLGIPYDPKKYDVWSMGVVLYVMVTGCMPFDDSD
IAGLPRRQKRGVLYPEGLELSERCKALIAELLQFSPSARPSAGQVARNCWLRAGDSGRNGRKGRKEGREGRE
GREGREGKEGKEGKGRKKRKEREGRKGRKGKEGKRDKRKVQLTHHPFIILQSLKLPRDTTYSHPFQPAYWNF
YSHLVANISFYICFTIKDLESQ

NOV1 sequences were initially identified by searching a proprietary sequence file database for DNA sequences which translate into proteins with similarity to a protein family of interest. NOV1 was identified as having suitable similarity. NOV1 was analyzed further to identify any open reading frames encoding novel full length proteins, as well as, novel splice forms of TSK-1. This was done by extending the identified NOV1 using suitable sequences from additional proprietary assemblies, publicly available EST sequences and public genomic sequences. A Genomic clone AC011448 was identified as having regions with 100% identity to the NOV1 and was selected for analysis because this identity implied that this clone contained the sequence of the genomic locus for NOV1.

The genomic clones were analysed by Genscan and Grail to identify exons and putative coding sequences/open reading frames. This clone was also analyzed by TblastN, BlastX and other homology programs to identify regions translating to proteins with similarity to the original protein/protein family of interest. Expressed sequences from both public and proprietary databases were also added when available to further define and complete the gene sequence. The DNA sequence was then manually corrected for apparent inconsistencies thereby obtaining the sequences encoding the full-length protein.

The TSK-1 disclosed in this invention (NOV1) belongs to genomic DNA [AC011448 from GenbankNEW]. Within this GenbankNew entry was a note showing that the sequence was from Chromosome 19. Therefore we assign the chromosomal locus of NOV1 as Chromosome 19. Further, the TSK-1 disclosed in this invention (NOV1) is expressed in testis.

A BLASTX search was performed against public protein databases. The disclosed NOV1 protein (SEQ ID NO:2) has good identity with TSK-1-like proteins. For example, the full amino acid sequence of the protein of the invention was found to have 146 of 360 amino acid residues (40%) identical to, and 208 of 360 residues (57%) similar to, the 364 amino acid residue TSK-1 protein from Mus musculus (SPTREMBL-ACC:Q61241; E=3.4 e-66). Public amino acid databases include the GenBank databases, SwissProt, PDB and PIR.

It was also found that NOV1 had homology to the amino acid sequences shown in the BLASTP data listed in Table 1C.

TABLE 1C
BLAST results for NOV1

| Gene Index/ | | Length | Identity | Positives | |
|---|---|---|---|---|---|
| Identifier | Protein/Organism | (aa) | (%) | (%) | Expect |
| gi\|14042966\| | serine/threonine | 273 | 272/272 | 272/272 | 1e−152 |
| ref\| | protein kinase | | (100%) | (100%) | |
| NP_114426.1\| | SSTK [Homo | | | | |
| | sapiens] | | | | |
| gi\|13540326\| | serine/threonine | 273 | 272/272 | 272/272 | 1e−152 |
| gb\|AAK29414.1\| | kinase FKSG82 | | (100%) | (100%) | |
| AF348077_1 | [Homo sapiens] | | | | |
| (AF348077) | | | | | |
| gi\|13898617\| | serine/threonine | 273 | 272/272 | 272/272 | 1e−152 |
| gb\|AAK48827.1\| | protein kinase | | (100%) | (100%) | |
| AF329483_1 | SSTK [Homo | | | | |
| (AF329483) | sapiens] | | | | |
| gi\|14030781\| | serine/threonine | 273 | 264/272 | 270/272 | 1e−148 |
| ref\| | protein kinase | | (97%) | (99%) | |
| NP_114393.1\| | SSTK [Mus | | | | |
| | musculus] | | | | |
| gi\|13898619\| | serine/threonine | 273 | 264/272 | 270/272 | 1e−148 |
| gb\|AAK48828.1\| | protein kinase | | (97%) | (99%) | |
| AF329484_1 | SSTK [Mus | | | | |
| (AF329484) | musculus] | | | | |

The homology of these and other sequences is shown graphically in the ClustalW analysis shown in Table ID. In the ClustalW alignment of the NOV1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be mutated to a much broader extent without altering protein structure or function.

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for NOV1 as disclosed in Tables 1E-1G, were collected from the Conserved Domain Database (CDD) with Reverse Position Specific BLAST analyses. This BLAST analysis software samples domains found in the Smart and Pfam collections. For Tables 1E-1G and all successive DOMAIN sequence alignments, fully conserved single residues are indicated by black shading and “strong” semi-conserved residues are indicated by grey shading. The “strong” group of conserved amino acid residues may be any one of the following groups of amino acids: STA, EQK, NHQK, NDEQ, QHRK, MILV, MILF, HY, FYW.

Table 1E-1G lists the domain description from DOMAIN analysis results against NOV1. This indicates that the NOV1 sequence has properties similar to those of other proteins known to contain this domain.

[image]

[image]

[image]

BLAST results include sequences from the Patp database, which is a proprietary database that contains sequences published in patents and patent publications. Patp results include those listed in Table 1H.

TABLE 1H
Patp alignments of NOV1

| | | | | Smallest |
|---|---|---|---|---|
| | | | | Sum |
| Sequences producing High-scoring | | Reading | High | Prob. |
| Segment Pairs: | | Frame | Score | P (N) |
| Patp: AAB65686 | Novel Protein Kinase | +1 | 1429 | 1.8e−145 |
| | Homo Sapiens, 273 aa | | | |
| Patp: AAB42167 | Human ORFX ORF1931 | +1 | 910 | 1.8e−90 |
| | polypeptide, 210 aa | | | |

For example, a BLAST against patp: AAB65686 (WO00/073469), a 273 amino acid protein kinase from Homo sapiens, produced good identity, E=1.8e-145. Additionally, a BLAST against patp: AAB42167, a 210 Human ORFX polypeptide sequence (WO00/058473), also produced good identity, E=1.8E-90.

Protein kinases are involved in intracellular signal transduction pathways. They are broadly classified into serine/threonine kinases and tyrosine kinases, which can be further divided into families and subfamilies based on similarity within the catalytic domain.

Two studies have isolated and identified members of the testis specific serine/threonine protein kinase family. Bielke et al. (1994) isolated a cDNA fragment encoding a new member of the Ser/Thr (serine/threonine) family of protein kinases using degenerate oligos corresponding to two highly conserved motifs within the protein kinase catalytic domain and a PCR-based cloning strategy. Expression analysis revealed that the fragment recognized two transcripts (1.6 and 1.4 kb) exclusively in testis. Using this fragment as a probe, Bielke et al. (1994) cloned a full-length cDNA from a mouse testis cDNA library. The sequence has a 1092-bp open reading frame encoding a protein of 364 amino acids. The N-terminally localized kinase catalytic domain has all the conserved motifs found in other Ser/Thr kinases. Northern blot analysis using the full-length sequence as a probe revealed that the cloned gene corresponds to the 1.6-kb transcript, suggesting the existence of at least two testis-specific novel Ser/Thr kinases. Bielke et al. (1994) proposed the name testis-specific kinase-1 (TSK-1) for the identified/described gene. A GenEMBL databank search revealed highest homology to the human gene encoding rac protein kinase-beta and the group of yeast Ser/Thr kinases encoded by SNF-1, nm-1, KIN-1 and KIN-2.

In a more recent study, Rosok et al. (1999) isolated a novel full-length cDNA from a human fetal liver cDNA library using a subtractive PCR cloning strategy and degenerate primers based on conserved amino acid regions in the catalytic domain of serine/threonine kinases. Rosok et al. (1999) designated the cDNA testis-specific kinase-2 (TESK2) because it encodes a putative 555-amino acid protein with a kinase domain that is 65% identical to that of testis-specific kinase-1 (TESK1), which exhibits dual specific protein kinase activity on both serine/threonine and tyrosine; it also shows 42% and 39% identity to LIMK1 and LIMK2, respectively. Northern blot analysis revealed a single TESK2 mRNA species of approximately 3.0 kb, predominantly expressed in testis and prostate. Rosok et al. (1999) also found that the rat homolog was first expressed in the testis after day 30 of postnatal development in round spermatids. The authors suggested that TESK2 plays an important role in spermatogenesis.

The above defined information for this invention suggests that this novel TSK-1-like protein (NOV1) may function as a member of a TSK-1 family. Therefore, the expression nucleic acids and proteins of NOV1 are useful in potential therapeutic applications implicated in various TSK-1-related pathologies and/or disorders. For example, a cDNA encoding the TSK-1-like protein may be useful in gene therapy, and the TSK-1-like protein may be useful when administered to a subject in need thereof. The novel nucleic acid encoding NOV1 protein, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The NOVX nucleic acids and proteins of the invention are useful in potential therapeutic applications implicated in various diseases and disorders described below and/or other pathologies and disorders. For example, but not limited to, a cDNA encoding the TSK-1-like protein may be useful in gene therapy, and the TSK-1-like protein may be useful when administered to a subject in need thereof. By way of nonlimiting example, the compositions of the present invention will have efficacy for treatment of patients suffering from Spermatogenesis, Male Reproductive Health, Fertility and/or other pathologies/disorders. The novel nucleic acid encoding the TSK-1-like protein, and the TSK-1-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

Further, the protein similarity information, expression pattern, and map location for NOV1 suggests that NOV1 may have important structural and/or physiological functions characteristic of the TSK-1 family. Therefore, the nucleic acids and proteins of the invention are useful in potential diagnostic and therapeutic applications and as a research tool. Potential therapeutic uses for the compositions of the invention included, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

These materials are further useful in the generation of antibodies that bind immunospecifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 10 to 30. In another embodiment, a NOV1 epitope is from about amino acids 35 to 70. In additional embodiments, NOV1 epitopes are from amino acids 90 to 110, 120-170, 180-190, 210-230, 250-335 and from amino acids 340 to 365. These novel proteins can be used in assay systems for functional analysis of various human disorders, which will help in understanding of pathology of the disease and development of new drug targets for various disorders. NOV2

NOV2 includes three novel beta thymosin-like proteins disclosed below. The disclosed proteins have been named NOV2a, NOV2b and NOV2c.

NOV2a

A novel nucleic acid was identified on chromosome 9 by ThlastN using CuraGen Corporation's sequence file for beta thymosin or homolog as run against the Genomic Daily Files made available by GenBank or from files downloaded from the individual sequencing centers. The nucleic acid sequence was predicted from the genomic file GB ACCNO:ba518k17 by homology to a known beta thymosin or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added, when available, to further define and complete the gene sequence. The DNA sequence was then manually corrected for apparent inconsistencies thereby obtaining the sequences encoding the full-length protein. In particular, nucleotide 121905 was spliced to nucleotide 121758 in preparing the ba518k17_A sequence.

The novel nucleic acid of 147 nucleotides (also referred to as ba518 k17_A) encoding a novel beta thymosin-like protein is shown in Table 2A. An open reading frame was identified beginning with an ATG initiation codon at nucleotides 6-8 and ending with a TGA codon at nucleotides 135-137. A putative untranslated region upstream from the initiation codon and downstream from the termination codon is underlined in Table 2A, and the start and stop codons are in bold letters.

TABLE 2A
NOV2a Nucleotide Sequence

(SEQ ID NO:3)

AGAAAATGGCACACAAACTAGACCTGGAAGAAATTGCCAGCTTGGATAAGGCCAAGCTGAAGGCCACAGAG
ATGCAGAAGAACACTCTGATGACCAAAGAGACCACAGAGCAGGAGAAGTGGAGTGAAATTTCCTGAGAGCC
TCGAG

In a search of public sequence databases, it was found, for example, that the nucleic acid sequence (NOV2a) has 126 of 150 bases (84%) identical to a human beta thymosin mRNA (GENBANK-ID: S54005|acc:S54005) (E=7.0 e-17). Public nucleotide databases include all GenBank databases and the GeneSeq patent database.

The disclosed NOV2a polypeptide (SEQ ID NO:4) encoded by SEQ ID NO:3 is 43 amino acid residues and is presented using the one-letter code in Table 2B. The NOV2a protein was analyzed for signal peptide prediction and cellular localization. SignalP, Psort and Hydropathy results predict that NOV2a does not appear to contain a predicted signal peptide and that NOV2a is likely to be localized in the cytoplasm with a certainty of 0.4500.

TABLE 2B
Encoded NOV2a protein sequence.

(SEQ ID NO:4)

MAHKLDLEEIASLDKAKLKATEMQKNTLMTKETTEQEKWSEIS

The full amino acid sequence of the NOV2a protein of the invention was found to have 33 of 44 amino acid residues (75%) identical to, and 34 of 44 residues (77%) positive with, the 44 amino acid residue Thymosin beta-10 protein from rat (ptnr: PIR-ID:A27266; E=7.2 e-09)(Table 2C). The global sequence homology is 79% amino acid similarity and 77% amino acid identity. In addition, this protein contains the thymosin protein domain (as defined by Interpro# IPR001152) at amino acid positions 2 to 41. Public amino acid databases include the GenBank databases, SwissProt, PDB and PIR.

TABLE 2C
BLASTX results for NOV2a

| | | | | Smallest | |
|---|---|---|---|---|---|
| | | | | Sum | |
| Sequences producing High-scoring | | Reading | High | Prob. | |
| Segment Pairs: | | Frame | Score | P (N) | N |
| ptnr: PR-ID: A27266 | thymosin beta- | +3 | 137 | 7.2e−09 | 1 |
| | 10 - rat | | | | |

NOV2b

In the present invention, the target sequence identified above, Accession Number ba518 k17_A, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy to provide the sequence reported below, which is designated Accession Number 518 k17_A1.

A disclosed NOV2b (also referred to as 518 k17_A1) nucleic acid of 147 nucleotides is shown in Table 2D. An open reading frame was identified beginning with an ATG initiation codon at nucleotides 6-8 and ending with a TGA codon at nucleotides 135-137. A putative untranslated region upstream from the initiation codon and downstream from the termination codon is underlined in Table 2D, and the start and stop codons are in bold letters.

TABLE 2D
NOV2b Nucleotide Sequence

(SEQ ID NO:5)

AGAAAATGGCACACAAACTAGACCTGGAAGAAATTGCCAGCTTGGATAAGGCCAAGCTGAAGGCCACAGAG
ATGCAGAAGAACACTCTGATGACCAAAGAGACCACAGAGCAGGAGAAGTGGAGTGAAATTTCCTGAGAGCC
TCGAG

The disclosed NOV2b polypeptide (SEQ ID NO:6) encoded by SEQ ID NO:5 is 43 amino acid residues and is presented using the one-letter code in Table 2E. The NOV2b protein was analyzed for signal peptide prediction and cellular localization. SignalP, Psort and Hydropathy results predict that NOV2b does not appear to contain a predicted signal peptide and that NOV2b is likely to be localized in the cytoplasm with a certainty of 0.4500. NOV2b has a molecular weight of 4979.7 Daltons.

TABLE 2E
Encoded NOV2b protein sequence.

(SEQ ID NO:6)

MAHKLDLEEIASLDKAKLKATEMQKNTLMTKETTEQEKWSEIS

The amino acid sequence of NOV2b had high homology to other proteins as shown in Table 2F.

TABLE 2F
BLASTX results for NOV2b

| | | | | Smallest | |
|---|---|---|---|---|---|
| | | | | Sum | |
| | | Reading | High | Prob. | |
| Sequences producing High-scoring Segment Pairs: | | Frame | Score | P (N) | N |
| ptnr: PIR-ID: A27266 | thymosin beta-10 - rat | +3 | 137 | 1.8e−08 | 1 |
| ptnr: SWISSPROT-ACC: P13472 | THYMOSIN BETA-10 - Homo sapi . . . | +3 | 132 | 6.0e−08 | 1 |
| ptnr: TREMBLNEW-ACC: BAA96493 | THYMOSIN BETA B - Cyprinus . . . | +3 | 115 | 3.8e−06 | 1 |
| ptnr: SWISSPROT-ACC: P21752 | THYMOSIN BETA-9 AND BETA-8 - . . . | +3 | 114 | 4.9e−06 | 1 |
| ptnr: SPTREMBL-ACC: Q9PT32 | THYMOSIN BETA - Oncorhynchus . . . | +3 | 113 | 6.2e−06 | 1 |
| ptnr: TREMBLNEW-ACC: CAB76965 | PUTATIVE THYMOSIN BETA-10 . . . | +3 | 113 | 6.2e−06 | 1 |
| ptnr: SWISSPROT-ACC: P21753 | THYMOSIN BETA-9 - Sus scrofa . . . | +3 | 112 | 7.9e−06 | 1 |
| ptnr: SWISSPROT-ACC: P26351 | THYMOSIN BETA-11 - Oncorhync . . . | +3 | 108 | 2.1e−05 | 1 |
| ptnr: PIR-ID: S21282 | thymosin beta-11 - rainbow trout | +3 | 108 | 2.1e−05 | 1 |
| ptnr: SPTREMBL-ACC: 076538 | THYMOSIN BETA - Strongylocent . . . | +3 | 107 | 2.7e−05 | 1 |
| ptnr: PIR-ID: A59005 | thymosin beta - sea urchin (Arbacia . . . | +3 | 106 | 3.4e−05 | 1 |
| ptnr: PIR-ID: JQ1489 | thymosin beta-4 - African clawed frog | +3 | 104 | 5.6e−05 | 1 |
| ptnr: PIR-ID: B59005 | thymosin beta - scallop (Argopecten . . . | +3 | 103 | 7.1e−05 | 1 |
| ptnr: SPTREMBL-ACC: Q9W7M8 | BETA-THYMOSIN - Brachydanio r . . . | +3 | 102 | 9.1e−05 | 1 |
| ptnr: SWISSPROT-ACC: P26352 | THYMOSIN BETA-12 - Oncorhync . . . | +3 | 101 | 0.00012 | 1 |
| ptnr: PIR-ID: S22426 | thymosin beta-12 - rainbow trout | +3 | 101 | 0.00012 | 1 |
| ptnr: TREMBLNEW-ACC: CAB94229 | DJ1071L10.1 (THYMOSIN/INTE . . . | +3 | 100 | 0.00015 | 1 |
| ptnr: SWISSPROT-ACC: P20065 | THYMOSIN BETA-4 - Mus muscul . . . | +3 | 99 | 0.00019 | 1 |

Possible SNPs found for NOV2b are listed in Table 2G.

TABLE 2G
SNPs

| Base | Base | Base |
|---|---|---|
| Position | Before | After |
| 238 | A | C(2) |
| 241 | C | A(2) |
| 251 | A | C(3) |
| 253 | A | G(3) |
| 254 | C | G(3) |
| 255 | C | T(3) |
| 266 | C | T(2) |
| 572 | C | T(2) |
| 673 | G | T(3) |
| 748 | C | T(2) |

NOV2c

In the present invention, the target sequence identified above, Accession Number ba518k17_A, was subjected to the exon linking process to confirm the sequence. PCR primers were designed by starting at the most upstream sequence available, for the forward primer, and at the most downstream sequence available for the reverse primer. In each case, the sequence was examined, walking inward form the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a wide range of cDNA libraries. The resulting amplicon was gel purified, cloned and sequenced to high redundancy to provide the sequence reported below, which is designated Accession Number 518k17_A.

A disclosed NOV2c (also referred to as 518 k17_A) nucleic acid of 147 nucleotides is shown in Table 2H. An open reading frame was identified beginning with an ATG initiation codon at nucleotides 6-8 and ending with a TGA codon at nucleotides 135-137. A putative untranslated region upstream from the initiation codon and downstream from the termination codon is underlined in Table 2H, and the start and stop codons are in bold letters.

TABLE 2H
NOV2c Nucleotide Sequence

(SEQ ID NO:7)

AGAAAATGGCACACAAACTAGACCTGGAAGAAATTGCCAGCTTGGATAAGGCCAAGCTGAAGGCCACAGAG
ATGCAGAAGAACACTCTGATGACCAAAGAGACCACAGAGCAGGAGAAGTGGAGTGAAATTTCCTGAGAGCC
TCGAG

The disclosed NOV2c polypeptide (SEQ ID NO:8) encoded by SEQ ID NO:7 is 43 amino acid residues and is presented using the one-letter code in Table 2I. The NOV2c protein was analyzed for signal peptide prediction and cellular localization. SignalP, Psort and Hydropathy results predict that NOV2c does not appear to contain a predicted signal peptide and that NOV2c is likely to be localized in the cytoplasm with a certainty of 0.4500. NOV2c has a molecular weight of 4979.7 Daltons.

TABLE 2I
Encoded NOV2c protein sequence.

(SEQ ID NO:8)

MAHKLDLEEIASLDKAKLKATEMQKNTLMTKETTEQEKWSEIS

The amino acid sequences of NOV2c had high homology to other proteins as shown in Table 2J.

TABLE 2J
BLASTX results for NOV2c

| | | | | Smallest | |
|---|---|---|---|---|---|
| | | | | Sum | |
| | | Reading | High | Prob. | |
| Sequences producing High-scoring Segment Pairs: | | Frame | Score | P (N) | N |
| ptnr: PIR-ID: A27266 | thymosin beta-10 - rat | +3 | 137 | 1.7e−08 | 1 |
| ptnr: SWISSPROT-ACC: P13472 | THYMOSIN BETA-10 - Homo sapi . . . | +3 | 132 | 5.9e−08 | 1 |
| ptnr: TREMBLNEW-ACC: BAA96493 | THYMOSIN BETA B - Cyprinus . . . | +3 | 115 | 3.7e−06 | 1 |
| ptnr: SWISSPROT-ACC: P21752 | THYMOSIN BETA-9 AND BETA-8 - . . . | +3 | 114 | 4.8e−06 | 1 |
| ptnr: SPTREMBL-ACC: Q9PT32 | THYMOSIN BETA - Oncorhynchus . . | +3 | 113 | 6.1e−06 | 1 |
| ptnr: TREMBLNEW-ACC: CAB76965 | PUTATIVE THYMOSIN BETA-10 . . . | +3 | 113 | 6.1e−06 | 1 |
| ptnr: SWISSPROT-ACC: P21753 | THYMOSIN BETA-9 - Sus scrofa . . . | +3 | 112 | 7.8e−06 | 1 |
| ptnr: SWISSPROT-ACC: P26351 | THYMOSIN BETA-11 - Oncorhync . . . | +3 | 108 | 2.1e−05 | 1 |
| ptnr: PIR-ID: S21282 | thymosin beta-11 - rainbow trout | +3 | 108 | 2.1e−05 | 1 |
| ptnr: SPTREMBL-ACC: 076538 | THYMOSIN BETA - Strongylocent . . . | +3 | 107 | 2.6e−05 | 1 |
| ptnr: PIR-ID: A59005 | thymosin beta - sea urchin (Arbacia . . . | +3 | 106 | 3.4e−05 | 1 |
| ptnr: PIR-ID: JQ1489 | thymosin beta-4 - African clawed frog | +3 | 104 | 5.5e−05 | 1 |
| ptnr: PIR-ID: B59005 | thymosin beta - scallop (Argopecten . . . | +3 | 103 | 7.0e−05 | 1 |
| ptnr: SPTREMBL-ACC: Q9W7M8 | BETA-THYMOSIN - Brachydanio r . . . | +3 | 102 | 8.9e−05 | 1 |
| ptnr: SWISSPROT-ACC: P26352 | THYMOSIN BETA-12 - Oncorhync . . . | +3 | 101 | 0.00011 | 1 |
| ptnr: PIR-ID: S22426 | thymosin beta-12 - rainbow trout | +3 | 101 | 0.00011 | 1 |
| ptnr: TREMBLNEW-ACC: CAB94229 | DJ1071L10.1 (THYMOSIN/INTE . . . | +3 | 100 | 0.00015 | 1 |
| ptnr: SWISSPROT-ACC: P20065 | THYMOSIN BETA-4 - Mus muscul . . . | +3 | 99 | 0.00019 | 1 |
| ptnr: TREMBLNEW-ACC: AAC52490 | THYMOSIN B4 - Mus musculus . . . | +3 | 99 | 0.00019 | 1 |

NOV2a, 2b and 2c are related to each other as shown in the alignment listed in Table 2K.

[image]

It was also found that NOV2a had homology to the amino acid sequences shown in the BLASTP data listed in Table 2L.

TABLE 2L
BLAST results for NOV2a

| Gene Index/ | Protein/ | Length | Identity | Positives | |
|---|---|---|---|---|---|
| Identifier | Organism | (aa) | (%) | (%) | Expect |
| gi\|339697\| | thymosin | 49 | 33/44 | 34/44 | 6.1 |
| gb\|AAA36746.1\| | beta-10 | | (75%) | (77%) | |
| (M92383) | [Homo | | | | |
| | sapiens] | | | | |

The homology of these sequences is shown graphically in the ClustalW analysis shown in Table 2M.

[image]

Other BLAST results include sequences from the Patp database, which is a proprietary database that contains sequences published in patents and patent publications. Patp results include those listed in Table 2N.

TABLE 2N
Patp alignments of NOV2

| | | | | Smallest |
|---|---|---|---|---|
| | | | | Sum |
| Sequences producing High-scoring | | Reading | High | Prob. |
| Segment Pairs: | | Frame | Score | P (N) |
| Patp: AAR96932 | Thymosin beta 10 - | +3 | 132 | 4.9e−8 |
| | synthetic, 43 aa | | | |
| Patp: AAY80267 | Thymosin beta 4 pep- | +3 | 132 | 4.9e−8 |
| | tide isoform, 43 aa | | | |

For example, a BLAST against patp:AAR96932, a 43 amino acid synthetic thymosin beta 10 protein (WO96/11016), produced good identity, E=4.9e-8. Additionally, a BLAST against patp:AAY80267, a thymosin beta 4 peptide isoform (Theta10) (WO00/06190), a 43 amino acid polypeptide, also produced good identity, E=4.9e-8.

Thymosin-beta-4 (T-beta-4) induces the expression of terminal deoxynucleotidyl transferase activity in vivo and in vitro, inhibits the migration of macrophages, and stimulates the secretion of hypothalamic luteinizing hormone-releasing hormone. Clauss et al. (1991) noted that the protein was originally isolated from a partially purified extract of calf thymus, thymosin fraction 5, which induced differentiation of T cells and was partially effective in some immuno-compromised animals. Further studies demonstrated that the molecule is ubiquitous in all tissues and cell lines analyzed. It is found in highest concentrations in spleen, thymus, lung, and peritoneal macrophages. Li et al. (1996) stated that T-beta-4 is an actin monomer sequestering protein that may have a critical role in modulating the dynamics of actin polymerization and depolymerization in nonmuscle cells. Its regulatory role is consistent with the many examples of transcriptional regulation of T-beta-4 and of tissue-specific expression. Lymphocytes have a unique T-beta-4 transcript relative to the ubiquitous transcript found in many other tissues and cells. In a separate study, Clauss et al. (1991) stated that rat T-beta-4 is synthesized as a 44-amino acid propeptide which is processed into a 43-amino acid peptide by removal of the first methionyl residue and does not have a signal peptide. Comparison studies have shown that human T-beta-4 has a high degree of homology to rat T-beta-4; the coding regions differ by only 9 nucleotides, and these are all silent base changes.

Gondo et al. (1987) isolated a cDNA encoding T-beta-4 using differential screening of a cDNA library prepared from leukocytes of an acute lymphocytic leukemia patient. Utilizing Northern blot analysis, they studied the expression of the 830-nucleotide T-beta-4 mRNA in various primary myeloid and lymphoid malignant cell lines and in hemopoietic cell lines. Gondo et al. (1987) stated that the pattern of T-beta-4 gene expression suggests that it may be involved in an early phase of the host defense mechanism.

In other studies, Clauss et al. (1991) isolated a cDNA clone for the human interferon-inducible gene 6-26 (Friedman et al., 1984) and showed that its sequence was identical to that for the human T-beta-4. By use of a panel of human rodent somatic cell hybrids, Clauss et al. (1991) showed that the 6-26 cDNA recognized 7 genes, members of a multigene family, present on chromosomes 1, 2, 4, 9, 11, 20, and X. These genes are symbolized TMSL1, TMSL2, etc., respectively. Separately, Li et al. (1996) established that in the mouse there is a single Tmsb4 gene and that the lymphoid-specific transcript is generated by extending the ubiquitous exon 1 with an alternate downstream splice site. By interspecific backcross mapping, they located the mouse gene, which they symbolized Ptmb4, to the distal region of the mouse X chromosome, linked to Btk and Gja6. Thus, the human gene could be predicted to reside on the X chromosome in the general region of Xq21.3-q22, where BTK is located. By analysis of somatic cell hybrids, Lahn and Page (1997) mapped the T-beta-4, or TB4X, gene to the X chromosome. They noted that a homologous gene, TB4Y, is present on the Y chromosome.

Bao et al. (1996) found a novel member of the beta thymosin protein family expressed in a metastatic prostate carcinoma cell line. Prostate carcinoma is the most prevalent form of cancer in males and the second leading cause of cancer death among older males. The use of the serum prostate-specific antigen (PSA) test permits early detection of human prostate cancer; however, early detection has not been accompanied by an improvement in determining which tumors may progress to the metastatic stage. The process of tumor metastasis is a multistage event involving local invasion and destruction of extracellular matrix; intravasation into blood vessels, lymphatics or other channels of transport; survival in the circulation; extravasation out of the vessels into the secondary site; and growth in the new location. Common to many components of the metastatic process is the requirement for tumor cell motility. A well-characterized series of cell lines that showed varying metastatic potential was developed from the Dunning rat prostate carcinoma. Mohler et al. (1988) and Partin et al. (1989) showed a direct correlation between cell motility and metastatic potential in the Dunning cell lines. In studies comparing gene expression in poorly and highly motile metastatic cell lines derived from Dunning rat prostate carcinoma using differential mRNA display, Bao et al. (1996) found a novel member of the beta thymosin family of actin-binding molecules, named thymosin-beta-15 (T-beta-15), which was found to deregulate motility in prostate cells directly. In addition, it was expressed in advanced human prostate cancer specimens, but not in normal human prostate or benign prostatic hyperplasia, suggesting its potential use as a new marker for prostate carcinoma progression. Bao et al. (1996) also found that T-beta-15 levels correlated positively with the Gleason tumor grade. Coffey (1996) pointed out that the upregulation of T-beta-15 as a positive motility factor and the down regulation of the motility suppressor KAI1 provide the ‘yin and yang’ for metastasis; thus, he speculated that these pathways may provide a new target for therapy.

T-beta-4 has also been implicated in the acceleration of wound healing. Angiogenesis is an essential step in the repair process that occurs after injury. In our studies, we investigated whether the angiogenic thymic peptide, T-beta-4, enhanced wound healing in a rat full thickness wound model. Addition of T-beta-4 topically or intraperitoneally increased reepithelialization by 42% over saline controls at 4 d and by as much as 61% at 7 d post-wounding. Treated wounds also contracted at least 11% more than controls by day 7. Increased collagen deposition and angiogenesis were observed in the treated wounds. We also found that T-beta-4 stimulated keratinocyte migration in the Boyden chamber assay. After 4-5 h, migration was stimulated 2-3-fold over migration with medium alone when as little as 10 pg of T-beta-4 was added to the assay. These results suggest that T-beta-4 is a potent wound healing factor with multiple activities that may be useful in the clinic.

The above defined information for this invention suggests that this beta thymosin-like protein may function as a member of a “beta thymosin family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in cancer including but not limited to prostate cancer, immunological and autoimmune disorders (i.e. hyperthyroidism), angiogenesis and wound healing, modulation of apoptosis, neurodegenerative and neuropsychiatric disorders, age-related disorders, and other pathological disorders involving spleen, thymus, lung, and peritoneal macrophages and/or other pathologies and disorders. For example, a cDNA encoding the beta thymosin-like protein may be useful in gene therapy, and the beta thymosin-like protein may be useful when administered to a subject in need thereof. By way of nonlimiting example, the compositions of the present invention will have efficacy for treatment of patients suffering from cancer including but not limited to prostate cancer, immunological and autoimmune disorders (ie hyperthyroidism), angiogenesis and wound healing, modulation of apoptosis, neurodegenerative and neuropsychiatric disorders, age-related disorders, and other pathological disorders involving spleen, thymus, lung, and peritoneal macrophages. The novel nucleic acid encoding beta thymosin-like protein, and the beta thymosin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding the beta thymosin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV2 proteins have multiple hydrophilic regions, each of which can be used as an immunogen. These novel proteins can be used in assay systems for functional analysis of various human disorders, which will help in understanding of pathology of the disease and development of new drug targets for various disorders.

NOV3

NOV3 includes two novel connexin-like proteins disclosed below. The disclosed proteins have been named NOV3a and NOV3b.

NOV3a

A novel nucleic acid was identified on chromosome 6 by TblastN using CuraGen Corporation's sequence file for connexin or homolog as run against the Genomic Daily Files made available by GenBank or from files downloaded from the individual sequencing centers. The nucleic acid sequence was predicted from the genomic file Sequencing Center accession number: ba63k6_A by homology to a known connexin or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The novel nucleic acid of 1750 nucleotides (also referred to as GM_ba63k6_A) encoding a novel connexin-like protein is shown in FIG. 3A. An open reading frame was identified beginning with an ATG initiation codon at nucleotides 55-57 and ending with a TAA codon at nucleotides 1684-1686. A putative untranslated region upstream from the initiation codon and downstream from the termination codon is underlined in FIG. 3A, and the start and stop codons are in bold letters.

TABLE 3A
NOV3a Nucleotide Sequence

(SEQ ID NO:9)

TGGGTGCTATTAACCTCAGTGTTTTCTGTATATTTCAGACATTAGTCTTTAACCATGGGGGACTGGAACT
TATTGGGTGGCATCCTAGAGGAAGTTCACTCCCACTCAACCATAGTGGGGAAAATCTGGCTGACCATCCT
CTTCATCTTCCGAATGCTGGTACTTCGTGTGGCTGCTGAGGATGTCTGGGATGATGAACAGTCAGCATTT
GCCTGCAACACCCGGCAGCCAGGTTGCAACAATATCTGTTATGATGATGCATTCCCTATCTCTTTGATCA
GGTTCTGGGTTTTACAGATCATCTTTGTGTCTTCTCCTTCTTTGGTCTATATGGGCCATGCACTTTATAG
GCTCAGGGCCTTTGAGAAAGACAGGCAGAGGAAAAAGTCACACCTTAGAGCCCAGATGGAGAATCCAGAT
CTTGACTTGGAGGAGCAGCAAAGAATAGATAGGGAACTGAGGAGGTTAGAGGAGCAGAAGAGGATCCATA
AAGTCCCTCTGAAAGGATGTCTGCTGCGTACTTATGTCTTACACATCTTGACCAGATCTGTGCTGGAAGT
AGGATTCATGATAGGCCAATATATTCTCTATGGGTTTCAAATGCACCCCCTTTACAAATGCACTCAACCT
CCTTGCCCCAATGCGGTGGATTGCTTTGTATCCAGGCCCACTGAGAAGACAATTTTCATGCTTTTTATGC
ACAGCATTGCAGCCATTTCCTTGTTACTCAATATACTGGAAATATTTCATCTAGGCATCAGAAAAATTAT
GAGGACACTTTATAAGAAATCCAGCAGTGAGGGCATTGAGGATGAAACAGGCCCTCCATTCCATTTGAAG
AAATATTCTGTGGCCCAGCAGTGTATGATTTGCTCTTCATTGCCTGAAAGAATCTCTCCACTTCAAGCTA
ACAATCAACAGCAAGTCATTCGAGTTAATGTGCCAAAGTCTAAAACCATGTGGCAAATCCCACAGCCAAG
GCAACTTGAAGTAGACCCTTCCAATGGGAAAAAGGACTGGTCTGAGAAGGATCAGCATAGCGGACAGCTC
CATGTTCACAGCCCGTGTCCCTGGGCTGGCAGTGCTGGAAATCAGCACCTGGGACAGCAATCAGACCATT
CCTCATTTGGCCTGCAGAATACAATGTCTCAGTCCTGGCTAGGTACAACTACGGCTCCTAGAAACTGTCC
ATCCTTTGCAGTAGGAACCTGGGAGCAGTCCCAGGACCCAGAACCCTCAGGTGAGCCTCTCACAGATCTT
CATAGTCACTGCAGAGACAGTGAAGGCAGCATGAGAGAGAGTGGGGTCTGGATAGACAGATCTCGCCCAG
GCAGTCGCAAGGCCAGCTTTCTGTCCAGATTGTTGTCTGAAAAGCGACATCTGCACAGTGACTCAGGAAG
CTCTGGTTCTCGGAATAGCTCCTGCTTGGATTTTCCTCACTGGGAAAACAGCCCCTCACCTCTGCCTTCA
GTCACTGGGCACAGAACATCAATGGTAAGACAGGCAGCCCTACCGATCATGGAACTATCACAAGAGCTGT
TCCATTCTGGATGCTTTCTTTTTCCTTTCTTTCTTCCTGGGGTGTGTATGTATGTTTGTGTTGACAGAGA
GGCAGATGGAGGGGGAGATTATTTATGGAGAGATAAAATTATTCATTCGATACATTCAGTTAAATTCAAT
TCATAAAATAGACTTAGAAAAATCTTATTATATCAATCGCTCTTATAAGTGCTGGGCATGTAAATGGGTA

In a search of public sequence databases, it was found, for example, that the disclosed NOV3a nucleic acid sequence has 1274 of 1514 bases (84%) identical to a Mus musculus connexin mRNA (GENBANK-ID: AJ010741)(E=4.7e-245). The nucleic acid also has 226 of 335 bases (67%) identical to a 1308 bp human gap juntion protein alpha (GJA3) gene (GENBANK-ID:AF075290jacc:AF075290) (E=3.1e-36). Public nucleotide databases include all GenBank databases and the GeneSeq patent database.

The disclosed NOV3a polypeptide (SEQ ID NO: 10) encoded by SEQ ID NO:9 is 543 amino acid residues and is presented using the one-letter code in Table 3B. The NOV3a protein was analyzed for signal peptide prediction and cellular localization. SignalP, Psort and Hydropathy results predict that NOV3a has a signal peptide with most likely cleavage site pos. 41 and 42, at: VAA-ED, and that NOV3a is likely to be localized in the plasma membrane with a certainty of 0.6000.

TABLE 3B
Encoded NOV3a protein sequence.

(SEQ ID NO:10)

MGDWNLLGGILEEVHSHSTIVGKIWLTILFIFRMLVLRVAAEDVWDDEQSAFACNTRQPGCNNICYDDAFP
ISLIRFWVLQIIFVSSPSLVYMGHALYRLRAFEKDRQRKKSHLRAQMENPDLDLEEQQRIDRELRRLEEQK
RIHKVPLKGCLLRTYVLHILTRSVLEVGFMIGQYILYGFQMHPLYKCTQPPCPNAVDCFVSRPTEKTIFML
FMHSIAAISLLLNILEIFHLGIRKIMRTLYKKSSSEGIEDETGPPFHLKKYSVAQQCMICSSLPERISPLQ
ANNQQQVIRVNVPKSKTMWQIPQPRQLEVDPSNGKKDWSEKDQHSGQLHVHSPCPWAGSAGNQHLGQQSDH
SSFGLQNTMSQSWLGTTTAPRNCPSFAVGTWEQSQDPEPSGEPLTDLHSHCRDSEGSMRESGVWIDRSRPG
SRKASFLSRLLSEKRHLHSDSGSSGSRNSSCLDFPHWENSPSPLPSVTGHRTSMVRQAALPIMELSQELFH
SGCFLFPFFLPGVCMYVCVDREADGGGDYLWRDKIIHSIHSVKFNS

A BLASTX search was performed against public protein databases. The full amino acid sequence of the protein of the invention was found to have 393 of 485 amino acid residues (81%) identical to, and 422 of 485 residues (87%) positive with, the 505 amino acid residues connexin protein from Mus musculus (ptnr: SPTREMBL-ACC: Q9WUS4; E=2.9e-211). The protein also has 138 of 258 residues (53%) identical to, and 188 of 258 residues (72%) positive with a 435 residue human gap junction protein (connexin) (SPTREMBL-ACC:Q9Y6H8; E=2.8e-71)(Table 3C). The global sequence homology (as defined by GAP global sequence alignment with the full length sequence of this protein) is 84% amino acid similarity and 81% amino acid identity. In addition, this protein contains the connexin (IPR000500) protein domain (as defined by Interpro) at amino acid positions 1 to 233.

TABLE 3C
BLASTX results for NOV3a

| | | | | Smallest | |
|---|---|---|---|---|---|
| | | | | Sum | |
| Sequences producing High-scoring | | Reading | High | Prob. | |
| Segment Pairs: | | Frame | Score | P (N) | N |
| ptnr: SPTREMBL- | Connexin 57 - | +3 | 731 | 2.0e−71 | 1 |
| ACC: Q9WUS4 | Mus musculus | | | | |
| ptnr: SPTREMBL- | Connexin - | +3 | 702 | 2.4e−68 | 1 |
| ACC: Q9Y6H8 | Homo Sapiens | | | | |

NOV3b

In the present invention, the target sequence identified previously, Accession Number GMba63k6_A, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number CG54734-02.

A disclosed NOV3b (also referred to as CG54734-02) nucleic acid of 1641 nucleotides is shown in Table 3D. An open reading frame was identified beginning with an ATG initiation codon at nucleotides 5-7 and ending with a TAA codon at nucleotides 1634-1636. A putative untranslated region upstream from the initiation codon and downstream from the termination codon is underlined in Table 3D, and the start and stop codons are in bold letters.

TABLE 3D
NOV3b Nucleotide Sequence

(SEQ ID NO:11)

TACCATGGGGGACTGGAACTTATTGGGTGGCATCCTAGAGGAAGTTCACTCCCACTCAACCATAGTGGGGA
AAATCTGGCTGACCATCCTCTTCATCTTCCGAATGCTGGTACTTCGTGTGGCTGCTGAGGATGTCTGGGAT
GATGAACAGTCAGCATTTGCCTGCAACACCCGGCAGCCAGGTTGCAACAATATCTGTTATGATGATGCATT
CCCTATCTCTTTGATCAGGTTCTGGGTTTTACAGATCATCTTTGTGTCTTCTCCTTCTTTGGTCTATATGG
GCCATGCACTTTATAGGCTCAGGGCCTTTGAGAAAGACAGGCAGAGGAAAAAGTCACACCTTAGAGCCCAG
ATGGAGAATCCAGATCTTGACTTGGAGGAGCAGCAAAGAATAGATAGGGAACTGAGGAGGTTAGAGGAGCA
GAAGAGGATCCATAAAGTCCCTCTGAAAGGATGTCTGCTGCGTACTTATGTCTTACACATCTTGACCAGAT
CTGTGCTGGAAGTAGGATTCATGATAGGCCAATATATTCTCTATGGGTTTCAAATGCACCCCCTTTACAAA
TGCACTCAACCTCCTTGCCCCAATGCGGTGGATTGCTTTGTATCCAGGCCCACTGAGAAGACAATTTTCAT
GCTTTTTATGCACAGCATTGCAGCCATTTCCTTGTTACTCAATATACTGGAAATATTTCATCTAGGCATCA
GAAAAATTATGAGGACACTTTATAAGAAATCCAGCAGTGAGGGCATTGAGGATGAAACAGGCCCTCCATTC
CATTTGAAGAAATATTCTGTGGCCCAGCAGTGTATGATTTGCTCTTCATTGCCTGAAAGAATCTCTCCACT
TCAAGCTAACAATCAACAGCAAGTCATTCGAGTTAATGTGCCAAAGTCTAAAACCATGTGGCAAATCCCAC
AGCCAAGGCAACTTGAAGTAGACCCTTCCAATGGGAAAAAGGACTGGTCTGAGAAGGATCAGCATAGCGGA
CAGCTCCATGTTCACAGCCCGTGTCCCTGGGCTGGCAGTGCTGGAAATCAGCACCTGGGACAGCAATCAGA
CCATTCCTCATTTGGCCTGCAGAATACAATGTCTCAGTCCTGGCTACGTACAACTACGGCTCCTAGAAACT
GTCCATCCTTTGCAGTAGGAACCTGGGAGCAGTCCCAGGACCCAGAACCCTCAGGTGAGCCTCTCACAGAT
CTTCATAGTCACTGCAGAGACAATGAAGGCAGCATGAGAGAGAGTGGGGTCTGGATAGACAGATCTCGCCC
AGGCAGTCGCAAGGCCAGCTTTCTGTCCAGATTGTTGTCTGAAAAGCGACATCTGCACAGTGACTCAGGAA
GCTCTGGTTCTCGGAATAGCTCCTGCTTGGATTTTCCTCACTGGGAAAACAGCCCCTCACCTCTGCCTTCA
GTCACTGGGCACAGAACATCAATGGTAAGACGGGCAGCCCTACCGATCATGGAACTATCACAAGAGCTGTT
CCATTCTGGATGCTTTCTTTTTCCTTTCTTTCTTCCTGGGGTGTGTATGTATGTTTGTGTTGACAGAGAGG
CAGATGGAGGGGGAGATTATTTATGGAGAGATAAAATTATTCATTCGATACATTCAGTTAAATTCAATTCA
TAAAATAA

In a search of public sequence databases, it was found, for example, that the disclosed NOV3b nucleic acid sequence has 1239 of 1467 bases (84%) identical to a Mus musculus connexin 57 mRNA (GENBANK-ID: AJ010741.1)(E=1.7e-239). Public nucleotide databases include all GenBank databases and the GeneSeq patent database.

The disclosed NOV3b polypeptide (SEQ ID NO: 12) encoded by SEQ ID NO:11 is 543 amino acid residues and is presented using the one-letter code in Table 3E. The NOV3b protein was analyzed for signal peptide prediction and cellular localization. SignalP, Psort and Hydropathy results predict that NOV3b has a signal peptide with most likely cleavage site pos. 41 and 42, at: VAA-ED, and that NOV3b is likely to be localized in the plasma membrane with a certainty of 0.6000.

TABLE 3E
Encoded NOV3b protein sequence.

(SEQ ID NO:12)

MGDWNLLGGILEEVHSHSTIVGKIWLTILFIFRMLVLRVAAEDVWDDEQSAFACNTRQPGCNNICYDDAFPI
SLIRFWVLQIIFVSSPSLVYMGHALYRLRAFEKDRQRKKSHLRAQMENPDLDLEEQQRIDRELRRLEEQKRI
HKVPLKGCLLRTYVLHILTRSVLEVGFMIGQYILYGFQMHPLYKCTQPPCPNAVDCFVSRPTEKTIFMLFMH
SIAAISLLLNILEIFHLGIRKIMRTLYKKSSSEGIEDETGPPFHLKKYSVAQQCMICSSLPERISPLQANNQ
QQVIRVNVPKSKTMWQIPQPRQLEVDPSNGKKDWSEKDQHSGQLHVHSPCPWAGSAGNQHLGQQSDHSSFGL
QNTMSQSWLGTTTAPRNCPSFAVGTWEQSQDPEPSGEPLTDLHSHCRDNEGSMRESGVWIDRSRPGSRKASF
LSRLLSEKRHLHSDSGSSGSRNSSCLDFPHWENSPSPLPSVTGHRTSMVRRAALPIMELSQELFHSGCFLFP
FFLPGVCMYVCVDREADGGGDYLWRDKIIHSIHSVKFNS

The full amino acid sequence of the protein of the invention was found to have 392 of 485 amino acid residues (80%) identical to, and 422 of 485 amino acid residues (87%) similar to, the 505 amino acid residue connexin 57 protein from Mus musculus (ptnr:SPTREMBL-ACC:Q9WUS4)(E=1.1e-211).

Possible SNPs found for NOV3b are listed in Table 3F.

TABLE 3F
SNPs

| Consensus | | |
|---|---|---|
| Position | Depth | Base Change |
| 83 | 16 | G > A |
| 1242 | 21 | A > G |
| 1337 | 17 | T > C |

It was also found that NOV3a had homology to the amino acid sequences shown in the BLASTP data listed in Table 3G.

TABLE 3G
BLAST results for NOV3a

| Gene Index/ | Protein/ | Length | Identity | Positives | |
|---|---|---|---|---|---|
| Identifier | Organism | (aa) | (%) | (%) | Expect |
| gi\|14009611\| | connexin | 543 | 508/543 | 510/543 | 0.0 |
| gb\|AAK51676.1\| | 62 | | (93%) | (93%) | |
| AF2967661 | [Homo | | | | |
| (AF296766) | sapiens] | | | | |
| gi\|6753990\| | gap | 505 | 364/485 | 394/485 | 0.0 |
| ref\|NP_034419.1\| | junction | | (75%) | (81%) | |
| | membrane | | | | |
| | channel | | | | |
| | protein | | | | |
| | alpha 10; | | | | |
| | connexin-57 | | | | |
| | [Mus | | | | |
| | musculus] | | | | |
| gi\|10946367\| | connexin | 498 | 170/277 | 210/277 | 1e−89 |
| gb\|AAG24878.1\| | 55.5 | | (61%) | (75%) | |
| (AF304048) | [Danio | | | | |
| | rerio] | | | | |
| gi\|13540537\| | connexin | 515 | 161/246 | 196/246 | 2e−88 |
| ref\|NP_110399.1\| | 59; | | (65%) | (79%) | |
| | gap | | | | |
| | junction | | | | |
| | alpha 10 | | | | |
| | [Homo | | | | |
| | sapiens] | | | | |
| gi\|12719964\| | gap | 433 | 128/245 | 167/245 | 4e−66 |
| ref\|XP_001660.2\| | junction | | (52%) | (67%) | |
| | protein, | | | | |
| | alpha | | | | |
| | 8, 50kD | | | | |
| | (connexin | | | | |
| | 50) | | | | |
| | [Homo | | | | |
| | sapiens] | | | | |

The homology of these sequences is shown graphically in the ClustalW analysis shown in

[image]

[image]

The homologies shown above are shared by NOV3b insofar as NOV3b and NOV3a are homologous.

Tables 3I and 3J list the domain description from DOMAIN analysis results against NOV3a. This indicates that the NOV3a sequence has properties similar to those of other proteins known to contain this domain.

[image]

The connexins are a family of integral membrane proteins that oligomerise to form intercellular channels that are clustered at gap junctions. These channels are specialized sites of cell-cell contact that allow the passage of ions, intracellular metabolites and messenger molecules (with molecular weights of 1-2 kD) from the cytoplasm of one cell to its apposing neighbors. They are found in almost all vertebrate cell types, and somewhat similar proteins have been cloned from plant species. Invertebrates utilize a different family of molecules, innexins that share a similar predicted secondary structure to the vertebrate connexins, but have no sequence identity to them.

Vertebrate gap junction channels are thought to participate in diverse biological functions. For instance, in the heart they permit the rapid cell-cell transfer of action potentials, ensuring coordinated contraction of the cardiomyocytes. They are also responsible for neurotransmission at specialized ‘electrical’ synapses. In non-excitable tissues, such as the liver, they may allow metabolic cooperation between cells. In the brain, gap junctions extensively couples glial cells; this allows waves of intracellular Ca²⁺ to propagate through nervous tissue, and may contribute to their ability to spatially-buffer local changes in extracellular K⁺ concentration.

The connexin protein family is encoded by at least 13 genes in rodents, with many homologues cloned from other species. They show overlapping tissue expression patterns, most tissues expressing more than one connexin type. Their conductances, permeability to different molecules, phosphorylation and voltage-dependence of their gating, have been found to vary. Possible communication diversity is increased further by the fact that gap junctions may be formed by the association of different connexin isoforms from apposing cells. However, in vitro studies have shown that not all possible combinations of connexins produce active channels.

Hydropathy analysis predicts that all cloned connexins share a common transmembrane (TM) topology. Each connexin is thought to contain 4 TM domains, with two extracellular and three cytoplasmic regions. This model has been validated for several of the family members by in vitro biochemical analysis. Both N- and C-termini are thought to face the cytoplasm, and the third TM domain has an amphipathic character, suggesting that it contributes to the lining of the formed-channel. Amino acid sequence identity between the isoforms is −50-80%, with the TM domains being well conserved. Both extracellular loops contain characteristically conserved cysteine residues, which likely form intramolecular disulphide bonds. By contrast, the single putative intracellular loop (between TM domains 2 and 3) and the cytoplasmic C-terminus are highly variable among the family members. Six connexins are thought to associate to form a hemi-channel, or connexon. Two connexons then interact (likely via the extracellular loops of their connexins) to form the complete gap junction channel.

Gap junctions were first characterized by electron microscopy as regionally specialized structures on plasma membranes of contacting adherent cells. These structures were shown to consist of cell-to-cell channels. Connexin proteins are purified from fractions of enriched gap junctions from different tissues differ. The connexins are designated by their molecular mass. Another system of nomenclature divides gap junction proteins into 2 categories, alpha and beta, according to sequence similarities at the nucleotide and amino acid levels. For example, CX43 is designated alpha-i gap junction protein, whereas CX32 and CX26 are called beta-1 and beta-2 gap junction proteins, respectively. This nomenclature emphasizes that CX32 and CX26 are more homologous to each other than either of them is to CX43.

Willecke et al. (1990) used rat connexin gene probes in Southern blot analysis of human-mouse somatic cell hybrids to map the CX26 gene to chromosome 13. By means of somatic cell hybrids, Hsieh et al. (1991) assigned the GJB2 gene to chromosome 13 in man and chromosome 14 in the mouse. Haefliger et al. (1992) showed that the rat homologs of the CX26 and CX46 genes are tightly linked on chromosome 14. By isotopic in situ hybridization, Mignon et al. (1996) mapped GJB2to 13q11-q12 and confirmed the assignment to mouse chromosome 14.

Various studies have been carried out to investigate the role(s) of altered genes or proteins from the connexin family. Kelsell et al. (1997) studied a pedigree containing individuals with autosomal dominant deafness and identified a mutation in the CX26 gene: a101T-C transition resulting in a met34-to-thr amino acid substitution. CX26 mutations resulting in premature stop codons were also found in 3autosomal recessive nonsyndromic sensorineural deafness pedigrees, genetically linked to 13q11-q12, where the CX26 gene is localized. Immunohistochemical staining of human cochlear cells for CX26 demonstrated high levels of expression. Kelley et al. (1998) presented evidence that the 101T-C missense mutation identified by Kelsell et al. (1997) in individuals with autosomal dominant nonsyndromic deafness is not sufficient to cause hearing loss.

Carrasquillo et al. (1997) performed linkage analysis in 2 interrelated inbred kindreds in a single Israeli-Arab village containing more than 50 individuals with nonsyndromic recessive deafness. Genetic mapping demonstrated that a gene located at 13q11 (DFNB1) segregated with the deafness in these 2 kindreds. Haplotype analysis, using 8 microsatellite markers spanning 15 cM in 13q11, suggested the segregation of 2 different mutations in this extended kindred; affected individuals were homozygotes for either haplotype or compound heterozygotes. Carrasquillo et al. (1997) identified 2 distinct mutations, trp77 to arg and 35delG, in the CX26 gene, both of which were predicted to inactivate connexin 26. The recombination of marker alleles involving polymorphisms in 13q11, at known map distances from the mutations, allowed them to estimate the age of the mutations to be 3 to 5 generations (75 to 125 years). The study demonstrated that in small populations with high rates of consanguinity, as compared with large outbred populations, recessive mutations may have very recent origin and show allelic diversity. They pointed to the same phenomenon being observed for Hurler syndrome with 3 unique mutations and for metachromatic leukodystrophy with 5 distinct mutations, discovered among the Druze and Muslim Arab villages in Israel. In light of these findings, the authors commented that it is likely that homozygosity mapping studies in highly inbred communities may be compromised, as may be studies of mapping by linkage disequilibrium, unless the possibility of mutational diversity is taken into account.

Lench et al. (1998) studied the role of CX26 mutations in singleton (sporadic) cases of nonsyndromal sensorineural deafness. Such mutations were identified in 4 of 43 U.K. and 2 of 25 Belgian patients. Thus, about 10% of families presenting with a child sporadically affected with this disorder can be offered definitive mendelian recurrence risks. This was said to be the first genetic test available for screening such children.

Kelley et al. (1998) analyzed 58 multiplex families each having at least 2 affected children diagnosed with autosomal recessive nonsyndromic deafness. Mutations in both alleles of GJB2 were observed in 20 of the 58 families. A 30delG allele occurred in 33 of the 116 chromosomes, for a frequency of 0.284. This mutation was observed in 2 of 192 control chromosomes, for an estimated gene frequency of 0.01+/−0.007. The homozygous frequency of the 30delG allele was then estimated at 0.0001, or 1 in 10,000. Given that the frequency of all childhood hearing impairment is 1 in 1,000 and that half of that is genetic, the specific mutation 30delG is responsible for 10% of all childhood hearing loss and for 20% of all childhood hereditary hearing loss. Six novel mutations were also observed in the affected population.

In more recent studies, Murgia et al. (1999) studied 53 unrelated individuals with non syndromic sensorineural hearing impairment and carried out CX26 mutation analysis. Mutations were found in 53% of cases, in 35.3% of those in whom autosomal recessive inheritance was thought likely and in 60% of the presumed sporadic cases. Three novel mutations were found. The hearing deficit varied from mild to profound even within the same family. Among patients with profound hearing loss, 35.5% were found to have a mutation; among those severely impaired, 20%; and among those moderately impaired, 33.3%. Rabionet et al. (2000) analyzed the GJB2 gene in 576 families/unrelated patients with recessive or sporadic deafness from Italy and Spain, 193 of them being referred as autosomal recessive and the other 383 as apparently sporadic. Of the 1,152 unrelated GJB2 chromosomes, 37% had GJB2 mutations. A total of 23 different mutations were detected. Mutation 35delG was the most common, accounting for 82% of all GJB2 deafness alleles. It represented 88% of the alleles in Italian patients and only 55% in Spanish cases. Sobe et al. (2000) sequenced the entire coding region of the GJB2 gene in 75 hearing-impaired children and adults in Israel. Age at onset in the screened population was both prelingual and postlingual, with hearing loss ranging from moderate to profound. Almost 39%of all persons tested harbored GJB2 mutations, most of which were 35delG and 167delT. A novel mutation, involving both a deletion and an insertion, 51dell2insA, was identified in a family originating from Uzbekistan. All GJB2 mutations were associated with prelingual hearing loss, although severity ranged from moderate to profound, with variability even among hearing-impaired sibs. No significant difference in hearing levels was found between individuals with 35delG and 167delT mutations.

The above defined information for this invention suggests that these connexin-like protein (NOV3a and 3b) may function as members of the “connexin family”. Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in mutilating palmoplantar keratoderma (PPK), X-linked Charcot-Marie-Tooth neuropathy, hereditary peripheral neuropathy, hereditary non-syndromic sensorineural deafness, vohwinkel's syndrome an autosomal dominant disease characterized by hyperkeratosis, constriction on finger and toes and congenital deafness and other pathologies and disorders. For example, a cDNA encoding the connexin-like protein may be useful in gene therapy, and the connexin-like protein may be useful when administered to a subject in need thereof. By way of nonlimiting example, the compositions of the present invention will have efficacy for treatment of patients suffering from Clouston syndrome and deafness, mutilating palmoplantar keratoderma (PPK), X-linked Charcot-Marie-Tooth neuropathy, hereditary peripheral neuropathy. The novel nucleic acid encoding connexin-like protein, and the connexin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acids encoding the connexin-like proteins of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. The disclosed NOV3 proteins have multiple hydrophilic regions, each of which can be used as an immunogen. In one embodiment, a contemplated NOV3 epitope is from about amino acids 40 to 70. In another embodiment, a NOV3 epitope is from about amino acids 85 to 145. In additional embodiments, NOV3 epitopes are from about amino acids 175 to 200, 225-270, 280-480 and from about amino acids 510 to 530. These novel proteins can be used in assay systems for functional analysis of various human disorders, which will help in understanding of pathology of the disease and development of new drug targets for various disorders.

NOV4

NOV4 includes four novel hepatoma derived growth factor-like proteins disclosed below. The disclosed proteins have been named NOV4a, NOV4b, NOV4c and NOV4d.

NOV4a

A novel nucleic acid of 2031 nucleotides (also referred to as 85731808_EXT) encoding a novel Hepatoma Derived Growth Factor-like protein is shown in FIG. 4A. An open reading frame was identified beginning with an ATG initiation codon at nucleotides 1-3 and ending with a TGA codon at nucleotides 2029-2031. The start and stop codons are in bold letters in FIG. 4A.

TABLE 4A
NOV4a Nucleotide Sequence.

(SEQ ID NO:13)

ATGCCAAACGCCTTTAAACCCGGGGACTTGGTTTTCCCTAAAATTAAGGGCTACCCTCAATGGCCTTCCAG
GATCGACGACATCGCGGATGGCGCCGTGAAGCCCCCACCCAACAAGTACCCCATCTTTTTCTTTGGCACAC
ACGAAACGGCCTTCCTGGGACCCAAGGACCTGTTCCCCTACGACAAATGTAAAGACAAGTACGGGAAGCCC
AACAAGAGGAAAGGCTTCAATGAAGGGCTGTGGGAGATCCAGAACAACCCCCACGCCAGCTACAGCGCCCC
TCCGCCAGTGAGCTCCTCCGACAGCGAGGCCCCCGAGGCCAACCCCGCCGACGGCAGTGACGCTGACGAGG
ACGATGAGGACCGGGGGGTCATGGCCGTCACAGCGGTAACCGCCACAGCTGCCAGCGACAGGATGGAGAGC
GACTCAGACTCAGACAAGAGTAGCGACAACAGTGGCCTGAAGAGGAAGACGCCTGCGCTAAAGGTATCGGT
CTCGAAACGAGCCCGAAAGGCCTCCAGCGACCTGGATCAGGCCAGCGTGTCCCCATCCGAAGAGGAGAACT
CGGAAAGCTCATCTGAGTCGGAGAAGACCAGCGACCAGGACTTCACACCTGAGAAGAAAGCAGCGGTCCGG
GCGCCACGGAGGGGCCCTCTGGGGGGACGGAAAAAAAAGAAGGCGCCATCAGCCTCCGACTCCGACTCCAA
GGCCGATTCGGACGGGGCCAAGCCTGAGCCGGTGGCCATGGCGCGGTCGGCGTCCTCCTCCTCCTCTTCCT
CCTCCTCCTCCGACTCCGATGTGTCTGTGAAGAAGCCTCCGAGGGGCAGGAAGCCAGCGGAGAAGCCTCTC
CCGAAGCCGCGAGGGCGGAAACCGAAGCCTGAACGGCCTCCGTCCAGCTCCAGCAGTGACAGTGACAGCGA
CGAGGTGGACCGCATCAGTGAGTGGAAGCGGCGGGACGAGGCGCGGAGGCGCGAGCTGGAGGCCCGGCGGC
GGCGAGAGCAGGAGGAGGAGCTGCGGCGCCTGCGGGAGCAGGAGAAGGAGGAGAAGGAGCGGAGGCGCGAG
CGGGCCGACCGCGGGGAGGCTGAGCGGGGCAGCGGCGGCAGCAGCGGGGACGAGCTCAGGGAGGACGATGA
GCCCGTCAAGAAGCGGGGACGCAAGGGCCGGGGCCGGGGTCCCCCGTCCTCCTCTGACTCCGAGCCCGAGG
CCGAGCTGGAGAGAGAGGCCAAGAAATCAGCGAAGAAGCCGCAGTCCTCAAGCACAGAGCCCGCCAGGAAA
CCTGGCCAGAAGGAGAAGAGAGTGCGGCCCGAGGAGAAGCAACAAGCCAGGCCCGTGAAGGTGGAGCGGAC
CCGGAAGCGGTCCGAGGGCTTCTCGATGGACAGGAAGGTAGAGAAGAAGAAAGAGCCCTCCGTGGAGGAGA
AGCTGCAGAAGCTGCACAGTGAGATCAAGTTTGCCCTAAAGGTCGACAGCCCGGACGTGAAGAGGTGCCTG
AATGCCCTAGAGGAGCTGGGAACCCTGCAGGTGACCTCTCAGATCCTCCAGAAGAACACAGACGTGGTGGC
CACCTTGAAGAAGATTCGCCGTTACAAAGCGAACAAGGACGTAATGGAGAAGGCAGCAGAAGTCTATACCC
GGCTCAAGTCGCGGGTCCTCGGCCCAAAGATCGAGGCGGTGCAGAAAGTGAACAAGGCTGGGATGGAGAAG
GAGAAGGCCGAGGAGAAGCTGGCCGGGGAGGAGCTGGCCGGGGAGGAGCTGGCCGGGGAGGAGGCCCCCCA
GGAGAAGGCGGAGGACAAGCCCAGCACCGATCTCTCAGCCCCAGTGAATGGCGAGGCCACATCACAGAAGG
GGGAGAGCGCAGAGGACAAGGAGCACGAGGAGGGTCGGGACTCGGAGGAGGGGCCAAGGTGTGGCTCCTCT
GAAGACCTGCACGAGAGCGTACGGGAGGGTCCCGACCTGGACAGGCCTGGGAGCGACCGGCAGGAGCGCGA
GAGGGCACGGGGGGACTCGGAGGCCCTGGACGAGGAGAGCTGA

In a search of public sequence databases, it was found, for example, that the nucleic acid sequence (NOV4a) has 1268 of 1775 bases (71%) identical to a Mus musculus Hepatoma Derived Growth Factor-like protein mRNA (GENBANK-ID:D63850) (E=2.5 e-174). It is also 70% similar over 364 bp to a separate, but partially overlapping, fragment of the same gene.

The NOV4a polypeptide (SEQ ID NO:14) encoded by SEQ ID NO:13 is 676 amino acid residues and is presented using the one-letter amino acid code in Table 4B. The NOV4a protein was analyzed for signal peptide prediction and cellular localization. SignalP, Psort and Hydropathy profile indicate that NOV4a does not have a signal peptide and is likely to be localized in the nucleus with a certainty of 0.9866. However, the protein predicted here is similar to the “Hepatoma Derived Growth Factor-Like Protein Family”, some members of which end up outside the cell and exhibit growth factor activity. Therefore it is likely that NOV4a is available at the appropriate sub-cellular localization and hence accessible for the therapeutic uses described in this application.

TABLE 4B
NOV4a protein sequence

(SEQ ID NO:14)

MPNAFKPGDLVFPKIKGYPQWPSRIDDIADGAVKPPPNKYPIFFFGTHETAFLGPKDLFPYDKCKDKYGKPNKR
KGFNEGLWEIQNNPHASYSAPPPVSSSDSEAPEANPADGSDADEDDEDRGVMAVTAVTATAASDRMESDSDSDK
SSDNSGLKRKTPALKVSVSKRARKASSDLDQASVSPSEEENSESSSESEKTSDQDFTPEKKAAVRAPRRGPLGG
RKKKKAPSASDSDSKADSDGAKPEPVAMARSASSSSSSSSSSDSDVSVKKPPRGRKPAEKPLPKPRGRKPKPER
PPSSSSSDSDSDEVDRISEWKRRDEARRRELEARRRREQEEELRRLREQEKEEKERRRERADRGEAERGSGGSS
GDELREDDEPVKKRGRKGRGRGPPSSSDSEPEAELEREAKKSAKKPQSSSTEPARKPGQKEKRVRPEEKQQARP
VKVERTRKRSEGFSMDRKVEKKKEPSVEEKLQKLHSEIKFALKVDSPDVKRCLNALEELGTLQVTSQILQKNTD
VVATLKKIRRYKANKDVMEKAAEVYTRLKSRVLGPKIEAVQKVNKAGMEKEKAEEKLAGEELAGEELAGEEAPQ
EKAEDKPSTDLSAPVNGEATSQKGESAEDKEHEEGRDSEEGPRCGSSEDLHESVREGPDLDRPGSDRQERERAR
GDSEALDEES

The Hepatoma Derived Growth Factor-like protein (NOV4a) maps to chromosome 19 and is expressed in at least the following tissues: lung, blood, lymphocyte, bone marrow, colon, brain, pancreas, pituitary, testis, ovaries, prostate, and uterus.

The full amino acid sequence of the NOV4a protein was found to have 548 of 676 amino acid residues (81%) identical to, and 601 of 676 residues (88%) similar to, the 669 amino acid residue Hepatoma Derived Growth Factor-like protein from Mus musculus (SPTREMBL-ACC:035540; E=1.3 e-284) (Table 4C).

TABLE 4C
BLASTX results for NOV4a

| | | | | Smallest | |
|---|---|---|---|---|---|
| | | | | Sum | |
| Sequences producing High-scoring | | Reading | High | Prob. | |
| Segment Pairs: | | Frame | Score | P (N) | N |
| ptnr: SPTREMBL- | Hepatoma- | +1 | 2742 | 1.3e−284 | 1 |
| ACC: 035540 | Derived GF - | | | | |
| | Mus | | | | |

NOV4a, as well as 4b, 4c and 4d, sequences were initially identified by searching a proprietary sequence file database for DNA sequences which translate into proteins with similarity to Hepatoma Derived Growth Factor-Like Proteins. NOV4a was identified as having suitable similarity. NOV4a was analyzed further to identify any open reading frames encoding novel full length proteins as well as novel splice forms of these genes. This was done by extending the identified NOV4a using suitable sequences from additional proprietary assemblies, publicly available EST sequences and public genomic sequences. A Genomic clone (AC011498) HTG Homo sapiens chromosome 19 clone CTB-50L17 was identified as having regions with 100% identity to the NOV4a and was selected for analysis because this identity implied that this clone contained the sequence of the genomic locus for NOV4a.

The genomic clone was analysed by Genscan and Grail to identify exons and putative coding sequences/open reading frames. This clone was also analyzed by TblastN, BlastX and other homology programs to identify regions translating to proteins with similarity to the original protein/protein family of interest. Expressed sequences from both public and proprietary databases were also added when available to further define and complete the gene sequence. The DNA sequence was then manually corrected for apparent inconsistencies thereby obtaining the sequences encoding the full-length protein.

NOV4b

In the present invention, the target sequence identified above, Accession Number 85731808_EXT, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such suitable sequences were then employed as the forward and reverse primers in a PCR amplification based on a library containing a wide range of cDNA species. The resulting amplicon was gel purified, cloned and sequenced to high redundancy to provide the sequence reported below, which is designated Accession Number 21143463.0.45.

A disclosed NOV4b (also referred to as 21143463.0.45) nucleic acid of 2004 nucleotides is shown in FIG. 4D. An open reading frame was identified beginning with an ATG initiation codon at nucleotides 1-3 and ending with a TGA codon at nucleotides 2002-2004. The start and stop codons are in bold letters in FIG. 4D.

TABLE 4D
NOV4b Nucleotide Sequence.

(SEQ ID NO:15)

ATGCCACACGCCTTCAAGCCCGGGGACTTGGTGTTCGCTAAGATGAAGGGCTACCCTCACTGGCCTGCCAG
GGTGGACGACATCGCGGATGGCGCCGTGAAGCCCCCACCCAACAAGTACCCCATCTTTTTCTTTGGCACAC
ACGAAACGGCCTTCCTGGGACCCAAGGACCTGTTCCCCTACGACAAATGTAAAGACAAGTACGGGAAGCCC
AACAAGAGGAAAGGCTTCAATGAAGGGCTGTGGGAGATCCACAACAACCCCCACGCCAGCTACAGCGCCCC
TCCGCCAGTGAGCTCCTCCGACAGCGAGGCCCCCGAGGCCAACCCCGCCGACGGCAGTGACGCTGACGAGG
ACGATGAGGACCGGGGGGTCATGGCCGTCACAGCGGTAACCGCCACAGCTGCCAGCGACAGGATGGAGAGC
GACTCAGACTCAGACAAGAGTAGCGACAACAGTGGCCTGAAGAGGAAGACGCCTGCGCTAAAGGTATCGGT
CTCGAAACGAGCCCGAAAGGCCTCCAGCGACCTGGATCAGGCCAGCGTGTCCCCATCCGAAGAGGAGAACT
CGGAAAGCTCATCTGAGTCGGAGAAGACCAGCGACCAGGACTTCACACCTGAGAAGAAAGCAGCGGTCCGG
GCGCCACGGAGGGGCCCTCTGGGGGGACGGAAAAAAAAGAAGGCGCCATCAGCCTCCGACTCCGACTCCAA
GGCCGATTCGGACGGGGCCAAGCCTGAGCCGGTGGCCATGGCGCGGTCGGCGTCCTCCTCCTCCTCTTCCT
CCTCCTCCTCCGACTCCGATGTGTCTGTGAAGAAGCCTCCGAGGGGCAGGAAGCCAGCGGAGAAGCCTCTC
CCGAAGCCGCGAGGGCGGAAACCGAAGCCTGAACGGCCTCCGTCCAGCTCCAGCAGTGACAGTGACAGCGA
CGAGGTGGACCGCATCAGTGAGTGGAAGCGGCGGGACGAGGCGCGGAGGCGCGAGCTGGAGGCCCGGCGGC
GGCGAGAGCAGGAGGAGGAGCTGCGGCGCCTGCGGGAGCAGGAGAAGGAGGAGAAGGAGCGGAGGCGCGAG
CGGGCCGCTGAGCGGGGCAGCGGCGGCAGCAGCGGGGACGAGCTCAGGGAGGACGATGAGCCCGTCAAGAA
GCGGGGACGCAAGGGCCGGGGCCGGGGTCCCCCGTCCTCCTCTGACTCCGAGCCCGAGGCCGAGCTGGAGA
GAGAGGCCAAGAAATCAGCGAAGAAGCCGCAGTCCTCAAGCACAGAGCCCGCCAGGAAACCTGGCCAGAAG
GAGAAGAGAGTGCGGCCCGAGGAGAAGCAACAAGCCAAGCCCGTGAAGGTGGAGCGGACCCGGAAGCGGTC
CGAGGGCTTCTCGATGGACAGGAAGGTAGAGAAGAAGAAAGAGCCCTCCGTGGAGGAGAAGCTGCAGAAGC
TGCACAGTGAGATCAAGTTTGCCCTAAAGGTCGACAGCCCGGACGTGAAGAGGTGCCTGAATGCCCTAGAG
GAGCTGGGAACCCTGCAGGTGACCTCTCAGATCCTCCAGAAGAACACAGACGTGGTGGCCACCTTGAAGAA
GATTCGCCGTTACAAAGCGAACAAGGACGTAATGGAGAAGGCAGCAGAAGTCTATACCCGGCTCAAGTCGC
GGGTCCTCGGCCCAAAGATCGAGGCGGTGCAGAAAGTGGACAAGGCTGGGATGGAGAAGGAGAAGGCCGAG
GAGAAGCTGGCCGGGGAGGAGCTGGCCGGGGAGGAGGCCCCCCAGGAGAAGGCGGAGGACAAGCCCAGCAC
CGATCTCTCAGCCCCAGTGAATGGCGAGGCCACATCACAGAAGGGGGAGAGCGCAGAGGACAAGGAGCACG
AGGAGGGTCGGGACTCGGAGGAGGGGCCAAGGTGTGGCTCCTCTGAAGACCTGCACGAGAGCGTACGGGAG
GGTCCCGACCTGGACAGGCCTGGGAGCGACCGGCAGGAGCGCGAGAGGGCACGGGGGGACTCGGAGGCCCT
GGACGAGGAGAGCTGA

The NOV4b polypeptide (SEQ ID NO:16) encoded by SEQ ID NO:15 is 667 amino acid residues and is presented using the one-letter amino acid code in Table 4E. The NOV4b protein was analyzed for signal peptide prediction and cellular localization. SignalP, Psort and Hydropathy profile indicate that NOV4b does not have a signal peptide and is likely to be localized in the nucleus with a certainty of 0.9867. However, the protein predicted here is similar to the “Hepatoma Derived Growth Factor-Like Protein Family”, some members of which end up outside the cell and exhibit growth factor activity. Therefore it is likely that NOV4b is available at the appropriate sub-cellular localization and hence accessible for the therapeutic uses described in this application. NOV4b has a molecular weight of 73827.3 Daltons.

TABLE 4E
NOV4b protein sequence

(SEQ ID NO:16)

MPHAFKPGDLVFAKMKGYPHWPARVDDIADGAVKPPPNKYPIFFFGTHETAFLGPKDLFPYDKCKDKYGKPNKR
KGFNEGLWEIQNNPHASYSAPPPVSSSDSEAPEANPADGSDADEDDEDRGVMAVTAVTATAASDRMESDSDSDK
SSDNSGLKRKTPALKVSVSKRARKASSDLDQASVSPSEEENSESSSESEKTSDQDFTPEKKAAVRAPRRGPLGG
RKKKKAPSASDSDSKADSDGAKPEPVAMARSASSSSSSSSSSDSDVSVKKPPRGRKPAEKPLPKPRGRKPKPER
PPSSSSSDSDSDEVDRISEWKRRDEARRRELEARRRREQEEELRRLREQEKEEKERRRERAAERGSGGSSGDEL
REDDEPVKKRGRKGRGRGPPSSSDSEPEAELEREAKKSAKKPQSSSTEPARKPGQKEKRVRPEEKQQAKPVKVE
RTRKRSEGFSMDRKVEKKKEPSVEEKLQKLHSEIKFALKVDSPDVKRCLNALEELGTLQVTSQILQKNTDVVAT
LKKIRRYKANKDVMEKAAEVYTRLKSRVLGPKIEAVQKVDKAGMEKEKAEEKLAGEELAGEEAPQEKAEDKPST
DLSAPVNGEATSQKGESAEDKEHEEGRDSEEGPRCGSSEDLHESVREGPDLDRPGSDRQERERARGDSEALDEE
S

The Hepatoma Derived Growth Factor-like protein (NOV4b) is expressed in at least the following tissues: lung, blood, lymphocyte, bone marrow, colon, brain, pancreas, pituitary, testis, ovaries, liver, prostate, heart, adrenal gland, spleen, thyroid and uterus.

The amino acid sequence of NOV4b had high homology to other proteins as shown in Table 4F.

TABLE 4F
BLASTX results for NOV4b

| | | | | Smallest | |
|---|---|---|---|---|---|
| | | | | Sum | |
| | | Reading | High | Prob. | |
| Sequences producing High-scoring Segment Pairs: | | Frame | Score | P (N) | N |
| ptnr: SPTREMBL-ACC: 035540 | HEPATOMA-DERIVED GROWTH FACTO . . . | +1 | 2774 | 6.6e−288 | 1 |
| ptnr: PIR-ID: JC7168 | lens epithelium-derived growth fact . . . | +1 | 522 | 4.3e−52 | 2 |
| ptnr: SPTREMBL-ACC: 075475 | LENS EPITHELIUM-DERIVED GROWT . . . | +1 | 518 | 1.1e−51 | 2 |
| ptnr: SPTREMBL-ACC: Q9UER6 | TRANSCRIPTIONAL COACTIVATOR P . . . | +1 | 517 | 1.4e−51 | 2 |
| ptnr: TREMBLNEW-ACC: AAF25871 | LENS EPITHELIUM-DERIVED GR . . . | +1 | 503 | 2.9e−47 | 1 |
| ptnr: SPTREMBL-ACC: 095368 | TRANSCRIPTIONAL COACTIVATOR P . . . | +1 | 502 | 3.8e−47 | 1 |

NOV4c

A disclosed NOV4c (also referred to as 21143463_A.0.45_EXT) nucleic acid of 2004 nucleotides is shown in FIG. 4G. An open reading frame was identified beginning with an ATG initiation codon at nucleotides 1-3 and ending with a TGA codon at nucleotides 2002-2004. The start and stop codons are in bold letters in FIG. 4G.

TABLE 4G
NOV4c Nucleotide Sequence.

(SEQ ID NO:17)

ATGCCACACGCCTTCAAGCCCGGGGACTTGGTGTTCGCTAAGATGAAGGGCTACCCTCACTGGCCTGCCAG
GGTGGACGACATCGCGGATGGCGCCGTGAAGCCCCCACCCAACAAGTACCCCATCTTTTTCTTTGGCACAC
ACGAAACGGCCTTCCTGGGACCCAAGGACCTGTTCCCCTACGACAAATGTAAAGACAAGTACGGGAAGCCC
AACAAGAGGAAAGGCTTCAATGAAGGGCTGTGGGAGATCCAGAACAACCCCCACGCCAGCTACAGCGCCCC
TCCGCCAGTGAGCTCCTCCGACAGCGAGGCCCCCGAGGCCAACCCCGCCGACGGCAGTGACGCTGACGAGG
ACGATGAGGACCGGGGGGTCATGGCCGTCACAGCGGTAACCGCCACAGCTGCCAGCGACAGGATGGAGAGC
GACTCAGACTCAGACAAGAGTAGCGACAACAGTGGCCTGAAGAGGAAGACGCCTGCGCTAAAGGTATCGGT
CTCGAAACGAGCCCGAAAGGCCTCCAGCGACCTGGATCAGGCCAGCGTGTCCCCATCCGAAGAGGAGAACT
CGGAAAGCTCATCTGAGTCGGAGAAGACCAGCGACCAGGACTTCACACCTGAGAAGAAAGCAGCGGTCCGG
GCGCCACGGAGGGGCCCTCTGGGGGGACGGAAAAAAAAGAAGGCGCCATCAGCCTCCGACTCCGACTCCAA
GGCCGATTCGGACGGGGCCAAGCCTGAGCCGGTGGCCATGGCGCGGTCGGCGTCCTCCTCCTCCTCTTCCT
CCTCCTCCTCCGACTCCGATGTGTCTGTGAAGAAGCCTCCGAGGGGCAGGAAGCCAGCGGAGAAGCCTCTC
CCGAAGCCGCGAGGGCGGAAACCGAAGCCTGAACGGCCTCCGTCCAGCTCCAGCAGTGACAGTGACAGCGA
CGAGGTGGACCGCATCAGTGAGTGGAAGCGGCGGGACGAGGCGCGGAGGCGCGAGCTGGAGGCCCGGCGGC
GGCGAGAGCAGGAGGAGGAGCTGCGGCGCCTGCGGGAGCAGGAGAAGGAGGAGAAGGAGCGGAGGCGCGAG
CGGGCCGCTGAGCGGGGCAGCGGCGGCAGCAGCGGGGACGAGCTCAGGGAGGACGATGAGCCCGTCAAGAA
GCGGGGACGCAAGGGCCGGGGCCGGGGTCCCCCGTCCTCCTCTGACTCCGAGCCCGAGGCCGAGCTGGAGA
GAGAGGCCAAGAAATCAGCGAAGAAGCCGCAGTCCTCAAGCACAGAGCCCGCCAGGAAACCTGGCCAGAAG
GAGAAGAGAGTGCGGCCCGAGGAGAAGCAACAAGCCAAGCCCGTGAAGGTGGAGCGGACCCGGAAGCGGTC
CGAGGGCTTCTCGATGGACAGGAAGGTAGAGAAGAAGAAAGAGCCCTCCGTGGAGGAGAAGCTGCAGAAGC
TGCACAGTGAGATCAAGTTTGCCCTAAAGGTCGACAGCCCGGACGTGAAGAGGTGCCTGAATGCCCTAGAG
GAGCTGGGAACCCTGCAGGTGACCTCTCAGATCCTCCAGAAGAACACAGACGTGGTGGCCACCTTGAAGAA
GATTCGCCGTTACAAAGCGAACAAGGACGTAATGGAGAAGGCAGCAGAAGTCTATACCCGGCTCAAGTCGC
GGGTCCTCGGCCCAAAGATCGAGGCGGTGCAGAAAGTGGACAAGGCTGGGATGGAGAAGGAGAAGGCCGAG
GAGAAGCTGGCCGGGGAGGAGCTGGCCGGGGAGGAGGCCCCCCAGGAGAAGGCGGAGGACAAGCCCAGCAC
CGATCTCTCAGCCCCAGTGAATGGCGAGGCCACATCACAGAAGGGGGAGAGCGCAGAGGACAAGGAGCACG
AGGAGGGTCGGGACTCGGAGGAGGGGCCAAGGTGTGGCTCCTCTGAAGACCTGCACGAGAGCGTACGGGAG
GGTCCCGACCTGGACAGGCCTGGGAGCGACCGGCAGGAGCGCGAGAGGGCACGGGGGGACTCGGAGGCCCT
GGACGAGGAGAGCTGA

In a search of public sequence databases, it was found, for example, that the nucleic acid sequence (NOV4c) has 1570 of 1985 bases (79%) identical to a Mus musculus cDNA encoding HET-B which has homology to HDGF mRNA (GENBANK-ID: E14401|acc:E14401)(E=4.8 e-258).

The NOV4c polypeptide (SEQ ID NO:18) encoded by SEQ ID NO:17 is 667 amino acid residues and is presented using the one-letter amino acid code in Table 4H. The NOV4c protein was analyzed for signal peptide prediction and cellular localization. SignalP, Psort and Hydropathy profile indicate that NOV4c does not have a signal peptide and is likely to be localized in the nucleus. However, the protein predicted here is similar to the “Hepatoma Derived Growth Factor-Like Protein Family”, some members of which end up outside the cell and exhibit growth factor activity. Therefore it is likely that NOV4c is available at the appropriate sub-cellular localization and hence accessible for the therapeutic uses described in this application.

TABLE 4H
NOV4c protein sequence

| | |
|---|---|
| MPHAFKPGDLVFAKMKGYPHWPARVDDIADGAVKPPPNKYPIFFFGTHETAFLGPKDLFPYDKCKDK | (SEQ ID NO:18) |
| YGKPNKRKGFNEGLWEIQNNPHASYSAPPPVSSSDSEAPEANPADGSDADEDDEDRGVMAVTAVTAT | |
| AASDRMESDSDSDKSSDNSGLKRKTPALKVSVSKRARKASSDLDQASVSPSEEENSESSSESEKTSD | |
| QDFTPEKKAAVRAPRRGPLGGRKKKKAPSASDSDSKADSDGAKPEPVAMARSASSSSSSSSSSDSDV | |
| SVKKPPRGRKPAEKPLPKPRGRKPKPERPPSSSSSDSDSDEVDRISEWKRRDEARRRELEARRRREQ | |
| EEELRRLREQEKEEKERRRERAAERGSGGSSGDELREDDEPVKKRGRKGRGRGPPSSSDSEPEAELE | |
| REAKKSAKKPQSSSTEPARKPGQKEKRVRPEEKQQAKPVKVERTRKRSEGFSMDRKVEKKKEPSVEE | |
| KLQKLHSEIKFALKVDSPDVKRCLNALEELGTLQVTSQILQKNTDVVATLKKIRRYKANKDVMEKAA | |
| EVYTRLKSRVLGPKIEAVQKVDKAGMEKEKAEEKLAGEELAGEEAPQEKAEDKPSTDLSAPVNGEAT | |
| SQKGESAEDKEHEEGRDSEEGPRCGSSEDLHESVREGPDLDRPGSDRQERERARGDSEALDEES | |

The Hepatoma Derived Growth Factor-like protein (NOV4c) maps to chromosome 19 and is expressed in at least the following tissues: adrenal gland, fetal brain, spleen, thyroid and small intestine and additionally from literature sources: testis, ovaries and liver. The full amino acid sequence of the NOV4c protein was found to have 552 of 667 amino acid residues (82%) identical to, and 603 of 667 residues (90%) similar to, the 669 amino acid residue Hepatoma Derived Growth Factor-like protein from Mus musculus (SPTREMBL-ACC:035540; E=6.6 e-288)(Table 4I).

TABLE 4I
BLASTX results for NOV4c

| | | | | Smallest | |
|---|---|---|---|---|---|
| | | | | Sum | |
| Sequences producing High-scoring | | Reading | High | Prob. | |
| Segment Pairs: | | Frame | Score | P (N) | N |
| ptnr: SPTREMBL- | Hepatoma- | +1 | 2774 | 6.6e−288 | 1 |
| ACC: 035540 | Derived GF - | | | | |
| | Mus | | | | |

NOV4d

A disclosed NOV4d (also referred to as 117477333_EXT) nucleic acid of 2307 nucleotides is shown in FIG. 4J. An open reading frame was identified beginning with an ATG initiation codon at nucleotides 72-74 and ending with a TGA codon at nucleotides 2085-2087. A putative untranslated region upstream from the initiation codon and downstream from the termination codon is underlined in FIG. 4J, and the start and stop codons are in bold letters.

TABLE 4J
NOV4d Nucleotide Sequence.

(SEQ ID NO:19)

GCCGCCGCCGCCGCCGCAGCCGCTACCGCCGCTGCAGCCGCTTTCCGCGGCCTGGGCCTCTCGCCGTCAGC
ATGCCACACGCCTTCAAGCCCGGGGACTTGGTGTTCGCTAAGATGAAGGGCTACCCTCACTGGCCTGCCAG
GATCGACGACATCGCGGATGGCGCCGTGAAGCCCCCACCCAACAAGTACCCCATCTTTTTCTTTGGCACAC
ACGAAACAGCCTTCCTGGGACCCAAGGACCTGTTCCCCTACGACAAATGTAAAGACAAGTACGGGAAGCCC
AACAAGAGGAAAGGCTTCAATGAAGGGCTGTGGGAGATCCAGAACAACCCCCACGCCAGCTACAGCGCCCC
TCCGCCAGTGAGCTCCTCCGACAGCGAGGCCCCCGAGGCCAACCCCGCCGACGGCAGTGACGCTGACGAGG
ACGATGAGGACCGGGGGGTCATGGCCGTCACAGCGGTAACCGCCACAGCTGCCAGCGACAGGATGGAGAGC
GACTCAGACTCAGACAAGAGTAGCGACAACAGTGGCCTGAAGAGGAAGACGCCTGCGCTAAAGATGTCGGT
CTCGAAACGAGCCCGAAAGGCCTCCAGCGACCTGGATCAGGCCAGCGTGTCCCCATCCGAAGAGGAGAACT
CGGAAAGCTCATCTGAGTCGGAGAAGACCAGCGACCAGGACTTCACACCTGAGAAGAAAGCAGCGGTCCGG
GCGCCACGGAGGGGCCCTCTGGGGGGACGGAAAAAAAAGAAGGCGCCATCAGCCTCCGACTCCGACTCCAA
GGCCGATTCGGACGGGGCCAAGCCTGAGCCGGTGGCCATGGCGCGGTCGGCGTCCTCCTCCTCCTCTTCCT
CCTCCTCCTCCGACTCCGATGTGTCTGTGAAGAAGCCTCCGAGGGGCAGGAAGCCAGCGGAGAAGCCTCTC
CCGAAGCCGCGAGGGCGGAAACCGAAGCCTGAACGGCCTCCGTCCAGCTCCAGCAGTGACAGTGACAGCGA
CGAGGTGGACCGCATCAGTGAGTGGAAGCGGCGGGACGAGGCGCGGAGGCGCGAGCTGGAGGCCCGGCGGC
GGCGAGAGCAGGAGGAGGAGCTGCGGCGCCTGCGGGAGCAGGAGAAGGAGGAGAAGGAGCGGAGGCGCGAG
CGGGCCGACCGCGGGGAGGCTGAGCGGGGCAGCGGCGGCAGCAGCGGGGACGAGCTCAGGGAGGACGATGA
GCCCGTCAAGAAGCGGGGACGCAAGGGCCGGGGCCGGGGTCCCCCGTCCTCCTCTGACTCCGAGCCCGAGG
CCGAGCTGGAGAGAGAGGCCAAGAAATCAGCGAAGAAGCCGCAGTCCTCAAGCACAGAGCCCGCCAGGAAA
CCTGGCCAGAAGGAGAAGAGAGTGCGGCCCGAGGAGAAGCAACAAGCCAAGCCCGTGAAGGTGGAGCGGAC
CCGGAAGCGGTCCGAGGGCTTCTCGATGGACAGGAAGGTAGAGAAGAAGAAAGAGCCCTCCGTGGAGGAGA
AGCTGCAGAAGCTGCACAGTGAGATCAAGTTTGCCCTAAAGGTCGACAGCCCGGACGTGAAGAGGTGCCTG
AATGCCCTAGAGGAGCTGGGAACCCTGCAGGTGACCTCTCAGATCCTCCAGAAGAACACAGACGTGGTGGC
CACCTTGAAGAAGATTCGCCGTTACAAAGCGAACAAGGACGTAATGGAGAAGGCAGCAGAAGTCTATACCC
GGCTCAAGTCGCGGGTCCTCGGCCCAAAGATCGAGGCGGTGCAGAAAGTGAACAAGGCTGGGATGGAGAAG
GAGAAGGCCGAGGAGAAGCTGGCCGGGGAGGAGCTGGCCGGGGAGGAGGCCCCCCAGGAGAAGGCGGAGGA
CAAGCCCAGCACCGATCTCTCAGCCCCAGTGAATGGCGAGGCCACATCACAGAAGGGGGAGAGCGCAGAGG
ACAAGGAGCACGAGGAGGGTCGGGACTCGGAGGAGGGGCCAAGGTGTGGCTCCTCTGAAGACCTGCACGAC
AGCGTACGGGAGGGTCCCGACCTGGACAGGCCTGGGAGCGACCGGCAGGAGCGCGAGAGGGCACGGGGGGA
CTCGGAGGCCCTGGACGAGGAGAGCTGAGCCGCGGGCAGCCAGGCCCAGCCCCCGCCCGAGCTCAGGCTGC
CCCTCTCCTTCCCCGGCTCGCAGGAGAGCAGAGCAGAGAACTGTGGGGAACGCTGTGCTGTTTGTATTTGT
TCCCTTGGGTTTTTTTTTCCTGCCTAATTTCTGTGATTTCCAACCAACATGAAATGACTATAAATGGTTTT
TTAATGAAAAAAAAAAAAAAAGAAAAAAAAAGATT

In a search of public sequence databases, it was found, for example, that the nucleic acid sequence (NOV4d) has 1052 of 1378 bases (76%) identical to a Mus musculus Hepatoma Derived Growth Factor-like protein mRNA (GENBANK-ID:D63850)(E=4.8 e-163).

The NOV4d polypeptide (SEQ ID NO:20) encoded by SEQ ID NO:19 is 671 amino acid residues and is presented using the one-letter amino acid code in Table 4K. The NOV4d protein was analyzed for signal peptide prediction and cellular localization. SignalP, Psort and Hydropathy profile indicate that NOV4d does not have a signal peptide and is likely to be localized in the nucleus. However, the protein predicted here is similar to the “Hepatoma Derived Growth Factor-Like Protein Family”, some members of which end up outside the cell and exhibit growth factor activity. Therefore it is likely that NOV4d is available at the appropriate sub-cellular localization and hence accessible for the therapeutic uses described in this application.

TABLE 4K
NOV4d protein sequence

(SEQ ID NO:20)

MPHAFKPGDLVFAKMKGYPHWPARIDDIADGAVKPPPNKYPIFFFGTHETAFLGPKDLFPYDKCKDKYGKPNKR
KGFNEGLWEIQNNPHASYSAPPPVSSSDSEAPEANPADGSDADEDDEDRGVMAVTAVTATAASDRMESDSDSDK
SSDNSGLKRKTPALKMSVSKRARKASSDLDQASVSPSEEENSESSSESEKTSDQDFTPEKKAAVRAPRRGPLGG
RKKKKAPSASDSDSKADSDGAKPEPVAMARSASSSSSSSSSSDSDVSVKKPPRGRKPAEKPLPKPRGRKPKPER
PPSSSSSDSDSDEVDRISEWKRRDEARRRELEARRRREQEEELRRLREQEKEEKERRRERADRGEAERGSGGSS
GDELREDDEPVKKRGRKGRGRGPPSSSDSEPEAELEREAKKSAKKPQSSSTEPARKPGQKEKRVRPEEKQQAKP
VKVERTRKRSEGFSMDRKVEKKKEPSVEEKLQKLHSEIKFALKVDSPDVKRCLNALEELGTLQVTSQILQKNTD
VVATLKKIRRYKANKDVMEKAAEVYTRLKSRVLGPKIEAVQKVNKAGMEKEKAEEKLAGEELAGEEAPQEKAED
KPSTDLSAPVNGEATSQKGESAEDKEHEEGRDSEEGPRCGSSEDLHDSVREGPDLDRPGSDRQERERARGDSEA
LDEES

The Hepatoma Derived Growth Factor-like protein (NOV4d) maps to chromosome 19 and is expressed in at least the following tissues: brain, heart, lung, lung carcinoids, exocrine pancreas, breast, teratocarcinoma, ovarian tumor, prostate, colon, colon carcinoma, esophagus, foreskin, germ cells, uterus, genitourinary tract, thyroid, blood, spleen, tonsil, hematopoietic and lymphatic systems and bone marrow.

The full amino acid sequence of the NOV4d protein was found to have 563 of 671 amino acid residues (82%) identical to, and 603 of 671 residues (89%) similar to, the 669 amino acid residue Hepatoma Derived Growth Factor-like protein from Mus musculus (SPTREMBL-ACC:035540; E=2.7 e-288).

The amino acid sequence of NOV4d had high homology to other proteins as shown in Table 4L.

TABLE 4L
BLASTX results for NOV4d

| | | | | Smallest | |
|---|---|---|---|---|---|
| | | | | Sum | |
| | | Reading | High | Prob. | |
| Sequences producing High-scoring Segment Pairs: | | Frame | Score | P (N) | N |
| ptnr: SPTREMBL-ACC: 035540 | HEPATOMA-DERIVED GROWTH FACTO . . . | +3 | 2777 | 2.7e−288 | 1 |
| ptnr: PIR-ID: JC7168 | lens epithelium-derived growth fact . . . | +3 | 522 | 5.8e−52 | 2 |
| ptnr: SPTREMBL-ACC: 075475 | LENS EPITHELIUM-DERIVED GROWT . . . | +3 | 518 | 1.8e−51 | 2 |

NOV4a, 4b, 4c and 4d are related to each other as shown in the alignment listed in Table 4M.

[image]

[image]

It was also found that NOV4a had homology to the amino acid sequences shown in the BLASTP data listed in Table 4N.

TABLE 4N
BLAST results for NOV4a

| Gene Index/ | | Length | Identity | Positives | |
|---|---|---|---|---|---|
| Identifier | Protein/Organism | (aa) | (%) | (%) | Expect |
| gi\|12653923\| | Similar to | 670 | 220/272 | 222/272 | 1e−82 |
| gb\|AAH00755.1\| | hepatoma-derived | | (80%) | (80%) | |
| AAH00755 | growth factor, | | | | |
| (BC000755) | related protein 2 | | | | |
| | [Homo sapiens] | | | | |
| gi\|13277669\| | Similar to | 678 | 167/256 | 197/256 | 9e−64 |
| gb\|AAH03741.1\| | hepatoma-derived | | (65%) | (76%) | |
| AAH03741 | growth factor, | | | | |
| (BC003741) | related protein 2 | | | | |
| | [Mus musculus] | | | | |
| gi\|6680201\| | hepatoma-derived | 669 | 167/256 | 197/256 | 2e−63 |
| ref\|NP_032259.1\| | growth factor, | | (65%) | (76%) | |
| | related protein 2 | | | | |
| | [Mus musculus] | | | | |
| gi\|13957749\| | hepatoma-derived | 669 | 163/246 | 186/246 | 6e−62 |
| gb\|AAK50635.1\| | growth factor 3 | | (66%) | (75%) | |
| AF355102_1 | [Rattus | | | | |
| (AF355102) | norvegicus] | | | | |

The homology of these sequences is shown graphically in the ClustalW analysis shown in Table 4O.

[image]

[image]

The homologies shown above are shared by NOV4b, 4c and 4d insofar as they are themselves homologous to NOV4a as shown in Table 4M.

Table 4P lists the domain description from DOMAIN analysis results against NOV4a. This indicates that the NOV4a sequence has properties similar to those of other proteins known to contain this domain.

[image]

Other BLAST results include sequences from the Patp database, which is a proprietary database that contains sequences published in patents and patent publications. Patp results include those listed in Table 4Q.

TABLE 4Q
Patp alignments of NOV4

| | | | | Smallest |
|---|---|---|---|---|
| | | | | Sum |
| Sequences producing High-scoring | | Reading | High | Prob. |
| Segment Pairs: | | Frame | Score | P (N) |
| Patp: AAY99426 | Human PRO1604 | +1 | 3379 | 0.0 |
| | (UNQ785), | | | |
| | Homo sapiens, 671 aa | | | |
| Patp: AAB66175 | unidentified protein, | +1 | 3379 | 0.0 |
| | 671 aa | | | |

For example, a BLAST against patp:AAY99426, a 671 amino acid polypeptide (WO00/012708) from Homo sapiens, produced good identity, E=0.0. Additionally, a BLAST against patp:AAB66175, a 671 unidentified protein (WO00/78961), also produced good identity, E=0.0.

Hepatoma-Derived Growth Factor (HDGF) is a basis Fibroblast Growth Factor-like growth factor secreted by human hepatomas that acts as an endothelial cell mitogen (Klagsbrun et al., 1986). It is expressed in proliferating smooth muscle cells and may be involved in vascular development and atherosclerosis (Everett et al., 2000). HDGF has also been implicated in renal development (Oliver et al., 1998).

Many groups have attempted to elucidate the specific function(s) of HDGF; as its role in vascular growth is currently unknown. Everett et al. (2000) demonstrated that HDGF mRNA is expressed in smooth muscle cells (SMCs), most prominently in proliferating SMCs. Exogenous HDGF and endogenous overexpression of HDGF stimulated a significant increase in SMC number and DNA synthesis. Moreover, it was shown that HDGF colocalizes with the proliferating cell nuclear antigen (PCNA) in SMCs in human atherosclerotic carotid arteries, suggesting that HDGF helps regulate SMC growth during development and in response to vascular injury. Cilley et al. (2000) while studying lung development identified HDGF as a differentially expressed gene enhanced by tracheal ligation using an in vitro murine fetal lung model with airway ligation. The authors concluded that genes enhanced by airway pressure or tracheal ligation are mitogenic for fibroblasts, correlate with cell proliferation, regulate cell proliferation and differentiation, and may play a role in growth in distal lung and type II cell differentiation. Oliver et al., 1998 while studying the potential secretion of endothelial mitogens by metanephrogenic mesenchymal cells using a endothelial mitogenic assay and sequential chromatography isolated HDGF. The authors found that HDGF was widely distributed in the renal anlage at early stages of development but soon concentrated at sites of active morphogenesis and, except for some renal tubules, disappeared from the adult kidney. HDGF was most abundant at sites of nephron morphogenesis and in ureteric bud cells while in the adult kidney transcripts disappeared except for a small population of distal tubules. Thus, the authors concluded that HDGF is an endothelial mitogen that is present in embryonic kidney, and its expression is synchronous with nephrogenesis.

Along with HDGF, there is a family of proteins with significant homology to HDGF in the amino terminal region (hath region) which are termed HDGF-related proteins (HRPs) (Izumoto et al., 1997)(Ikegame et al., 1999). There are several family members with varied tissue expression. HRP-1 is expressed only in the developing germ cells of the testis and may be involved in spermatogenesis. These findings suggest that the testis-specific HRP-1 gene may play an important role in the phase around meiotic cell division (Kuroda et al., 1999). HRP-2, like HDGF, is expressed predominantly in the testis and skeletal muscle, with intermediate levels in heart, brain, lung, liver and kidney and minimally in the spleen. {RP-1 is a highly acidic protein (26% acidic) and also has a putative NLS. HRP-2 protein carries a mixed charge cluster (Izumoto et al., 1997). HRP-3 cDNA contained 203 amino acids without a signal peptide for secretion. HRP-3 has a putative bipartite nuclear localizing signal (NLS) sequence and as a result is known to translocate to the nucleus. The HRP-3 gene was mapped to chromosome 15, region q25 by FISH analysis. Further, HRP-3 is expressed predominantly in the testis and brain, to an intermediate extent in the heart, and with lower levels in the ovaries, kidneys, spleen, and liver in humans (Ikegame et al., 1999).

The expression pattern, map location and protein similarity information for the invention(s) suggest that these NOV4 proteins may function as members of the HDGF family. Therefore, the nucleic acids and proteins of the invention are useful in potential therapeutic applications implicated, for example but not limited to, in various pathologies/disorders as described below and/or other pathologies/disorders. Potential therapeutic uses for the invention(s) are, for example but not limited to, the following: Protein therapeutic, small molecule drug target, antibody target (therapeutic, diagnostic, drug targeting/cytotoxic antibody), diagnostic and/or prognostic marker, gene therapy (gene delivery/gene ablation), research tools, and tissue regeneration in vitro and in vivo (regeneration for all these tissues and cell types composing these tissues and cell types derived from these tissues).

The nucleic acids and proteins of the invention are useful in potential therapeutic applications implicated in various diseases and disorders described below and/or other pathologies and disorders. For example, but not limited to, a cDNA encoding the HDGF-like protein may be useful in gene therapy, and the HDGF-like protein may be useful when administered to a subject in need thereof. By way of nonlimiting example, the compositions of the present invention will have efficacy for treatment of patients suffering from Adrenoleukodystrophy, Congenital Adrenal Hyperplasia, Hyperthyroidism, Inflammatory bowel disease, Diverticular disease, Hemophilia, Hypercoagulation, Idiopathic thrombocytopenic purpura, Immunodeficiencies, Graft vesus host, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, Neuroprotection, Fertility, Cirrhosis, Transplantation. The novel nucleic acid encoding the HDGF-like protein, and the HDGF-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acids encoding HDGF-like proteins, and the HDGF-like proteins of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods and other diseases, disorders and conditions of the like. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below.

For example, the disclosed NOV4 proteins have multiple hydrophilic regions, each of which can be used as an immunogen. In one embodiment, a contemplated NOV4 epitope is from about amino acids 10 to 50. In another embodiment, a NOV4 epitope is from about amino acids 55 to 125. In additional embodiments, NOV4 epitopes are from about amino acids 130 to 500 and from about amino acids 520 to 680. These novel proteins can be used in assay systems for functional analysis of various human disorders, which will help in understanding of pathology of the disease and development of new drug targets for various disorders.

NOV5

NOV5a

This invention describes the following novel Cortexin-like proteins and nucleic acids encoding them: 21647246_EXT. These sequences were initially identified by searching CuraGen's Human SeqCalling database for DNA sequences, which translate into proteins with similarity to Cortexin-Like Proteins. SeqCalling assembly 21647246 was identified as having suitable similarity. SeqCalling assembly 21647246 was analyzed further to identify any open reading frames encoding novel full length proteins as well as novel splice forms of these genes. The SeqCalling assembly was extended using one or more sequences taken from additional SeqCalling assemblies, publicly available EST sequences and public genomic sequences. Public ESTs and additional CuraGen SeqCalling assemblies were identified by the CuraTools™ program SeqExtend. Such fragments were included in the DNA sequence extension for SeqCalling assembly 21647246 only when the extent of identity in the putative overlap region was high. The extent of identity may be, for example, about 90% or higher, preferably about 95% or higher, and even more preferably close to or equal to 100%. These inclusions, if used, are described below.

The following genomic clones were identified as having regions with 100% identity to the SeqCalling assembly 21647246. They were selected for analysis because this identity indicates that these clones identify the genomic locus for SeqCalling assembly 21647246.

Genomic clone acc:AC010336 Homo sapiens chromosome 19 clone CITB-E1_{—}3193013, WORKING DRAFT SEQUENCE, 64 unordered pieces—Homo sapiens was analyzed by Genscan and Grail to identify exons and putative coding sequences. This clone was also analyzed by ThlastN, BlastX and other programs to identify genomic regions translating to proteins with similarity to the original protein or protein family of interest.

The results of these analyses were integrated and manually corrected for apparent inconsistencies that may have arisen, for example, from miscalled bases in the original fragments used. The sequences obtained encode the full-length proteins disclosed herein. When necessary, the process to identify and analyze cDNAs, ESTs and genomic clones was reiterated to derive the full-length sequence. This invention describes the resulting full-length DNA sequence and the full-length protein sequence, which they encode.

The disclosed NOV5a nucleic acid of 249 nucleotides (also referred to as CuraGen Acc. No. 21647246_EXT) encoding a novel Cortexin-like protein is shown in Table 5A. An ORF begins with an ATG initiation codon at nucleotides 1-3 and ends with a TGA codon at nucleotides 247-249. The start and stop codons are in bold letters in Table 5A.

TABLE 5A
NOV5a Nucleotide Sequence

(SEQ ID NO:21)

ATGAGCGCGACGTGGACGCTGTCGCCGGAGCCCCTGCCGCCGTCGACGGGGCCCCCGGTGGGCGCGGGCC
TGGACGCGGAGCAGCGCACGGTGTTCGCCTTCGTGCTCTGCCTGCTCGTGGTGCTGGTGCTGTTGATGGT
GCGCTGCGTGCGCATCCTGCTCGACCCCTACAGCCGCATGCCCGCCTCGTCCTGGACCGACCACAAGGAG
GCGCTCGAGCGCGGGCAGTTCGACTACGCGTTOGTGTGA

The NOV5a protein encoded by SEQ ID NO:21 has 82 amino acid residues and is presented using the one-letter code in Table SB. The Psort profile for NOV5a predicts that this sequence has a signal peptide and is likely to be localized at the plasma membrane with a certainty of 0.700, it may also localize to the microbody (peroxisome) (certainity of 0.2462); endoplasmic reticulum (membrane) (certainity of 0.200); and mitochondrial inner membrane (certainty of 0.1000). The most likely cleavage site for a peptide is between amino acids 49 and 50, i.e., at the slash in the amino acid sequence VRC-VR based on the SignalP result.

The disclosed Cortexin-like protein maps to chromosome 19. Moreover, the disclosed protein is expressed in at least the following tissues: cortex (brain), hippocampus (brain), and nervous system.

TABLE 5B
Encoded NOV5a protein sequence

(SEQ ID NO:22)

MSATWTLPEPFLPPSTGPPVGAGLDAEQRTVFAFVLCLLVVLVLLMVRCVRTLLDPYSRMPASSWTDHKEAL
ERCQFDYALV

NOV5b

In the present invention, the target sequence identified previously, Accession Number 21647246_EXT (NOV5a), was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number 21647246_dal (NOV5b) which is 100% homologous to the previously identified sequence (Accession Number 21647246_EXT).

The NOV5b sequence of the invention was derived by laboratory cloning of cDNA fragments covering the full length and/or part of the DNA sequence of the invention, and/or by in silico prediction of the full length and/or part of the DNA sequence of the invention from public human sequence databases.

The laboratory cloning was performed using one or more of the methods summarized below:

Exon Linking:

The cDNA coding for the sequence was cloned by polymerase chain reaction (PCR) using the following primers: ATGAGCGCGACGTGGACG (SEQ ID NO:101) and TCACACCAACGCGTAGTCGAACT (SEQ ID NO: 102 on the following pools of human cDNAs: Pool 1—Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus.

Primers were designed based on in silico predictions for the full length or part (one or more exons) of the DNA/protein sequence of the invention or by translated homology of the predicted exons to closely related human sequences or to sequences from other species. Usually multiple clones were sequenced to derive the sequence which was then assembled similar to the SeqCalling process. In addition, sequence traces were evaluated manually and edited for corrections if appropriate.

Physical Clone:

The PCR product derived by exon linking was cloned into the pCR2.1 vector from Invitrogen. The bacterial clone 120906::21647246.698361.M22 has an insert covering the entire open reading frame cloned into the pCR2.1 vector from Invitrogen. Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

The DNA sequence and protein sequence for a novel Cortexin-like gene or one of its splice forms was obtained solely by exon linking and is reported here as NOV5b.

In the following positions, one or more consensus positions (Cons. Pos.) of the nucleotide sequence have been identified as SNPs. “Depth” rerepresents the number of clones covering the region of the SNP. The Putative Allele Frequency (Putative Allele Freq.) is the fraction of all the clones containing the SNP. A dash (“-”), when shown, means that a base is not present. The sign “>” means “is changed to”.

Cons.Pos.: 24 Depth: 48 Change: G>A

Cons.Pos.: 39 Depth: 48 Change: A>C

Cons.Pos.: 40 Depth: 48 Change: G>A

Cons.Pos.: 63 Depth: 57 Change: C>T

Cons.Pos.: 68 Depth: 57 Change: A>C

Cons.Pos.: 72 Depth: 57 Change: A>G

Cons.Pos.: 110 Depth: 57 Change: A>G

Cons.Pos.: 146 Depth: 56 Change: C>G

Cons.Pos.: 170 Depth: 56 Change: T>C

Cons.Pos.: 177 Depth: 56 Change: G>C

Cons.Pos.: 297 Depth: 54 Change: T>C

Cons.Pos.: 309 Depth: 54 Change: C>T

Cons.Pos.: 439 Depth: 66 Change: T>C

Cons.Pos.: 445 Depth: 66 Change: A>G

Cons.Pos.: 479 Depth: 64 Change: G>A

Cons.Pos.: 488 Depth: 61 Change: T>C

Cons.Pos.: 492 Depth: 60 Change: T>C

Cons.Pos.: 511 Depth: 61 Change: C>T

Cons.Pos.: 524 Depth: 63 Change: C>T

Cons.Pos.: 539 Depth: 62 Change: C>T

Cons.Pos.: 626 Depth: 38 Change: C>T

Cons.Pos.: 708 Depth: 43 Change: T>G

Cons.Pos.: 720 Depth: 44 Change: T>C

Cons.Pos.: 753 Depth: 44 Change: C>G

Cons.Pos.: 852 Depth: 44 Change: G>T

Cons.Pos.: 872 Depth: 44 Change: A>G

Cons.Pos.: 878 Depth: 44 Change: T>C

Cons.Pos.: 904 Depth: 44 Change: C>T

Cons.Pos.: 956 Depth: 46 Change: C>T

Cons.Pos.: 980 Depth: 46 Change: A>G

As shown in Tables 5C and 5D, the disclosed NOV5a and NOV5b nucleic acid sequences and amino acid sequences are are 100% identical. As used herein, any reference to NOV5 encompasses both NOV5a and NOV5b.

[image]

The disclosed nucleic acid sequence for NOV5 has 229 of 249 bases (91%) identical and 229 of 249 (91%) residues positives to a Rattus norvegicus neuron-specific cortexin mRNA (acc:L15011)(E=4.2e-42).

Moreover, the full NOV5 amino acid sequence of the protein of the invention was found to have 46 of 82 amino acid residues (56%) identical to, and 46 of 82 residues (56%) positive with, the 82 amino acid residue rat cortexin. (gi|7292251sp|P412371|CTXN_RAT CORTEXIN; gi|543376|pir}}JH0814 (cortexin-rat)(E=2e-15).

The disclosed NOV5 protein has good identity with a cortexin-like protein. The identity information used for ClustalW analysis is presented in Table 5E.

TABLE 5E
BLAST results for NOV5

| Gene Index/ | Protein/ | Length | Identity | Positives | |
|---|---|---|---|---|---|
| Identifier | Organism | (aa) | (%) | (%) | Expect |
| gi\|729225\| | cortexin | 82 | 46/82 | 46/82 | 2e−15 |
| sp\|P41237\| | Rattus | | (56%) | (56%) | |
| CTXN_RAT CORTEXIN; | norvegicus | | | | |
| gi\|543376\| | | | | | |
| pir\|\|JH08 | | | | | |
| 14 cortexin | | | | | |

This information is presented graphically in the Clustal W sequence alignment given in Table 5F (with NOV5 being shown on line 1) comparing NOV5 with a related protein sequence.

[image]

The NOV5 proteins predicted here are similar to the “Cortexin-Like Protein Family”, some members of which end up localized at the cell surface where they exhibit activity. Therefore, it is likely that thes enovel Cortexin-like proteins are available at the appropriate sub-cellular localization and hence accessible for the therapeutic uses described herein.

Nucleotide sequence analysis of a cDNA clone of a rat cortex-enriched mRNA identifies a novel integral membrane protein of 82 amino acids. The encoded protein is named cortexin to reflect its enriched expression in cortex. The amino acid sequence of rat cortexin and its mouse homologue are nearly identical (98% similarity), and both contain a conserved single membrane-spanning region in the middle of each sequence. Northern blot analysis shows that cortexin mRNA is brain-specific, cortex-enriched, and present at significant levels in fetal brain, with peak expression in postnatal rodent brain. In situ hybridization studies detect cortexin mRNA primarily in neurons of rodent cerebral cortex, but not in cells of the hindbrain or white matter regions. The function of cortexin may be particularly important to neurons of both the developing and adult cerebral cortex. See J. Neurochem 61(2):756-59 (1993).

A clinical trial of cortexin, a new peptide bioregulator of cerebral functions, in combined therapy of dyscirculatory encephalopathy (DE) stage I-II was made in 76 patients. They were divided into two groups: a control group of 31 patients on standard therapy and the study group of 45 patients on standard therapy with adjuvant cortexin delivered via nasal electrophoresis (NE). The effect was estimated by clinical symptoms, psychophysiological tests, computed EEG, quantitative parameters of rehabilitation. Cortexin NE produced a positive effect on psychoemotional state, neurological status, intellectual-mnestic and CNS functions. Adjuvant cortexin aroused efficiency of rehabilitation in DE stage I and II by 22.7%. The response of intellectual-mnestic and CNS functions was the highest. Cortexin improves attention, perception, memory, thinking, cortical neurodynamic processes. It is well tolerated and has no side effects. Cortexin is recommended as a drug of choice in combined treatment of patients with DE stage I-II. See Klin Med. (Mosk) 77(4):42-45 (1999).

The effect of cortexin and epithalamin on the cell growth rate was investigated in the organotypic tissue culture of dorsal root ganglia (DRG), and of cortex and subcortical structures of 10-11-day old chick embryos. Cortexin in concentrations of 20 and 100 ng/ml is active, inducing a more intensive neurite outgrowth in DRG, compared to the control. Epithalamin was active in concentrations 20 and 200 ng/ml. Cortexin (100 ng/ml) was active in the cortex tissue culture, but inhibited the neurite growth in the subcortical structures culture. The stimulation of this culture to development was evident after using 200 ng/ml epithalamin. The neurite stimulating effect of cortexin and epithalamin is presumably associated with neurotrophic factors. See Tsitologiia 39(7):571-76 (1997).

The expression pattern, map location and protein similarity information for the invention suggest that this Cortexin-like protein may function as a member of the Cortexin-like protein family. Therefore, the nucleic acids and proteins of the invention are useful in potential therapeutic applications implicated, for example but not limited to, in various pathologies /disorders as described below and/or other pathologies/disorders. Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention are useful in potential therapeutic applications implicated in various diseases and disorders described below and/or other pathologies and disorders. For example, but not limited to, a cDNA encoding the Cortexin-like protein may be useful in gene therapy, and the Cortexin-like protein may be useful when administered to a subject in need thereof. By way of nonlimiting example, the compositions of the present invention will have efficacy for treatment of patients suffering from Von Hippel-Lindau (VHL) syndrome, Alzheimer's disease, stroke, tuberous sclerosis, hypercalceimia, Parkinson's disease, Huntington's disease, cerebral palsy, epilepsy, Lesch-Nyhan syndrome, multiple sclerosis, ataxia-telangiectasia, leukodystrophies, behavioral disorders, addiction, anxiety, pain, memory/perception/attention disorders, and/or neuroprotection. The novel nucleic acid encoding the a Cortexin-like protein, and the a Cortexin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding cortexin-like protein, and the cortexin-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein has multiple hydrophilic regions, each of which can be used as an immunogen. In one embodiment, a contemplated NOV5 epitope is from about amino acids 12 to 28. In another embodiment, a NOV5 epitope is from about amino acids 58 to 77. This novel protein also has value in development of powerful assay system for functional analysis of various human disorders, which will help in understanding of pathology of the disease and development of new drug targets for various disorders.

NOV6

This invention describes novel SN-like proteins, and nucleic acids encoding them, designated 27926453_EXT1 (NOV6). These sequences were initially identified by searching CuraGen's Human SeqCalling database for DNA sequences, which translate into proteins with similarity to a protein family of interest. SeqCalling assembly 27926453_EXT1 was identified as having suitable similarity. SeqCalling assembly 27926453_EXT1 has 1 component. In a search of CuraGen's human expressed sequence assembly database, trimmed assembly s3aq: 27926453 (699 nucleotides) was identified as having homology to this predicted gene sequence (FIG. 3B). SeqCalling assembly 27926453_EXT1 was analyzed further to identify open reading frame(s) encoding for a novel full length protein(s) and novel splice forms of these SN. This was done by extending the SeqCalling assembly using suitable additional SeqCalling assemblies, publicly available EST sequences as well public genomic sequence. Public ESTs and additional CuraGen SeqCalling assemblies were identified by the CuraTools™ program SeqExtend. They were included in the DNA sequence extension for SeqCalling assembly 27926453_EXT1 only when sufficient identical overlap was found. These inclusions are described below:

Genomic clone AL109804 was identified as having regions with 100% identity to the SeqCallling assembly 27926453_EXT1 and was selected for analysis. This identity implies that this clone provides the genomic locus for SeqCallling assembly 27926453_EXT1.

The genomic clones were analysed by Genscan and Grail to identify exons and putative coding sequences/open reading frames. This clone was also analyzed by TblastN, BlastX and other homology programs to identify regions translating to proteins with similarity to the original protein/protein family of interest.

The results of these analyses were integrated and manually corrected for apparent inconsistencies, thereby obtaining the sequences encoding the full-length protein. When necessary, the process to identify and analyse cDNAs/ESTs and genomic clones was reiterated to derive the full length sequence. This invention describes this full-length DNA sequence and the full-length protein sequence which it encodes. These nucleic acids and protein sequences for each splice form are referred to here as NOV6 (27926453_EXT1).

Specifically, CuraGen's SeqCalling Assembly 27926453_EXT1 is made up of 154 fragments, which are trimmed to a 699 bp contig. SeqCalling Assembly 27926453_EXT1 is found in bone marrow, thyroid, lymph node, pancreas, placenta, fetal liver, heart, prostate, spleen, salivary gland, mammary gland, thalamus, adrenal gland, and kidney. SeqCalling assembly 27926453_EXT1 showed initial homology, by searching with BLASTX, to a Mus musculus (mouse) protein: SIALOADHESIN PRECURSOR (SER) (SWISSNEW-ACC:Q62230, 1694 aa). Using BlastN, this SeqCalling Assembly was identical at the nucleotide level to a GenBank genomic sequence: GENBANKNEW-ID: HS1009E24jacc: AL109804 Homo sapiens chromosome 20 clone RP5-1009E24, * * * SEQUENCING IN PROGRESS * * *, 10 unordered pieces—Homo sapiens, 195588 bp. AL109804 was processed with GenScan and the predicted coding regions were analyzed using BlastX, BlastN and TBlastN to find exons with homologies to M. musculus SN. The genomic clone matched identically to NOV6. AL109804 was used to extend 27926453_EXT1. This was accomplished by using the protein sequence of SWISSNEW-5 ACC:Q62230 and Curatool's TblastN against the GBNEW database. Intron/exon junctions were determined by manual inspection and corrected for apparent inconsistencies. BlastX of this sequence showed the correct full-length protein, 27926453_EXT1. The base pair (bp) regions used from the genomic clone were: 179126-179161, 179532-179912, 180219-180510, 182165-182428, 182802-183053, 184609-184911, 186797-187066, 188129-188455, 188911-189169, 189366-110 189670, 191333-191596, 191721-192032, 192554-192806, 193123-193392, 193484-193735, 194054-194358, 140705-140757 which was subsequently corrected for apparent inconsistencies.

The disclosed novel NOV6 nucleic acid of 5103 nucleotides (Accession Number 27926453 EXT1) is shown in Table 6A. An open reading begins with an ATG initiation codon at nucleotides 1-3 and ends with a TGA codon at nucleotides 5101-5103. The start and stop codons are in bold letters.

TABLE 6A
NOV6 Nucleotide Sequence

(SEQ ID NO:23)

ATGGCCTTCTTGCCCAAGCTTCTCCTCCTGGCCTCAGCCGTTCTTCCCCCAGGCCAGCCCTCATGGGGCG
TCTCCAGTCCCCACGACGTGCAGGGTGTGAAGGCGTCTTGCCTGCTTATCCCCTGCATCTTCAGCTTCCC
TGCCGACGTGGAGGTGCCCCACGGCATCACGGCCATCTGGTACTACCACTACTCGGGCCAGCGGCAGGTC
GTGAGCCACTCGGCGGACCCCAAGCTCGTGGAGGCCCGCTTCCGCGGCCGCACCCAGTTCATGCGGAACC
CCGAGCACAGGGTGTGCAACCTGCTGCTCAAGCACCTGCACCCCGACGACTCTGGTTCCTACAACTTCCC
CTTCGAGATCAGTGAGGTCAACCGCTGGTCAGATGTCAAAGGCACCTTGGTCACACTAACAGGTGATCCC
AGGGTGCCCACCATTGCCTCCCCGGTGGAGCTTCTCGAGGGCACAGAGGTGGACTTCAACTGCTCCACTC
CCTACGPATGCCTGCAGGAGCAGCTCAGACTGCAGTGGCAAGGCCAGGACCCTGCTCGCTCTGTCACCTT
CAACACCCAGAACTTTGAGCCCACCGGCGTCGGCCACCTGGACACCCTCCACATGGCCATGTCCTGGCAG
GACCACGGCCGGATCCTGCGCTCCCAGCTCTCCGTGGCCAATCACAGCGCTCAGAGCGACATTCACCTCC
AAGTGAAGTGTGCCCCCAAGGGTGTGAAGATCCTCCTCAGCCCCTCCCGGAGGAACATCCTTCCAGGTGA
GCTGGTCACACTCACCTGCCAGGTGAACAGCAGCTACCCTGCACTCAGTTCCATTAAGTGGCTCAAGGAT
GGGGTACGCCTCCAAACCAACACTGGTGTGCTGCACCTGCCCCAGGCAGCCTGGAGCGATGCTGGCGTCT
ACACCTGCCAAGCTGAGAACGGCGTGGGCTCTTTCGTCTCACCCCCCATCACCCTCCACATCTTCGTGGC
TGAGGTCCAGGTGAGCCCAGCAGGTCCCATCCTGGAGAACCAGACAGTGACACTAGTCTGCAACACACCC
AATGAGCCACCCAGTCATCTCCCCTACAGCTGGTACAAGAACCATGTCCTGCTGGAGGATGCCCACTCCC
ATACCCTCCGGCTGCACTTGGCCACTAGGGCTGATACTGGCTTCTACTTCTGTGAGGTGCAGAACGTCCA
TGGCAGCGAGCGCTCGGGCCCTGTCAGCGTGGTAGTCACAGACCCGCCTCTCACTCCAGTCCTGACAGCC
TTCCTGGACACCCAGGCGGGACTTCTGGGCATCCTTCACTGCTCTCTGGTCAGTGAGCCCCTGGCCACAC
TGGTCCTGTCACATGGGGGTCATATCCTGGCCTCCACCTCCGGCGACAGTCATCACAGCCCACCCTTCAG
TGGTACCTCTCCTCCCAACTCCCTGCGCCTGGAGATCCGAGACCTGGAGCAAACTCACAGTGGGGACTAC
AAGTCCTCAGCCACCAACTCCCTTGGAAATGCAACCTCCACCCTGGACTTCCATGCCAATCTCGCCCGTC
TCCTCATCACCCCGGCAGCCGAGGTGGTGGAAGGACAGGCAGTGACACTGAGCTGCAGAAGCGCCCTAAG
CCCCACACCTGATGCCCGCTTCTCCTGGTACCTGAATGGAGCCCTGCTTCACGAGGGTCCCGGCAGCAGC
CTCCTGCTCCCCGCGGCCTCCAGCACTGACGCCGGCTCATACCACTGCCGCGCCCGGGACGGCCACAGTG
CCAGTGCCCCCTCTTCGCCAGCTGTTCTCACTCTGCTCTGTGAGCAGCCACCACGACAACCAACATTCAC
CACCAGCCTCGACCTTGATGCCGCTCGCGCCGGGGCTGGACGGCGAGGCCTCCTTTTGTGCCGTGTCGAC
AGCGACCCCCCCCCCAGGCTGCAGCTGCTCCACAAGGACCGTGTTGTCGCCACTTCCCTGCCATCACGGG
GTGGCTGCAGCACCTGTGGGGGCTCTTCCCCACGCATCAAGGTCACCAAAGCCCCCAACTTGCTGCGTGT
CGAGATTCACAACCCTTTCCTGGAAGAGGAGGGCTTGTACCTCTGTGAGGCCAGCAATCCCCTGGGCAAC
CCCTCCACCTCAGCCACCTTCAATGGCCAGGCCACTCTCCTGGCCATTGCACCATCACACACACTTCAGC
AGGGCACAGAACCCAACTTGACTTGCAACGTGAGCCGGGAAGCTGCTGGCAGCCCTGCTAACTTCTCCTG
GTTCCGAAATGGGGTGCTGTGGGCCCAGGGTCCCCTGGAGACCGTGACACTGCTGCCCGTCGCCAGAACT
GATGCTGCOCTTTACGCCTGCCCCATCCTGACTGAGCCTGGTGCCCAGCTCTCCACTCCCGTGCTCCTGA
GTGTACTCTATCCCCCGGACCGTCCAAAGCTGTCAGCCCTCCTAGACATCGGCCAGGGCCACATGGCTCT
GTTCATCTGCACTGTGGACAGCCGCCCCCTGGCCTTGCTCGCCTTGTTCCATGGCGAGCACCTCCTGGCC
ACCAGCCTGGGTCCCCAGGTCCCATCCCATGGTCCGTTCCAGGCTAAAGCTGAGCCCAACTCCCTGAAGT
TAGAGGTCCGAGAACTGGGCCTTGGGCACTCTGGCAGCTACCGCTGTCAGGCCACAAATGTTCTTCGATC
ATCCAACACCTCACTCTTCTTCCACGTCCGAGGTGCCTGGGTCCAGCTGTCACCATCACCTGAGCTCCAA
GAGGGCCAGGCTGTGGTCCTGAGCTGCCAGGTACACACAGGAGTCCCAGACGGGACCTCATATCGTTGGT
ATCGGGATGGCCAGCCCCTCCAGGAGTCCACCTCGCCCACGCTCCGCTTTGCAGCCATAACTTTGACACA
AGCTGGGGCCTATCATTGCCAAGCCCAGGCCCCAGCCTCAGCCACCACGAGCCTAGCTGCACCCATCAGC
CTCCACGTGTCCTGTAAGGATGCCCCACGCCACGTCACACTCACTACCCTGATGGACACAGGCCCTGGAC
GACTGGGCCTCCTCCTGTGCCGTGTGGACAGTGACCCTCCGGCCCAGCTGCGGCTGCTCCACGGGGATCG
CCTTGTGGCCTCCACCCTACAAGGTGTGGGGGGACCCGAAGGCAGCTCTCCCAGGCTGCATGTGGCTGTG
GCCCCCAACACACTGCGTCTGGAGATCCACCGGGCTATGCTGGAGGATGAGGGTGTCTATATCTGTGAGG
CCTCCAACACCCTGGGCCAGGCCTCGCCCGCAGCTGACTTCGACGCTCAAAGCTGTGAATGPGCAGCTGT
GGCCCGGGGCTACCGTGCGGGAGGGGCAGCTGGTGAACCTGACCTGCCTTGTGTGGACCACTCACCCGGC
CCAGCTCACCTACACATGGTACCAGGATGGGCAGCAGCGCCTGGATCCCCACTCCATCCCCCTCCCCAAC
GTCACAGTCAGGGATGCCACCTCCTACCGCTGCGGTCTGGGCCCCCCTGGTCGCGCACCCCGCCTCTCCA
GACCTATCACCTTCGACGTCCTCACGCGCCCCGCAACCTGCGCCTGACCTACCTCCTCGAGAGCCATGGC
GGGCAGCTGGCCCTGGTACTGTCCACTGTGGACAGCCCCCCGCCCCCCCAGCTGCCCCTCAGCCACGCCG
GTCCCCTCTTGGCCTCCTCGACAGCAGCCTCTGTCCCCAACACCCTGCGCCTGGAGCTGCGAGGGCCACA
GCCCACGGATGAGGGTTTCTACACCTCCTCTGCCCGCAGCCCTCTGGGCCAGGCCAACACGTCCCTGGAG
CTGCGGCTGGAGGTGCGGGTGATCCTGGCTCCGGAGGCTGCCGTGCCTGAACGTGCCCCCATCACAGTGA
CCTGTGCGGACCCTGCTCCCCACGCACCCACACTCTATACTTCGTACCACAACCGTCGTTGGCTGCAGGA
GGGTCCAGCTGCCTCACTCTCATTCCTCCTCGCCACGCGGGCTCATGCAGGCCCCTACTCTTGCCAGGCC
CAGGATGCCCAGGGCACCCCCAGCTCCCCTCCTGCTGCCCTCCAAGTCCTCTGTGCCCCTCAGGACCCTG
TCCTGTCCTCCTTCCGGGACTCCAGGGCCAGATCCATGGCTGTGATACAGTGCACTGTGGACAGTGAGCC
ACCTGCTGAGCTGGCCCTATCTCATCATGCCAAGGTGCTCGCCACGAGCAGCGGGGTCCACACCTTGCCA
TCAGGGACAGGCCATGTCCAGGTGGCCCGAAACGCCCTACGCCTGCAGGTGCAAGATGTGCCTGCAGGTG
ATGACACCTATGTTTGCACAGCCCAAAACTTGCTGCCCTCAATCAGCACCATCGGGCGGTTGCAGGTAGA
AGGTGAGTGGCGCGTGGTGGCAGAGCCTGGCCTGGACGTGCCTGACGGCCCTGCCCTGAACCTCAGCTGC
CGCCTCCTGGGAGGCCCTGGGCCTGTGGGCAACTCCACCTTTGCATGGTTCTGGAATGACCGGCGGCTGC
ACGCGGAGCCTGTGCCCACTCTCGCCTTCACCCACGTGGCTCGTGCTCAAGCTGGGATGTACCACTGCCT
GGCTGAGCTCCCCACTGGGGCTGCTGCCTCTGCTCCAGTCATCCTCCGTGTGCTCTACCCTCCCAAGACG
CCCACCATGATGGTCTTCGTGGAGCCTGAGGGTGGCCTCCGGGGCATCCTGGATTGCCGAGTGGACAGCG
AGCCGCTCCCCAGCCTGACTCTCCACCTTGCCAGTCGACTGGTGGCCTCCAGTCAGCCCCAGGGTGCTCC
TGCAGAGCCACACATCCATGTCCTGGCTTCCCCCAATGCCCTGAGGGTGGACATCGAGGCGCTGAGGCCC
AGCGACCAAGGGGAATACATCTGTTCTGCCTCAAATGTCCTGGGCCCTGCCTCTACCTCCACCTACTTTG
GGGTCAGAGCCCTGCACCGCCTGCATCAGTTCCAGCAGCTCCTCTGGGTCCTGGGACTGCTGGTGGGCCT
CCTGCTCCTGCTGTTCGGCCTGGGGCCCTGCTACACCTGGACAAGGAGGCCTGTTTGTAAGCAGAGCATG
GGCGAGAATCCAGGGCGGGCATCGTCTTTCCATTTACTGCCTCTAGCTCGGTCTTCAAGGTA

The NOV6 protein encoded by SEQ ID NO:23 has 1700 amino acid residues, and is presented using the one-letter code in Table 6B (SEQ ID NO:24). The SignalP, Psort and/or Hydropathy profile for NOV6a predict that NOV6 has a signal peptide is likely to be localized at the plasma membrane (certainty=0.4600). NOV6 is also likely to be localized to the endoplasmic reticulum (membrane) (certainty=0.1000); endoplasmic reticulum (lumen) (certainty=0.1000); and outside (certainty=0.1000). The predicted protein is similar to the “SN Family”, some members of which have secreted and membrane localization and can be presented at the plasma membrane. Therefore, it is likely that this novel SN is available at the appropriate subcellular localization and hence accessible for the therapeutic uses described herein. A likely signal peptide cleavage site is indicated at the slash in the sequence GQA-SW, between amino acids 20 and 21 in Table 6B.

TABLE 6B
Encoded NOV6 protein sequence

(SEQ ID NO:24)

MGFLPKLLLLASAVLPPGQASWGVSSPQDVQGVKGSCLLIPCIFSFPADVEVPDGILTAIWYYDYSGQR
QVVSHSADPKLVEARFRGRTEFMGNPEHRVCNLLLKDLQPEDSGSYNFREFEISEVNRWSDVKGTLVTV
TGDPRVPTIASPVELLEGTEVDFNCSTPYVCLQEQVRLQWQGQDPARSVTFNSQKFEPTGVGHLETLH
MAMSWQDHGRILRCQLSVANHRAQSEIHLQVKCAPKGVKILLSPSGRNILPGELVTLTCQVNSSYPAV
SSIKWLKDGVRLQTKTGVLHLPQAAWSDAGVYTCQAENGVGSLVSPPTSLHIFVAEVQVSPAGPILSN
QTVTLVCNTPNEAPSDLRYSWYKNHVLLEDAHSHTLRLHLATRADTGFYFCEVQNVHGSERSGPVSVV
VTDPPLTPVLTAFLETQAGLVGILHCSVVSEPLATLVLSHGGHILASTSGDSDHSPRFSGTSGPNSLR
LEIRDLEETDSGEYKCSATNSLGNATSTLDFHANVARLLISPAAEVVEGQAVTLSCRSGLSPTPDARF
SWYLNGALLHEGPGSSLLLPAASSTDAGSYHCRARDGHSASGPSSPAVLTVLCEQPPRQPTFTTRLDL
DAAGAGAGRRGLLLCRVDSDPPARLQLLHKDRVVATSLPSGGGCSTCGGCSPRMKVTKAPNLLRVETH
NPLLEEEGLYLCEASNALGNASTSATFNGQATVLAIAPSHTLQEGTEANLTCNVSREAAGSPANFSWF
RNGVLWAQGPLETVTLLPVARTDAALYACRILTEAGAQLSTPVLLSVLYPPDRPKLSALLDMGQGHMA
LFICTVDSRPLALLALFHGEELLATSLGPQVPSHGRFQAKAEANSLKLEVRELGLGDSGSYRCEATNV
LGSSNTSLFFQVRGAWVQVSPSPELQEGQAVVLSCQVHTGVPEGTSYRWYRDGQPLQESTSATLRFAA
ITLTQAGAYHCQAQAPGSATTSLAAPISLHVSCKDAPRHVTLTTLMDTGPGRLGLLLCRVDSDPPAQL
RLLHGDRLVASTLQGVGGPEGSSPRLHVAVAPNTLRLEIHGAMLEDEGVYTCEASNTLGQASASADFD
AQSCECAGVARGYRAGGAAGEPDLPCVDHSPGPAHLHMVPGWAAAPGCPLHPPAQRHSQGCHLLPLRC
GPPWSGTPPLQTYHLGRPHAPRNLRLTYLLESHGGQLALVLCTVDSRPPAQLALSHAGRLLASSTAAS
VPNTLRLELRGPQPRDEGFYSCSARSPLGQANTSLELRLEVRVILAPEAAVPEGAPITVTCADPAAHA
PTLYTWYHNGRWLQEGPAASLSFLVATRAHAGAYSCQAQDAQGTRSSRPAALQVLCAPQDAVLSSFRD
SRARSMAVIQCTVDSEPPAELALSHDGKVLATSSGVHSLASGTGHVQVARNALRLQVQDVPAGDDTYV
CTAQNLLGSISTIGRLQVEGEWRVVAEPGLDVPEGAALNLSCRLLGGPGPVGNSTFAWFWNDRRLHAE
PVPTLAFTHVARAQAGMYHCLAELPTGAAASAPVMLRVLYPPKTPTMMVFVEPEGGLRGILDCRVDSE
PLASLTLHLGSRLVASSQPQGAPAEPHIHVLASPNALRVDTEALRPSDQGEYICSASNVLGPASTSTY
FGVRALHRLHQFQQLLWVLGLLVGLLLLLLGLGACYTWRRRRVCKQSNGENPGRASSFHLLPLAGSSR.

The disclosed NOV6 nucleic acid sequence belongs to genomic DNA [Acc.NO.: AL109804 from GenbankNEW]. Within this GenbankNew entry is a note showing that the sequence was from Chromosome 1. Therefore, the likely chromosomal locus of the disclosed NOV6 nucleic acid is Chromosome 1.

NOV6 is expressed in at least the following tissues: bone marrow, thyroid, lymph node, pancreas, placenta, fetal liver, heart, prostate, spleen, salivary gland, mammary gland, thalamus, adrenal gland, and kidney.

The disclosed NOV6 protein (SEQ ID NO:24) has good identity with sialoadhesin proteins. The identity information used for ClustalW analysis is presented in Table 6C.

TABLE 6C
BLAST results for NOV6

| Gene Index/ | Protein/ | Length | Identity | Positives | |
|---|---|---|---|---|---|
| Identifier | Organism | (aa) | (%) | (%) | Expect |
| gi\|6919968\| | Sialoadhesin | 1694 | 1097/1678 | 1256/1678 | 0.0 |
| sp\|Q62230\| | (Mus | | (65%) | (74%) | |
| SN_MOUSE | musculus) | | | Gaps = | |
| SIALOADHESIN | | | | 31/1678 | |
| PRECURSOR (SER); | | | | (1%) | |
| gi\|1083506\| | | | | | |
| pir\|\| | | | | | |
| S50065; | | | | | |
| gi\|557254\| | | | | | |
| emb\|CAA85290.1\| | | | | | |
| (Z36293) | | | | | |
| gi\|13489095\| | dJ1009E24.1.1 | 1709 | 1495/1680 | 1503/1680 | 0.0 |
| ref\|NP_075556.1; | (sialoadhesin | | (88%) | (88%) | |
| gi\|11493365\| | (isoform 1)) | | | Gaps = | |
| emb\|CAC17543.1 | (Homo | | | 32/1680 | |
| (AL109804) | sapiens) | | | (1%) | |
| gi\|557250\| | sialoadhesin | 1598 | 1010/1530 | 1153/1530 | 0.0 |
| emb\|CAA85268.1\| | (Mus | | (66%) | (75%) | |
| (Z36233) | musculus) | | | Gaps = | |
| | | | | 31/1530 | |
| | | | | (2%) | |
| gi\|12656130\| | sialoadhesin | 1709 | 1494/1680 | 1502/1680 | 0.0 |
| gb\|AAK00757.1\| | (Homo | | (88%) | (88%) | |
| AF230073_1 | sapiens) | | | Gaps = | |
| (AF230073) | | | | 32/1680 | |
| | | | | (1%) | |
| gi\|10440438\| | FLJ00055 | 977 | 840/955 | 846/955 | 0.0 |
| dbj\|BAB15752.1\| | protein | | (87%) | (87%) | |
| (AK024462) | (Homo | | | Gaps = | |
| | sapiens) | | | 16/955 | |
| | | | | (1%) | |
| gi\|6755584\| | sialoadhesin | 1695 | 1103/1678 | 1259/1678 | 0.0 |
| ref\|NP_035556.1; | (Mus | | (75%) | (74%) | |
| gi\|2768750\| | musculus) | | | Gaps = | |
| gb\|AAB95641.1\| | | | | 30/1678 | |
| (U92843) | | | | (1%) | |
| gi\|13653900\| | sialoadhesin | 1620 | 1422/1607 | 1430/1607 | 0.0 |
| ref\|XP_016245.1\| | (Homo | | (88%) | (88%) | |
| | sapiens) | | | Gaps = | |
| | | | | 32/1607 | |
| | | | | (1%) | |
| gi\|11493364\| | dJ1009E24.1.2 | 462 | 440/445 | 440/445 | 0.0 |
| emb\|CAC17542.1\| | (sialoadhesin | | (98%) | (98%) | |
| (AL109804) | (isoform 2)) | | | Gaps = | |
| | (Homo | | | 2/445 | |
| | sapiens) | | | (0%) | |
| gi\|10440432\| | FLj00051 | 462 | 439/445 | 439/445 | 0.0 |
| dbj\|BAB15749.1\| | protein | | (98%) | (98%) | |
| (AK024459); | (Homo | | | Gaps = | |
| gi\|10440472\| | sapiens); | | | 2/445 | |
| dbj\|BAB15769.1\| | FLJ00073 | | | (0%) | |
| (AK024479) | protein | | | | |
| | (Homo | | | | |
| | sapiens) | | | | |

This information is presented graphically in the multiple sequence alignment given in Table 6D (with NOV6 being shown on line 1) as a ClustalW analysis comparing NOV6 with related protein sequences.

[image]

[image]

[image]

[image]

[image]

The presence of identifiable domains in NOV6 was determined by searches using algorithms such as PROSITE, Blocks, Pfam, ProDomain, Prints and then determining the Interpro number by crossing the domain match (or numbers) using the Interpro website (http:www.ebi.ac.uk/interpro/).

DOMAIN results for NOV6 were collected from the Conserved Domain Database (CDD) with Reverse Position Specific BLAST. This BLAST samples domains found in the Smart and Pfam collections. The results are listed in Table 6E with the statistics and domain description. The results indicate that this protein contains at least one immunoglobulin domain. The presence of these identifiable domains is shown in Table 6F.

TABLE 6E
Domain Results for NOV6

| | | Score | |
|---|---|---|---|
| Domain Identifier | Domain Name | (Bits) | E Value |
| gnl\|Smart\|IG | Immunoglobulin | 53.1 | 1e−07 |
| gnl\|Smart\|IG | Immunoglobulin | 48.5 | 3e−06 |
| gnl\|Smart\|IG | Immunoglobulin | 43.9 | 7e−05 |
| gnl\|Smart\|IG | Immunoglobulin | 40.0 | 0.001 |
| gnl\|Smart\|IG | Immunoglobulin | 38.9 | 0.002 |
| gnl\|Smart\|IG | Immunoglobulin | 38.9 | 0.002 |
| gnl\|Smart\|IG | Immunoglobulin | 37.4 | 0.006 |
| gnl\|Smart\|IG_like | Immunoglobulin like; IG domains | 52.8 | 1e−07 |
| | that cannot be classified into . . . | | |
| gnl\|Smart\|IG_like | Immunoglobulin like; IG domains | 50.1 | 1e−06 |
| | that cannot be classified into . . . | | |
| gnl\|Smart\|IG_like | Immunoglobulin like; IG domains | 47.4 | 6e−06 |
| | that cannot be classified into . . . | | |
| gnl\|Smart\|IG_like | Immunoglobulin like; IG domains | 43.9 | 7e−05 |
| | that cannot be classified into . . . | | |
| gnl\|Smart\|IG_like | Immunoglobulin like; IG domains | 41.6 | 3e−04 |
| | that cannot be classified into . . . | | |
| gnl\|Smart\|IG_like | Immunoglobulin like; IG domains | 40.8 | 6e−04 |
| | that cannot be classified into . . . | | |
| gnl\|Smart\|IG_like | Immunoglobulin like; IG domains | 39.3 | 0.002 |
| | that cannot be classified into . . . | | |
| gnl\|Smart\|IG_like | Immunoglobulin like; IG domains | 38.5 | 0.003 |
| | that cannot be classified into . . . | | |
| gnl\|Smart\|IG_like | Immunoglobulin like; IG domains | 38.1 | 0.004 |
| | that cannot be classified into . . . | | |
| gnl\|Smart\|IG_like | Immunoglobulin like; IG domains | 37.7 | 0.005 |
| | that cannot be classified into . . . | | |
| gnl\|Smart\|IG_like | Immunoglobulin like; IG domains | 37.7 | 0.005 |
| | that cannot be classified into . . . | | |
| gnl\|Smart\|IG_like | Immunoglobulin like; IG domains | 37.7 | 0.005 |
| | that cannot be classified into . . . | | |
| gnl\|Smart\|IG_like | Immunoglobulin like; IG domains | 37.7 | 0.005 |
| | that cannot be classified into . . . | | |
| gnl\|Smart\|IGc2 | Immunoglobulin C-2 Type | 38.9 | 0.002 |
| gnl\|Pfam\|pfam00047 | Ig, Immunoglobulin domain | 37.7 | 0.005 |
| gnl\|Pfam\|pfam00047 | Ig, Immunoglobulin domain | 37.4 | 0.006 |

TABLE 6F
DOMAIN results for NOV6

gnl|Smart|IG, Immunoglobulin
CD-Length=63 residues, 100.0% aligned
Score=53.1 bits (126), Expect=1E−07

| | | | | |
|---|---|---|---|---|
| Query: | 254 | LPGELVTLTCQVNSSYPAVSSIKWLKDGVRLQ-----TKTGVLHLPQAAWSDAGVYTCQA | 308 | (SEQ ID NO:109) |
| Sbjct: | 1 | LEGSSVTLTCPASGDPVP--NITWLKDGKPLPESRVVASGSTLTIKNVSLEDSGLTCVA | 58 | (SEQ ID NO:110) |
| Query: | 309 | ENGVG 313 | | |
| Sbjct: | 59 | RNSAG 63 | | |

gnl|Smart|IG, Immunoglobulin
CD-Length=63 residues, 93.7% aligned
Score=48.5 bits (114), Expect=3e−06

| | | | | |
|---|---|---|---|---|
| Query: | 910 | QEGQAVVLSCQVHTGVPEGTSYRWYRDGQPLQES----TSATLRFAAITLTQAGAYHCQA | 965 | (SEQ ID NO:111) |
| Sbjct: | 1 | LEGESVTLTCPA-SGDEVPN-ITWLKDGKRLPESRVVASGSLTIKNVSLEDSGLYTCVA | 58 | (SEQ ID NO:112) |
| Query: | 966 | Q 966 | | |
| Sbjct: | 59 | R 59 | | |

gnl|Smart|IG, Immunoglobulin
CD-Length=63 residues, 98.4% aligned
Score=43.9 bits (102), Expect=7e−05

| | | | | |
|---|---|---|---|---|
| Query: | 1277 | EGAPITVTCADPAAHAPTLYTWYHNGRVLQEGPA----ASLSFLVATRAHAHAGAYSCQAQD | 1332 | (SEQ ID NO:113) |
| Sbjct: | 2 | EGESVTLTCPASGDPVPNI-TWLKDGKPLPESRVVASGSTLLTIKNVSLEDSGLYTCVARN | 60 | (SEQ ID NO:114) |
| Query: | 1333 | AQG 1335 | | |
| Sbjct: | 61 | SAG 63 | | |

gnl|Smart|IG, Immunoglobulin
CD-Length=63 residues, 100.0% aligned
Score=40.0 bits (92), Expect=0.001

| | | | | |
|---|---|---|---|---|
| Query: | 338 | LENQTVTLVCNTPNEAPSDLRYSWYKNHVILLEDAHSH----TLRLHLATRADTGFYFCE | 392 | (SEQ ID NO:115) |
| Sbjct: | 1 | LEGESVTLTCPASGDPVPN---ITWLKDGKPLPESRVVASGSTLTIKNVSLEDSGLYTCV | 57 | (SEQ ID NO:116) |
| Query: | 393 | VQNVHG 398 | | |
| Sbjct: | 58 | ARNSAG 63 | | |

gnl|Smart|IG, Immunoglobulin
CD-Length=63 residues, 98.4% aligned
Score=38.9 bits (89), Expect=0.002

| | | | | |
|---|---|---|---|---|
| Query: | 1462 | EGAALNLSCRLLGGPGPVGNSTFAWFWNDRRLHAEPV----PTLAFTHVARAQAGMYHCL | 1517 | (SEQ ID 140:117) |
| Sbjct: | 2 | EGESVTLTCPASGDPVP----NITWLKDGKPLPESRVVASGSTLTIKNVSLEDSGLYTCV | 57 | (SEQ ID 140:118) |
| Query: | 1518 | LELPTG 1523 | | |
| Sbjct: | 58 | ARESAG 63 | | |

gnl|Smart|IG, Immunoglobulin
CD-Length=63 residues, 98.4% aligned
Score=38.9 bits (89), Expect=0.002

| | | | | |
|---|---|---|---|---|
| Query: | 524 | EGQAVTLSCRSGLSPTPDARFSWYLNGALLHE----GPGSSLLLPAASSTDAGSYHCRAR | 579 | (SEQ ID 140:119) |
| Sbjct: | 2 | EGESVPLTCPASGDPVPN--ITWLKDGKPLPESRVVASGSTLTIKNVSLEDSGLYTCVAR | 59 | (SEQ ID NO:120) |
| Query: | 580 | DGHS 583 | | |
| Sbjct: | 60 | NSAG 63 | | |

gnl|Smart|IG, Immunoglobuiin
CD-Length=63 residues, 100.0% aligned
Score=37.4 bits (85), Expect=0.006

| | | | | |
|---|---|---|---|---|
| Query: | 723 | QEGTEANLTCNVSREAAGSP-ANFSWFRNGVLWAQGPLE----TVTLLPVARTQSGLYTC | 777 | (SEQ ID NO:121) |
| Sbjct: | 1 | LEGESVTLTC----PASGDPVPNITWLKDGKPLPESRVVASGSTLTIKNVSLEDSGLYTC | 56 | (SEQ ID NO:122) |
| Query: | 778 | RILTEAG 784 | | |
| Sbjct: | 57 | VARNSAG 63 | | |

gnl|Smart|IG like, Immunoglobulin like; IG domains
that cannot be classified into one of IGv1, TGc1, TGc2, IG.
CD-Length=86 residues, 93.0% aligned
Score=52.8 bits (125), Expect=1e−07

| | | | | |
|---|---|---|---|---|
| Query: | 248 | PSGRNILPGELVTLTCQVNSSYPAVSSIKWLKD-----------VRLQTKTGVLHLPQA | 296 | (SEQ ID NO:123) |
| Sbjct: | 1 | PPSVTVKEGESVTLSCEASGGNPPP--TVTWYKQGGKLLAESGRFSVSRSGGNSTLTISNV | 58 | (SEQ ID NO:124) |
| Query: | 297 | AWSDAGVAYTCQAENGVGSLVSP | 318 | |
| Sbjct: | 59 | TPEDSGTYTCAATNGSGSASSG | 80 | |

gnl|Smart|IG like, Immunoglobulin like; IC domains
that cannot be classified into one of IGv1, TGc1, IGc2, IG.
CD-Length=86 residues, 77.9% aligned
Score=50.1 bits (118), Expect=1e−06

| | | | | |
|---|---|---|---|---|
| Query: | 432 | LHCSVVSEPLATLVLSHGGHILASTSGDSDHSPRFSGTSGPNSLRLELRDLEETDGEYK | 491 | (SEQ ID NO:125) |
| Sbjct: | 14 | LSCEASGNPPPTVTWYKQGGKLLAESG------RFSVSRSGGNSTLTLNVTPEDSGTYT | 67 | (SEQ ID NQ:126) |
| Query: | 492 | CSATNSLC-NATST 504 | | |
| Sbjct: | 68 | CAATNGSGSASSG 80 | | |

gnl|Smart|IG like, Immunoglobulin like; IG domains
that cannot be classified into one of IGv1, IGc1, IGc2, IG.
CD-Length+322 86 residues, 84.9% aligned
Score=47.4 bits (111), Expect=6e−06

| | | | | |
|---|---|---|---|---|
| Query: | 339 | ENQTVTLVCNTPNEAPSDLRYSEYKN----------HVLLEDAHSHLRLHLATRADTGF | 388 | (SEQ ID NQ:127) |
| Sbjct: | 8 | EGESVTLSCEASGNPPP--TVTWYKQGGKLLAESGRFSVSRSGGNSILTISNVIPEDSGT | 65 | (SEQ ID NO:128) |
| Query: | 389 | YFCEVQNVHGSERSG 403 | | |
| Sbjct: | 66 | YTCAATNGSGSASSG 80 | | |

gnl|Smart|IG like, Immunoglobulin like; IG domains
that cannot be classified into one of IGv1, IGv1, IGc2, IG.
CD-Length=86 residues, 72.1% aligned
Score=43.9 bits (102), Expect=7e−05

| | | | | |
|---|---|---|---|---|
| Query: | 1008 | LLCRVDSDPPAQLRLLHGDRLVASTLQGVGGPEGSSPRLHVAVAPNTLRLEIHGAMLEDE | 1067 | (SEQ ID NO:129) |
| Sbjct: | 14 | LSCEASGNPPPTVTWYKQG----------GKLLAESGRFSVSRSGGNSTLTISNTVPEDS | 63 | (SEQ ID NO:130) |
| Query: | 1068 | GVYICEASNTLG 1079 | | |
| Sbjct: | 64 | GTYTAATNGSG 75 | | |

gnl|Smart|IG like, Immunoglobulin like; IG domains
that cannot be classified into one of IGv1, IGc1, IGc2, IG.
CD-Length=86 residues, 100.0% aligned
Score=41.6 bits (96), Expect=3e−04

| | | | | |
|---|---|---|---|---|
| Query: | 904 | SPSPELQEGQAVVLSCQVHTGVPEGTSYRWYRDGQPL---------QESTSATLRFAAI | 953 | (SEQ ID 140:131) |
| Sbjct: | 1 | PPSVTVKEGESVTLSCEA-SGNPP-PTVTWYKQGGKLLAESGRFSVSRSGGNSTLTISNV | 58 | (SEQ ID 140:132) |
| Query: | 954 | TLTQAGAYHTQAQAPGSATTSLAAPISLHVS 984 | | |
| Sbjct: | 59 | TPEDSGTYTCAAT---NGSGSASSGVTLTVL 86 | | |

gnl|Smart|IG like, Immunoglobulin like; IC domains
that cannot be classified into one of IGv1, IGc1, ICc2, IC.
CD-Length=86 residues, 88.4% aligned
Score=40.8 bits (94), Expect=6e−04

| | | | | |
|---|---|---|---|---|
| Query: | 1191 | GQLALVLCTVDSRPPAQLLSH-AGRLLIASSTAAV---PNTLRIELRGPQPRDEGFYSC | 1246 | (SEQ ID NO: 133) |
| Sbjct: | 9 | GESVTLSCEASCNPPPTVTWYKQGCKLLAESGRFSVSRSGGNSTLTISNVTPEDSGTYTC | 68 | (SEQ ID 140:134) |
| Query: | 1247 | SARSPLGQANTSLELR 1262 | | |
| Sbjct: | 69 | AATNGSGSASSGVTLT 84 | | |

gnl|Smart|IC like, Immunoglobulin like; IC domains
that cannot be classified into one of IGv1, IGc1, ICc2, IG.
CD-Length=86 residues, 100.0% aligned
Score=39.3 bits (90), Expect=0.002

| | | | | |
|---|---|---|---|---|
| Query: | 517 | SPAAEVVEGQAVTLSCRSGLSPTPDARFSWYLNGA----------LLHEGPGSSLLLPAA | 566 | (SEQ ID NO: 135) |
| Sbjct: | 1 | PPSVTVKEGESVTLSCE--ASGNPPPTVTWYKQGGKLLAESGRFSVSRSGGNSTLTISNV | 58 | (SEQ ID NO:136) |
| Query: | 567 | SSTDAGSYHCRARDGHSASGPSSPAVLTVL 596 | | |
| Sbjct: | 59 | TPEDSGTYTCAATNGSGSA--SSGVTLTVL 86 | | |

gnl|Smart|IC like, Immunoglobulin like; IG domains
that cannot be classified into one of ICv1, IGc1, ICc2, IC.
CD-Length=86 residues, 86.0% aligned
Score=38.5 bits (88), Expect=0.003

| | | | | |
|---|---|---|---|---|
| Query: | 1275 | VPEGAPITVTCADPAAHAPTLYTWYHNG----------RWLQEGPAASLSFLVATRAHAG | 1324 | (SEQ ID 140:137) |
| Sbjct: | 6 | VKECESVTLSCEASGNPPPTV-TWYKQCCKLLAESCRFSVSRSGGNSTLTISNVTPEDSG | 64 | (SEQ ID 140:138) |
| Query: | 1325 | AYSCQAQDAQCTRSS 1339 | | |
| Sbjct: | 65 | TYTCAATNCSCSASS 79 | | |

gnl⊕Smart|IG like, Immunoglobulin like; IG domains
that cannot be classified into one of IGv1, IGc1, IGc2, IG.
CD-Length=86 residues, 98.8% aligned
Score=38.1 bits (87), Expect=0.004

| | | | | |
|---|---|---|---|---|
| Query: | 718 | PSHTLQEGTEANLTCNVSREAAGSPANFSWFRNGVLWAQGPLE----------TVTLLPV | 767 | (SEQ ID NO:139) |
| Sbjct: | 2 | PSVTVKEGESVTLSCEAS---GNPPPTVTWYKQCCKLLAESGRFSVSRSGCNSTLTISNV | 58 | (SEQ ID NO:140) |
| Query: | 768 | ARTDAALYACRILTEAGAQLSTPVLLSVD 796 | | |
| Sbjct: | 59 | TPEDSGTYTCAATNGSG-SASSCVTLTVL 86 | | |

gnl|Smart|IC like, Immunoglobulin like; IC domains
that cannot be classified into one of IGv1, IGc1, ICc2, IG.
CD-Length=86 residues, 77.9% aligned
Score=37.7 bits (86), Expect=0.005

| | | | | |
|---|---|---|---|---|
| Query: | 627 | CRVDSDPPARLQLLHKDRVVATSLPSGGGCSTCGGCSPRMKVTKAPNLLRVELHXPLLEE | 686 | (SEQ ID NO:141( |
| Sbjct: | 16 | CEASGNPPPTVTWYKQCCKLLAE-------------SGRFSVSRSGGNSTLTTSXVTPED | 62 | (SEQ ID 140:142) |
| Query: | 687 | EGLYLCEASNALCNASTSAT 706 | | |
| Sbjct: | 63 | SGTYTCAATNGSGSASSGVT 82 | | |

gnl|Smart|lG like, Immunoglobulin like; IG domains
that cannot be classified into one of IGv1, IGc1, IGc2, IG.
CD-Length=86 residues, 79.1% aligned
Score 37.7 bits (86), Expect=0.005

| | | | | |
|---|---|---|---|---|
| Query: | 1557 | LDCRVDSEPLASLTLHLGSRLVASSQPQGAFAEPHIHVLASFNALRVDTEALRPSDQGEY | 1616 | (SEQ ID NO:143) |
| Sbjct: | 14 | LSCEASGNPPPTVTWYKQG-------GKLLAESGRFSVSRSGGNSTLTISNVTPEDSGTY | 66 | (SEQ ID NO:144) |
| Query: | 1617 | ICSASNVLGPASTST 1631 | | |
| Sbjct: | 67 | TCAATNGSGSASSGV 81 | | |

gnl|Smart|IG like, Immunoglobulin like; IG domains
that cannot be classified into one of IGv1, IGc1, IGc2, IG.
CD-Length=86 residues, 89.5% aligned
Score=37.7 bits (86), Expect=0.005

| | | | | |
|---|---|---|---|---|
| Query: | 813 | GHMALFICTVDSRPLALLALFHGEHLLATSLGPQVPSHGREQAKAEANSLKLEVRELGLG | 872 | (SEQ ID NO:145) |
| Sbjct: | 9 | GESVTLSCEASGNPPPTVTWYKQG-------GKLLAESGRESVSRSGGNSTLTISNVTPE | 61 | (SEQ ID NO:146) |
| Query: | 873 | DSGSYRCEATNVLZSSNTSLFFQV 896 | | |
| Sbjct: | 62 | DSGTYTCAACNGSCSASSGVTLTV 85 | | |

gnl|Smart|IG like, Immunoglobulin like; IC domains
that cannot be classified into one of IGv1, IGc1, IGc2, IC.
CD-Length=86 residues, 94.2% aligned
Score=37.7 bits (86), Expect=0.005

| | | | | |
|---|---|---|---|---|
| Query: | 1460 | VPECAALNLSCRLLCCPCPVCNSTFAWFWNDRRLHAEP----------VPTLAFTHVARA | 1509 | (SEQ ID NO:147) |
| Sbjct: | 6 | VKEGESVTLSCEASGNPPP----TVTWYKQGGKLLAESCRFSVSRSCGNSTLTISNVTPE | 61 | (SEQ ID NO: 148) |
| Query: | 1510 | QAGNYHCLAELFTCAAASAPVMLRVL 1535 | | |
| Sbjct: | 62 | DSGTYTCAAT-NGSGSASSGVTLTVL 86 | | |

gnl|Smart|IGc2, Immunoglobulin C-2 Type
CD-Length=74 residues, 89.2% aligned
Score=38.9 bits (89), Expect=0.002

| | | | | |
|---|---|---|---|---|
| Query: | 259 | VTLTCQVNSSYPAVSSIKWLEDGVRLQTKTG----------------VLHLFQAAWSDAG | 302 | (SEQ ID NO:149) |
| Sbjct: | 2 | ATLVCLVTGFYPFDITVTWLKNGQEVTSGVETTDFLKEKDGTYSLSSYLTVS-ATWESGD | 60 | (SEQ ID NO:150) |
| Query: | 303 | VYTCQAE 309 | | |
| Sbjct: | 61 | TYTCRVT 67 | | |

gnl|Pfam|pfam00047, ig, Immunoglobulin domain.
CD-Length 68 residues, 100.0% aligned
Score=37.7 bits (86), Expect=0.005

| | | | | |
|---|---|---|---|---|
| Query: | 256 | GELVTLTCQVNSSYPAVSSIKWLKDGVRLQTKTGV----------------LHLPQAAWS | 299 | (SEQ ID NQ:151) |
| Sbjct: | 1 | GESVTLTCSV-SGYPPDPTVTWLRNGKGIELLGSSESRVTSGGRFSISSLSLTISSVTPE | 59 | (SEQ ID NO:152) |
| Query: | 300 | DAGVYTCQA 308 | | |
| Sbjct: | 60 | DSGTYTCVV 68 | | |

gnl|Pfam|pfam00047, ig, Inmunoglobulin domain.
CD-Length 68 residues, 100.0% aligned
Score=37.4 bits (85), Expect=0.006

| | | | | |
|---|---|---|---|---|
| Query: | 340 | NQTVTLVCNTPNEAPSDLRYSWYKNHVLLE---------------DAHSHTLRLHLATRA | 384 | (SEQ ID NO:153) |
| Sbjct: | 1 | GESVTLTCSVSG-YPPDPTVTWLRNGKGIELLGSSESRVTSGGRFSISSLSLTISSVTPE | 59 | (SEQ ID NO:154) |
| Query: | 385 | DTCFYFCEV 393 | | |
| Sbjct: | 60 | DSCTYTCVV 68 | | |

Other BLAST results include sequences from the Patp database, which is a proprietary database that contains sequences published in patents and patent publications. Patp results include those listed in Table 6G.

TABLE 6G
Patp alignments of NOV6

| | | | | Smallest |
|---|---|---|---|---|
| | | | | Sum |
| Sequences producing High-scoring | | Reading | High | Prob. |
| Segment Pairs: | | Frame | Score | P (N) |
| Patp: AAB41901 | Human ORFX polypep- | +1 | 1217 | 8.1e−123 |
| | tide sequence, 235 aa | | | |

For example, a BLAST against patp: AAB41901, a 235 amino acid Human ORFX polypeptide sequence (WO/0058473), produced good identity, E=8.1e-123 over the region from amino acid 3 to amino acid 235.

Crocker et.al., EMBO J 10(7):1661-69 (1991); PMID: 2050106, UI: 91266893, examined macrophage subpopulations in the mouse that express a lectin-like receptor, sialoadhesin (originally named sheep erythrocyte receptor (“SER”)), which selectively recognizes sialoglyco-conjugates and is likely to be involved in cellular interactions of stromal macrophages in haematopoietic and lymphoid tissues. Crocker et al. further described the purification and ligand specificity of sialoadhesin isolated from mouse spleen. Purified sialoadhesin, a glycoprotein of 185 kd apparent Mw, agglutinated sheep or human erythrocytes at nanomolar concentrations in a sialic acid-dependent manner. Low angle shadowing and electron microscopy showed that sialoadhesin consists of a globular head region of approximately 9 nm and an extended tail of approximately 35 nm.

To investigate the specificity for sialic acid, the interaction of sialoadhesin with derivatized human erythrocytes, glycoproteins, and glycolipids was examined. Sialoadhesin specifically recognizes the oligosaccharide sequence Neu5Ac alpha 2—3Gal beta 1—3GalNAc in either sialoglycoproteins or gangliosides. These findings imply that specific sialoglycoconjugates carrying this structure may be involved in cellular interactions between stromal macrophages and subpopulations of haematopoietic cells and lymphocytes.

The extracellular region of sialoadhesin is composed of seventeen immunoglobulin-like domains, of which the amino-terminal two are highly-related structurally and functionally to the amino-terminal domains of CD22, myelin associated glycoprotein and CD33. These proteins, collectively known as the sialoadhesin family, are able to mediate sialic acid-dependent binding with distinct specificities for both the type of sialic acid and its linkage to subterminal sugars.

Additionally, Crocker et.al. (Glycoconj J 14(5):601-09 (1997)) reviewed their recent studies on sialoadhesin and suggested how this molecule may contribute to a range of macrophage functions, both under normal conditions as well as during inflammatory reactions. (See also, Crocker et.al., EMBO J 13(19):4490-503 (1994), which reports the molecular cloning of murine sialoadhesin and show that it is a new member of the immunoglobulin (Ig) superfamily with 17 Ig-like domains. COS cells transfected with a cDNA encoding full-length sialoadhesin bound mouse bone marrow cells in a sialic acid-dependent manner). Alternatively spliced cDNAs, predicting soluble forms of sialoadhesin containing the first three or 16 Ig-like domains of sialoadhesin, were expressed in COS cells and the respective proteins purified. When immobilized on plastic, the 16-domain form bound cells in a sialic acid-dependent manner, suggesting that sialoadhesin can function in both secreted and membrane-bound forms.

The most similar proteins in the database were CD22, myelin-associated glycoprotein, Schwann cell myelin protein and CD33. Like sialoadhesin, CD22 mediates sialic acid-dependent cell adhesion. The sequence similarity of sialoadhesin to CD22 and related members of the Ig superfamily indicates the existence of a novel family of sialic acid binding proteins involved in cell-cell interactions.

Stromal macrophages in lymphohemopoietic tissues express novel macrophage-restricted plasma membrane receptors involved in nonphagocytic interactions with other hemopoietic cells. One such receptor with lectin-like specificity for sialylated glycoconjugates on sheep erythrocytes and murine hemopoietic cells has been characterized immunochemically and termed sialoadhesin. Sialoadhesin expression during mouse development was examined to learn more about its regulation and function. See Morris et.al., Dev Immunol 2(1):7-17 (1992). PMID: 1521065, UT: 92393348. Immunocytochemical, resetting, and Western blot studies show that sialoadhesin is first detected on fetal liver macrophages on day 18 of development, 7 days after numerous F4/80+ macrophages are found within erythroblastic islands. In spleen and bone marrow, sialoadhesin appears between day 18 and birth, in parallel with myeloid development. Strongly labeled macrophages in the marginal zone of spleen, characteristic of adult lymphoid tissues, appeared gradually between 1-4 weeks after birth, as the white pulp became enlarged. Isolation of fetal liver macrophages at day 14 confirmed that sialoadhesin was not involved in the binding of erythroblasts, which is mediated by a distinct cation-dependent receptor.

Sialoadhesin could be expressed by isolated fetal liver macrophages after cultivation in adult mouse serum, a known source of inducer activity, but was not dependent on the presence of this inducer, unlike adult-derived macrophages. Fetal plasma contained inducing activity on day 13, but adult levels were not reached until 2 weeks postnatally. These studies show that sialoadhesin is differentially regulated compared with the erythroblast receptor and F4/80 antigen, that it is not required for fetal erythropoiesis, and that its induction on stromal macrophages is delayed until the onset of myeloid and lymphoid development. Sialoadhesin provides a marker to study maturation and functions of macrophages during ontogeny of the lymphohemopoietic system. See generally, Morris et.al., Dev Immunol 2(1):7-17 (1992). PMID: 1521065, UI: 92393348.

The disclosed NOV6 protein of the invention has homology to the murine SN. The murine SN has characteristic properties, as mentioned in the above. The disclosed NOV6 protein of the invention therefore is predicted to have characteristic properties homologous to the murine SN. The expression pattern, map location, and protein similarity information for the invention(s) suggest that NOV6 may function as an SN family member.

The nucleic acids and proteins of the invention are useful in potential therapeutic applications implicated in various diseases and disorders and/or other pathologies and disorders. For example, a cDNA encoding the SN-like protein may be useful in gene therapy, and the SN-like protein may be useful when administered to a subject in need thereof. By way of nonlimiting example, the compositions of the present invention will have efficacy for treatment of patients suffering from from Von Hippel-Lindau (VHL) syndrome, Cirrhosis, Transplantation, Hemophilia, Hypercoagulation, Idiopathic thrombocytopenic purpura, autoimmume disease, allergies, immunodeficiencies, transplantation, Graft vesus host, Diabetes, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan syndrome, Alzheimer's disease, Stroke, Tuberous sclerosis, Parkinson's disease, Huntington's disease, Cerebral palsy, Epilepsy, Multiple sclerosis, Ataxia-telangiectasia, Leukodystrophies, Behavioral disorders, Addiction, Anxiety, Pain, Xerostomia, Neuroprotection, Diabetes, Autoimmune disease, Renal artery stenosis, Interstitial ephritis, Glomerulonephritis, Polycystic kidney disease, Systemic lupus erythematosus, Renal tubular acidosis, Adrenoleukodystrophy, Congenital Adrenal Hyperplasia, Cardiomyopathy, Atherosclerosis, Hypertension, Congenital heart defects, Aortic stenosis, Atrial septal defect (ASD), Atrioventricular (A-V) canal defect, Ductus arteriosus, Pulmonary stenosis, Subaortic stenosis, Ventricular septal defect VSD), valve diseases, Scleroderma, Obesity, Transplantation, Hyperthyroidism , Hypothyroidism, Fertility, Pancreatitis and/or other diseases/pathologies. The novel nucleic acid encoding the SN-like protein, and the SN-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

Potential therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues). The nucleic acids and proteins of the invention are useful in potential therapeutic applications implicated in various diseases and disorders described below and/or other pathologies and disorders.

The nucleic acids and proteins of the invention are useful in potential diagnostic and therapeutic applications implicated in various diseases and disorders described above and/or other pathologies. Moreover, the polypeptides can be used as immunogens to produce antibodies specific for the invention, and as vaccines. They can also be used to screen for potential agonist and antagonist compounds. The novel nucleic acid encoding a sialoadhesin-like protein, and the sialoadhesin-like protein of the invention, or fragments thereof, may further be useful in diagnostic applications, wherein the presence or amount of the nucleic acid or the protein are to be assessed.

These materials are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below.

NOV7

NOV7 is a novel protein encoded by a genomic DNA sequence that bears sequence similarity to Trio. Trio is a phosphoprotein identified in humans (See WO 97/35979) that is suggested to be a central organizer of multiple signaling pathways, to be involved in the activation of oncogenes such as c-fos, and to induce the transformation of cells. Trio has been found to be expressed in several tissues.

Trio is a complex protein possessing two guanine nucleotide exchange factor domains, each with adjacent pleckstrin homology and SH3 domains, a protein serine/threonine kinase domain with an adjacent immunoglobulin-like domain and multiple spectrin-like domains. Guanine nucleotide exchange factors, which promote the exchange of GDP for GTP, positively regulate Rho family GTPases and therefore participate in diverse cellular processes, including cell motility and cell growth. There is evidence that expression of fragments of Trio induces haptotactic cell migration and anchorage-independent growth (See Seipel, et al., J Cell Sci 112 (Pt 12):1825-34 (1999)).

The disclosed NOV7 is an alternative splice form of Trio. This variant was identified in SeqCalling assembly 3327669. SeqCalling is a differential expression and sequencing procedure that normalizes mRNA species in a sample and is disclosed in U.S. Ser. No. 09/417,386, filed Oct. 13, 1999, incorporated herein by reference in its entirety. SeqCalling assembly 3327669 was extended to provide clone 105180778 (NOV7). As noted NOV7 was found to be an alternative splice form of TRIO (Acc. No. U42390).

The disclosed NOV7 nucleic acid is shown in Table 7A.

TABLE 7A
NOV7 Nucleotide Sequence

(SEQ ID NO:25)

GGTGCGGAAGGTGAGGTTTTATATATATATCATTAAGACAACCCCAGGGGCGGAAAAAAGCTCCATCAACCACATTGAGA
CGGTGATGCAGCAGCTGGACGAGGCGCAGTCGCAGATGGAGGAGCTCTTCCAGGAGCGCAAGATCAAGCTGGAGCTCTTC
CTGCAGCTGCGCATCTTCGAGAGGGACGCCATCGACGTGAGTGTCCCGCGGCTGGCGCCTGCCTGCCTGTGGGAGCCCTT
GGCTTCCTCCACACCACCGGCGCCCTCTTGTCTCTGCCCTGCTGACGTCCTGTGTCCTCACCCACACCCCAACCCTTTGC
ACCAGGAGGGGGTGTGGGAGGGAGAGAGGGTCCCTGGAGGCTGGAATTGGATCATCCCTGGGAGGCTGGGGTATGCTCAG
CCCTGGACCCTAATGTTGGAAACTATAGAGCAGGCAGCTCGCGTGGCAGGACCAGAGCACAGCAAGCGCTGAGCGGAACA
GAAGGAAGCCAGCACAGCTTTCTCTCGGCCTTGGGGTGTTAAAACCTCGATTGGGTTGCACACAGCACTCCCTTGTCCTC
AGCTTAGGCACAGGCATCAGCTTCAGGCAGCAAGTCTTCCTCTCTTCCTCCCTGTCGTCTTCTCATCCTTCCCACTCTCC
TCCTGTCCTCACCCTCCCCTTTTCTCTCTCTCTCCACCTTCTCTCTCTCCTACCTTTTCTCTTCCTTGCTTCCTCCCTTT
CTCTTTOCATCTTTCTCTCTCTTGCCCTTCTCTCTTCTAGAGTGGGAATAATCCTGACTTGTGGTCGTAGCATTTTAGGT
AACAAAAAACACAATTTCCCCCCACAAAGAGCTGTTCTTAGATTATCCATCATTCTTACCTAAACGTGGACTTTTGCATC
TTATATGTGAACAGGAGAAAGAATTTACAGGTGTCTTTTATCTTTCTTCAACAAAAAGAGAGTGGTTCTGGGTTCTGTGC
TTAGGAGGAGCTCTCTTTTTTGATACCCCCCAACTTGCAGCAAATAGCCATACA

The NOV7 protein encoded by SEQ ID NO:25 is presented using the one-letter code in Table 7B (SEQ ID NO:26).

TABLE 7B
Encoded NOV7 protein sequence

(SEQ ID NO:26)

VRKVRFYTYIIKTTPGAEKSSINHIETVMQQLDEAQSQMEELFQERKIKLELFLQLRIFERDAIDVSVPRLAPACLWE
PLASSTAFAPSCLCPAEVLCPHPHPNPLHQEGVWEGERVPGGWNWIIPGRLGYAQFWTLMLETIEQAARVAGAEHSKR.

A comparison of NOV7 (SEQ ID NO:26) with Trio (AF091395) (SEQ ID NO:103) is provided in Table 7C.

[image]

NOV7 has been analyzed for tissue expression profiles. The quantitative expression of various clones was assessed in normal and tumor tissue samples by real time quantitative PCR (TAQMAN®) as described in Example 1, infra.

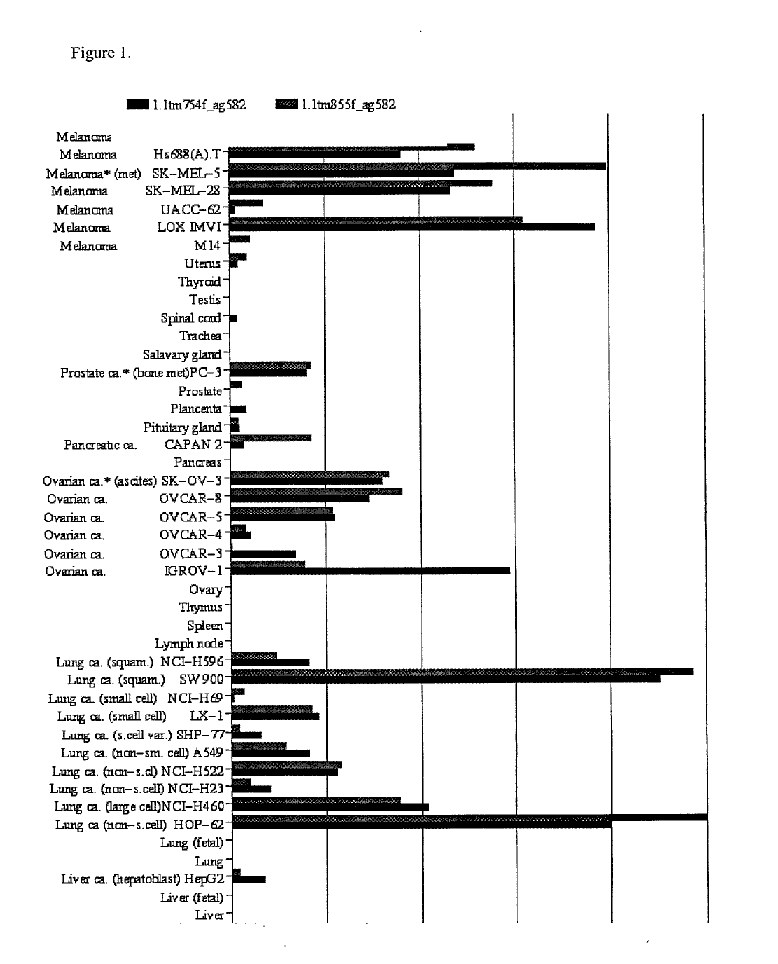

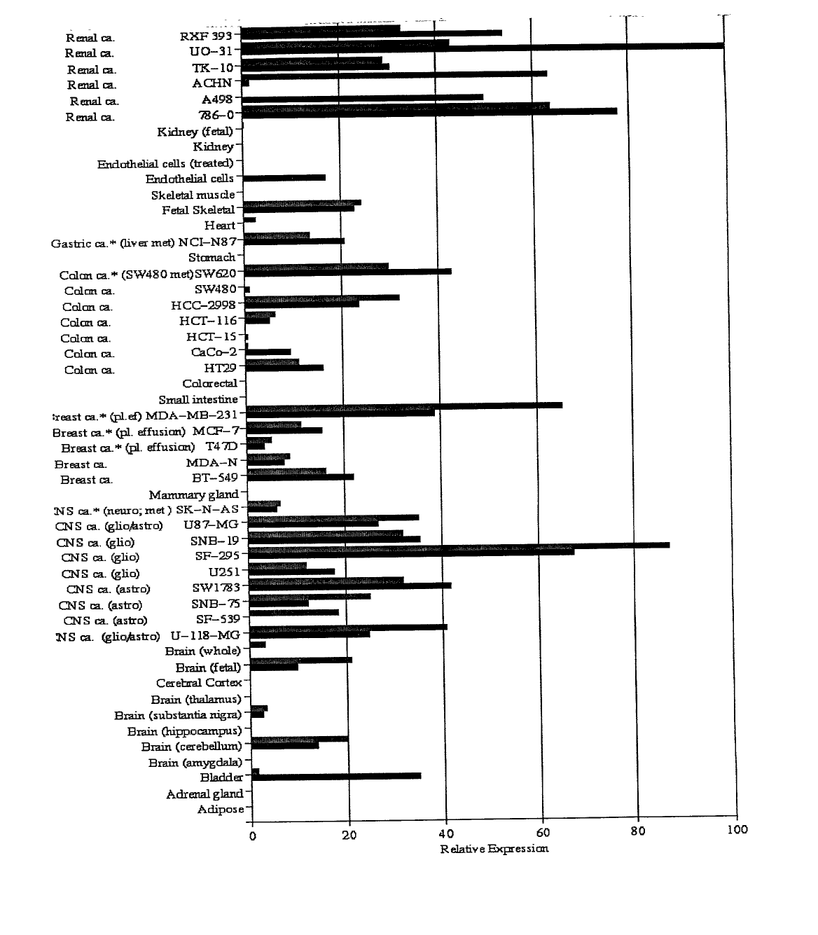

FIG. 1 shows a TaqMan tissue profile result. Two replicates of the same experiment are shown in gray and black bars. It is seen that the alternative splice form is overexpressed in cell lines derived from all major carcinomas groups, melanoma, ovary, lung, kidney, breast, brain. There is no expression, or very low expression, in most normal tissues.

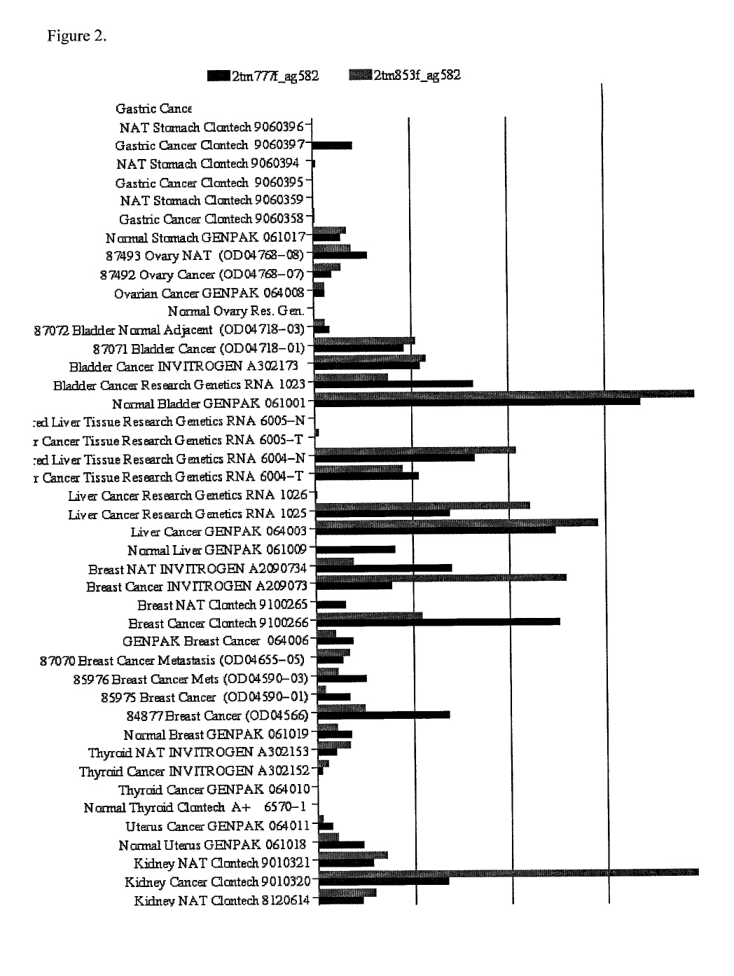

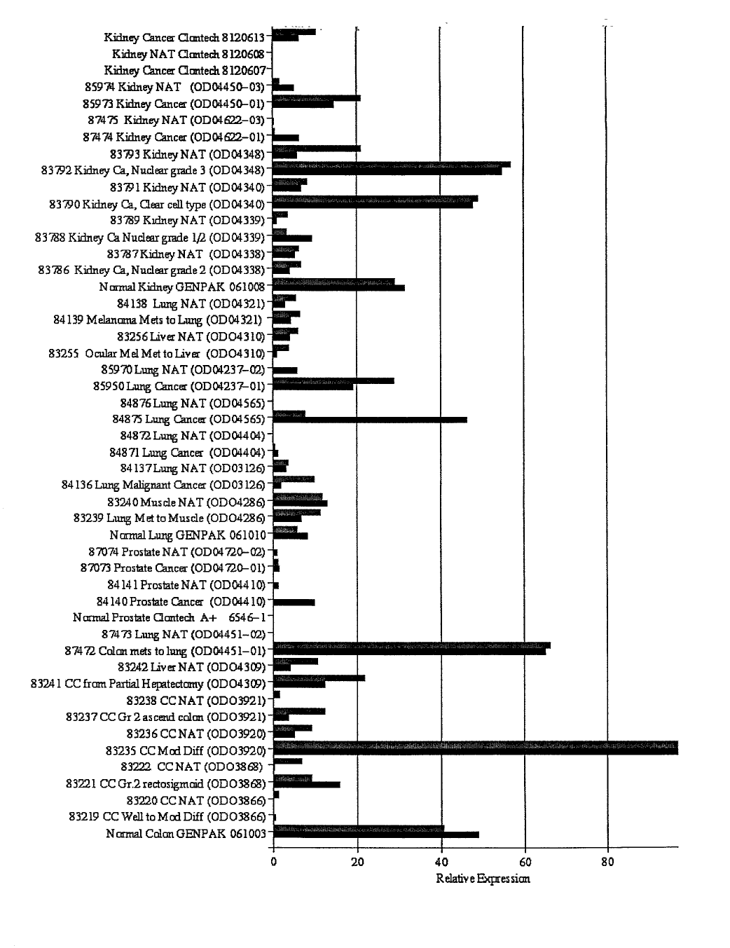

FIG. 2 provides replicate TaqMan profiles in a broader range of cancer cells that were derived from surgical specimens. Frequently these are juxtaposed with normal adjacent tissue (NAT) obtained at the same time by the operating surgeon. FIG. 2 shows that in colon, lung and kidney carcinomas, the Trio alternative splice form (NOV7) is overexpressed in the tumor compared to the normal adjacent tissue.

The disclosed NOV7 protein (SEQ ID NO:26) has identity with Trio phosphoproteins. The identity information used for ClustalW analysis is presented in Table 7D.

TABLE 7D
Identity Information Used for Clustal W Analysis

| Gene Index/ | | Length | Identity | Positives | |
|---|---|---|---|---|---|
| Identifier | Protein/Organism | (aa) | (%) | (%) | Expect |
| gi\|13646706\| | Triple functional | 1201 | 46/56 | 49/56 | 2e−17 |
| ref\|XP_003639.3\| | domain (PTPRF | | (82%) | (87%) | |
| | interacting) | | | | |
| | (Homo sapiens) | | | | |
| gi\|8928460\| | Triple Functional | 3038 | 44/56 | 48/56 | 1e−16 |
| sp\|O75962\| | Domain Protein | | (78%) | (85%) | |
| TRIO_HUMAN; | (PTPRF | | | | |
| gi\|3644048\| | Interacting | | | | |
| gb\|AAC43042.1\| | Protein); | | | | |
| (AF091395) | Trio isoform | | | | |
| | (Homo sapien) | | | | |
| gi\|6005922\| | Triple functional | 2861 | 44/56 | 48/56 | 1e−16 |
| ref\|NP_09049.1\|; | domain (PTPRF | | (78%) | (85%) | |
| gi\|3522970\| | interacting) | | | | |
| gb\|AAC34245.1\| | (Homo sapiens) | | | | |
| (U42390) | | | | | |
| gi\|7767545\|gb\| | Kalirin-7c | 1654 | 35/48 | 44/48 | 1e−08 |
| AAF69144.1\| | isoform | | (72%) | (90%) | |
| AF230644_1 | (Rattus | | | | |
| (AF230644) | norvegicus) | | | | |
| gi\|13645537\| | huntingtin- | 1564 | 35/48 | 44/48 | 1e−08 |
| ref\|XP_003026.2\| | associated | | (72%) | (90%) | |
| | protein | | | | |
| | interacting | | | | |
| | protein | | | | |
| | (Homo sapiens) | | | | |

This information is presented graphically in the multiple sequence alignment given in Table 7E (with NOV7 being shown on line 1) as a ClustalW analysis comparing NOV7 with related protein sequences.

[image]

[image]

[image]

[image]

[image]

[image]

[image]

Other BLAST results include sequences from the Patp database, which is a proprietary database that contains sequences published in patents and patent publications. Patp results include those listed in Table 7F.

TABLE 7F
Patp alignments of NOV7

| | | | | Smallest |
|---|---|---|---|---|
| | | | | Sum |
| Sequences producing High-scoring | | Reading | High | Prob. |
| Segment Pairs: | | Frame | Score | P (N) |
| Patp: AAW27227 | Human TRIO phospho- | +2 | 216 | 9.8e−13 |
| | protein, 2861 aa | | | |

For example, a BLAST against patp: AAW27227, a 2861 amino acid TRIO phosphoprotein (WO97/35979), produced good identity, E=9.8e-13).

The similarity information for the NOV7 protein and nucleic acid disclosed herein suggest that NOV7 may have important structural and/or physiological functions characteristic of Trio. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon. The novel nucleic acid encoding NOV7, and the disclosed NOV7 protein, or fragments thereof, may further be useful in diagnostic applications, wherein the presence or amount of the nucleic acid or the protein are to be assessed.

The disclosed NOV7 polypeptides can be used as immunogens to produce vaccines. The novel nucleic acid encoding NOV-like protein, and the NOV-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods. For example the disclosed NOV7 protein has multiple hydrophilic regions, each of which can be used as an immunogen. These novel proteins can also be used to develop assay system for functional analysis. These antibodies may be generated according to methods known in the art, using prediction from hydrophobicity charts, as described in the “Anti-NOVX Antibodies” section below.

NOV8

The present invention discloses a novel protein encoded by a cDNA and/or genomic DNA and proteins similar to it, namely, proteins bearing sequence similarity to Stra6.

NOV8a

This invention describes the following novel Stra6-like proteins and nucleic acids encoding them: 3277789_EXT (NOV8a). These sequences were initially identified by searching CuraGen's Human SeqCalling database for DNA sequences, which translate into proteins with similarity to Stra6-Like Proteins. SeqCalling is a differential expression and sequencing procedure that normalizes mRNA species in a sample, and is disclosed in U.S. Ser. No. 09/417,386, filed Oct. 13, 1999, incorporated herein by reference in its entirety. SeqCalling assembly 3277789 was identified as having suitable similarity. NOV8a was analyzed further to identify any open reading frames encoding novel full length proteins as well as novel splice forms of these genes. The SeqCalling assembly was extended using one or more sequences taken from additional SeqCalling assemblies, publicly available EST sequences and public genomic sequences. Public ESTs and additional CuraGen SeqCalling assemblies were identified by the CuraTools™ program SeqExtend. Such fragments were included in the DNA sequence extension for SeqCalling assembly 3277789 only when the extent of identity in the putative overlap region was high. The extent of identity may be, for example, about 90% or higher, preferably about 95% or higher, and even more preferably close to or equal to 100%. These inclusions, if used, are described below.

Genomic clone (acc:AC023545 HTG Homo sapiens chromosome 15 clone RP1 1-665J16 map 15, WORKING DRAFT SEQUENCE, 28 unordered pieces—Homo sapiens) was analyzed by Genscan and Grail to identify exons and putative coding sequences. This clone was also analyzed by TblastN, BlastX and other programs to identify genomic regions translating to proteins with similarity to the original protein or protein family of interest. It was identified as having regions with 100% identity to the SeqCalling assembly 3277789.

The results of these analyses were integrated and manually corrected for apparent inconsistencies that may have arisen, for example, from miscalled bases in the original fragments used. The sequences obtained encode the full-length proteins disclosed herein. When necessary, the process to identify and analyze cDNAs, ESTs and genomic clones was reiterated to derive the full-length sequence.

The disclosed NOV8a nucleic acid of 1962 nucleotides (also referred to as 3277789_EXT) is shown in Table 8A. An open reading begins with an ATG initiation codon at nucleotides 1-3 and ends with a TGA codon at nucleotides 1960-1962.

TABLE 8A
NOV8a Nucleotide Sequence

(SEQ ID NO:27)

ATGTCGTCCCAGCCAGCAGGGAACCAGACCTCCCCCCGGGCCACAGAGGACTACTCCTATGGCAGCTGGT
ACATCGATGAGCCCCAGGGGGGCGAGGAGCTCCAGCCAGAGGGGGAAGTGCCCTCCTGCCACACCAGCAT
ACCACCCGGCCTGTACCACGCCTGCCTGGCCTCGCTGTCAATCCTTGTGCTGCTCCTCCTGGCCATCCTG
GTGAGGCGCCGCCAGCTCTGGCCTGACTGTGTGCGTGGCACGCCCGGCCTGCCCAGCCCTGTGGATTTCT
TGGCTGGGGACAGGCCCCGGGCAGTGCCTGCTGCTGTITTCATGCTCCTCCTGAGCTCCCTCTGTTTCCT
GCTCCCCGACGAGGACGCATTGCCCTTCCTGACTCTCGCCTCAGCACCCAGCGGGGCCTGGAAGATACTG
GGACTGTTCTATTATGCTGCCCTCTACTACCCTCTGGCTGCCTGTGCCACGGCTGGCCACACACCTGCAC
ACCTGCTCGGCAGCACGCTGTCCTGGGCCCACCTTGGCGTCCAGGTCTGGCAGAGCGCAGAGTGTCCCCA
GGTGCCCAAGATCTACAAGTACTACTCCCTGCTGGCCTCCCTGCCTCTCCTGCTGGGCCTCGGATTCCTG
AGCCTTTGGTACCCTGTGCAGCTGGTGAGAAGCTTCAGCCGTAGCACAGGAGCAGGCTCCCAGGGGCTGC
AGAGCACCTACTCTGACCAATATCTGAGGAACCTCCTTTGCAGGAAGAAGCTGGGAAGCTGCAGCTACCA
CACCTCCAAGCATGGCTTCCTGTCCTGGGCCCGCGTCTGCTTGAGACACTGCATCTACACTCCACAGCCA
GGATTCCATCTCCCGCTGAAGCTGGTGCTTTCAGCTACACTGACAGGGACGGCCATTTACCAGGTAGCCC
TGCTGCTGCTGGTGGGCGTGGTACCCACTATCCAGAAGGTGAGGGCAGGGGTCACCACGGATGTCTCCTA
CCTGCTGGCCCGCTTTGGAATCGTGCTCTCCGAGGACAAGCAGGAGGTGGTCGAGCTGGTGAAGCACCAT
CTGTGGGCTCTGGAAGTGTGCTACATCTCACCCTTGGTCTTGTCCTGCTTACTCACCTTCCTGGTCCTGA
TGCGCTCACTGGTGACACACAGGACCAACCTTCGAGCTCTGCACCGAGGAGCTGCCCTGGACTTGAGTCC
CTTGCATCGGAGTCCCCATCCCTCCCGCCAAGCCATATTCTGTTGGATGAGCTTCAGTGCCTACCAGACA
GCCTTTATCTGCCTTGGTCTCCTGGTGCAGCAGATCATCTTCTTCCTGGGAACCACGGCCCTGGCCTTCC
TGGTGCTCATGCCTCTGCTCCATGGCAGGAACCTCCTGCTCTTCCGTTCCCTGGAGTCCTCATGGCCCTG
GCTTGTGATCCTGCAGAACATGGCAGCCCATTGGGTCTTCCTGGAGACTCATGATGGACACCCACAGCTG
ACCAACCGGCGAGTGCTCTATGCAGCCACCTTTCTTCTCTTCCCCCTCAATGTGCTGGTGGGTCCCATGG
TGGCCACCTGCCGAGTGCTCCTCTCTGCCCTCTACAACGCCATCCACCTTGGCCAGATGGACCTCAGCCT
GCTGCCACCGAGAGCCGCCACTCTCGACCCAGGCTACTACACGTACCGACCCTTCTTGAAGATTGAAGTC
AGCCAGTCGCATCCAGCCATGACAGCCTTCTGCTCCCTGCTCCTGCAAGCGCAGAGCCTCCTACCCAGGA
CCATGGCAGCCCCCCAGGACAGCCTCAGACCAGGGGAGGAAGACGAAGATATGCAGCTGCTACAGACAAA
GGACTCCATGGCCAAGGGAGCTAGGCCCGGGGCCAGCCGCGGCAGGGCTCGCTGGGGTCTGGCCTACACG
CTGCTGCACAACCCAACCCTGCAGGTCTTCCGCAAGACGGCCCTGTTGGGTGCCAATGGTGCCCAGCCCT
GA

The NOV8a protein encoded by SEQ ID NO:27 has 653 amino acid residues, and is presented using the one-letter code in Table 8B (SEQ ID NO:28). The SignalP, Psort and/or Hydropathy profile for NOV8 predict that NOV8 has a signal peptide and is likely to be localized within the plasma membrane with a certainty of 0.6000. It is also likely localized at the Golgi body (certainty=0.4000); endoplasmic reticulum (membrane) (certainty=0.3000); and microbody (peroxisome) (certainity=0.3000). The disclosed NOV8a protein is predicted to have a signal peptide that is likely cleaved between positions 8 and 9 (i.e., at the slash in the sequence AGN-QT).

TABLE 8B
Encoded NOV8a protein sequence

(SEQ ID NO:28)

MSSQPAGNQTSPGATEDYSYGSWYIDEFQGGEELQFEGEVPSCETSTPPCLYHACLASLSILVLLLLAML
VRRRQLWPDCVRGRPGLPSPVDFLAGDRPRAVPAAVFMVLLSSLCLLLPDEDALPFLTLASAPSGAWKTL
CLFYYAALYYPLAACATAGHTAAHLLGSTLSWAHLGVQVWQRAECPQVPKIYKYYSLLASLPLLLGLGFL
SLWYPVQLVRSFSRRTGAGSQGLQSSYSEEYLRNLLCRKKLGSCSYHTSKHGFLSWARVCLRHCIYTFQP
GFHLPLKLVLSATLTGTAIYQVALLLLVCVVPTIQKVRAGVTTDVSYLLACFGTVLSEDKQEVVELVKHH
LWALEVCYISALVLSCLLTFLVLMRSLVTHRTNLRALHRGAALDLSPLHRSPHPSRQATFCWNSFSAYQT
AFICLGLLVQQIIFFLGTTALAFLVLMPVLHGRNLLLFRSLESSWFWLVILQNMAAHWVFLETHDGHPQL
TNRRVLYAATFLLFPLNVLVGAMVATWRVLLSALYNAIHLGQMDLSLLPPRAATLDPGYYTYRNFLKIEV
SQSHPAMTAFCSLLLQAQSLLPRTMAAPQDSLRPGEEDEGMQLLQTKDSMAKGARPGASRGRARWGLAYT
LLHNPTLQVFRKTALLGANGAQP

The disclosed Stra6-like protein (NOV8a) maps to chromosome 15. Additionally, the disxlosed NOV8a protein is expressed in at least the following tissues: testis, bone, muscle, and blood-organ barriers. The protein disclosed herein is similar to the “Stra6-Like Protein Family”, some members of which end up localized at the cell surface where they exhibit activity. Therefore, it is likely that this novel Stra6-Like Protein is available at the appropriate sub-cellular localization and hence accessible for the therapeutic uses described herein.

NOV8b

In the present invention, the target sequence identified previously, Accession Number 3277789_Ext or CG52276-01 (NOV8a), was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated Accession Number CG52276-03 (NOV8b). NOV8b is a splice variant form and differs from the previously identified sequence (NOV8a) in having 9 additional internal amino acids and one amino acid change at position 59 S->P.

The sequence of the invention was derived by laboratory cloning of cDNA fragments covering the full length and/or part of the DNA sequence of the invention, and/or by in silico prediction of the full length and/or part of the DNA sequence of the invention from public human sequence databases.

The laboratory cloning was performed using one or more of the methods summarized below:

SeqCallingTM Technology:

cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Exon Linking:

The cDNA coding for the sequence was cloned by polymerase chain reaction (PCR) using the following primers: GGTCAAAGGAGAAGGGCCAGAGAAT (SEQ ID NO:63) and TTTTCTCAGGACCAAGTTTATTGCAGG (SEQ ID NO:64) on the following pool of human cDNAs: Pool 1—Adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus.

Primers were designed based on in silico predictions for the full length or part (one or more exons) of the DNA/protein sequence of the invention or by translated homology of the predicted exons to closely related human sequences or to sequences from other species. Usually multiple clones were sequenced to derive the sequence which was then assembled similar to the SeqCalling process. In addition, sequence traces were evaluated manually and edited for corrections if appropriate.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

The DNA sequence and protein sequence for a novel Retinoic Acid-Responsive Protein-like gene or one of its splice forms was obtained solely by exon linking and is reported here as NOV8b.

The disclosed NOV8b nucleic acid of 2012 bp (SEQ ID NO:29) is shown in Table 8C. An open reading frame was identified beginning at nucleotides 24-26 and ending at nucleotides 2010-2012. The start (ATG) and stop (TGA) codons of the open reading frame are highlighted in bold type. Putative untranslated regions, if any, are underlined.

TABLE 8C
NOV8b Nucleotide Sequence

| | |
|---|---|
| GGTCAAAGGAGAAGGGCCAGAGAATGTCGTCCCAGCCAGCAGGGAACCAGACCTCCCCCG | (SEQ ID NO:29) |
| GGGCCACAGAGGACTACTCCTATGGCAGCTGGTACATCGATGAGCCCCAGGGGGGCGAGG | |
| AGCTCCAGCCAGAGGGGGAAGTGCCCTCCTGCCACACCAGCATACCACCCGGCCTGTACC | |
| ACGCCTGCCTGGCCCCACTGTCAATCCTTGTGCTGCTGCTCCTGGCCATGCTGGTGAGCC | |
| GCCGCCAGCTCTCGCCTGACTGTGTGCGTGGCAGGCCCGGCCTGCCCAGCCCTGTGGATT | |
| TCTTGGCTGGGGACAGGCCCCGGGCAGTGCCTGCTGCTGTTTTCATGGTCCTCCTGAGCT | |
| CCCTGTGTTTGCTGCTCCCCGACGAGGACGCATTGCCCTTCCTGACTCTCGCCTCAGCAC | |
| CCAGCCAAGATGGGAAAACTGAGGCTCCAAGAGGGGCCTGGAAGATACTGGGACTGTTCT | |
| ATTATGCTGCCCTCTACTACCCTCTGGCTGCCTGTGCCACGGCTGGCCACACAGCTGCAC | |
| ACCTGCTCGGCAGCACGCTGTCCTGGGCCCACCTTGGGGTCCAGGTCTGGCAGAGGGCAG | |
| AGTGTCCCCAGGTCCCCAAGATCTACAAGTACTACTCCCTGCTGGCCTCCCTGCCTCTCC | |
| TGCTGGGCCTCGGATTCCTGAGCCTTTGGTACCCTGTGCAGCTGGTGAGAAGCTTCAGCC | |
| GTAGGACAGGAGCAGGCTCCCAGGGGCTCCAGAGCAGCTACTCTCAGCAATATCTGAGCA | |
| ACCTCCTTTGCAGGAAGAAGCTGGGAAGCTGCAGCTACCACACCTCCAAGCATGGCTTCC | |
| TGTCCTGGGCCCGCGTCTGCTTGAGACACTCCATCTACACTCCACAGCCAGGATTCCATC | |
| TCCCGCTGAAGCTGGTGCTTTCAGCTACACTGACAGGGACGGCCATTTACCAGGTAGCCC | |
| TGCTGCTGCTGGTGGGCGTGGTACCCACTATCCAGAAGGTGAGGGAAGCGGTCACCACGG | |
| ATGTCTCCTACCTGCTGGCCGGCTTTGGAATCGTGCTCTCCGAGGACAAGCAGGAGCTGG | |
| TGGAGCTGGTGAAGCACCATCTGTGGGCTCTGGAAGTGTGCTACATCTCAGCCTTGGTCT | |
| TGTCCTGCTTACTCACCTTCCTGGTCCTCATGCGCTCACTGGTGACACACAGGACCAACC | |
| TTCGAGCTCTGCACCGAGGAGCTGCCCTGGACTTGAGTCCCTTGCATCGGAGTCCCCATC | |
| CCTCCCGCCAAGCCATATTCTGTTGGATGAGCTTCAGTGCCTACCAGACAGCCTTTATCT | |
| GCCTTGGTCTCCTGGTGCAGCAGATCATCTTCTTCCTGGGAACCACGGCCCTGGCCTTCC | |
| TGGTGCTCATGCCTGTGCTCCATGGCAGGAACCTCCTGCTCTTCCGTTCCCTGGAGTCCT | |
| CATGCCCCTGGCTTGTGATCCTGCAGAACATGGCACCCCATTGGGTCTTCCTGGAGACTC | |
| ATGATGGACACCCACAGCTGACCAACCGGCGAGTGCTCTATGCAGCCACCTTTCTTCTCT | |
| TCCCCCTCAATGTGCTGGTGGGTGCCATGGTGGCCACCTGCCGAGTGCTCCTCTCTGCCC | |
| TCTACAACGCCATCCACCTTGGCCAGATGGACCTCAGCCTGCTGCCACCGAGAGCCGCCA | |
| CTCTCGACCCAGGCTACTACACGTACCGAAACTTCTTGAAGATTGAAGTCAGCCAGTCGC | |
| ATCCAGCCATGACAGCCTTCTGCTCCCTGCTCCTGCAAGCGCAGAGCCTCCTACCCAGGA | |
| CCATGGCAGCCCCCCAGGACAGCCTCAGACCAGGGGACGAAGACGAACGTATGCAGCTGC | |
| TACAGACAAAGGACTCCATGGCCAAGGAGCTAGGCCCGGGGGCCAGCCGCGGCAGGGCTC | |
| GCTGGGGTCTGGCCTACACGCTGCTGCACAACCCAACCCTGCAGGTCTTCCGCAAGACGG | |
| CCCTGTTGGGTGCCAATGGTGCCCAGCCCTGA | |

The NOV8b protein encoded by SEQ ID NO:29 has 662 amino acid residues, and is presented using the one-letter code in Table 8D (SEQ ID NO:30). The SignalP, Psort and Hydropathy profile for the Retinoic Acid-Responsive Protein-like protein predict that this sequence has a signal peptide and is likely to be localized at the plasma membrane with a certainty of 0.6000. NOV8b is also likely localized to the Golgi body (certainty=0.4000); endoplasmic reticulum (membrane) (certainty=0.3000); and microbody (peroxisome) (certainty=0.3000). The first 8 amino acids are more likely to be cleaved as a signal peptide based on the SignalP result (i.e., between the slash in the sequence AGN-QT).

TABLE 8D
Encoded NOV8b protein sequence

| | |
|---|---|
| MSSQPAGNQTSPGATEDYSYGSWYIDEPQGGEELQPEGEVPSCHTSIPPGLYHACLAPLS | (SEQ ID NO:30) |
| ILVLLLLAMLVRRRQLWPDCVRGRPGLPSPVDFLAGDRPRAVPAAVFMVLLSSLCLLLPD | |
| EDALPFLTLASAPSQDGKTEAPRGAWKILGLFYYAALYYPLAACATAGHTAAHLLGSTLS | |
| WAHLGVQVWQRAECPQVPKIYKYYSLLASLPLLLGLGFLSLWYPVQLVRSFSRRTGAGSQ | |
| GLQSSYSEEYLRNLLCRKKLGSCSYHTSKHGFLSWARVCLRHCIYTPQPGFHLPLKLVLS | |
| ATLTGTAIYQVALLLLVGVVPTIQKVRAGVTTDVSYLLAGFGIVLSEDKQEVVELVKHHL | |
| WALEVCYISALVLSCLLTFLVLMRSLVTHRTNLRALHRGAALDLSPLHRSPHPSRQAIFC | |
| WMSFSAYQTAFICLGLLVQQIIFFLGTTALAFLVLMPVLHGRNLLLFRSLESSWPWLVIL | |
| QNMAAHWVFLETHDGHPQLTNRRVLYAATFLLFPLNVLVGAMVATWRVLLSALYNAIHLG | |
| QMDLSLLPPRAATLDPGYYTYRNFLKIEVSQSHPAMTAFCSLLLQAQSLLPRTMAAPQDS | |
| LRPGEEDEGMQLLQTKDSMAKCARPGASRGRARWGLAYTLLHNPTLQVFRKTALLGANGA | |
| QE. | |

The disclosed NOV8b disclosed in this invention is expressed in at least the following tissues: Brain, Cervix, Heart, Kidney, Lymph node, Lymphoid tissue, Ovary, Pituitary Gland, Placenta, Retina, Temporal Lobe, Thyroid, Uterus, Whole Organism. This information was derived by determining the tissue sources of the sequences that were included in the invention including but not limited to SeqCalling sources, Public EST sources, Literature sources, and/or RACE sources.

NOV8c

The sequence of Acc. No. CG52276-04 (NOV8c) was derived by laboratory cloning of cDNA fragments, by in silico prediction of the sequence. cDNA fragments covering either the full length of the DNA sequence, or part of the sequence, or both, were cloned. In silico prediction was based on sequences available in Curagen's proprietary sequence databases or in the public human sequence databases, and provided either the full-length DNA sequence, or some portion thereof.

Exon Linking:

The cDNA coding for the CG52276-04 sequence was cloned by the polymerase chain reaction (PCR) using the primers: 5′ GTCAAAGGAGAAGGGCCAGAGAAT 3′ (SEQ ID NO:65) and 5′ TTTTCTCAGGACCAAGTTTATTGCAGG 3′ (SEQ ID NO:66). Primers were designed based on in silico predictions of the full length or some portion (one or more exons) of the cDNA/protein sequence of the invention. These primers were used to amplify a cDNA from a pool containing expressed human sequences derived from the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus.

Multiple clones were sequenced and these fragments were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical Clone:

The PCR product derived by exon linking, covering the entire open reading frame, was cloned into the pCR2.1 vector from Invitrogen to provide clone 90816::3277789.698482.C4.

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

The DNA sequence and protein sequence for a novel Retinoic Acid Responsive-like gene were obtained by exon linking and are reported here as NOV8c (CuraGen Acc. No. CG52276-04).

The disclosed NOV8c nucleic acid of 2620 bp (SEQ ID NO:31) is shown in Table 8E. An open reading frame was identified beginning at nucleotides 24-26 and ending at nucleotides 2025-2027. The start (ATG) and stop (TGA) codons of the open reading frame are highlighted in bold type. Putative untranslated regions, if any, are underlined.

TABLE 8E
NOV8c Nucleotide Sequence

| | |
|---|---|
| GGTCAAAGGAGAAGGGCCAGAGAATGTCGTCCCAGCCAGCAGGGAACCAGACCTCCCCCG | (SEQ ID NO:31) |
| GGGCCACAGAGGACTACTCCTATGGCAGCTGGTACATCGATGAGCCCCAGGGGGGCGAGG | |
| AGCTCCAGCCAGAGGGGGAAGTGCCCTCCTGCCACACCAGCATACCACCCGGCCTGTACC | |
| ACGCCTGCCTGGCCTCGCTCTCAATCCTTGTGCTGCTGCTCCTGGCCATGCTGGTGAGGC | |
| GCCGCCAGCTCTGGCCTCACTGTGTCCGTGGCAGGCCCGGCCTGCCCAGCCCTGTGGATT | |
| TCTTGCCTGGGGACAGGCCCCGGGCAGTGCCTGCTGCTGTTTTCATGATCCTCCTGAGCT | |
| CCCTGTGTTTCCTGCTCCCCGACCAGGACGCATTGCCCTTCCTGACTCTCGCCTCAGCAC | |
| CCAGCCAAGATCGGAAAACTGAGCCTCCAAGACGGGCCTGGAAGATACTGGGACTGTTCT | |
| ATTATGCTGCCCTCTACTACCCTCTGGCTCCCTGTGCCACGGCTGGCCACACAGCTGCAC | |
| ACCTGCTCGGCAGCACGCTGTCCTGGGCCCACCTTGGGGTCCAGGTCTGGCAGAGGGCAG | |
| AGTGTCCCCAGGTGCCCAAGATCTACAAGTACTACTCCCTGCTGGCCTCCCTGCCTCTCC | |
| TGCTGGGCCTCGGATTCCTGAGCCTTTGGTACCCTGTGCAGCTGGTGAGAAGCTTCAGCC | |
| GTAGGACAGGAGCAGGCTCCAAGGGGCTGCAGAGCAGCTACTCTGAGGAATATCTGAGGA | |
| ACCTCCTTTGCAGGAAGAAGCTGGGAAGCAGCTACCACACCTCCAAGCATGGCTTCCTGT | |
| CCTGGGCCCGCGTCTGCTTGAGACACTGCATCTACACTCCACAGCCACGATTCCATCTCC | |
| CGCTGAAGCTGGTCCTTTCAGCTACACTGACAGGGACGGCCATTTACCAGGTGGCCCTGC | |
| TGCTGCTGGTGGGCGTGGTACCCACTATCCAGAAGGTGAGGGCAGGGGTCACCACGGATG | |
| TCTCCTACCTGCPGGCCGGCTTTGGAATCGTGCTCTCCGAGGACAAGCAGGACGTGGTGG | |
| AGCTGGTGAAGCACCATCTGTGGCCTCTGGAAGTGTGCTACATCTCAGCCTTGGTCTTGT | |
| CCTGCTTACTCACCTTCCTGGTCCTGATGCGCTCACTGCTGACACACAGGACCAACCTTC | |
| GAGCTCTCCACCGAGGAGCTGCCCTGGACTTGAGTCCCTTGCATCGGACTCCCCATCCCT | |
| CCCGCCAAGCCATATTCTGTTGGATGAGCTTCAGTGCCTACCAGACAGCCTTTATCTGCC | |
| TTGGGCTCCTGGTGCAGCAGATCATCTTCTTCCTGGGAACCACGGCCCTGGCCTTCCTGG | |
| TGCTCATGCCTCTGCTCCATGGCAGGAACCTCCTGCTCTTCCCTTCCCTGGAGTCCTCGT | |
| GGCCCTTCTGGCTGACTTTGGCCCTGGCTGTGATCCTGCAGAACATGGCAGCCCATTGGG | |
| TCTTCCTGGAGACTCATGATGGACACCCACAGCTGACCAACCGGCGAGTGCTCTATGCAG | |
| CCACCTTTCTTCTCTTCCCCCTCAATGTGCTGGTGGGTGCCATGGTCCCCACCTGGCGAG | |
| TGCTCCTCTCTGCCCTCTACAACGCCATCCACCTTGGCCAGATGCACCTCAGCCTGCTGC | |
| CACCGAGAGCCGCCACTCTCGACCCCGGCTACTACACGTACCGAAACTTCTTGAAGATTG | |
| AAGTCAGCCAGTCGCATCCAGCCATCACAGCCTTCTGCTCCCTGCTCCTGCAAGCGCACA | |
| GCCTCCTACCCAGGACCATGGCAGCCCCCCAGGACAGCCTCAGACCAGGGGACGAAGACG | |
| AAGGGATGCAGCTGCTACAGACAAAGGACTCCATGGCCAAGGGAGCTAGCCCCGCGGCCA | |
| GCCGCGGCAGGGCTCGCTGGGGTCTGGCCTACACGCTGCTGCACAACCCAACCCTGCAGG | |
| TCTTCCGCAAGACGGCCCTGTTGGGTGCCAATGGTGCCCAGCCCTGAGGGCAGGGAAGGT | |
| CAACCCACCTGCCCATCTGTGCTGAGGCATGTTCCTGCCTACCATCCTCCTCCCTCCCCG | |
| GCTCTCCTCCCAGCATCACACCAGCCATGCAGCCAGCAGGTCCTCCGGATCACTGTGGTT | |
| GGGPGGAGGTCTGTCTGCACTGGGAGCCTCAGGAGCGCTCTGCTCCACCCACTTGGCTAT | |
| GGGAGAGCCAGCAGGGGTTCTGGAGAAAAAAAACTGGTGGGTTAGGGCCTTGGTCCAGGA | |
| GCCAGTTGAGCCAGGGCACCCACATCCAGGCGTCTCCCTACCCTGGCTCTGCCATCACCC | |
| TTGAAGGGCCTCGATGAAGCCTTCTCTGGAACCACTCCAGCCCAGCTCCACCTCAGCCTT | |
| GGCCTTCACGCTGTGGAACCAGCCAAGGCACTTCCTCACCCCCTCAGCGCCACGGACCTC | |
| TCTGGGGAGTGGCCGGAAAGCTCCCGCGCCTCTGGCCTGCAGGCCAGCCCAAGTCATGAC | |
| TCAGACCAGGTCCCACACTGAGCTGCCCACACTCGAGAGCCAGATATTTTTGTAGTTTTT | |
| ATCCCTTTGGCTATTATGAAAGAGGTTAGTGTGTTCCCTG | |

The NOV8c protein encoded by SEQ ID NO:31 has 667 amino acid residues, and is presented using the one-letter code in Table 8F (SEQ ID NO:32). The SignalP, Psort and Hydropathy profile for the Retinoic Acid-Responsive Protein-like protein predict that this sequence has a signal peptide and is likely to be localized at the plasma membrane with a certainty of 0.6000. NOV8b is also likely localized to the Golgi body (certainty=0.4000); endoplasmic reticulum (membrane) (certainty=0.3000); and microbody (peroxisome) (certainty=0.3000). The first 8 amino acids are more likely to be cleaved as a signal peptide based on the SignalP result (i.e., between the slash in the sequence AGN-QT).

TABLE 8F
Encoded NOV8c protein sequence

| | |
|---|---|
| MSSQPAGNQTSPGATEDYSYGSWYIDEPQGGEELQPEGEVPSCHTSIPPGLYHACLASLS | (SEQ ID NO:30) |
| ILVLLLLAMLVRRRQLWPDCVRGRPGLPSPVDFLAGDRPRAVPAAVFMILLSSLCLLLPD | |
| EDALPFLTLASAPSQDGKTEAPRGAWKILGLFYYAALYYPLAACATAGHTAAHLLGSTLS | |
| WAHLGVQVWQRAECPQVPKIYKYYSLLASLPLLLGLGFLSLWYPVQLVRSFSRRTGAGSK | |
| GLQSSYSEEYLRNLLCRKKGLSSYHTSKHGFLSWARVCLRHCIYTPQPGFHLPLKLVLSA | |
| TLTGTAIYQVALLLLVGVVPTIQKVRAGVTTDVSYLLAGFGIVLSEDKQEVVELVKHHLW | |
| ALEVCYISALVLSCLLTFLVLMRSLVTHRTNLRALHRGAALDLSPLHRSPHPSRQAIFCW | |
| MSFSAYQTAFICLGLLVQQIIFFLGTTALAFLVLMPVLHGRNLLLFRSLESSWPFWLTLA | |
| LAVTLQNNAAHWVFLETHDGHPQLTNRRVLYAATFLLFPLNVLVGAMVATWRVLLSALYN | |
| AIHLGQMDLSLLPPRAATLDPGYYTYRNFLKIEVSQSHPAMTAFCSLLLQAQSLLPRTMA | |
| APQDSLRPGEEDEGMQLLQTKDSMAKGARPGASRGRARWGLAYTLLHNPTLQVFRKTALL | |
| GANGAQP. | |

The disclosed NOV8b disclosed in this invention is expressed in at least the following tissues: Heart, Thyroid, Lymphoid tissue, Lymph node, Brain, Pituitary Gland, Temporal Lobe, Cervix, Ovary. Expression information was derived from the tissue sources of the sequences that were included in the derivation of the sequence of NOV8c (CuraGen Acc. No. CG52276-04). NOV8c maps to chromosome 15.

As used herein, any reference to NOV8 encompasses NOV8a, NOV8b, and NOV8c. A comparison of the NOV8 nucleic acid sequences is given in Table 8G. A comparison of the NOV8 amino acid sequences is given in Table 8H.

TABLE 8G
Comparison of NOV8 Nucleic Acid Sequences

| | |
|---|---|
| | 10 20 30 40 50 60 |
| | ....\|....\|....\|....\|....\|....\|....\|....\|....\|....\|....\|....\| |
| NOV8a | ----------------------- |
| NOV8b | |
| NOV8c | |
| | 70 60 90 100 110 120 |
| | ....\|....\|....\|....\|....\|....\|....\|....\|....\|....\|....\|....\| |
| NOV8a | |
| NOV8b | |
| NOV8c | |
| | 130 140 150 160 170 180 |
| | ....\|....\|....\|....\|....\|....\|....\|....\|....\|....\|....\|....\| |
| NOV8a | |
| NOV8b | |
| NOV8c | |
| | 190 200 210 220 230 240 |
| | ....\|....\|....\|....\|....\|....\|....\|....\|....\|....\|....\|....\| |
| NOV8a | |
| NOV8b | C A |
| NOV8c | |
| | 250 260 270 280 290 300 |
| | ....\|....\|....\|....\|....\|....\|....\|....\|....\|....\|....\|....\| |
| NOV8a | |
| NOV8b | |
| NOV8c | |
| | 310 320 330 340 350 360 |
| | ....\|....\|....\|....\|....\|....\|....\|....\|....\|....\|....\|....\| |
| NOV8a | |
| NOV8b | |
| NOV8c | |
| | A |
| | 370 380 390 400 410 420 |
| | ....\|....\|....\|....\|....\|....\|....\|....\|....\|....\|....\|....\| |
| NOV8a | |
| NOV8b | |
| NOV8c | |
| | 430 440 450 460 470 480 |
| | ....\|....\|....\|....\|....\|....\|....\|....\|....\|....\|....\|....\| |
| NOV8a | |
| NOV8b | |
| NOV8c | |
| | 490 500 510 520 530 540 |
| | ....\|....\|....\|....\|....\|....\|....\|....\|....\|....\|....\|....\| |
| NOV8a | |
| NOV8b | |
| NOV8c | |
| | 550 560 570 580 590 600 |
| | ....\|....\|....\|....\|....\|....\|....\|....\|....\|....\|....\|....\| |
| NOV8a | |
| NOV8b | |
| NOV8c | |
| | 610 620 630 640 650 660 |
| | ....\|....\|....\|....\|....\|....\|....\|....\|....\|....\|....\|....\| |
| NOV8a | |
| NOV8b | |
| NOV8c | |
| | 670 680 690 700 710 720 |
| | ....\|....\|....\|....\|....\|....\|....\|....\|....\|....\|....\|....\| |
| NOV8a | |
| NOV8b | |
| NOV8c | |
| | 730 740 750 760 770 780 |
| | ....\|....\|....\|....\|....\|....\|....\|....\|....\|....\|....\|....\| |
| NOV8a | |
| NOV8b | |
| NOV8c | A |
| | 790 800 810 820 830 840 |
| | ....\|....\|....\|....\|....\|....\|....\|....\|....\|....\|....\|....\| |
| NOV8a | |
| NOV8b | |
| NOV8c | |
| | 850 860 870 880 890 900 |
| | ....\|....\|....\|....\|....\|....\|....\|....\|....\|....\|....\|....\| |
| NOV8a | |
| NOV8b | |
| NOV8c | |
| | 910 920 930 940 950 960 |
| | ....\|....\|....\|....\|....\|....\|....\|....\|....\|....\|....\|....\| |
| NOV8a | |
| NOV8b | |
| NOV8c | G |
| | 970 980 990 1000 1010 1020 |
| | ....\|....\|....\|....\|....\|....\|....\|....\|....\|....\|....\|....\| |
| NOV8a | |
| NOV8b | |
| NOV9c | |
| | 1030 1040 1050 1060 1070 1080 |
| | ....\|....\|....\|....\|....\|....\|....\|....\|....\|....\|....\|....\| |
| NOV8a | |
| NOV8b | |
| NOV8c | |
| | 1090 1100 1110 1120 1130 1140 |
| | ....\|....\|....\|....\|....\|....\|....\|....\|....\|....\|....\|....\| |
| NOV8a | |
| NOV8b | |
| NOV8c | |
| | 1150 1160 1170 1180 1190 1200 |
| | ....\|....\|....\|....\|....\|....\|....\|....\|....\|....\|....\|....\| |
| NOV8a | |
| NOV8b | |
| NOV8c | |
| | 1210 1220 1230 1240 1250 1260 |
| | ....\|....\|....\|....\|....\|....\|....\|....\|....\|....\|....\|....\| |
| NOV8a | |
| NOV8b | |
| NOV8c | |
| | 1270 1280 1290 1300 1310 1320 |
| | ....\|....\|....\|....\|....\|....\|....\|....\|....\|....\|....\|....\| |
| NOV8a | |
| NOV8b | |
| NOV8c | |
| | 1330 1340 1350 1360 1370 1380 |
| | ....\|....\|....\|....\|....\|....\|....\|....\|....\|....\|....\|....\| |
| NOV8a | |
| NOV8b | |
| NOV8c | G |
| | 1390 1400 1410 1420 1430 1440 |
| | ....\|....\|....\|....\|....\|....\|....\|....\|....\|....\|....\|....\| |
| NOV8a | |
| NOV8b | |
| NOV8c | |
| | 1450 1460 1470 1480 1490 1500 |
| | ....\|....\|....\|....\|....\|....\|....\|....\|....\|....\|....\|....\| |
| NOV8a | --- --------------- |
| NOV8b | --- --------------- |
| NOV8c | G TCT GACTTTGGCCCTGGC |
| | 1510 1520 1530 1540 1560 1570 |
| | ....\|....\|....\|....\|....\|....\|....\|....\|....\|....\|....\|....\| |
| NOV8a | |
| NOV8b | |
| NOV8c | |
| | 1570 1580 1590 1600 1610 1620 |
| | ....\|....\|....\|....\|....\|....\|....\|....\|....\|....\|....\|....\| |
| NOV8a | |
| NOV8b | |
| NOV8c | |
| | 1630 1640 1650 1660 1670 1680 |
| | ....\|....\|....\|....\|....\|....\|....\|....\|....\|....\|....\|....\| |
| NOV8a | |
| NOV8b | |
| NOV8c | |
| | 1690 1700 1710 1720 1730 1740 |
| | ....\|....\|....\|....\|....\|....\|....\|....\|....\|....\|....\|....\| |
| NOV8a | |
| NOV8b | |
| NOV8c | C |
| | 1750 1760 1770 1780 1790 1800 |
| | ....\|....\|....\|....\|....\|....\|....\|....\|....\|....\|....\|....\| |
| NOV8a | |
| NOV8b | |
| NOV8c | |
| | 1810 1820 1830 1840 1850 1860 |
| | ....\|....\|....\|....\|....\|....\|....\|....\|....\|....\|....\|....\| |
| NOV8a | |
| NOV8b | |
| NOV8c | |
| | 1870 1880 1890 1900 1910 1920 |
| | ....\|....\|....\|....\|....\|....\|....\|....\|....\|....\|....\|....\| |
| NOV8a | |
| NOV8b | |
| NOV8c | G |
| | 1930 1940 1950 1960 1970 1980 |
| | ....\|....\|....\|....\|....\|....\|....\|....\|....\|....\|....\|....\| |
| NOV8a | |
| NOV8b | |
| NOV8c | |
| | 1990 2000 2010 2020 2030 2040 |
| | ....\|....\|....\|....\|....\|....\|....\|....\|....\|....\|....\|....\| |
| NOV8a | ---------- |
| NOV8b | ---------- |
| NOV8c | GGGCAGGGAA |
| | 2050 2060 2070 2080 2090 2100 |
| | ....\|....\|....\|....\|....\|....\|....\|....\|....\|....\|....\|....\| |
| NOV8a | ------------------------------------------------------------ |
| NOV8b | ------------------------------------------------------------ |
| NOV8c | GGTCAACCCACCTGCCCATCTGTGCTGAGGCATGTTCCTGCCTACCATCCTCCTCCCTCC |
| | 2110 2120 2130 2140 2150 2160 |
| | ....\|....\|....\|....\|....\|....\|....\|....\|....\|....\|....\|....\| |
| NOV8a | ------------------------------------------------------------ |
| NOV8b | ------------------------------------------------------------ |
| NOV8c | CCGGCTCTCCTCCCAGCATCACACCAGCCATGCAGCCAGCAGGTCCTCCGGATCACTGTG |
| | 2170 2180 2190 2200 2210 2220 |
| | ....\|....\|....\|....\|....\|....\|....\|....\|....\|....\|....\|....\| |
| NOV8a | ------------------------------------------------------------ |
| NOV8b | ------------------------------------------------------------ |
| NOV8c | GTTGGGTGGAGGTCTGTCTGCACTGGGAGCCTCAGGAGGGCTCTGCTCCACCCACTTGGC |
| | 2230 2240 2250 2260 2270 2280 |
| | ....\|....\|....\|....\|....\|....\|....\|....\|....\|....\|....\|....\| |
| NOV8a | ------------------------------------------------------------ |
| NOV8b | ------------------------------------------------------------ |
| NOV8c | TATGGGAGAGCCAGCAGGGGTTCTGGAGAAAAAAAACTGGTGGGTTACGGCCTTCGTCCA |
| | 2290 2300 2310 2320 2340 2350 |
| | ....\|....\|....\|....\|....\|....\|....\|....\|....\|....\|....\|....\| |
| NOV8a | ------------------------------------------------------------ |
| NOV8b | ------------------------------------------------------------ |
| NOV8c | GGAGCCAGTTGAGCCAGGGGCAGCCACATCCAGGCGTCTCCCTACCCTGGCTCTGCCATCA |
| | 2350 2360 2370 2380 2390 2400 |
| | ....\|....\|....\|....\|....\|....\|....\|....\|....\|....\|....\|....\| |
| NOV8a | ------------------------------------------------------------ |
| NOV8b | ------------------------------------------------------------ |
| NOV8c | GCCTTGAAGGGCCTCGATGAAGCCTTCTCTGGAACCACTCCAGCCCAGCTCCACCTCAGC |
| | 2410 2420 2430 2440 2450 2460 |
| | ....\|....\|....\|....\|....\|....\|....\|....\|....\|....\|....\|....\| |
| NOV8a | ------------------------------------------------------------ |
| NOV8b | ------------------------------------------------------------ |
| NOV8c | CTTGGCCTTCACGCTGTGGAAGCAGCCAAGGCACTTCCTCACCCCCTCAGCGCCACGGAC |
| | 2470 2480 2490 2500 2510 2520 |
| | ....\|....\|....\|....\|....\|....\|....\|....\|....\|....\|....\|....\| |
| NOV8b | ------------------------------------------------------------ |
| NOV8c | CTCTCTGGG0AGTGGCCGGAAAGCTCCCGGGCCTCTGGCCTGCAGGGCAGCCCAAGTCAT |
| | 2530 2540 2550 2560 2570 2580 |
| | ....\|....\|....\|....\|....\|....\|....\|....\|....\|....\|....\|....\| |
| NOV8a | ------------------------------------------------------------ |
| NOV8b | ------------------------------------------------------------ |
| NOV8c | GACTCAGACCAGGTCCCACACTGAGCTGCCCACACTCGAGAGCCAGATATTTTTCTAGTT |
| | 2590 2600 2610 2620 |
| | ....\|....\|....\|....\|....\|....\|....\|....\|... |
| NOV8a | ------------------------------------------- |
| NOV8b | ------------------------------------------- |
| NOV8c | TTTATGCCTTTGGCTATTATGAAAGAGGTTAGTGTGTTCCCTG |

[image]

[image]

BLAST analysis produced the significant results listed in Table 8I. The disclosed NOV8 proteins have good identity with a number of Stra6-like and retinoic acid responsive-like proteins.

TABLE 8I
BLAST results for NOV8

| Gene Index/ | | Length | Identity | Positives | |
|---|---|---|---|---|---|
| Identifier | Protein/Organism | (aa) | (%) | (%) | Expect |
| gi\|13560966\| | STRA6 isoform 1 | 667 | 535/668 | 536/668 | 0.0 |
| gb\|AAK30289.1\| | (Homo sapiens) | | (80%) | (80%) | |
| AF352728_1 | | | | Gaps = | |
| (AF352728) | | | | 16/668 | |
| | | | | (2%) | |
| gi\|13560968\| | STRA6 isoform 2 | 658 | 522/668 | 527/668 | 0.0 |
| gb\|AAK30290.1\| | (Homo sapiens) | | (78%) | (78%) | |
| AF352729_1 | | | | Gaps = | |
| (AF352729) | | | | 25/668 | |
| | | | | (3%) | |
| gi\|11641295\| | hypothetical | 560 | 462/561 | 463/561 | 0.0 |
| ref\|NP_071764.1\|; | protein FLJ12541 | | (82%) | (82%) | |
| gi\|10434086\| | similar to Stra6; | | | Gaps = | |
| dbj\|BAB14122.1\| | unnamed protein | | | 16/561 | |
| (AK022603) | product | | | (2%) | |
| | (Homo sapiens) | | | | |
| gi\|6678171\| | stimulated by | 670 | 396/672 | 447/672 | 0.0 |
| ref\|NP_033317.1\|; | retinoic acid | | (58%) | (65%) | |
| gi\|3126975\| | gene 6; | | | Gaps = | |
| gb\|AAC16016.1\| | retinoic acid- | | | 21/672 | |
| (AF062476) | responsive | | | (3%) | |
| | protein; STRA6 | | | | |
| | (Mus musculus) | | | | |
| gi\|13651719\| | hypothetical | 188 | 174/180 | 174/180 | 4e−86 |
| ref\|XP_016576.1\| | protein FLJ12541 | | | (96%) | |
| | similar to Stra6 | | | Gaps = | |
| | (Homo sapiens) | | | 6/180 | |
| | | | | (3%) | |

This information is presented graphically in the multiple sequence alignment given in Table 8J (with NOV8a being shown on line 1, NOV8b being shown on line 2, and NOV8c being shown on line 3) as a ClustalW analysis comparing NOV8 with related protein sequences.

[image]

[image]

[image]

Other BLAST results include sequences from the Patp database, which is a proprietary database that contains sequences published in patents and patent publications. Patp results include those listed in Table 8K.

TABLE 8K
Patp alignments of NOV8

| | | | | Smallest |
|---|---|---|---|---|
| | | | | Sum |
| Sequences producing High-scoring | | Reading | High | Prob. |
| Segment Pairs: | | Frame | Score | P (N) |
| Patp: AAB53256 | Human colon cancer | +1 | 807 | 6.1e−83 |
| | antigen protein, 178 aa | | | |
| Patp: AAW88559 | Secreted protein encoded | +1 | 441 | 1.5e−40 |
| | by gene 26, 88 aa | | | |

For example, a BLAST against patp:AAB53256, a 178 amino acid Human colon cancer antigen protein (WO/55351), produced good identity, E=6.1e-83). Moreover, a BLAST against patp:AAW88559, a 88 amino acid secreted protein encoded by gene 26 clone HTDAF28 (WO98/54963) from Homo sapiens, also produced good identity, E=1.5e-40.

Retinoic acid plays important roles in development, growth and differentiation by regulating the expression of target genes. A new retinoic acid-inducible gene, Stra6, has been identified in P19 embryonal carcinoma cells using a subtractive hybridization cDNA cloning technique. Stra6 codes for a very hydrophobic membrane protein of a new type, which does not display similarities with previously characterized integral membrane proteins. Stra6, which exhibits a specific pattern of expression during development and in the adult, is strongly expressed at the level of blood-organ barriers. Interestingly, in testis Sertoli cells, Stra6 has a spermatogenic cycle-dependent expression, which is lost in testes of RAR alpha null mutants where Stra6 is expressed in all tubules. The Stra6 protein may be a component of an as yet unidentified transport machinery. See generally Mech Dev 63(2):173-86 (1997); PMID: 9203140, UI: 97346723.

Using a differential substractive hybridization cloning procedure Stra6 has recently been identified as a novel retinoic acid-induced gene in murine P19 embryonal carcinoma cells. The putative amino acid sequence of Stra6 shows no similarity with previously characterised proteins. The pattern of expression of Stra6 transcripts during mouse limb development as revealed by in situ hybridization has been reported. In 8.5-9.0 days post-coitum (dpc) embryos, Stra6 was expressed in the lateral plate mesenchyme prior to limb bud outgrowth. By 9.5 dpc, expression was restricted to the proximal and dorsal forelimb bud mesoderm. Over the next 2 gestational days, Stra6 expression was specific of the dorsal mesoderm of the undifferentiated forelimb and hindlimb buds with the exception of their distal-most region or progress zone. A novel proximal-ventral expression domain appeared, however, by 11.0-11.5 dpc. Stra6 also remained expressed in the flank mesoderm. From 11.5-13.5 dpc, Stra6 expression was restricted to the superficial mesenchyme surrounding the chondrogenic blastemas, and progressively extended until the distal extremities of the limbs upon disappearance of the progress zone. Progressive restriction of Stra6 expression to perichondrium and developing muscles was seen at 13.5-14.5 dpc. Upon the initiation of endochondral ossification (15.5-16.5 dpc), Stra6 expression was limited to the area of perichondrium opposing cells of high metabolic and proliferative activity (the elongation zone). This suggests that Stra6 may play a role in early dorsoventral limb patterning and later in the control of endochondral ossification. See generally Dev Genet 19(1):66-73 (1996); PMID: 8792610, UI: 96384726.

Disruption of retinoic acid receptor (RAR) gamma in F9 embryonal carcinoma cells leads to aberrent differentiation and reduced activation of expression of several all-trans-retinoic acid (RA)-induced genes. The expression of several additional RA-responsive genes in RAR alpha- and RAR gamma-null F9 cells was analyzed. The RA-induced activation of Cdx1, Gap43, Stra4, and Stra6 was specifically impaired in RAR gamma-null cells, supporting the idea that each RAR may regulate distinct subsets of target genes. To further investigate the role of RAR gamma in F9 cell differentiation, “rescue” cell lines reexpressing RAR gamma 2 or overexpressing either RAR alpha 1 or RAR beta 2 were established in RAR gamma-null cells. Reexpression of RAR gamma or overexpression of RAR alpha restored both target-gene activation and the differentiation potential. In contrast, over-expression of RAR beta only poorly restored differentiation, although it could replace RAR gamma for the activation of target genes. See generally, Proc Natl Acad Sci USA 92(17):7854-58 (1995); PMID: 7644503, UI: 95372377.

Carcinogenesis involves inactivation or subversion of the normal controls of proliferation, differentiation, and apoptosis (Hurst et.al. Adv Exp Med Biol 462:449-67 (1999)). However, these controls are robust, redundant, and interlinked at the gene expression levels, regulation of mRNA lifetimes, transcription, and recycling of proteins. One of the central systems of control of proliferation, differentiation and apoptosis is retinoid signaling. The hRAR alpha nuclear receptor occupies a central position with respect to induction of gene transcription in that when bound to appropriate retinoid ligands, its homodimers and heterodimers with hRXR alpha regulate the transcription of a number of retinoid-responsive genes. These include genes in other signaling pathways, so that the whole forms a complex network. It has been shown that simple, cause-effect interpretations in terms of hRAR alpha gene transcription being the central regulatory event would not describe the retinoid-responsive gene network.

A set of cultured bladder-derived cells representing different stages of bladder tumorigenesis formed a model system. It consisted of two immortalized bladder cell lines (HUC-BC and HUC-PC), one squamous cell carcinoma cell line (SCaBER), one papilloma line (RT4), and 4 transitional cell carcinomas (TCC-Sup, 5637, T24, J82) of varying stages and grades. This set of cells was used to model the range of behaviors of bladder cancers. Relative gene expression before (constitutive) and after treatment with 10 microM all-trans-retinoic acid (aTRA) was measured for androgen and estrogen receptor; a set of genes involved with retinoid metabolism and action, hRAR alpha and beta, hRXR alpha and beta CRBP, CRABP I and II; and for signaling genes that are known to be sensitive to retinoic acid, EGFR, cytokine MK, ICAM I and transglutaminase. The phenotype for inhibition of proliferation and for apoptotic response to both aTRA and the synthetic retinoid 4-HPR was determined. Transfection with a CAT-containing plasmid containing an aTRA-sensitive promoter was used to determine if the common retinoic acid responsive element (RARE)-dependent pathway for retinoid regulation of gene expression was active. Each of the genes selected is known from previous studies to react to aTRA in a certain way, either by up- or down-regulation of the message and protein. A complex data set not readily interpretable by simple cause and effect was observed.

While all cell lines expressed high levels of the mRNAs for hRXR alpha and beta that were not altered by treatment with exogenous aTRA, constitutive and stimulated responses of the other genes varied widely among the cell lines. For example, CRABP I was not expressed by J82, T24, 5637 and RT4, but was expressed at low levels that did not change in SCaBER and at moderate levels that decreased, increased, or decreased sharply in HUC-BC, TCC-Sup and HUC-PC, respectively. The expression of hRAR alpha, which governs the expression of many retinoid-sensitive genes, was expressed at moderate to high levels in all cell lines, but in some it was sharply upregulated (TCC-Sup, HUC-PC and J82), remained constant (5637 and HUC-BC), or was down-regulated (SCaBER, T24 and RT4). The phenotypes for inhibition of proliferation showed no obvious relationship to the expression of any single gene, but cell lines that were inhibited by a TRA (HUC-BC and TCC-Sup) were not sensitive to 4-HPR, and vice versa. One line (RT4) was insensitive to either retinoid. Transfection showed very little retinoid-stimulated transfection of the CAT reporter gene with RT4 or HUC-PC. About 2-fold enhancement transactivation was observed with SCaBER, HUC-BC, J82 and T24 cells and 3-8 fold with 5637, TCC-Sup cells. In HUC-BC, a G to T point mutation was found at position 606 of the hRAR alpha gene. This mutation would substitute tyrosine for asparagine in a highly conserved domain.

These data indicate that retinoid signaling is probably a frequent target of inactivation in bladder carcinogenesis. The presence and functionality of retinoid signaling pathways in human urinary bladder carcinoma and SV40-immortalized uroepithelial cell lines has been examined. (See Waliszewski, et.al.; Mol Cell Endocrinol 148(1-2):55-65 (1999)). Only two of eight cell lines were proliferation-inhibited by 10 microM of either all-trans or 13-cis-retinoic acid. Transactivation of the CAT gene under control of a retinoid-responsive element demonstrated functionality of the signaling pathway in both sensitive cell lines and four of six resistant cell lines. Relative RT-PCR analysis of a panel of retinoid-responsive and inducible genes demonstrated changes in expression levels of all the genes in response to-retinoic acid treatment together with numerous aberrations dysregulations.

Retinoid signaling may be a target for inactivation during tumorigenesis by uncoupling gene expression, proliferation and differentiation. Therefore retinoids are more likely to be effective for chemoprevention than for treatment of bladder carcinomas. The proliferative effects of retinoids were examined in the MC-26 and LoVo colon adenocarcinoma cell lines (See Stewart, et.al. Exp Cell Res 233(2):321-29 (1997)). The proliferation of the LoVo cell line was not altered in the presence of the retinoids all trans-retinoic acid (atRA) and 9-cis-retinoic acid (9-cis-RA). Both retinoids, however, stimulated the growth, as measured by cell proliferation, of MC-26 cells. atRA and 9-cis-RA were equipotent in increasing MC-26 cell proliferation, suggesting that the growth stimulation is mediated by one or more retinoic acid receptors (RARs). To determine the RAR, which might be responsible for this growth stimulatory effect, the RAR subtypes which were present in both cell lines were characterized. mRNA for the RAR alpha, RAR beta, and RAR gamma were detected in the MC-26 cell. Of the RARs present in MC-26 cells, the RAR alpha does not mediate the growth stimulatory effects of retinoids, for a selective RAR alpha antagonist was unable to prevent the retinoid-induced increase in MC-26 cell growth. RAR alpha, RAR beta, and RAR gamma mRNA are also expressed in the LoVo cell line; the lack of growth-stimulation by retinoids in LoVo cells, therefore, does not seem to be due to the absence of RARs.

The results obtained in these experiments demonstrate that the growth response elicited by retinoids can vary between colon cancer cells and that the differences in response may not be solely determined by the RAR subtypes which are expressed in a colon cancer cell line.

Retinoic acids (RAs), well characterized regulators of proliferation and differentiation, partly re-differentiate follicular thyroid carcinoma cell lines (FTC-1 33, FTC-238, and HTC-TSHr) as well as SV40-transfected immortalized thyroid cell lines (ori3 and 7751) (See Schmutzler et.al Exp Clin Endocrinol Diabetes 104 Suppl 4:16-19 (1996)). This is indicated by the stimulation of type I 5′-deiodinase and other differentiation markers. As demonstrated by RT-PCR, electrophoretic mobility shift, and [3H]-retinoic acid binding assays, thyroid carcinoma cell lines express RA receptor mRNAs and functional ligand- and DNA-binding receptor proteins able to mediate RA-dependent signal transduction. Together, these properties make these thyroid-derived cell lines useful in vitro models for studying the effects of an RA re-differentiation therapy of thyroid cancer.

The chemotherapeutic agent retinoic acid (RA) inhibits the proliferation and invasion of many tumor types (See Vo et.al; Anticancer Res 18(1A):217-24 (1998)). RA chemotherapy in head and neck squamous cell carcinoma (SCC) patients reduces recurrence and induces regression of premalignant lesions. The effects of RA are mediated by both cytoplasmic and nuclear proteins. In the nucleus, a family of ligand-dependent transcription factors, the retinoic acid receptors (RAR) and the retinoid X receptors (RXR), regulate target gene response to RA. In the cytoplasm, the cellular retinoic acid binding proteins I and II (CRABP) regulate intracellular RA concentration, transport, and metabolism. Alterations in CRABP expression have been shown to affect target gene response and the phenotype of cancer cells.

To elucidate the role of these proteins in mediating the RA response, target gene expression and malignant phenotype in SCC25 cells expressing an antisense CRABP II construct was examined. RA induced CRABP II mRNA levels 2 fold in SCC25 cells by transcriptional upregulation. Expression of the antisense construct reduced CRABP II expression to undetectable levels. Inhibition of CRABP II expression resulted in significant downregulation of RA responsive genes. These reductions were the result of decreased transcription from RA responsive promoters. Surprisingly, clones expressing the antisense CRABP construct were less sensitive to RA mediated inhibition of proliferation. These clones were also less invasive in an in vitro invasion assay, likely due to downregulation of matrix metalloproteinase activity.

CRABP II affects the transcription of RA responsive genes which regulate proliferation and invasion of head and neck SCCs. All-trans-retinoic acid (trans-RA) and other retinoids exert anticancer effects through two types of retinoid receptors, the RA receptors (RARs) and retinoid X receptors (RXRs) (See Wu et.al.; Mol Cell Biol 17(11):6598-608 (1997)). Previous studies demonstrated that the growth-inhibitory effects of trans-RA and related retinoids are impaired in certain estrogen-independent breast cancer cell lines due to their lower levels of RAR alpha and RARbeta. In this study, we evaluated several synthetic retinoids for their ability to induce growth inhibition and apoptosis in both trans-RA-sensitive and trans-RA-resistant breast cancer cell lines. RXR-selective retinoids, particularly in combination with RAR-selective retinoids, could significantly induce RARbeta and inhibit the growth and induce the apoptosis of trans-RA-resistant, RAR alpha-deficient MDA-MB-23 1 cells but had low activity against trans-RA-sensitive ZR-75-1 cells that express high levels of RAR alpha. Using gel retardation and transient transfection assays, the effects of RXR-selective retinoids on MDA-MB-231 cells were most likely mediated by RXR-nur77 heterodimers that bound to the RA response element in the RARbeta promoter and activated the RARbeta promoter in response to RXR-selective retinoids. In contrast, growth inhibition by RAR-selective retinoids in trans-RA-sensitive, RAR alpha-expressing cells most probably occurred through RXR-RAR alpha heterodimers that also bound to and activated the RARbeta promoter. In MDA-MB-231 clones stably expressing RAR alpha, both RARbeta induction and growth inhibition by RXR-selective retinoids were suppressed, while the effects of RAR-selective retinoids were enhanced. Together, the results demonstrate that activation of RXR can inhibit the growth of trans-RA-resistant MDA-MB-231 breast cancer cells and suggest that low cellular RAR alpha may regulate the signaling switch from RAR-mediated to RXR-mediated growth inhibition in breast cancer cells. The protein similarity information, expression pattern, and map location for the Retinoic Acid-Responsive Protein-like protein and nucleic acid disclosed herein suggest that this Retinoic Acid-Responsive Protein may have important structural and/or physiological functions characteristic of the retionoic acid-responsive protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) biological defense weapon.

The nucleic acids and proteins of the invention are useful in potential diagnostic and therapeutic applications implicated in various diseases and disorders described below and/or other pathologies. For example, the compositions of the present invention will have efficacy for treatment of patients suffering from: Inflamation, Autoimmune disorders, Aging, cancer, Cardiomyopathy, Atherosclerosis,Hypertension, Congenital heart defects, Aortic stenosis ,Atrial septal defect (ASD),Atrioventricular (A-V) canal defect, Ductus arteriosus, Pulmonary stenosis, Subaortic stenosis, Ventricular septal defect (VSD), valve diseases, Tuberous sclerosis, Scleroderma, Obesity, Transplantation, Diabetes, Von Hippel-Lindau (VHL) syndrome, Pancreatitis, Obesity, Endometriosis, Fertility, Hemophilia, Hypercoagulation, Idiopathic thrombocytopenic purpura, Immunodeficiencies, Graft vesus host, Autoimmune disease, Renal artery stenosis, Interstitial nephritis, Glomerulonephritis, Polycystic kidney disease, Systemic lupus erythematosus, Renal tubular acidosis, IgA nephropathy, Hypercalceimia, Lesch-Nyhan as well as other diseases, disorders and conditions.

The novel nucleic acid encoding the NOV8 proteins, and the NOV8 proteins of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

The novel nucleic acid encoding NOV8 proteins, and the NOV8 proteins of the invention, or fragments thereof, may further be useful in diagnostic applications, wherein the presence or amount of the nucleic acid or the protein are to be assessed. These materials are further useful in the generation of antibodies that bind immuno-specifically to the novel NOV8 substances for use in therapeutic or diagnostic methods. These antibodies may be generated according to methods known in the art, using prediction from hydrophobicity charts, as described in the “Anti-NOVX Antibodies” section below. For example the disclosed NOV8a and NOV8b proteins have multiple hydrophilic regions, each of which can be used as an immunogen. For example, hydrophilic regions are found from about amino acid 5 to about amino acid 50; from about amino acid 80 to about amino acid 100; from about amino acid 180 to about amino acid 190; from about amino acid 210 to about amino acid 280; from about amino acid 345 to about amino acid 350; from about amino acid 390 to about amino acid 410; from about amino acid 490 to about amino acid 500; from about amino acid 540 to about amino acid 560; and from about amino acid 595 to about amino acid 630. These novel proteins can also be used to develop assay system for functional analysis.

NOV9

NOV9 includes a novel protein encoded by a genomic DNA sequence and proteins simiar to it, namely new proteins bearing sequence similarity to a protein encoded by thyroid regulated gene (TRG-like protein).

A novel nucleic acid was identified on chromosome 14 by TblastN using CuraGen Corporation's sequence file for TRG or homolog as run against the Genomic Daily Files made available by GenBank or from files downloaded from the individual sequencing centers. The nucleic acid sequence was predicted from the genomic file Genbank accession number: AL049870 by homology to a known TRG or homolog. Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequence designated SC 108341967_A (NOV9) encoding the full-length protein.

In a search of CuraGen Corporation's proprietary human expressed sequence assembly database, assembly 108341967 (552 nucleotides) was identified as having >95% homology to this predicted gene sequence. SeqCalling is a differential expression and sequencing procedure that normalizes mRNA species in a sample, and is disclosed in U.S. Ser. No. 09/417,386, filed Oct. 13, 1999, incorporated herein by reference in its entirety. This database is composed of the expressed sequences (as derived from isolated mRNA) from more than 96 different tissues. The mRNA is converted to cDNA and then sequenced. These expressed DNA sequences are then pooled in a database and those exhibiting a defined level of homology are combined into a single assembly with a common consensus sequence. The consensus sequence is representative of all member components. Since the disclosed NOV9 nucleic acid has >95% sequence identity with the CuraGen assembly, the nucleic acid of the invention represents an expressed gene sequence. This DNA assembly has 10 components.

A disclosed novel NOV9 nucleic acid is 1476 nucleotides long and is shown in Table 9A (SEQ ID NO:33). An ORF begins with an ATG initiation codon at nucleotides 1-3 and ends with a TAA codon at nucleotides 1474-1476. The start and stop codons are in bold letters in Table 9A.

TABLE 9A
NOV9 Nucleotide Sequence

(SEQ ID NO:33)

ATGAAGCAACTGCCTTTCCCACAGAAGTCAAAGACTTGACCAAGAGAATCTGCACTGTTCTTATGGCCA
CTGCCCAAATGAAGGAGCATGAGAACACCCTGAAATGCTAACTGATCTCCAATGTAGCTTAGCCAAGTC
CTATGCAAGTATCCCAGAGCTTAGGAAAACCTGGCTTGATAGCATGGCCAACATTCATGTAAAAAATGGA
GATTTTTCAGAGGCTGCAATGTGTTATGTCCATGCAGCAGCTCTAGTTGCAGAGTTTCTTAAAAGTACCT
ACTGGAAAAAGACCCAGAAGCTTCTTGGGACTTGTTTATACCATCCATGCAGCAGCTCTAGTTGCACACT
TTCTTCATTGAAAAAAAAATTTCCTAATGGATGTTCACCCTTCAAGAAAATTACTCCCAATATAGTTGAA
GAAGGAGCAGTGAAAGAACATGCTGGGATGATGGATGTCCATTATAGTGAAGAAGTTTTGCTCGACTTGC
TAGAACAATGTGTGGATAGCTTATGCAAGGCAAAACTTTATGAAATAATTTCTGAGATTTCCAAGTTGAT
CATTCCAATTTATGAGAAACATCCTGACTTTGAGAAACTTACTCAAGTTTATAGAACTCTTCAGGGAGCT
TACACAAATTATTCTGGAAAGTTATGCATACAAAAAAAAAAGAGAATTTTTTAGGCACTTTCTTCAGGTT
GCCTTTATGGCCAGTCTTTTTTTGAAGAAGATGGAAAGGAGTACATCTATAAAGAACCAAAGCTCACTGG
CCTCTCAGAAATTTCCCTGAGACTTGTTAAACTTTATGGTGAAAAATTTGGTATGGCGAATGTCAAAAAA
ATTCAGGATACAGACGAGGTAAATACCAAAGAGTTTGATCCAAAATATGCTCATATACAACTTACTTATG
TGAAGCCTTACTTTGATGACAAAGAACTCACAGAAAGAAACACCGAGTTTGGAAGAAATCATAATATCAG
CAGATTTGTTTTTGAGGCTCCTTACACTTTATCAGGCAAAAAGCAGGGTTGTACAGAAGAACAGTGCAAA
TGCCGTACAATCTTGACAACCTCAAAGTCATTTCCCTATGTGAAGAGGAGGATTCCTATTAACTGTGAAC
AGCAGATTAATTTAAAACCAATTGATGTTGCCACTGATGAAATAAAAGATAAAACTGCAGATCTGCAAAA
GCTTTCCTCCTCTGTTTATGTGGACATGATTCAACTCCAACTTAAATTGCAGGGCTGTGTTTCCATGCAG
GTCAATGCTGGTCCATTAGCATATGCAGGGGCTTTCTTAAATGATAGCCAAGCTAGCAAGTATCCACCTA
GGAAAGTGAGTGAGTTGAAAGACATGTTTAGGAAATCCATACAAGCATGCAGCATTGCACTTGAACTAAA
TGAGTGGCTAATTAAAGAACATCAAGTTGAGTACCATGAAGGGCTAAAGTCAAATTTCACACACCTGGTA
AAATAA

A disclosed NOV9 protein encoded by SEQ ID NO:33 has 491 amino acid residues, and is presented using the one-letter code in Table 9B (SEQ ID NO:34). The SignalP, Psort and/or Hydropathy profile for NOV9 predict that NOV9 has a signal peptide and is likely to be localized in the mitochondrial matrix space with a certainty of 0.4555. Using SIGNALP analysis, it is predicted that the protein of the invention has a signal peptide with most likely cleavage site between residues 49 and 50 in the sequence CSLAKSYA-SI.

NOV9 was found to be expressed in at least the following tissues: testis, uterus, nervous system, lymphatic system, and muscle.

TABLE 9B
Encoded NOV9 protein sequence

| | |
|---|---|
| MKATAFPTEVKDLTKRICTVLMATAQMKEHEKDPEMLTDLQCSLAKSYASIPELRKTWLDSMAN | (SEQ ID NO:34) |
| IHVKNGDFSEAANCYVHAAALVAEFLKSTYWKKTQKLLGTCLYHPCSSSSCRVSSLKKKFPNGC | |
| SPFKKITPNIVEEGAVKEDAGMMDVHYSEEVLLELLEQCVDSLWKAKLYEIISEI3KLIIPIYE | |
| KHPEFEKLTQVYRTLQGAYTKTLESYAYKKKREFFRHFLQSCLYGQSFFEEDGKEYTYKEPKLT | |
| GLSEISLRLVKLYGEKFGMANVKKIQDTDEVNTKEFDPKYAHIQVTYVKPYFDDKELTERKTEF | |
| GRNHNTSRFVFEAPYTLSGKKQGCTEEQCKCRTILTTSKSFPYVKRRIPINCEQQINLKPTDVA | |
| TDEIKDKTADLQKLCSSVYVDMIQLQLKLQGCVSMQVNAGPLAYAGAFLNDSQASKYPPRKVSE | |
| LKDMFRKSIQACSIALELNEWLIKEDQVEYHEGLKSNFRDVVK. | |

The disclosed NOV9 protein (SEQ ID NO:34) has good identity with members of th TRG family. The identity information used for ClustalW analysis is presented in Table 9C.

TABLE 9C
BLAST results for NOV9

| Gene Index/ | | Length | Identity | Positives | | |
|---|---|---|---|---|---|---|
| Identifier | Protein/Organism | (aa) | (%) | (%) | Expect | Gaps |
| gi\|12657106\| | bA155N3.2.1 | 1539 | 278/510 | 343/510 | e−140 | 30/510 |
| emb\|CAC27814.1\| | (KIAA1058) | | (54%) | (66%) | | (5%) |
| (AL161420) | (Homo sapiens) | | | | | |
| gi\|5689453\| | KIAA1058 protein | 1534 | 278/510 | 343/510 | e−140 | 30/510 |
| dbj\|BAA83010.1\| | (Homo sapiens) | | (54%) | (66%) | | (5%) |
| (AB028981) | | | | | | |
| gi\|7513980\| | gene trg protein - | 738 | 274/497 | 333/497 | e−137 | 16/497 |
| pir\|\|I60486; | rat (fragment); | | (55%) | (66%) | | (3%) |
| gi\|550420\| | trg | | | | | |
| emb\|CAA48220.1 | (Rattus | | | | | |
| (X68101) | norvegicus) | | | | | |
| gi\|8923210\| | hypothetical | 500 | 266/493 | 334/493 | e−131 | 8/493 |
| ref\|NP_060188.1\|; | protein FLJ20220; | | (53%) | (66%) | | (1%) |
| gi\|7020173\| | unnamed protein | | | | | |
| dbj\|BAA91022.1 | product | | | | | |
| (AK000227) | (Homo sapiens) | | | | | |
| gi\|7022394\| | unnamed protein | 415 | 191/368 | 253/368 | 7e−93 | 3/368 |
| dbj\|BAA91583.1\| | product | | (51%) | (67%) | | (0%) |
| (AK001253) | (Homo sapiens) | | | | | |

This information is presented graphically in the multiple sequence alignment given in Table 9D (with NOV9 being shown on line 1) as a ClustalW analysis comparing NOV9 with related protein sequences.

[image]

[image]

[image]

[image]

[image]

[image]

Other BLAST results include sequences from the Patp database, which is a proprietary database that contains sequences published in patents and patent publications. Patp results include those listed in Table 9E.

TABLE 9E
Patp alignments of NOV9

| | | | | Smallest |
|---|---|---|---|---|
| | | | | Sum |
| Sequences producing High-scoring | | Reading | High | Prob. |
| Segment Pairs: | | Frame | Score | P (N) |
| Patp: AAB64378 | Amino acid sequence | +1 | 1634 | 1.2e−208 |
| | of human intracellular | | | |
| | signaling, Homo sapiens, | | | |
| | 747 aa | | | |

For example, a BLAST against patp:AAB64378, a 747 amino acid human intracellular signaling molecule (INTRA 10) (WO00/77040) from Homo sapiens, produced good identity, E=1.2e-208.

The pattern of expression of this gene and its family members, and its similarity to the TRG family of genes suggests that it may function as a TRG family protein Therefore, the novel nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The nucleic acids and proteins of the invention are useful in potential therapeutic applications implicated in hypo- and hyperthyroidism, disorders of the thyroid, cancer including but not limited to thyroid-related cancers, and/or other pathologies and disorders. For example, a cDNA encoding the TRG-like protein may be useful in gene therapy, and the TRG-like protein may be useful when administered to a subject in need thereof. By way of nonlimiting example, the compositions of the present invention will have efficacy for treatment of patients suffering from hypo- and hyperthyroidism, disorders of the thyroid, cancer including but not limited to thyroid-related cancers. The novel nucleic acid encoding TRG-like protein, and the TRG-like protein of the invention, or fragments thereof, may further be useful in diagnostic applications, wherein the presence or amount of the nucleic acid or the protein are to be assessed. These materials are further useful in the generation of antibodies that bind immunospecifically to the novel substances of the invention for use in therapeutic or diagnostic methods.

These materials are further useful in the generation of antibodies that bind immuno-specifically to the novel NOV9 substances for use in therapeutic or diagnostic methods. These antibodies may be generated according to methods known in the art, using prediction from hydrophobicity charts, as described in the “Anti-NOVX Antibodies” section below.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, 25, 27, 29, 31, or 33, or a complement of this aforementioned nucleotide sequence, can be isolated using standard molecular biology techniques and the sequence information provided herein. Using all or a portion of the nucleic acid sequence of SEQ ID NOS 1, 3, 5, 7, 9, 11, 13, 15, 17, 19, 21, 23, 25, 27, 29, 31, or 33 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.) A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, 25,27,29, 31, and 33, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, 25, 27, 29, 31, or 33, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS: 1, 3, 5, 7, 9, 11, 13, 15, 17, 19, 21, 23, 25, 27, 29, 31, or 33 is one that is sufficiently complementary to the nucleotide sequence shown SEQ ID NOS:1, 3,5,7,9, 11, 13, 15, 17, 19,21,23,25,27,29,31, or 33 that it can hydrogen bond with little or no mismatches to the nucleotide sequence shown SEQ ID NOS: 1, 3, 5, 7, 9, 11, 13, 15, 17, 19, 21, 23, 25, 27, 29, 31, or 33, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, 25, 27, 29, 31, or 33, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, 25, 27, 29, 31, or 33; or an anti-sense strand nucleotide sequence of SEQ ID NOS: 1, 3, 5, 7, 9, 11, 13, 15, 17, 19, 21, 23, 25, 27, 29, 31, or 33; or of a naturally occurring mutant of SEQ ID NOS: 1, 3, 5, 7, 9, 11, 13, 15, 17, 19, 21, 23, 25, 27, 29, 31, or 33.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted. “A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, 25, 27, 29, 31, or 33, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, 25, 27, 29, 31, or 33 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, 25, 27, 29, 31, or 33. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 26, 28, 30, 32, or 34.

In addition to the human NOVX nucleotide sequences shown in SEQ ID NOS: 1, 3, 5, 7, 9, 11, 13, 15, 17, 19, 21, 23, 25, 27, 29, 31, or 33, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, 25, 27, 29, 31, or 33 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, 25, 27, 29, 31, or 33. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2× SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, 25, 27, 29, 31, or 33, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, 25, 27, 29, 31, or 33, or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, 25, 27, 29, 31, or 33, or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, 25, 27, 29, 31, or 33, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS:2, 4, 6, 8, 10, 12, 14, 16, 18, 20, 22, 24, 26, 28, 30, 32, or 34. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, 23, 25, 27, 29, 31, or 33 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS:2, 4, 6, 8, 10, 12, 14, 16, 18, 20, 22, 24, 26, 28, 30, 32, and 34. Preferably, the protein encoded by the nucleic acid molecule is at least about 60% homologous to SEQ ID NOS: 2, 4, 6, 8, 10, 12, 14, 16, 18, 20, 22, 24, 26, 28, 30, 32, and 34; more preferably at least about 70% homologous SEQ ID NOS:2, 4, 6, 8, 10, 12, 14, 16, 18, 20, 22, 24, 26, 28, 30, 32, or 34; still more preferably at least about 80% homologous to SEQ ID NOS:2, 4, 6, 8, 10, 12, 14, 16, 18, 20, 22, 24, 26, 28, 30, 32, or 34; even more preferably at least about 90% homologous to SEQ ID NOS:2, 4, 6, 8, 10, 12, 14, 16, 18, 20, 22, 24, 26, 28, 30, 32, or 34; and most preferably at least about 95% homologous to SEQ ID NOS:2, 4, 6, 8, 10, 12, 14, 16, 18, 20, 22, 24, 26, 28, 30, 32, or 34.

An isolated nucleic acid molecule encoding an NOVX protein homologous to the protein of SEQ ID NOS:2, 4, 6, 8, 10, 12, 14, 16, 18, 20, 22, 24, 26, 28, 30, 32, or 34 can be created by introducing one or more nucleotide substitutions, additions or deletions into the nucleotide sequence of SEQ ID NOS: 1, 3, 5, 7, 9, 11, 13, 15, 17, 19, 21, 23, 25, 27, 29, 31, or 33, such that one or more amino acid substitutions, additions or deletions are introduced into the encoded protein.

Mutations can be introduced into SEQ ID NOS: 1, 3, 5, 7, 9, 11, 13, 15, 17, 19, 21, 23, 25, 27, 29, 31, or 33 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, 25, 27, 29, 31, or 33, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof, (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, 25, 27, 29, 31, or 33, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS:2, 4, 6, 8, 10, 12, 14, 16, 18, 20, 22, 24, 26, 28, 30, 32, or 34, or antisense nucleic acids complementary to an NOVX nucleic acid sequence of SEQ ID NOS: 1, 3, 5, 7, 9, 11, 13, 15, 17, 19, 21, 23, 25, 27, 29, 31, or 33, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (ire., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, 25, 27, 29, 31, or 33). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS:2, 4, 6, 8, 10, 12, 14, 16, 18, 20, 22, 24, 26, 28, 30, 32, or 34. The invention also includes a mutant or variant protein any of whose residues may be changed from the corresponding residues shown in SEQ ID NOS:2, 4, 6, 8, 10, 12, 14, 16, 18, 20, 22, 24, 26, 28, 30, 32, or 34 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS:2, 4, 6, 8, 10, 12, 14, 16, 18, 20, 22, 24, 26, 28, 30, 32, or 34) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS:2, 4, 6, 8, 10, 12, 14, 16, 18, 20, 22, 24, 26, 28, 30, 32, or 34. In other embodiments, the NOVX protein is substantially homologous to SEQ ID NOS:2, 4, 6, 8, 10, 12, 14, 16, 18, 20, 22, 24, 26, 28, 30, 32, or 34, and retains the functional activity of the protein of SEQ ID NOS:2, 4, 6, 8, 10, 12, 14, 16, 18, 20, 22, 24, 26, 28, 30, 32, or 34, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS:2, 4, 6, 8, 10, 12, 14, 16, 18, 20, 22, 24, 26, 28, 30, 32, or 34, and retains the functional activity of the NOVX proteins of SEQ ID NOS:2, 4, 6, 8, 10, 12, 14, 16, 18, 20, 22, 24, 26, 28, 30, 32, or 34.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, 25, 27, 29, 31, or 33.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS:2, 4, 6, 8, 10, 12, 14, 16, 18, 20, 22, 24, 26, 28, 30, 32, or 34), whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline aphosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

The invention encompasses antibodies and antibody fragments, such as Fab or (Fab)2, that bind immunospecifically to any of the NOVX polypeptides of said invention.

An isolated NOVX protein, or a portion or fragment thereof, can be used as an immunogen to generate antibodies that bind to NOVX polypeptides using standard techniques for polyclonal and monoclonal antibody preparation. The full-length NOVX proteins can be used or, alternatively, the invention provides antigenic peptide fragments of NOVX proteins for use as immunogens. The antigenic NOVX peptides comprises at least 4 amino acid residues of the amino acid sequence shown SEQ ID NOS:2, 4, 6, 8, 10, 12, 14, 16, 18, 20, 22, 24, 26, 28, 30, 32, or 34 and encompasses an epitope of NOVX such that an antibody raised against the peptide forms a specific immune complex with NOVX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of NOVX that is located on the surface of the protein (e.g., a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc. Nat. Acad. Sci USA 78: 3824-3828; Kyte and Doolittle, 1982. J Mol. Biol. 157: 105-142, each incorporated herein by reference in their entirety).

As disclosed herein, NOVX protein sequences of SEQ ID NOS: 2, 4, 6, 8, 10, 12, 14, 16, 18, 20, 22, 24, 26, 28, 30, 32, and 34, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i.e., molecules that contain an antigen binding site that specifically-binds (immunoreacts with) an antigen, such as NOVX. Such antibodies include, but are not limited to, polyclonal, monoclonal, chimeric, single chain, F_{ab} and F_{(ab′)2} fragments, and an F_{ab} expression library. In a specific embodiment, antibodies to human NOVX proteins are disclosed. Various procedures known within the art may be used for the production of polyclonal or monoclonal antibodies to an NOVX protein sequence of SEQ ID NOS: 2, 4, 6, 8, 10, 12, 14, 16, 18, 20, 22, 24, 26, 28, 30, 32, and 34, or a derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly-expressed NOVX protein or a chemically-synthesized NOVX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against NOVX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of NOVX. A monoclonal antibody composition thus typically displays a single binding affinity for a particular NOVX protein with which it immunoreacts. For preparation of monoclonal antibodies directed towards a particular NOVX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an NOVX protein (see, e.g., U.S. Pat. No. 4,946,778). In addition, methods can be adapted for the construction of F_{ab} expression libraries (see, e.g., Huse, et al., 1989. Science 246: 1275-1281) to allow rapid and effective identification of monoclonal F_{ab} fragments with the desired specificity for an NOVX protein or derivatives, fragments, analogs or homologs thereof. Non-human antibodies can be “humanized” by techniques well known in the art. See, e.g., U.S. Pat. No. 5,225,539. Antibody fragments that contain the idiotypes to an NOVX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-NOVX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314 :446-449; Shaw, et al., 1988. J. Natl. Cancer Inst. 80: 1553-1559); Morrison(1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fuision expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSIoN TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banedji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, 25, 27, 29, 31, or 33 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, 23, 25, 27, 29, 31, or 33), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, 25, 27, 29, 31, or 33 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TFRATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermnann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, 25, 27, 29, 31, or 33, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, 25, 27, 29, 31, or 33 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, 25, 27, 29, 31, or 33, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qp Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7:5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6:1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofirans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLE 1

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources) and Panel 4 (containing cells and cell lines from normal cells and cells related to inflammatory conditions).

First, the RNA samples were normalized to constitutively expressed genes such as β-actin and GAPDH. RNA (50 ng total or 1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using P-actin and GAPDH TAQMAN® Assay Reagents (PE Biosystems; Catalog Nos. 4310881E and 4310884E, respectively) and TAQMAN® universal PCR Master Mix (PE Biosystems; Catalog No.4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR Conditions:

Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

Panel 1

In the results for Panel 1, the following abbreviations are used:

ca.=carcinoma,

*=established from metastasis,

met=metastasis,

s cell var=small cell variant,

non-s=non-sm=non-small,

squam=squamous,

pl.eff=pl effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s: 18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene ,La Jolla, Calif.) and thymus and kidney (Clontech) were employed. Total RNA from liver tissue from cirrhosis patients and kidney from lupus patients was obtained from BioChain (Biochain Institute, Inc., Hayward, CA). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 1100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 *g/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5,g/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 pM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-1 0 at 5 ng/ml was used to direct to Tr1. After 4-5 days, the activated Th 1, Th2 and Tr1 lymphocytes were washed once in DMEM and expanded for 4-7 days in DMEM 5% FCS (Hyclone), 100,M non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCDl06 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 ul DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

A summary of the TaqMan protocols used for each NOVX is provided in Table B.

TABLE B
Summary of TaqMan Protocols

| NOVX | Ag# |
|---|---|
| 1 | Ag3075 |
| 3a | Ag1247 |
| 3b | Ag1247 |
| 4a | AG1491/Ag676/ |
| | Ag1403 (identical) |
| | Ag721 Ag2835 |
| | Ag396 |
| 4b | Ag721, Ag1491, Ag1403, Ag2835 |
| 4c | Ag721, Ag1491, Ag1403, Ag2835 |
| 4d | Ag721, Ag1491, Ag1403, Ag2835 |
| 6 | Ag274 |
| 7 | Ag582 |
| 8a | Ag850 |
| 8b | Ag850 |
| 8c | Ag217 |
| 9 | Ag1249 |

NOV1

Expression of NOV1 was assessed using the primer-probe set Ag3075, described in Table C. Results of the RTQ-PCR runs are shown in Tables D, E and F.

TABLE C

Probe Name: Ag3O75

| | | | | |
|---|---|---|---|---|
| Primers | Sequences | TM | Length | Start Position |

| | | | | |
|---|---|---|---|---|
| Forward | 5′5-CAAACTTCTGAGCCAACTCG-3′ (SEQ ID | 58.8 | 20 | 153 |
| | NO: 76) | | | |
| Probe | FAM-5′-ATTGGAGAGGGCAGCTACTCCAAGGT-3′- | 69.1 | 26 | 193 |
| | TAMRA (SEQ ID NO:77) | | | |
| Reverse | 5′-TGTACTTCTTGGATGTGGCC-3′(SEQ ID | 58.6 | 20 | 225 |
| | NO:78) | | | |

TABLE D
Panel 1.3D

| | Relative | | Relative |
|---|---|---|---|
| | Expression (%) | | Expression (%) |
| | 1.3dx4tm5354f_{—} | | 1.3dx4tm5354f_{—} |
| Tissue Name | ag3075_b2 | Tissue Name | ag3075_b2 |
| Liver adenocarcinoma | 1.0 | Kidney (fetal) | 0.4 |
| Pancreas | 0.4 | Renal ca. 786-0 | 0.2 |
| Pancreatic ca. CAPAN 2 | 1.2 | Renal ca. A498 | 0.7 |
| Adrenal gland | 1.4 | Renal ca. RXF 393 | 0.6 |
| Thyroid | 1.5 | Renal ca. ACHN | 0.9 |
| Salivary gland | 0.2 | Renal ca. UO-31 | 0.7 |
| Pituitary gland | 0.9 | Renal ca. TK-10 | 0.2 |
| Brain (fetal) | 1.9 | Liver | 0.4 |
| Brain (whole) | 1.2 | Liver (fetal) | 1.4 |
| Brain (amygdala) | 1.2 | Liver ca. (hepatoblast) HepG2 | 0.3 |
| Brain (cerebellum) | 1.0 | Lung | 0.1 |
| Brain (hippocampus) | 1.5 | Lung (fetal) | 0.0 |
| Brain (substantia nigra) | 0.7 | Lung ca. (small cell) LX-1 | 0.0 |
| Brain (thalamus) | 2.0 | Lung ca. (small cell) NCI-H69 | 0.2 |
| Cerebral Cortex | 0.2 | Lung ca. (s. cell var.) SHP-77 | 1.5 |
| Spinal cord | 0.8 | Lung ca. (large cell) NCI-H460 | 1.5 |
| CNS ca. (glio/astro) U87-MG | 0.7 | Lung ca. (non-sm. cell) A549 | 1.3 |
| CNS ca. (glio/astro) U-118-MG | 3.0 | Lung ca. (non-s. cell) NCI-H23 | 0.3 |
| CNS ca. (astro) SW1783 | 0.5 | Lung ca (non-s. cell) HOP-62 | 0.4 |
| CNS ca.* (neuro; met) SK-N-AS | 1.5 | Lung ca. (non-s. cl) NCI-H522 | 0.6 |
| CNS ca. (astro) SF-539 | 0.8 | Lung ca. (squam.) SW 900 | 1.2 |
| CNS ca. (astro) SNB-75 | 1.1 | Lung ca. (squam.) NCI-H596 | 0.4 |
| CNS ca. (glio) SNB-19 | 1.3 | Mammary gland | 1.3 |
| CNS ca. (glio) U251 | 1.5 | Breast ca.* (pl. effusion) MCF-7 | 0.0 |
| CNS ca. (glio) SF-295 | 0.3 | Breast ca.* (pl. ef) MDA-MB- | 1.7 |
| | | 231 | |
| Heart (fetal) | 0.3 | Breast ca.* (pl. effusion) T47D | 0.8 |
| Heart | 0.5 | Breast ca. BT-549 | 0.3 |
| Fetal Skeletal | 0.0 | Breast ca. MDA-N | 0.4 |
| Skeletal muscle | 0.3 | Ovary | 0.5 |
| Bone marrow | 0.0 | Ovarian ca. OVCAR-3 | 1.0 |
| Thymus | 2.4 | Ovarian ca. OVCAR-4 | 0.8 |
| Spleen | 1.8 | Ovarian ca. OVCAR-5 | 0.5 |
| Lymph node | 2.0 | Ovarian ca. OVCAR-8 | 0.2 |
| Colorectal | 0.0 | Ovarian ca. IGROV-1 | 0.1 |
| Stomach | 2.7 | Ovarian ca.* (ascites) SK-OV-3 | 0.3 |
| Small intestine | 1.6 | Uterus | 0.4 |
| Colon ca. SW480 | 0.5 | Plancenta | 0.4 |
| Colon ca.* (SW480 met) SW620 | 0.3 | Prostate | 1.5 |
| Colon ca. HT29 | 0.4 | Prostate ca.* (bone met) PC-3 | 0.7 |
| Colon ca. HCT-116 | 0.0 | Testis | 100.0 |
| Colon ca. CaCo-2 | 0.4 | Melanoma Hs688(A).T | 0.4 |
| 83219 CC Well to Mod Diff | 0.2 | Melanoma* (met) Hs688(B).T | 0.2 |
| (ODO3866) | | | |
| Colon ca. HCC-2998 | 0.3 | Melanoma UACC-62 | 1.3 |
| Gastric ca.* (liver met) NCI-N87 | 3.2 | Melanoma M14 | 1.6 |
| Bladder | 0.2 | Melanoma LOX IMVI | 0.4 |
| Trachea | 0.6 | Melanoma* (met) SK-MEL-5 | 0.5 |
| Kidney | 0.2 | Adipose | 0.2 |

TABLE E
Panel 2D

| | Relative | | Relative |
|---|---|---|---|
| | Expression (%) | | Expression (%) |
| | 2dx4tm4819f_{—} | | 2dx4tm4819f_{—} |
| Tissue Name | ag3075_b2 | Tissue Name | ag3075_b2 |
| Normal Colon GENPAK | 11.1 | Kidney NAT Clontech 8120608 | 20.4 |
| 061003 | | | |
| 83219 CC Well to Mod Diff | 6.6 | Kidney Cancer Clontech | 54.3 |
| (ODO3866) | | 8120613 | |
| 83220 CC NAT (ODO3866) | 9.2 | Kidney NAT Clontech 8120614 | 25.5 |
| 83221 CC Gr.2 rectosigmoid | 3.0 | Kidney Cancer Clontech | 25.0 |
| (ODO3868) | | 9010320 | |
| 83222 CC NAT (ODO3868) | 0.8 | Kidney NAT Clontech 9010321 | 16.9 |
| 83235 CC Mod Diff | 15.2 | Normal Uterus GENPAK | 1.1 |
| (ODO3920) | | 061018 | |
| 83236 CC NAT (ODO3920) | 12.0 | Uterus Cancer GENPAK | 15.9 |
| | | 064011 | |
| 83237 CC Gr.2 ascend colon | 9.2 | Normal Thyroid Clontech A+ | 27.8 |
| (ODO3921) | | 6570-1 | |
| 83238 CC NAT (ODO3921) | 17.3 | Thyroid Cancer GENPAK | 7.6 |
| | | 064010 | |
| 83241 CC from Partial | 15.3 | Thyroid Cancer INVITROGEN | 12.4 |
| Hepatectomy (ODO4309) | | A302152 | |
| 83242 Liver NAT (ODO4309) | 13.7 | Thyroid NAT INVITROGEN | 27.7 |
| | | A302153 | |
| 87472 Colon mets to lung | 0.8 | Normal Breast GENPAK | 12.7 |
| (OD04451-01) | | 061019 | |
| 87473 Lung NAT (OD04451- | 8.4 | 84877 Breast Cancer | 10.7 |
| 02) | | (OD04566) | |
| Normal Prostate Clontech A+ | 58.6 | 85975 Breast Cancer | 86.1 |
| 6546-1 | | (OD04590-01) | |
| 84140 Prostate Cancer | 15.8 | 85976 Breast Cancer Mets | 100.0 |
| (OD04410) | | (OD04590-03) | |
| 84141 Prostate NAT | 17.5 | 87070 Breast Cancer Metastasis | 59.8 |
| (OD04410) | | (OD04655-05) | |
| 87073 Prostate Cancer | 22.9 | GENPAK Breast Cancer | 6.9 |
| (OD04720-01) | | 064006 | |
| 87074 Prostate NAT | 18.4 | Breast Cancer Res. Gen. 1024 | 12.6 |
| (OD04720-02) | | | |
| Normal Lung GENPAK 061010 | 29.5 | Breast Cancer Clontech | 22.4 |
| | | 9100266 | |
| 83239 Lung Met to Muscle | 11.5 | Breast NAT Clontech 9100265 | 7.2 |
| (ODO4286) | | | |
| 83240 Muscle NAT | 1.7 | Breast Cancer INVITROGEN | 13.6 |
| (ODO4286) | | A209073 | |
| 84136 Lung Malignant Cancer | 44.6 | Breast NAT INVITROGEN | 12.5 |
| (OD03126) | | A2090734 | |
| 84137 Lung NAT (OD03126) | 23.7 | Normal Liver GENPAK | 7.0 |
| | | 061009 | |
| 84871 Lung Cancer (OD04404) | 13.9 | Liver Cancer GENPAK 064003 | 3.8 |
| 84872 Lung NAT (OD04404) | 13.0 | Liver Cancer Research Genetics | 3.6 |
| | | RNA 1025 | |
| 84875 Lung Cancer (OD04565) | 1.1 | Liver Cancer Research Genetics | 8.1 |
| | | RNA 1026 | |
| 84876 Lung NAT (OD04565) | 7.2 | Paired Liver Cancer Tissue | 7.2 |
| | | Research Genetics RNA 6004-T | |
| 85950 Lung Cancer (OD04237- | 60.5 | Paired Liver Tissue Research | 11.5 |
| 01) | | Genetics RNA 6004-N | |
| 85970 Lung NAT (OD04237- | 7.1 | Paired Liver Cancer Tissue | 11.4 |
| 02) | | Research Genetics RNA 6005-T | |
| 83255 Ocular Mel Met to Liver | 15.0 | Paired Liver Tissue Research | 1.8 |
| (ODO4310) | | Genetics RNA 6005-N | |
| 83256 Liver NAT (ODO4310) | 6.8 | Normal Bladder GENPAK | 17.1 |
| | | 061001 | |
| 84139 Melanoma Mets to Lung | 6.9 | Bladder Cancer Research | 5.7 |
| (OD04321) | | Genetics RNA 1023 | |
| 84138 Lung NAT (OD04321) | 15.3 | Bladder Cancer INVITROGEN | 11.8 |
| | | A302173 | |
| Normal Kidney GENPAK | 34.7 | 87071 Bladder Cancer | 7.8 |
| 061008 | | (OD04718-01) | |
| 83786 Kidney Ca, Nuclear | 15.8 | 87072 Bladder Normal | 3.8 |
| grade 2 (OD04338) | | Adjacent (OD04718-03) | |
| 83787 Kidney NAT (OD04338) | 27.7 | Normal Ovary Res. Gen. | 3.7 |
| 83788 Kidney Ca Nuclear grade | 17.4 | Ovarian Cancer GENPAK | 24.3 |
| ½ (OD04339) | | 064008 | |
| 83789 Kidney NAT (OD04339) | 29.3 | 87492 Ovary Cancer | 38.6 |
| | | (OD04768-07) | |
| 83790 Kidney Ca, Clear cell | 3.2 | 87493 Ovary NAT (OD04768- | 6.3 |
| type (OD04340) | | 08) | |
| 83791 Kidney NAT (OD04340) | 22.4 | Normal Stomach GENPAK | 7.9 |
| | | 061017 | |
| 83792 Kidney Ca, Nuclear | 13.2 | Gastric Cancer Clontech | 1.8 |
| grade 3 (OD04348) | | 9060358 | |
| 83793 Kidney NAT (OD04348) | 17.2 | NAT Stomach Clontech | 4.7 |
| | | 9060359 | |
| 87474 Kidney Cancer | 18.5 | Gastric Cancer Clontech | 14.0 |
| (OD04622-01) | | 9060395 | |
| 87475 Kidney NAT (OD04622- | 7.5 | NAT Stomach Clontech | 16.4 |
| 03) | | 9060394 | |
| 85973 Kidney Cancer | 47.3 | Gastric Cancer Clontech | 15.0 |
| (OD04450-01) | | 9060397 | |
| 85974 Kidney NAT (OD04450- | 24.4 | NAT Stomach Clontech | 6.5 |
| 03) | | 9060396 | |
| Kidney Cancer Clontech | 21.5 | Gastric Cancer GENPAK | 15.0 |
| 8120607 | | 064005 | |

TABLE F
Panel 4D

| | Relative | | Relative |
|---|---|---|---|
| | Expression (%) | | Expression (%) |
| | 4dtm4708f_ag | | 4dtm4708f_ag |
| Tissue Name | 3075 | Tissue Name | 3075 |
| 93768_Secondary Th1_anti- | 33.9 | 93100_HUVEC | 0.0 |
| CD28/anti-CD3 | | (Endothelial)_IL-1b | |
| 93769_Secondary Th2_anti- | 43.5 | 93779_HUVEC | 11.2 |
| CD28/anti-CD3 | | (Endothelial)_IFN gamma | |
| 93770_Secondary Tr1_anti- | 35.1 | 93102_HUVEC | 7.2 |
| CD28/anti-CD3 | | (Endothelial)_TNF alpha + IFN | |
| | | gamma | |
| 93573_Secondary Th1_resting | 20.0 | 93101_HUVEC | 6.7 |
| day 4-6 in IL-2 | | (Endothelial)_TNF alpha + IL4 | |
| 93572_Secondary Th2_resting | 19.6 | 93781_HUVEC | 8.5 |
| day 4-6 in IL-2 | | (Endothelial)_IL-11 | |
| 93571_Secondary Tr1_resting | 21.2 | 93583_Lung Microvascular | 3.2 |
| day 4-6 in IL-2 | | Endothelial Cells_none | |
| 93568_primary Th1_anti- | 22.5 | 93584_Lung Microvascular | 18.6 |
| CD28/anti-CD3 | | Endothelial Cells_TNFa (4 | |
| | | ng/ml) and IL1b (1 ng/ml) | |
| 93569_primary Th2_anti- | 35.4 | 92662_Microvascular Dermal | 4.2 |
| CD28/anti-CD3 | | endothelium_none | |
| 93570_primary Tr1_anti- | 54.0 | 92663_Microsvasular Dermal | 3.7 |
| CD28/anti-CD3 | | endothelium_TNFa (4 ng/ml) | |
| | | and IL1b (1 ng/ml) | |
| 93565_primary Th1_resting dy | 73.7 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 41.5 | 93347_Small Airway | 9.9 |
| 4-6 in IL-2 | | Epithelium_none | |
| 93567_primary Tr1_resting dy | 42.3 | 93348_Small Airway | 21.3 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 8.8 |
| lymphocyte_anti-CD28/anti- | | SMC_resting | |
| CD3 | | | |
| 93352_CD45RO CD4 | 36.6 | 92669_Coronery Artery | 8.1 |
| lymphocyte_anti-CD28/anti- | | SMC_TNFa (4 ng/ml) and IL1b | |
| CD3 | | (1 ng/ml) | |
| 93251_CD8 Lymphocytes_anti- | 27.2 | 93107_astrocytes_resting | 9.4 |
| CD28/anti-CD3 | | | |
| 93353_chronic CD8 | 47.0 | 93108_astrocytes_TNFa (4 | 3.8 |
| Lymphocytes 2ry_resting dy 4- | | ng/ml) and IL1b (1 ng/ml) | |
| 6 in IL-2 | | | |
| 93574_chronic CD8 | 18.0 | 92666_KU-812 | 67.8 |
| Lymphocytes 2ry_activated | | (Basophil)_resting | |
| CD3/CD28 | | | |
| 93354_CD4_none | 23.8 | 92667_KU-812 | 80.7 |
| | | (Basophil)_PMA/ionoycin | |
| 93252_Secondary | 23.8 | 93579_CCD1106 | 31.2 |
| Th1/Th2/Tr1_anti-CD95 CH11 | | (Keratinocytes)_none | |
| 93103_LAK cells_resting | 17.9 | 93580_CCD1106 | 5.8 |
| | | (Keratinocytes)_TNFa and | |
| | | IFNg** | |
| 93788_LAK cells_IL-2 | 30.1 | 93791_Liver Cirrhosis | 7.1 |
| 93787_LAK cells_IL-2 + IL-12 | 54.0 | 93792_Lupus Kidney | 0.8 |
| 93789_LAK cells_IL-2 + IFN | 51.8 | 93577_NCI-H292 | 100.0 |
| gamma | | | |
| 93790_LAK cells_IL-2 + IL-18 | 45.7 | 93358_NCI-H292_IL-4 | 57.4 |
| 93104_LAK | 14.0 | 93360_NCI-H292_IL-9 | 77.4 |
| cells_PMA/ionomycin and IL- | | | |
| 18 | | | |
| 93578_NK Cells IL-2_resting | 28.1 | 93359_NCI-H292_IL-13 | 69.3 |
| 93109_Mixed Lymphocyte | 24.8 | 93357_NCI-H292_IFN gamma | 71.7 |
| Reaction_Two Way MLR | | | |
| 93110_Mixed Lymphocyte | 13.1 | 93777_HPAEC_- | 6.7 |
| Reaction_Two Way MLR | | | |
| 93111_Mixed Lymphocyte | 22.8 | 93778_HPAEC_IL-1 beta/TNA | 9.1 |
| Reaction_Two Way MLR | | alpha | |
| 93112_Mononuclear Cells | 6.0 | 93254_Normal Human Lung | 22.4 |
| (PBMCs)_resting | | Fibroblast_none | |
| 93113_Mononuclear Cells | 31.6 | 93253_Normal Human Lung | 10.7 |
| (PBMCs)_PWM | | Fibroblast_TNFa (4 ng/ml) and | |
| | | IL-1b (1 ng/ml) | |
| 93114_Mononuclear Cells | 13.4 | 93257_Normal Human Lung | 32.1 |
| (PBMCs)_PHA-L | | Fibroblast_IL-4 | |
| 93249_Ramos (B cell)_none | 33.9 | 93256_Normal Human Lung | 20.9 |
| | | Fibroblast_IL-9 | |
| 93250_Ramos (B | 37.6 | 93255_Normal Human Lung | 33.4 |
| cell)_ionomycin | | Fibroblast_IL-13 | |
| 93349_B lymphocytes_PWM | 64.6 | 93258_Normal Human Lung | 35.8 |
| | | Fibroblast_IFN gamma | |
| 93350_B lymphoytes_CD40L | 42.0 | 93106_Dermal Fibroblasts | 12.9 |
| and IL-4 | | CCD1070_resting | |
| 92665_EOL-1 | 92.7 | 93361_Dermal Fibroblasts | 54.0 |
| (Eosinophil)_dbcAMP | | CCD1070_TNF alpha 4 ng/ml | |
| differentiated | | | |
| 93248_EOL-1 | 42.6 | 93105_Dermal Fibroblasts | 5.8 |
| (Eosinophil)_dbcAMP/PMAiono- | | CCD1070_IL-1 beta 1 ng/ml | |
| mycin | | | |
| 93356_Dendritic Cells_none | 18.7 | 93772_dermal fibroblast_IFN | 14.9 |
| | | gamma | |
| 93355_Dendritic Cells_LPS | 23.5 | 93771_dermal fibroblast_IL-4 | 18.7 |
| 100 ng/ml | | | |
| 93775_Dendritic Cells_anti- | 18.0 | 93259_IBD Colitis 1** | 3.5 |
| CD40 | | | |
| 93774_Monocytes_resting | 3.1 | 93260_IBD Colitis 2 | 0.0 |
| 93776_Monocytes_LPS 50 | 3.6 | 93261_IBD Crohns | 3.5 |
| ng/ml | | | |
| 93581_Macrophages_resting | 21.8 | 735010_Colon_normal | 44.4 |
| 93582_Macrophages_LPS 100 | 8.3 | 735019_Lung_none | 29.3 |
| ng/ml | | | |
| 93098_HUVEC | 6.8 | 64028-1_Thymus_none | 48.3 |
| (Endothelial)_none | | | |
| 93099_HUVEC | 4.0 | 64030-1_Kidney_none | 37.9 |
| (Endothelial)_starved | | | |

Panel 1.3D Summary:

NOV1 is highly expressed in the testis, with expression levels being at least an order of magnitude lower in other tissues. Therefore this gene may be a marker for the testis and may be important in the regulation or dysregulation of spermatogenesis and fertility. Among other normal tissues, expression is detected at lower levels in fetal and adult brain; thyroid, adrenal and pituitary glands, thymus, spleen, lymph node, small intestine, fetal liver, mammary gland, prostate and spinal cord. In disease conditions, the highest expression is seen in a sample of gastric cancer, followed by CNS cancers, melanomas, lung, breast, pancreatic, liver and ovarian cancers. Therapeutics designed to this molecule may be effective in the treatment of infertility or cancer.

Panel 2D Summary:

This gene is expressed across a wide number of samples in panel 2D. Particularily it appears to be overexpressed in breast cancers relative to normal tissues as well as ovarian, kidney and lung cancers. Thus, inhibition of NOV1 function might be useful in the therapy of these or potentially other cancer types.

Panel 4D Summary:

The NOV1 transcript is expressed in many tissues regardless of treatment with the exception of colitis/inflammatory bowel disease (IBD) samples. Therapeutics designed to replace the protein encoded for by this transcript may reduce or eliminate inflammation due to inflammatory bowel diseases.

NOV3A

Expression of NOV3a was assessed using the primer-probe set Ag1247, described in Table G. Results of the RTQ-PCR runs are shown in Table H.

TABLE G

Probe Name: Agi 247

| | | | | |
|---|---|---|---|---|
| Primers | Sequences | TM | Length | Start Position |

| | | | | |
|---|---|---|---|---|
| Forward | 5′-GAATAGCTCCTGCTTGGATTTT-3′ (SEQ ID | 58.9 | 22 | 1413 |
| | NO:79) | | | |
| Probe | 5′-CTCACCTCTGCCTTCAGTCACTGGG-3′- | 69.6 | 25 | 1455 |
| | TAMPA (SEQ ID NO:80) | | | |
| Reverse | 5′-CTGCCTGTCTTACCATTGATGT-3′(SEQ ID | 59.1 | 22 | 1486 |
| | NO: 81) | | | |

TABLE H
Panel 4D

| | Relative | | Relative |
|---|---|---|---|
| | Expression (%) | | Expression (%) |
| | 4Dtm2106f_ag | | 4Dtm2106f_ag |
| Tissue Name | 1247 | Tissue Name | 1247 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 0.0 |
| day 4-6 in IL-2 | | Endothelial Cells_none | |
| 93568_primary Th1_anti- | 0.0 | 93584_Lung Microvascular | 0.0 |
| CD28/anti-CD3 | | Endothelial Cells_TNFa (4 | |
| | | ng/ml) and IL1b (1 ng/ml) | |
| 93569_primary Th2_anti- | 0.0 | 92662_Microvascular Dermal | 0.0 |
| CD28/anti-CD3 | | endothelium_none | |
| 93570_primary Tr1_anti- | 5.3 | 92663_Microsvasular Dermal | 0.0 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 0.0 |
| 4-6 in IL-2 | | epithelium_TNFa (4 ng/ml) and | |
| | | IL1b (1 ng/ml)** | |
| 93566_primary Th2_resting dy | 0.0 | 93347_Small Airway | 0.0 |
| 4-6 in IL-2 | | Epithelium_none | |
| 93567_primary Tr1_resting dy | 0.0 | 93348_Small Airway | 0.0 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 0.0 |
| lymphocyte_anti-CD28/anti- | | SMC_resting | |
| CD3 | | | |
| 93352_CD45RO CD4 | 7.6 | 92669_Coronery Artery | 0.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 8.0 |
| CD28/anti-CD3 | | | |
| 93353_chronic CD8 | 0.0 | 93108_astrocytes_TNFa (4 | 0.0 |
| Lymphocytes 2ry_resting dy 4- | | ng/ml) and IL1b (1 ng/ml) | |
| 6 in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.0 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and | |
| | | IFNg** | |
| 93788_LAK cells_IL-2 | 18.6 | 93791_Liver Cirrhosis | 16.8 |
| 93787_LAK cells_IL-2 + IL-12 | 0.0 | 93792_Lupus Kidney | 0.0 |
| 93789_LAK cells_IL-2 + IFN | 0.0 | 93577_NCI-H292 | 0.0 |
| gamma | | | |
| 93790_LAK cells_IL-2 + IL-18 | 8.6 | 93358_NCI-H292_IL-4 | 0.0 |
| 93104_LAK | 0.0 | 93360_NCI-H292_IL-9 | 0.0 |
| cells_PMA/ionomycin and IL- | | | |
| 18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.0 |
| Reaction_Two Way MLR | | alpha | |
| 93112_Mononuclear Cells | 0.0 | 93254_Normal Human Lung | 0.0 |
| (PBMCs)_resting | | Fibroblast_none | |
| 93113_Mononuclear Cells | 0.0 | 93253_Normal Human Lung | 0.0 |
| (PBMCs)_PWM | | Fibroblast_TNFa (4 ng/ml) and | |
| | | IL-1b (1 ng/ml) | |
| 93114_Mononuclear Cells | 0.0 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.0 |
| | | Fibroblast_IL-9 | |
| 93250_Ramos (B | 0.0 | 93255_Normal Human Lung | 0.0 |
| cell)_ionomycin | | Fibroblast_IL-13 | |
| 93349_B lymphocytes_PWM | 7.3 | 93258_Normal Human Lung | 0.0 |
| | | Fibroblast_IFN gamma | |
| 93350_B lymphoytes_CD40L | 0.0 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.0 |
| (Eosinophil)_dbcAMP/PMAiono- | | CCD1070_IL-1 beta 1 ng/ml | |
| mycin | | | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblast_IL-4 | 0.0 |
| 100 ng/ml | | | |
| 93775_Dendritic Cells_anti- | 0.0 | 93259_IBD Colitis 1** | 100.0 |
| CD40 | | | |
| 93774_Monocytes_resting | 0.0 | 93260_IBD Colitis 2 | 0.0 |
| 93776_Monocytes_LPS 50 | 0.0 | 93261_IBD Crohns | 0.0 |
| ng/ml | | | |
| 93581_Macrophages_resting | 0.0 | 735010_Colon_normal | 0.0 |
| 93582_Macrophages_LPS 100 | 0.0 | 735019_Lung_none | 11.8 |
| ng/ml | | | |
| 93098_HUVEC | 0.0 | 64028-1_Thymus_none | 0.0 |
| (Endothelial)_none | | | |
| 93099_HUVEC | 0.0 | 64030-1_Kidney_none | 0.0 |
| (Endothelial)_starved | | | |

Panel 4D Summary:

High expression of the NOV3a transcript is seen in colitis 1. The protein encoded for by this antigen may be important in the inflammatory process. Antagonistic antibodies or small molecule therapeutics may reduce or inhibit inflammation in the bowel due to IBD.

NOV4a

Expression of NOV4a was assessed using the primer-probe sets Ag 1491, Ag676 and Ag1403 (identical sequences), Ag721 and Ag2835, described in Tables I, J and K. Results of the RTQ-PCR runs are shown in Tables L, M, N and O.

TABLE I

Probe Name: Ag1491/Ag676/Ag14O3

| | | | | |
|---|---|---|---|---|
| Primers | Sequences | TM | Length | Start Position |

| | | | | |
|---|---|---|---|---|
| Forward | 5′-TAATGGAGAAGGCAGCAGAAG-3′ (SEQ ID | 59.6 | 21 | 1604 |
| | NO:82) | | | |
| Probe | TET-5′-TCTATACCCGGCTCAAGTCGCGG-3′-TAMRA | 702 | 23 | 1625 |
| | (SEQ ID NO:83) | | | |
| Reverse | 5′-CCCAGCCTTGTTCACTTTCT-3′(SEQ ID | 59.3 | 20 | 1676 |
| | NO: 84) | | | |

TABLE J
Probe Name: Ag721

| | | | | | Start |
|---|---|---|---|---|---|
| Primers | Sequences | | TM | Length | Position |
| Forward | 5′-ACCCAACAAGTACCCCATCTT-3′ | (SEQ ID NO:104) | 59.6 | 21 | 108 |
| Probe | FAM-5′-TTTCTTTGGCACACACGAAACGG-3′-TAMRA | (SEQ ID NO:105) | 68.2 | 23 | 129 |
| Reverse | 5′-TACATTTGTCGTAGGGGAACAG-3′ | (SEQ ID NO:106) | 59 | 22 | 172 |

TABLE K
Probe Name: Ag2835

| | | | | | Start |
|---|---|---|---|---|---|
| Primers | Sequences | | TM | Length | Position |
| Forward | 5′-GACCTTTAGGGCAAACTTGATC-3′ | (SEQ ID NO:107) | 59.1 | 22 | 757 |
| Probe TET-5′-ACTGTGCAGCTTCTGCAGCTTCTCCT-3′-TAMRA | (SEQ ID NO:108) | 69.8 | 26 | 781 | |
| Reverse | 5′-TGGACAGGAAGGTAGAGAAGAA-3′ | (SEQ ID NO:109) | 58.1 | 22 | 824 |

TABLE L
Panel 1.2

| | Relative | Relative | Relative |
|---|---|---|---|
| | Expression (%) | Expression (%) | Expression (%) |
| Tissue Name | 1.2tm1686f_ag1403* | 1.2tm895f_ag721 | 1.2tm2100t_ag1491 |
| Endothelial cells | 17.0 | 27.5 | 27.7 |
| Endothelial cells (treated) | 7.9 | 7.5 | 9.7 |
| Pancreas | 1.4 | 33.0 | 5.7 |
| Pancreatic ca. CAPAN 2 | 8.6 | 2.9 | 3.4 |
| Adrenal Gland (new lot*) | 25.7 | 15.5 | 23.2 |
| Thyroid | 1.2 | 24.1 | 2.2 |
| Salavary gland | 18.0 | 14.2 | 18.2 |
| Pituitary gland | 0.8 | 42.6 | 0.8 |
| Brain (fetal) | 2.6 | 12.4 | 0.9 |
| Brain (whole) | 8.9 | 27.7 | 2.1 |
| Brain (amygdala) | 10.6 | 8.8 | 6.5 |
| Brain (cerebellum) | 4.3 | 9.4 | 4.2 |
| Brain (hippocampus) | 27.5 | 16.6 | 20.6 |
| Brain (thalamus) | 16.0 | 9.3 | 11.3 |
| Cerebral Cortex | 38.4 | 23.0 | 42.6 |
| Spinal cord | 1.6 | 9.4 | 1.4 |
| CNS ca. (glio/astro) U87-MG | 17.1 | 14.9 | 20.4 |
| CNS ca. (glio/astro) U-118- | 16.7 | 12.9 | 19.8 |
| MG | | | |
| CNS ca. (astro) SW1783 | 8.4 | 5.8 | 7.5 |
| CNS ca.* (neuro; met) SK-N- | 24.1 | 38.7 | 18.3 |
| AS | | | |
| CNS ca. (astro) SF-539 | 8.7 | 9.8 | 6.8 |
| CNS ca. (astro) SNB-75 | 7.0 | 6.2 | 5.6 |
| CNS ca. (glio) SNB-19 | 12.5 | 9.9 | 16.0 |
| CNS ca. (glio) U251 | 6.9 | 5.7 | 6.4 |
| CNS ca. (glio) SF-295 | 32.5 | 13.2 | 17.7 |
| Heart | 63.3 | 18.8 | 62.8 |
| Skeletal Muscle (new lot*) | 95.3 | 100.0 | 100.0 |
| Bone marrow | 6.7 | 6.0 | 4.9 |
| Thymus | 2.0 | 7.5 | 1.7 |
| Spleen | 6.2 | 6.7 | 5.1 |
| Lymph node | 1.2 | 12.2 | 0.8 |
| Colorectal | 1.0 | 0.8 | 1.8 |
| Stomach | 3.7 | 14.5 | 5.4 |
| Small intestine | 21.0 | 19.1 | 22.8 |
| Colon ca. SW480 | 12.0 | 5.7 | 14.8 |
| Colon ca.* (SW480 | 15.6 | 15.7 | 17.4 |
| met) SW620 | | | |
| Colon ca. HT29 | 11.1 | 8.4 | 12.0 |
| Colon ca. HCT-116 | 29.9 | 13.5 | 28.1 |
| Colon ca. CaCo-2 | 15.8 | 12.2 | 12.2 |
| 83219 CC Well to Mod Diff | 1.2 | 0.7 | 1.3 |
| (ODO3866) | | | |
| Colon ca. HCC-2998 | 20.9 | 8.2 | 17.6 |
| Gastric ca.* (liver met) NCI-N87 | 5.6 | 7.5 | 6.5 |
| Bladder | 17.2 | 15.5 | 18.0 |
| Trachea | 1.1 | 7.7 | 0.5 |
| Kidney | 55.1 | 10.2 | 47.6 |
| Kidney (fetal) | 12.5 | 22.4 | 11.9 |
| Renal ca. 786-0 | 13.9 | 8.7 | 17.4 |
| Renal ca. A498 | 12.9 | 11.0 | 14.2 |
| Renal ca. RXF 393 | 4.3 | 2.7 | 5.6 |
| Renal ca. ACHN | 20.2 | 12.8 | 19.3 |
| Renal ca. UO-31 | 12.0 | 4.8 | 17.9 |
| Renal ca. TK-10 | 20.6 | 13.2 | 19.3 |
| Liver | 14.4 | 8.7 | 11.2 |
| Liver (fetal) | 10.7 | 8.5 | 7.8 |
| Liver ca. (hepatoblast) HepG2 | 17.2 | 4.5 | 17.9 |
| Lung | 1.0 | 6.5 | 0.8 |
| Lung (fetal) | 2.8 | 17.1 | 2.2 |
| Lung ca. (small cell) LX-1 | 18.4 | 10.4 | 15.8 |
| Lung ca. (small cell) NCI-H69 | 22.8 | 11.3 | 20.9 |
| Lung ca. (s. cell var.) SHP-77 | 6.7 | 6.2 | 5.8 |
| Lung ca. (large cell) NCI-H460 | 9.6 | 7.5 | 12.0 |
| Lung ca. (non-sm. cell) A549 | 7.0 | 6.2 | 7.2 |
| Lung ca. (non-s. cell) NCI-H23 | 31.0 | 11.3 | 19.5 |
| Lung ca (non-s. cell) HOP-62 | 35.8 | 16.5 | 23.7 |
| Lung ca. (non-s. cl) NCI-H522 | 100.0 | 53.2 | 85.9 |
| Lung ca. (squam.) SW 900 | 17.7 | 12.5 | 24.7 |
| Lung ca. (squam.) NCI-H596 | 33.7 | 16.4 | 37.1 |
| Mammary gland | 7.3 | 19.3 | 3.5 |
| Breast ca.* (pl. effusion) MCF-7 | 8.4 | 7.3 | 8.6 |
| Breast ca.* (pl. ef) MDA-MB-231 | 7.6 | 8.7 | 6.3 |
| Breast ca.* (pl. effusion) T47D | 9.5 | 10.7 | 12.0 |
| Breast ca. BT-549 | 7.9 | 10.1 | 6.8 |
| Breast ca. MDA-N | 31.9 | 15.4 | 18.8 |
| Ovary | 21.5 | 18.4 | 21.9 |
| Ovarian ca. OVCAR-3 | 17.0 | 21.2 | 20.9 |
| Ovarian ca. OVCAR-4 | 17.2 | 8.7 | 15.4 |
| Ovarian ca. OVCAR-5 | 33.7 | 22.4 | 35.6 |
| Ovarian ca. OVCAR-8 | 17.6 | 9.8 | 21.8 |
| Ovarian ca. IGROV-1 | 21.2 | 15.4 | 24.5 |
| Ovarian ca.* (ascites) SK-OV-3 | 45.7 | 29.9 | 48.6 |
| Uterus | 8.8 | 14.6 | 6.6 |
| Plancenta | 1.3 | 15.5 | 1.5 |
| Prostate | 29.3 | 18.8 | 25.0 |
| Prostate ca.* (bone met) PC-3 | 51.8 | 35.1 | 40.3 |
| Testis | 4.7 | 65.5 | 10.6 |
| Melanoma Hs688(A).T | 7.7 | 5.4 | 5.8 |
| Melanoma* (met) Hs688(B).T | 5.6 | 4.9 | 3.3 |
| Melanoma UACC-62 | 20.2 | 14.6 | 18.0 |
| Melanoma M14 | 10.2 | 4.9 | 11.0 |
| Melanoma LOX IMVI | 14.4 | 8.6 | 9.2 |
| Melanoma* (met) SK-MEL-5 | 10.7 | 8.1 | 8.8 |
| Adipose | 10.4 | 0.7 | 12.4 |

TABLE M
Panel 1.3D

| | Relative | Relative |
|---|---|---|
| | Expression (%) | Expression (%) |
| | 1.3Dtm3819t_ag | 1.3dtm4286f_ag |
| Tissue Name | 2835 | 721 |
| Liver adenocarcinoma | 17.0 | 13.7 |
| Pancreas | 7.0 | 4.5 |
| Pancreatic ca. CAPAN 2 | 3.5 | 5.0 |
| Adrenal gland | 6.8 | 5.8 |
| Thyroid | 14.9 | 11.6 |
| Salivary gland | 6.4 | 1.5 |
| Pituitary gland | 18.2 | 11.7 |
| Brain (fetal) | 9.3 | 8.5 |
| Brain (whole) | 19.5 | 13.0 |
| Brain (amygdala) | 19.8 | 11.6 |
| Brain (cerebellum) | 10.4 | 6.7 |
| Brain (hippocampus) | 100.0 | 62.4 |
| Brain (substantia nigra) | 5.7 | 2.9 |
| Brain (thalamus) | 15.1 | 11.1 |
| Cerebral Cortex | 58.6 | 32.3 |
| Spinal cord | 7.8 | 3.8 |
| CNS ca. (glio/astro) U87-MG | 21.8 | 15.9 |
| CNS ca. (glio/astro) U-118-MG | 69.3 | 74.7 |
| CNS ca. (astro) SW1783 | 16.3 | 14.8 |
| CNS ca.* (neuro; met) SK-N-AS | 96.6 | 84.1 |
| CNS ca. (astro) SF-539 | 15.4 | 15.5 |
| CNS ca. (astro) SNB-75 | 19.8 | 14.8 |
| CNS ca. (glio) SNB-19 | 8.5 | 9.7 |
| CNS ca. (glio) U251 | 7.6 | 11.2 |
| CNS ca. (glio) SF-295 | 17.1 | 23.0 |
| Heart (fetal) | 16.2 | 21.8 |
| Heart | 3.7 | 2.9 |
| Fetal Skeletal | 85.9 | 100.0 |
| Skeletal muscle | 11.6 | 6.2 |
| Bone marrow | 9.0 | 5.3 |
| Thymus | 12.0 | 8.9 |
| Spleen | 15.4 | 10.5 |
| Lymph node | 5.4 | 4.4 |
| Colorectal | 8.2 | 6.0 |
| Stomach | 12.3 | 9.3 |
| Small intestine | 20.3 | 9.1 |
| Colon ca. SW480 | 21.6 | 23.7 |
| Colon ca.* (SW480 met) SW620 | 10.0 | 14.4 |
| Colon ca. HT29 | 12.3 | 11.0 |
| Colon ca. HCT-116 | 17.8 | 16.8 |
| Colon ca. CaCo-2 | 17.8 | 15.9 |
| 83219 CC Well to Mod Diff | 9.9 | 6.9 |
| (ODO3866) | | |
| Colon ca. HCC-2998 | 20.0 | 16.7 |
| Gastric ca.* (liver met) | 12.2 | 9.3 |
| NCI-N87 | | |
| Bladder | 4.8 | 2.8 |
| Trachea | 24.5 | 17.7 |
| Kidney | 5.3 | 3.5 |
| Kidney (fetal) | 12.2 | 8.5 |
| Renal ca. 786-0 | 17.0 | 17.8 |
| Renal ca. A498 | 36.9 | 32.1 |
| Renal ca. RXF 393 | 3.9 | 3.6 |
| Renal ca. ACHN | 7.8 | 10.0 |
| Renal ca. UO-31 | 15.5 | 23.0 |
| Renal ca. TK-10 | 7.4 | 8.4 |
| Liver | 6.1 | 1.5 |
| Liver (fetal) | 14.5 | 6.5 |
| Liver ca. (hepatoblast) HepG2 | 10.0 | 12.0 |
| Lung | 19.6 | 8.8 |
| Lung (fetal) | 20.3 | 10.2 |
| Lung ca. (small cell) LX-1 | 5.3 | 5.6 |
| Lung ca. (small cell) NCI-H69 | 25.3 | 27.2 |
| Lung ca. (s. cell var.) SHP-77 | 23.0 | 29.1 |
| Lung ca. (large cell) NCI-H460 | 3.5 | 1.9 |
| Lung ca. (non-sm. cell) A549 | 5.4 | 3.8 |
| Lung ca. (non-s. cell) NCI-H23 | 18.0 | 18.3 |
| Lung ca (non-s. cell) HOP-62 | 9.2 | 13.2 |
| Lung ca. (non-s. cl) NCI-H522 | 14.1 | 15.7 |
| Lung ca. (squam.) SW 900 | 6.7 | 4.5 |
| Lung ca. (squam.) NCI-H596 | 13.4 | 9.1 |
| Mammary gland | 19.2 | 14.8 |
| Breast ca.* (pl. effusion) | 5.9 | 5.3 |
| MCF-7 | | |
| Breast ca.* (pl. ef) | 61.6 | 50.7 |
| MDA-MB-231 | | |
| Breast ca.* (pl. effusion) | 3.9 | 3.6 |
| T47D | | |
| Breast ca. BT-549 | 48.3 | 39.8 |
| Breast ca. MDA-N | 11.7 | 13.0 |
| Ovary | 42.9 | 43.8 |
| Ovarian ca. OVCAR-3 | 11.7 | 10.3 |
| Ovarian ca. OVCAR-4 | 2.5 | 4.4 |
| Ovarian ca. OVCAR-5 | 13.3 | 21.9 |
| Ovarian ca. OVCAR-8 | 16.4 | 16.3 |
| Ovarian ca. IGROV-1 | 4.7 | 4.8 |
| Ovarian ca.* (ascites) SK-OV-3 | 21.6 | 20.3 |
| Uterus | 15.6 | 9.7 |
| Plancenta | 12.9 | 7.8 |
| Prostate | 9.3 | 9.3 |
| Prostate ca.* (bone met) PC-3 | 17.6 | 15.6 |
| Testis | 56.3 | 59.0 |
| Melanoma Hs688(A).T | 9.4 | 5.7 |
| Melanoma* (met) Hs688(B).T | 4.6 | 3.6 |
| Melanoma UACC-62 | 2.9 | 2.9 |
| Melanoma M14 | 3.8 | 3.2 |
| Melanoma LOX IMVI | 18.0 | 20.9 |
| Melanoma* (met) SK-MEL-5 | 6.3 | 5.9 |
| Adipose | 5.7 | 3.3 |

TABLE N
Panels 2D and 3D

| Panel 2D | | Panel 3D | |
|---|---|---|---|
| | Relative | | Relative |
| | Expression (%) | | Expression (%) |
| | 2dtm4287f_ag | | 3dtm3957f_ag |
| Tissue Name | 721 | Tissue Name | 721 |
| Normal Colon GENPAK | 55.5 | 94905_Daoy_Medulloblastoma/ | 9.3 |
| 061003 | | Cerebellum_sscDNA | |
| 83219 CC Well to Mod Diff | 11.1 | 94906_TE671_Medulloblastom/ | 9.9 |
| (ODO3866) | | Cerebellum_sscDNA | |
| 83220 CC NAT (ODO3866) | 9.4 | 94907_D283 | 35.1 |
| | | Med_Medulloblastoma/Cerebel | |
| | | lum_sscDNA | |
| 83221 CC Gr.2 rectosigmoid | 27.9 | 94908_PFSK-1_Primitive | 30.1 |
| (ODO3868) | | Neuroectodermal/Cerebellum_{—} | |
| | | sscDNA | |
| 83222 CC NAT (ODO3868) | 5.5 | 94909_XF-498_CNS_sscDNA | 16.2 |
| 83235 CC Mod Diff | 33.4 | 94910_SNB- | 29.9 |
| (ODO3920) | | 78_CNS/glioma_sscDNA | |
| 83236 CC NAT (ODO3920) | 17.8 | 94911_SF- | 21.3 |
| | | 268_CNS/glioblastoma_sscDNA | |
| 83237 CC Gr.2 ascend colon | 32.5 | 94912_T98G_Glioblastoma_ssc | 21.5 |
| (ODO3921) | | DNA | |
| 83238 CC NAT (ODO3921) | 11.0 | 96776_SK-N- | 31.9 |
| | | SH_Neuroblastoma | |
| | | (metastasis)_sscDNA | |
| 83241 CC from Partial | 26.1 | 94913_SF- | 19.5 |
| Hepatectomy (ODO4309) | | 295_CNS/glioblastoma_sscDNA | |
| 83242 Liver NAT (ODO4309) | 19.2 | 94914_Cerebellum_sscDNA | 15.6 |
| 87472 Colon mets to lung | 17.9 | 96777_Cerebellum_sscDNA | 16.2 |
| (OD04451-01) | | | |
| 87473 Lung NAT (OD04451- | 14.0 | 94916_NCI- | 19.2 |
| 02) | | H292_Mucoepidermoid lung | |
| | | carcinoma_sscDNA | |
| Normal Prostate Clontech A+ | 26.4 | 94917_DMS-114_Small cell | 18.9 |
| 6546-1 | | lung cancer_sscDNA | |
| 84140 Prostate Cancer | 36.9 | 94918_DMS-79_Small cell | 100.0 |
| (OD04410) | | lung | |
| | | cancer/neuroendocrine_sscDNA | |
| 84141 Prostate NAT | 37.9 | 94919_NCI-H146_Small cell | 18.9 |
| (OD04410) | | lung | |
| | | cancer/neuroendocrine_sscDNA | |
| 87073 Prostate Cancer | 40.3 | 94920_NCI-H526_Small cell | 25.0 |
| (OD04720-01) | | lung | |
| | | cancer/neuroendocrine_sscDNA | |
| 87074 Prostate NAT | 46.3 | 94921_NCI-N417_Small cell | 15.3 |
| (OD04720-02) | | lung | |
| | | cancer/neuroendocrine_sscDNA | |
| Normal Lung GENPAK 061010 | 35.1 | 94923_NCI-H82_Small cell | 17.7 |
| | | lung | |
| | | cancer/neuroendocrine_sscDNA | |
| 83239 Lung Met to Muscle | 19.3 | 94924_NCI-H157_Squamous | 18.0 |
| (ODO4286) | | cell lung cancer | |
| | | (metastasis)_sscDNA | |
| 83240 Muscle NAT | 35.6 | 94925_NCI-H1155_Large cell | 41.8 |
| (ODO4286) | | lung | |
| | | cancer/neuroendocrine_sscDNA | |
| 84136 Lung Malignant Cancer | 38.7 | 94926_NCI-H1299_Large cell | 44.1 |
| (OD03126) | | lung | |
| | | cancer/neuroendocrine_sscDNA | |
| 84137 Lung NAT (OD03126) | 38.4 | 94927_NCI-H727_Lung | 18.2 |
| | | carcinoid_sscDNA | |
| 84871 Lung Cancer (OD04404) | 37.6 | 94928_NCI-UMC-11_Lung | 29.9 |
| | | carcinoid_sscDNA | |
| 84872 Lung NAT (OD04404) | 26.2 | 94929_LX-1_Small cell lung | 12.3 |
| | | cancer_sscDNA | |
| 84875 Lung Cancer (OD04565) | 15.2 | 94930_Colo-205_Colon | 5.1 |
| | | cancer_sscDNA | |
| 84876 Lung NAT (OD04565) | 13.8 | 94931_KM12_Colon | 17.6 |
| | | cancer_sscDNA | |
| 85950 Lung Cancer (OD04237- | 100.0 | 94932_KM20L2_Colon | 8.8 |
| 01) | | cancer_sscDNA | |
| 85970 Lung NAT (OD04237- | 26.2 | 94933_NCI-H716_Colon | 10.2 |
| 02) | | cancer_sscDNA | |
| 83255 Ocular Mel Met to Liver | 23.5 | 94935_SW-48_Colon | 6.5 |
| (ODO4310) | | adenocarcinoma_sscDNA | |
| 83256 Liver NAT (ODO4310) | 10.4 | 94936_SW1116_Colon | 10.7 |
| | | adenocarcinoma_sscDNA | |
| 84139 Melanoma Mets to Lung | 37.4 | 94937_LS 174T_Colon | 7.7 |
| (OD04321) | | adenocarcinoma_sscDNA | |
| 84138 Lung NAT (OD04321) | 23.2 | 94938_SW-948_Colon | 1.1 |
| | | adenocarcinoma_sscDNA | |
| Normal Kidney GENPAK | 50.3 | 94939_SW-480_Colon | 13.2 |
| 061008 | | adenocarcinoma_sscDNA | |
| 83786 Kidney Ca, Nuclear | 51.0 | 94940_NCI-SNU-5_Gastric | 18.9 |
| grade 2 (OD04338) | | carcinoma_sscDNA | |
| 83787 Kidney NAT (OD04338) | 46.0 | 94941_KATO III_Gastric | 16.8 |
| | | carcinoma_sscDNA | |
| 83788 Kidney Ca Nuclear grade | 72.2 | 94943_NCI-SNU-16_Gastric | 24.7 |
| ½ (OD04339) | | carcinoma_sscDNA | |
| 83789 Kidney NAT (OD04339) | 51.4 | 94944_NCI-SNU-1_Gastric | 12.4 |
| | | carcinoma_sscDNA | |
| 83790 Kidney Ca, Clear cell | 42.0 | 94946_RF-1_Gastric | 10.6 |
| type (OD04340) | | adenocarcinoma_sscDNA | |
| 83791 Kidney NAT (OD04340) | 36.3 | 94947_RF-48_Gastric | 13.1 |
| | | adenocarcinoma_sscDNA | |
| 83792 Kidney Ca, Nuclear | 23.0 | 96778_MKN-45_Gastric | 18.4 |
| grade 3 (OD04348) | | carcinoma_sscDNA | |
| 83793 Kidney NAT (OD04348) | 29.9 | 94949_NCI-N87_Gastric | 5.3 |
| | | carcinoma_sscDNA | |
| 87474 Kidney Cancer | 24.5 | 94951_OVCAR-5_Ovarian | 5.8 |
| (OD04622-01) | | carcinoma_sscDNA | |
| 87475 Kidney NAT (OD04622- | 7.6 | 94952_RL95-2_Uterine | 7.3 |
| 03) | | carcinoma_sscDNA | |
| 85973 Kidney Cancer | 32.1 | 94953_HelaS3_Cervical | 12.3 |
| (OD04450-01) | | adenocarcinoma_sscDNA | |
| 85974 Kidney NAT (OD04450- | 35.1 | 94954_Ca Ski_Cervical | 26.1 |
| 03) | | epidermoid carcinoma | |
| | | (metastasis)_sscDNA | |
| Kidney Cancer Clontech | 28.9 | 94955_ES-2_Ovarian clear cell | 19.6 |
| 8120607 | | carcinoma_sscDNA | |
| Kidney NAT Clontech 8120608 | 25.5 | 94957_Ramos/6h stim_″; | 9.3 |
| | | Stimulated with | |
| | | PMA/ionomycin 6h_sscDNA | |
| Kidney Cancer Clontech | 37.9 | 94958_Ramos/14h stim_″; | 9.3 |
| 8120613 | | Stimulated with | |
| | | PMA/ionomycin 14h_sscDNA | |
| Kidney NAT Clontech 8120614 | 34.6 | 94962_MEG-01_Chronic | 10.1 |
| | | myelogenous leukemia | |
| | | (megokaryoblast)_sscDNA | |
| Kidney Cancer Clontech | 55.1 | 94963_Raji_Burkitt's | 15.1 |
| 9010320 | | lymphoma_sscDNA | |
| Kidney NAT Clontech 9010321 | 49.0 | 94964_Daudi_Burkitt's | 16.3 |
| | | lymphoma_sscDNA | |
| Normal Uterus GENPAK | 10.3 | 94965_U266_B-cell | 17.3 |
| 061018 | | plasmacytoma/myeloma_sscDNA | |
| Uterus Cancer GENPAK | 52.1 | 94968_CA46_Burkitt's | 7.5 |
| 064011 | | lymphoma_sscDNA | |
| Normal Thyroid Clontech A+ | 34.6 | 94970_RL_non-Hodgkin's B- | 3.7 |
| 6570-1 | | cell lymphoma_sscDNA | |
| Thyroid Cancer GENPAK | 23.0 | 94972_JM1_pre-B-cell | 12.6 |
| 064010 | | lymphoma/leukemia_sscDNA | |
| Thyroid Cancer INVITROGEN | 25.0 | 94973_Jurkat_T cell | 23.0 |
| A302152 | | leukemia_sscDNA | |
| Thyroid NAT INVITROGEN | 31.4 | 94974_TF- | 26.2 |
| A302153 | | 1_Erythroleukemia_sscDNA | |
| Normal Breast GENPAK | 30.4 | 94975_HUT 78_T-cell | 16.8 |
| 061019 | | lymphoma_sscDNA | |
| 84877 Breast Cancer | 42.0 | 94977_U937_Histiocytic | 26.1 |
| (OD04566) | | lymphoma_sscDNA | |
| 85975 Breast Cancer | 90.1 | 94980_KU-812_Myelogenous | 33.9 |
| (OD04590-01) | | leukemia_sscDNA | |
| 85976 Breast Cancer Mets | 87.1 | 94981_769-P_Clear cell renal | 15.7 |
| (OD04590-03) | | carcinoma_sscDNA | |
| 87070 Breast Cancer Metastasis | 97.9 | 94983_Caki-2_Clear cell renal | 19.3 |
| (OD04655-05) | | carcinoma_sscDNA | |
| GENPAK Breast Cancer | 17.8 | 94984_SW 839_Clear cell renal | 21.5 |
| 064006 | | carcinoma_sscDNA | |
| Breast Cancer Res. Gen. 1024 | 61.1 | 94986_G401_Wilms' | 17.0 |
| | | tumor_sscDNA | |
| Breast Cancer Clontech | 41.8 | 94987_Hs766T_Pancreatic | 14.1 |
| 9100266 | | carcinoma (LN | |
| | | metastasis)_sscDNA | |
| Breast NAT Clontech 9100265 | 28.9 | 94988_CAPAN-1_Pancreatic | 4.8 |
| | | adenocarcinoma (liver | |
| | | metastasis)_sscDNA | |
| Breast Cancer INVITROGEN | 33.2 | 94989_SU86.86_Pancreatic | 10.7 |
| A209073 | | carcinoma (liver | |
| | | metastasis)_sscDNA | |
| Breast NAT INVITROGEN | 33.0 | 94990_BxPC-3_Pancreatic | 9.6 |
| A2090734 | | adenocarcinoma_sscDNA | |
| Normal Liver GENPAK | 13.8 | 94991_HPAC_Pancreatic | 13.7 |
| 061009 | | adenocarcinoma_sscDNA | |
| Liver Cancer GENPAK 064003 | 13.1 | 94992_MIA PaCa-2_Pancreatic | 4.0 |
| | | carcinoma_sscDNA | |
| Liver Cancer Research Genetics | 7.7 | 94993_CFPAC-1_Pancreatic | 18.7 |
| RNA 1025 | | ductal | |
| | | adenocarcinoma_sscDNA | |
| Liver Cancer Research Genetics | 7.9 | 94994_PANC-1_Pancreatic | 16.8 |
| RNA 1026 | | epithelioid ductal | |
| | | carcinoma_sscDNA | |
| Paired Liver Cancer Tissue | 16.8 | 94996_T24_Bladder carcinma | 14.6 |
| Research Genetics RNA 6004-T | | (transitional cell)_sscDNA | |
| Paired Liver Tissue Research | 16.5 | 94997_5637_Bladder | 17.0 |
| Genetics RNA 6004-N | | carcinoma_sscDNA | |
| Paired Liver Cancer Tissue | 18.4 | 94998_HT-1197_Bladder | 10.3 |
| Research Genetics RNA 6005-T | | carcinoma_sscDNA | |
| Paired Liver Tissue Research | 3.8 | 94999_UM-UC-3_Bladder | 8.8 |
| Genetics RNA 6005-N | | carcinma (transitional | |
| | | cell)_sscDNA | |
| Normal Bladder GENPAK | 36.9 | 95000_A204_Rhabdomyosarco | 17.6 |
| 061001 | | ma_sscDNA | |
| Bladder Cancer Research | 16.3 | 95001_HT- | 16.4 |
| Genetics RNA 1023 | | 1080_Fibrosarcoma_sscDNA | |
| Bladder Cancer INVITROGEN | 20.2 | 95002_MG-63_Osteosarcoma | 8.2 |
| A302173 | | (bone)_sscDNA | |
| 87071 Bladder Cancer | 60.7 | 95003_SK-LMS- | 33.7 |
| (OD04718-01) | | 1_Leiomyosarcoma | |
| | | (vulva)_sscDNA | |
| 87072 Bladder Normal | 24.0 | 95004_SJRH30_Rhabdomyo- | 13.4 |
| Adjacent (OD04718-03) | | sarcoma (met to bone | |
| | | marrow)_sscDNA | |
| Normal Ovary Res. Gen. | 28.5 | 95005_A431_Epidermoid | 6.8 |
| | | carcinoma_sscDNA | |
| Ovarian Cancer GENPAK | 42.0 | 95007_WM266- | 8.8 |
| 064008 | | 4_Melanoma_sscDNA | |
| 87492 Ovary Cancer | 55.9 | 95010_DU 145_Prostate | 0.0 |
| (OD04768-07) | | carcinoma (brain | |
| | | metastasis)_sscDNA | |
| 87493 Ovary NAT (OD04768- | 9.2 | 95012_MDA-MB-468_Breast | 10.6 |
| 08) | | adenocarcinoma_sscDNA | |
| Normal Stomach GENPAK | 18.0 | 95013_SCC-4_Squamous cell | 0.2 |
| 061017 | | carcinoma of tongue_sscDNA | |
| Gastric Cancer Clontech | 9.6 | 95014_SCC-9_Squamous cell | 0.3 |
| 9060358 | | carcinoma of tongue_sscDNA | |
| NAT Stomach Clontech | 15.5 | 95015_SCC-15_Squamous cell | 0.0 |
| 9060359 | | carcinoma of tongue_sscDNA | |
| Gastric Cancer Clontech | 21.5 | 95017_CAL 27_Squamous cell | 17.4 |
| 9060395 | | carcinoma of tongue_sscDNA | |
| NAT Stomach Clontech | 17.2 | | |
| 9060394 | | | |
| Gastric Cancer Clontech | 28.1 | | |
| 9060397 | | | |
| NAT Stomach Clontech | 8.9 | | |
| 9060396 | | | |
| Gastric Cancer GENPAK | 36.3 | | |
| 064005 | | | |

TABLE O
Panels 4D and 4.1D

| | Relative | |
|---|---|---|
| | Expression (%) | Relative |
| | 4.1dx4tm6096t_ag | Expression (%) |
| Tissue Name | 2835_b2 | 4Dtm2469f_ag721 |
| 93768_Secondary Th1_anti-CD28/anti-CD3 | 89.8 | 29.3 |
| 93769_Secondary Th2_anti-CD28/anti-CD3 | 90.4 | 28.1 |
| 93770_Secondary Tr1_anti-CD28/anti-CD3 | 89.4 | 27.9 |
| 93573_Secondary Th1_resting day 4-6 in IL-2 | 32.0 | 10.1 |
| 93572_Secondary Th2_resting day 4-6 in IL-2 | 28.8 | 6.8 |
| 93571_Secondary Tr1_resting day 4-6 in IL-2 | 32.5 | 13.5 |
| 93568_primary Th1_anti-CD28/anti-CD3 | 69.4 | 28.3 |
| 93569_primary Th2_anti-CD28/anti-CD3 | 72.6 | 28.3 |
| 93570_primary Tr1_anti-CD28/anti-CD3 | 78.5 | 44.1 |
| 93565_primary Th1_resting dy 4-6 in IL-2 | 24.5 | 48.3 |
| 93566_primary Th2_resting dy 4-6 in IL-2 | 17.4 | 26.8 |
| 93567_primary Tr1_resting dy 4-6 in IL-2 | 27.4 | 23.8 |
| 93351_CD45RA CD4 lymphocyte_anti-CD28/anti- | 69.5 | 21.3 |
| CD3 | | |
| 93352_CD45RO CD4 lymphocyte_anti-CD28/anti- | 62.4 | 30.8 |
| CD3 | | |
| 93251_CD8 Lymphocytes_anti-CD28/anti-CD3 | 67.5 | 24.7 |
| 93353_chronic CD8 Lymphocytes 2ry_resting dy 4-6 | 73.9 | 21.9 |
| in IL-2 | | |
| 93574_chronic CD8 Lymphocytes 2ry_activated | 36.0 | 16.7 |
| CD3/CD28 | | |
| 93354_CD4_none | 16.1 | 6.2 |
| 93252_Secondary Th1/Th2/Tr1_anti-CD95 CH11 | 67.3 | 18.6 |
| 93103_LAK cells_resting | 41.5 | 12.5 |
| 93788_LAK cells_IL-2 | 67.4 | 23.2 |
| 93787_LAK cells_IL-2 + IL-12 | 30.1 | 19.3 |
| 93789_LAK cells_IL-2 + IFN gamma | 29.3 | 40.6 |
| 93790_LAK cells_IL-2 + IL-18 | 26.4 | 27.7 |
| 93104_LAK cells_PMA/ionomycin and IL-18 | 17.3 | 5.5 |
| 93578_NK Cells IL-2_resting | 63.9 | 18.8 |
| 93109_Mixed Lymphocyte Reaction_Two Way MLR | 39.8 | 18.3 |
| 93110_Mixed Lymphocyte Reaction_Two Way MLR | 53.2 | 19.1 |
| 93111_Mixed Lymphocyte Reaction_Two Way MLR | 45.6 | 18.6 |
| 93112_Mononuclear Cells (PBMCs)_resting | 18.2 | 4.8 |
| 93113_Mononuclear Cells (PBMCs)_PWM | 35.9 | 53.6 |
| 93114_Mononuclear Cells (PBMCs)_PHA-L | 75.5 | 39.8 |
| 93249_Ramos (B cell)_none | 63.2 | 35.8 |
| 93250_Ramos (B cell)_ionomycin | 80.5 | 100.0 |
| 93349_B lymphocytes_PWM | 50.8 | 82.9 |
| 93350_B lymphoytes_CD40L and IL-4 | 75.0 | 51.0 |
| 92665_EOL-1 (Eosinophil)_dbcAMP differentiated | 80.2 | 27.4 |
| 93248_EOL-1 (Eosinophil)_dbcAMP/PMAionomycin | 71.6 | 27.7 |
| 93356_Dendritic Cells_none | 53.1 | 24.7 |
| 93355_Dendritic Cells_LPS 100 ng/ml | 29.2 | 17.6 |
| 93775_Dendritic Cells_anti-CD40 | 57.9 | 18.3 |
| 93774_Monocytes_resting | 28.2 | 13.0 |
| 93776_Monocytes_LPS 50 ng/ml | 27.2 | 7.5 |
| 93581_Macrophages_resting | 50.6 | 23.7 |
| 93582_Macrophages_LPS 100 ng/ml | 15.3 | 8.8 |
| 93098_HUVEC (Endothelial)_none | 63.8 | 48.0 |
| 93099_HUVEC (Endothelial)_starved | 91.8 | 82.9 |
| 93100_HUVEC (Endothelial)_IL-1b | 81.4 | 26.2 |
| 93779_HUVEC (Endothelial)_IFN gamma | 92.8 | 52.1 |
| 93102_HUVEC (Endothelial)_TNF alpha + IFN | 64.2 | 26.8 |
| gamma | | |
| 93101_HUVEC (Endothelial)_TNF alpha + IL4 | 73.0 | 36.3 |
| 93781_HUVEC (Endothelial)_IL-11 | 78.8 | 37.4 |
| 93583_Lung Microvascular Endothelial Cells_none | 100.0 | 31.4 |
| 93584_Lung Microvascular Endothelial Cells_TNFa | 71.0 | 26.4 |
| (4 ng/ml) and IL1b (1 ng/ml) | | |
| 92662_Microvascular Dermal endothelium_none | 90.5 | 47.6 |
| 92663_Microsvasular Dermal endothelium_TNFa (4 | 45.5 | 32.1 |
| ng/ml) and IL1b (1 ng/ml) | | |
| 93773_Bronchial epithelium_TNFa (4 ng/ml) and | 75.7 | 23.0 |
| IL1b (1 ng/ml)** | | |
| 93347_Small Airway Epithelium_none | 35.4 | 22.1 |
| 93348_Small Airway Epithelium_TNFa (4 ng/ml) and | 49.7 | 54.0 |
| IL1b (1 ng/ml) | | |
| 92668_Coronery Artery SMC_resting | 82.5 | 46.7 |
| 92669_Coronery Artery SMC_TNFa (4 ng/ml) and | 60.5 | 31.9 |
| IL1b (1 ng/ml) | | |
| 93107_astrocytes_resting | 59.6 | 34.2 |
| 93108_astrocytes_TNFa (4 ng/ml) and IL1b (1 ng/ml) | 42.9 | 25.2 |
| 92666_KU-812 (Basophil)_resting | 92.8 | 42.3 |
| 92667_KU-812 (Basophil)_PMA/ionoycin | 96.0 | 66.4 |
| 93579_CCD1106 (Keratinocytes)_none | 80.5 | 38.4 |
| 93580_CCD1106 (Keratinocytes)_TNFa and IFNg** | 71.3 | 12.0 |
| 93791_Liver Cirrhosis | 18.4 | 4.0 |
| 93792_Lupus Kidney | 47.4 | 4.4 |
| 93577_NCI-H292 | 75.0 | 47.0 |
| 93358_NCI-H292_IL-4 | 76.8 | 55.1 |
| 93360_NCI-H292_IL-9 | 60.0 | 53.6 |
| 93359_NCI-H292_IL-13 | 79.0 | 33.4 |
| 93357_NCI-H292_IFN gamma | 72.9 | 28.5 |
| 93777_HPAEC_- | 92.8 | 42.0 |
| 93778_HPAEC_IL-1 beta/TNA alpha | 87.1 | 31.0 |
| 93254_Normal Human Lung Fibroblast_none | 61.8 | 32.3 |
| 93253_Normal Human Lung Fibroblast_TNFa (4 | 61.1 | 14.7 |
| ng/ml) and IL-1b (1 ng/ml) | | |
| 93257_Normal Human Lung Fibroblast_IL-4 | 76.2 | 45.7 |
| 93256_Normal Human Lung Fibroblast_IL-9 | 67.4 | 37.6 |
| 93255_Normal Human Lung Fibroblast_IL-13 | 67.7 | 32.3 |
| 93258_Normal Human Lung Fibroblast_IFN gamma | 71.8 | 46.7 |
| 93106_Dermal Fibroblasts CCD1070_resting | 77.1 | 54.0 |
| 93361_Dermal Fibroblasts CCD1070_TNF alpha 4 | 47.3 | 61.1 |
| ng/ml | | |
| 93105_Dermal Fibroblasts CCD1070_IL-1 beta 1 | 47.9 | 25.0 |
| ng/ml | | |
| 93772_dermal fibroblast_IFN gamma | 95.6 | 15.8 |
| 93771_dermal fibroblast_IL-4 | 63.6 | 37.9 |
| 93259_IBD Colitis 1** | 9.5 | 1.2 |
| 93260_IBD Colitis 2 | 28.3 | 0.9 |
| 93261_IBD Crohns | 15.0 | 1.6 |
| 735010_Colon_normal | 34.4 | 19.6 |
| 735019_Lung_none | 63.0 | 17.4 |
| 64028-1_Thymus_none | 33.9 | 26.4 |
| 64030-1_Kidney_none | 89.8 | 45.4 |

Panel 1.2 Summary:

The three panels run with three probe/primer sets, two of which are identical, do not concur completely with one another. However, the pattern seen in all three runs is that expression of NOV4a is high in skeletal muscle. Among disease tissues, consistent expression is seen in a lung cancer specimen. Expression of this gene is at lower levels in a variety of other tissues.

Panel 1.3D Summary:

NOV4a encodes a protein with a growth factor domain which is expressed in a wide range of tissues and cell types. However, it shows its highest adult expression in the hippocampus (levels are higher in the fetal skeleton) followed by the testes and ovaries. Many growth factors have been shown to be beneficial in the process of compensatory synaptogenesis in the central nervous system in response to injury (in animal models of stroke, head trauma, spinal cord injury). The fact that this gene shows its highest expression in the adult brain suggests that it may have neuroprotective/neurotrophic effects. Furthermore, its specific region of highest expresion (the hippocampus) is a site of pronounced neurodegeneration in Alzheimer's disease, and to a lesser extent Parkinson's disease. Therefore, this molecule may be useful as a protein therapeutic in treating these diseases in addition to stroke, head trauma and spinal cord injury. In addition, this gene shows highest expression in fetal skeletal muscle when compared to adult skeletal muscle, potentially indicating a role in tissue regeneration. This is so given that in many instances fetal tissues are rapidly growing and developing whereas adult tissues are in homeostasis.

Panel 2D Summary:

The expression of this gene appears to be in virtually all samples in panel 2D. Of particular interest is the observation that there seems to be over-expression of this gene in samples of gastric and colon cancer relative to their adjacent margins. Thus, therapeutic targeting of this gene may be beneficial in these or other diseases.

Panel 3D Summary:

Expression of this gene is seen in almost all tissues and cell lines, except in a few samples of squamous cell carcinoma and prostate carcinoma. It is highest in lung cancers, followed by lower levels in CNS cancers, leiomyosarcoma and leukemias and lymphomas.

Panel 4D and 4.1D Summary:

This transcript as probed by Ag72 1 and Ag2835 is broadly expressed in many tissues and cell types regardless of treatment.

NOV6

Expression of NOV6 was assessed using the primer-probe set Ag274, described in Table P. Results of the RTQ-PCR runs are shown in Tables Q and R.

TABLE P
Probe Name: Ag274

| | | | | Start |
|---|---|---|---|---|
| Primers | Sequences | | Length | Position |
| Forward | 5′-CAGAACAGATGTATTCCCCTTGGT-3′ | (SEQ ID NO:85) | 24 | 194 |
| Probe | FAM-5′-CTCAGCGCCTCGATGTCCACCC-3′-TAMRA | (SEQ ID NO:86) | 22 | 226 |
| Reverse | 5′-CTGGCTTCCCCCAATGC-3′ | (SEQ ID NO:87) | 17 | 253 |

TABLE Q
Panels 2D and 3D

| Panel 2D | | Panel 3D | |
|---|---|---|---|
| | Rel. Expr., % | | Rel. Expr., % |
| | 2dtm6123f_ag | | 3dx4tm6102f_{—} |
| Tissue Name | 274 | Tissue Name | ag274_a2 |
| Normal Colon GENPAK | 23.3 | 94905_Daoy_Medulloblastoma/ | 14.7 |
| 061003 | | Cerebellum_sscDNA | |
| 83219 CC Well to Mod Diff | 13.2 | 94906_TE671_Medulloblastom/ | 51.2 |
| (ODO3866) | | Cerebellum_sscDNA | |
| 83220 CC NAT (ODO3866) | 10.0 | 94907_D283 | 0.0 |
| | | Med_Medulloblastoma/Cerebel- | |
| | | lum_sscDNA | |
| 83221 CC Gr.2 rectosigmoid | 6.0 | 94908_PFSK-1_Primitive | 0.0 |
| (ODO3868) | | Neuroectodermal/Cerebellum_{—} | |
| | | sscDNA | |
| 83222 CC NAT (ODO3868) | 6.0 | 94909_XF-498_CNS_sscDNA | 0.0 |
| 83235 CC Mod Diff | 3.1 | 94910_SNB- | 10.5 |
| (ODO3920) | | 78_CNS/glioma_sscDNA | |
| 83236 CC NAT (ODO3920) | 10.9 | 94911_SF- | 0.0 |
| | | 268_CNS/glioblastoma_sscDNA | |
| 83237 CC Gr.2 ascend colon | 10.2 | 94912_T98G_Glioblastoma_{—} | 10.3 |
| (ODO3921) | | sscDNA | |
| 83238 CC NAT (ODO3921) | 25.5 | 96776_SK-N- | 10.5 |
| | | SH_Neuroblastoma | |
| | | (metastasis)_sscDNA | |
| 83241 CC from Partial | 9.7 | 94913_SF- | 0.0 |
| Hepatectomy (ODO4309) | | 295_CNS/glioblastoma_sscDNA | |
| 83242 Liver NAT (ODO4309) | 48.3 | 94914_Cerebellum_sscDNA | 22.7 |
| 87472 Colon mets to lung | 14.4 | 96777_Cerebellum_sscDNA | 9.0 |
| (OD04451-01) | | | |
| 87473 Lung NAT (OD04451- | 17.8 | 94916_NCI- | 0.0 |
| 02) | | H292_Mucoepidermoid lung | |
| | | carcinoma_sscDNA | |
| Normal Prostate Clontech A+ | 11.0 | 94917_DMS-114_Small cell | 6.4 |
| 6546-1 | | lung cancer_sscDNA | |
| 84140 Prostate Cancer | 9.7 | 94918_DMS-79_Small cell | 32.9 |
| (OD04410) | | lung | |
| | | cancer/neuroendocrine_sscDNA | |
| 84141 Prostate NAT | 4.1 | 94919_NCI-H146_Small cell | 0.0 |
| (OD04410) | | lung | |
| | | cancer/neuroendocrine _sscDNA | |
| 87073 Prostate Cancer | 2.0 | 94920_NCI-H526_Small cell | 11.3 |
| (OD04720-01) | | lung | |
| | | cancer/neuroendocrine_sscDNA | |
| 87074 Prostate NAT | 4.4 | 94921_NCI-N417_Small cell | 0.0 |
| (OD04720-02) | | lung | |
| | | cancer/neuroendocrine_sscDNA | |
| Normal Lung GENPAK 061010 | 66.0 | 94923_NCI-H82_Small cell | 5.0 |
| | | lung | |
| | | cancer/neuroendocrine_sscDNA | |
| 83239 Lung Met to Muscle | 100.0 | 94924_NCI-H157_Squamous | 0.0 |
| (ODO4286) | | cell lung cancer | |
| | | (metastasis)_sscDNA | |
| 83240 Muscle NAT | 72.2 | 94925_NCI-H1155_Large cell | 25.2 |
| (ODO4286) | | lung | |
| | | cancer/neuroendocrine_sscDNA | |
| 84136 Lung Malignant Cancer | 35.4 | 94926_NCI-H1299_Large cell | 0.0 |
| (OD03126) | | lung | |
| | | cancer/neuroendocrine_sscDNA | |
| 84137 Lung NAT (OD03126) | 68.3 | 94927_NCI-H727_Lung | 0.0 |
| | | carcinoid_sscDNA | |
| 84871 Lung Cancer | 29.5 | 94928_NCI-UMC-11_Lung | 11.5 |
| (OD04404) | | carcinoid_sscDNA | |
| 84872 Lung NAT (OD04404) | 35.6 | 94929_LX-1_Small cell lung | 49.9 |
| | | cancer_sscDNA | |
| 84875 Lung Cancer (OD04565) | 12.4 | 94930_Colo-205_Colon | 0.0 |
| | | cancer_sscDNA | |
| 84876 Lung NAT (OD04565) | 36.1 | 94931_KM12_Colon | 22.7 |
| | | cancer_sscDNA | |
| 85950 Lung Cancer (OD04237- | 18.6 | 94932_KM20L2_Colon | 0.0 |
| 01) | | cancer_sscDNA | |
| 85970 Lung NAT (OD04237- | 22.4 | 94933_NCI-H716_Colon | 0.0 |
| 02) | | cancer_sscDNA | |
| 83255 Ocular Mel Met to Liver | 4.2 | 94935_SW-48_Colon | 9.7 |
| (ODO4310) | | adenocarcinoma_sscDNA | |
| 83256 Liver NAT (ODO4310) | 24.3 | 94936_SW1116_Colon | 6.1 |
| | | adenocarcinoma_sscDNA | |
| 84139 Melanoma Mets to Lung | 5.8 | 94937_LS 174T_Colon | 0.0 |
| (OD04321) | | adenocarcinoma_sscDNA | |
| 84138 Lung NAT (OD04321) | 29.7 | 94938_SW-948_Colon | 7.5 |
| | | adenocarcinoma_sscDNA | |
| Normal Kidney GENPAK | 12.2 | 94939_SW-480_Colon | 0.0 |
| 061008 | | adenocarcinoma_sscDNA | |
| 83786 Kidney Ca, Nuclear | 16.5 | 94940_NCI-SNU-5_Gastric | 0.0 |
| grade 2 (OD04338) | | carcinoma_sscDNA | |
| 83787 Kidney NAT | 6.2 | 94941_KATO III_Gastric | 66.1 |
| (OD04338) | | carcinoma_sscDNA | |
| 83788 Kidney Ca Nuclear grade | 23.2 | 94943_NCI-SNU-16_Gastric | 0.0 |
| ½ (OD04339) | | carcinoma_sscDNA | |
| 83789 Kidney NAT (OD04339) | 4.6 | 94944_NCI-SNU-1_Gastric | 6.3 |
| | | carcinoma_sscDNA | |
| 83790 Kidney Ca, Clear cell | 11.8 | 94946_RF-1_Gastric | 5.5 |
| type (OD04340) | | adenocarcinoma_sscDNA | |
| 83791 Kidney NAT (OD04340) | 5.8 | 94947_RF-48_Gastric | 10.4 |
| | | adenocarcinoma_sscDNA | |
| 83792 Kidney Ca, Nuclear | 56.6 | 96778_MKN-45_Gastric | 0.0 |
| grade 3 (OD04348) | | carcinoma_sscDNA | |
| 83793 Kidney NAT (OD04348) | 22.8 | 94949_NCI-N87_Gastric | 11.2 |
| | | carcinoma_sscDNA | |
| 87474 Kidney Cancer | 24.0 | 94951_OVCAR-5_Ovarian | 8.8 |
| (OD04622-01) | | carcinoma_sscDNA | |
| 87475 Kidney NAT | 2.6 | 94952_RL95-2_Uterine | 0.0 |
| (OD04622-03) | | carcinoma_sscDNA | |
| 85973 Kidney Cancer | 0.9 | 94953_HelaS3_Cervical | 3.4 |
| (OD04450-01) | | adenocarcinoma_sscDNA | |
| 85974 Kidney NAT | 2.6 | 94954_Ca Ski_Cervical | 4.3 |
| (OD04450-03) | | epidermoid carcinoma | |
| | | (metastasis)_sscDNA | |
| Kidney Cancer Clontech | 5.7 | 94955_ES-2_Ovarian clear cell | 9.8 |
| 8120607 | | carcinoma_sscDNA | |
| Kidney NAT Clontech 8120608 | 3.6 | 94957_Ramos/6h stim_″; | 5.6 |
| | | Stimulated with | |
| | | PMA/ionomycin 6h_sscDNA | |
| Kidney Cancer Clontech | 5.7 | 94958_Ramos/14h stim_″; | 100.0 |
| 8120613 | | Stimulated with | |
| | | PMA/ionomycin 14h_sscDNA | |
| Kidney NAT Clontech 8120614 | 3.5 | 94962_MEG-01_Chronic | 11.3 |
| | | myelogenous leukemia | |
| | | (megokaryoblast)_sscDNA | |
| Kidney Cancer Clontech | 61.1 | 94963_Raji_Burkitt's | 5.4 |
| 9010320 | | lymphoma_sscDNA | |
| Kidney NAT Clontech 9010321 | 24.1 | 94964_Daudi_Burkitt's | 5.1 |
| | | lymphoma_sscDNA | |
| Normal Uterus GENPAK | 5.4 | 94965_U266_B-cell | 7.5 |
| 061018 | | plasmacytoma/myeloma_sscDNA | |
| Uterus Cancer GENPAK | 5.1 | 94968_CA46_Burkitt's | 0.0 |
| 064011 | | lymphoma_sscDNA | |
| Normal Thyroid Clontech A+ | 5.9 | 94970_RL_non-Hodgkin's B- | 0.0 |
| 6570-1 | | cell lymphoma_sscDNA | |
| Thyroid Cancer GENPAK | 3.1 | 94972_JM1_pre-B-cell | 0.0 |
| 064010 | | lymphoma/leukemia_sscDNA | |
| Thyroid Cancer INVITROGEN | 3.3 | 94973_Jurkat_T cell | 0.0 |
| A302152 | | leukemia_sscDNA | |
| Thyroid NAT INVITROGEN | 13.5 | 94974_TF- | 0.0 |
| A302153 | | 1_Erythroleukemia_sscDNA | |
| Normal Breast GENPAK | 13.2 | 94975_HUT 78_T-cell | 14.9 |
| 061019 | | lymphoma_sscDNA | |
| 84877 Breast Cancer | 40.9 | 94977_U937_Histiocytic | 9.1 |
| (OD04566) | | lymphoma_sscDNA | |
| 85975 Breast Cancer | 29.5 | 94980_KU-812_Myelogenous | 39.6 |
| (OD04590-01) | | leukemia_sscDNA | |
| 85976 Breast Cancer Mets | 26.1 | 94981_769-P_Clear cell renal | 6.0 |
| (OD04590-03) | | carcinoma_sscDNA | |
| 87070 Breast Cancer Metastasis | 31.9 | 94983_Caki-2_Clear cell renal | 9.7 |
| (OD04655-05) | | carcinoma_sscDNA | |
| GENPAK Breast Cancer | 16.0 | 94984_SW 839_Clear cell renal | 16.4 |
| 064006 | | carcinoma_sscDNA | |
| Breast Cancer Res. Gen. 1024 | 11.3 | 94986_G401_Wilms' | 6.7 |
| | | tumor_sscDNA | |
| Breast Cancer Clontech | 3.7 | 94987_Hs766T_Pancreatic | 0.0 |
| 9100266 | | carcinoma (LN | |
| | | metastasis)_sscDNA | |
| Breast NAT Clontech 9100265 | 4.7 | 94988_CAPAN-1_Pancreatic | 11.4 |
| | | adenocarcinoma (liver | |
| | | metastasis)_sscDNA | |
| Breast Cancer INVITROGEN | 5.7 | 94989_SU86.86_Pancreatic | 0.0 |
| A209073 | | carcinoma (liver | |
| | | metastasis)_sscDNA | |
| Breast NAT INVITROGEN | 1.5 | 94990_BxPC-3_Pancreatic | 0.0 |
| A2090734 | | adenocarcinoma_sscDNA | |
| Normal Liver GENPAK | 8.4 | 94991_HPAC_Pancreatic | 0.0 |
| 061009 | | adenocarcinoma_sscDNA | |
| Liver Cancer GENPAK 064003 | 8.4 | 94992_MIA PaCa-2_Pancreatic | 4.5 |
| | | carcinoma_sscDNA | |
| Liver Cancer Research Genetics | 25.3 | 94993_CFPAC-1_Pancreatic | 7.4 |
| RNA 1025 | | ductal | |
| | | adenocarcinoma_sscDNA | |
| Liver Cancer Research Genetics | 6.4 | 94994_PANC-1_Pancreatic | 58.7 |
| RNA 1026 | | epithelioid ductal | |
| | | carcinoma_sscDNA | |
| Paired Liver Cancer Tissue | 40.1 | 94996_T24_Bladder carcinma | 0.0 |
| Research Genetics RNA 6004-T | | (transitional cell)_sscDNA | |
| Paired Liver Tissue Research | 17.8 | 94997_5637_Bladder | 11.2 |
| Genetics RNA 6004-N | | carcinoma_sscDNA | |
| Paired Liver Cancer Tissue | 10.2 | 94998_HT-1197_Bladder | 0.0 |
| Research Genetics RNA 6005-T | | carcinoma_sscDNA | |
| Paired Liver Tissue Research | 18.6 | 94999_UM-UC-3_Bladder | 10.9 |
| Genetics RNA 6005-N | | carcinma (transitional | |
| | | cell)_sscDNA | |
| Normal Bladder GENPAK | 60.7 | 95000_A204_Rhabdomyo- | 0.0 |
| 061001 | | sarcoma_sscDNA | |
| Bladder Cancer Research | 5.1 | 95001_HT- | 0.0 |
| Genetics RNA 1023 | | 1080_Fibrosarcoma_sscDNA | |
| Bladder Cancer INVITROGEN | 18.6 | 95002_MG-63_Osteosarcoma | 9.9 |
| A302173 | | (bone)_sscDNA | |
| 87071 Bladder Cancer | 28.5 | 95003_SK-LMS- | 0.0 |
| (OD04718-01) | | 1_Leiomyosarcoma | |
| | | (vulva)_sscDNA | |
| 87072 Bladder Normal | 48.6 | 95004_SJRH30_Rhabdomyo- | 3.8 |
| Adjacent (OD04718-03) | | sarcoma (met to bone | |
| | | marrow)_sscDNA | |
| Normal Ovary Res. Gen. | 0.0 | 95005_A431_Epidermoid | 0.0 |
| | | carcinoma_sscDNA | |
| Ovarian Cancer GENPAK | 16.2 | 95007_WM266- | 0.0 |
| 064008 | | 4_Melanoma_sscDNA | |
| 87492 Ovary Cancer | 16.4 | 95010_DU 145_Prostate | 5.5 |
| (OD04768-07) | | carcinoma (brain | |
| | | metastasis)_sscDNA | |
| 87493 Ovary NAT (OD04768- | 19.2 | 95012_MDA-MB-468_Breast | 10.8 |
| 08) | | adenocarcinoma_sscDNA | |
| Normal Stomach GENPAK | 6.4 | 95013_SCC-4_Squamous cell | 0.0 |
| 061017 | | carcinoma of tongue_sscDNA | |
| Gastric Cancer Clontech | 2.0 | 95014_SCC-9_Squamous cell | 0.0 |
| 9060358 | | carcinoma of tongue_sscDNA | |
| NAT Stomach Clontech | 5.1 | 95015_SCC-15_Squamous cell | 0.0 |
| 9060359 | | carcinoma of tongue_sscDNA | |
| Gastric Cancer Clontech | 8.4 | 95017_CAL 27_Squamous cell | 0.0 |
| 9060395 | | carcinoma of tongue_sscDNA | |
| NAT Stomach Clontech | 10.2 | | |
| 9060394 | | | |
| Gastric Cancer Clontech | 17.4 | | |
| 9060397 | | | |
| NAT Stomach Clontech | 5.3 | | |
| 9060396 | | | |
| Gastric Cancer GENPAK | 14.7 | | |
| 064005 | | | |

TABLE R
Panel 4D

| | Rel. Expr., % | Rel. Expr., % |
|---|---|---|
| | 4dx4tm5043f_{—} | 4dx4tm5056f_{—} |
| Tissue Name | ag274_b2 | ag274_b2 |
| 93768_Secondary Th1_anti-CD28/ | 0.0 | 0.0 |
| anti-CD3 | | |
| 93769_Secondary Th2_anti-CD28/ | 0.4 | 0.0 |
| anti-CD3 | | |
| 93770_Secondary Tr1_anti-CD28/ | 0.2 | 0.2 |
| anti-CD3 | | |
| 93573_Secondary Th1_resting day 4- | 0.0 | 0.0 |
| 6 in IL-2 | | |
| 93572_Secondary Th2_resting day 4- | 0.9 | 0.0 |
| 6 in IL-2 | | |
| 93571_Secondary Tr1_resting day 4- | 0.3 | 0.0 |
| 6 in IL-2 | | |
| 93568_primary Th1_anti-CD28/anti- | 0.4 | 0.0 |
| CD3 | | |
| 93569_primary Th2_anti-CD28/anti- | 0.3 | 0.3 |
| CD3 | | |
| 93570_primary Tr1_anti-CD28/anti- | 0.0 | 0.0 |
| CD3 | | |
| 93565_primary Th1_resting dy 4- | 0.4 | 0.5 |
| 6 in IL-2 | | |
| 93566_primary Th2_resting dy 4- | 0.6 | 0.1 |
| 6 in IL-2 | | |
| 93567_primary Tr1_resting dy 4- | 0.0 | 0.3 |
| 6 in IL-2 | | |
| 93351_CD45RA CD4 lymphocyte_{—} | 0.3 | 0.2 |
| anti-CD28/anti-CD3 | | |
| 93352_CD45RO CD4 lymphocyte_{—} | 0.0 | 0.0 |
| anti-CD28/anti-CD3 | | |
| 93251_CD8 Lymphocytes_anti-CD28/ | 0.0 | 0.0 |
| anti-CD3 | | |
| 93353_chronic CD8 Lymphocytes | 0.7 | 0.0 |
| 2ry_resting dy 4-6 in IL-2 | | |
| 93574_chronic CD8 Lymphocytes | 0.0 | 0.0 |
| 2ry_activated CD3/CD28 | | |
| 93354_CD4_none | 0.0 | 0.8 |
| 93252_Secondary Th1/Th2/Tr1_anti- | 0.2 | 0.0 |
| CD95 CH11 | | |
| 93103_LAK cells_resting | 13.3 | 12.6 |
| 93788_LAK cells_IL-2 | 0.4 | 0.4 |
| 93787_LAK cells_IL-2 + IL-12 | 0.5 | 1.6 |
| 93789_LAK cells_IL-2 + IFN gamma | 0.8 | 0.4 |
| 93790_LAK cells_IL-2 + IL-18 | 0.5 | 0.4 |
| 93104_LAK cells_PMA/ | 5.4 | 6.9 |
| ionomycin and IL-18 | | |
| 93578_NK Cells IL-2_resting | 0.5 | 0.1 |
| 93109_Mixed Lymphocyte Reaction_{—} | 44.4 | 38.7 |
| Two Way MLR | | |
| 93110_Mixed Lymphocyte Reaction_{—} | 8.0 | 6.9 |
| Two Way MLR | | |
| 93111_Mixed Lymphocyte Reaction_{—} | 0.4 | 0.5 |
| Two Way MLR | | |
| 93112_Mononuclear Cells (PBMCs)_{—} | 1.4 | 1.6 |
| resting | | |
| 93113_Mononuclear Cells (PBMCs)_{—} | 0.7 | 0.5 |
| PWM | | |
| 93114_Mononuclear Cells (PBMCs)_{—} | 2.6 | 3.2 |
| PHA-L | | |
| 93249_Ramos (B cell)_none | 0.0 | 0.3 |
| 93250_Ramos (B cell)_ionomycin | 0.0 | 0.7 |
| 93349_B lymphocytes_PWM | 0.0 | 0.0 |
| 93350_B lymphoytes_CD40L and | 0.3 | 0.6 |
| IL-4 | | |
| 92665_EOL-1 (Eosinophil)_dbcAMP | 0.2 | 0.4 |
| differentiated | | |
| 93248_EOL-1 (Eosinophil)_dbcAMP/ | 0.0 | 0.3 |
| PMAionomycin | | |
| 93356_Dendritic Cells_none | 7.9 | 6.4 |
| 93355_Dendritic Cells_LPS 100 ng/ml | 32.1 | 31.6 |
| 93775_Dendritic Cells_anti-CD40 | 3.8 | 3.6 |
| 93774_Monocytes_resting | 7.9 | 8.4 |
| 93776_Monocytes_LPS 50 ng/ml | 100.0 | 100.0 |
| 93581_Macrophages_resting | 3.6 | 3.5 |
| 93582_Macrophages_LPS 100 ng/ml | 95.3 | 88.3 |
| 93098_HUVEC (Endothelial)_none | 0.2 | 0.0 |
| 93099_HUVEC (Endothelial)_starved | 0.4 | 0.3 |
| 93100_HUVEC (Endothelial)_IL-1b | 0.5 | 0.0 |
| 93779_HUVEC (Endothelial)_IFN | 0.0 | 0.1 |
| gamma | | |
| 93102_HUVEC (Endothelial)_TNF | 0.3 | 0.1 |
| alpha + IFN gamma | | |
| 93101_HUVEC (Endothelial)_TNF | 0.0 | 0.2 |
| alpha + IL4 | | |
| 93781_HUVEC (Endothelial)_IL-11 | 0.2 | 0.0 |
| 93583_Lung Microvascular Endothelial | 0.0 | 0.3 |
| Cells_none | | |
| 93584_Lung Microvascular Endothelial | 0.0 | 0.3 |
| Cells_TNFa (4 ng/ml) and IL1b (1 ng/ | | |
| ml) | | |
| 92662_Microvascular Dermal | 0.0 | 0.0 |
| endothelium_none | | |
| 92663_Microsvasular Dermal | 0.0 | 0.2 |
| endothelium_TNFa (4 ng/ml) and | | |
| IL1b (1 ng/ml) | | |
| 93773_Bronchial epithelium_TNFa | 0.0 | 0.3 |
| (4 ng/ml) and IL1b (1 ng/ml)** | | |
| 93347_Small Airway Epithelium_none | 0.0 | 0.0 |
| 93348_Small Airway Epithelium_{—} | 0.0 | 0.1 |
| TNFa (4 ng/ml) and IL1b (1 ng/ml) | | |
| 92668_Coronery Artery SMC_resting | 0.2 | 0.0 |
| 92669_Coronery Artery SMC_TNFa | 0.0 | 0.0 |
| (4 ng/ml) and IL1b (1 ng/ml) | | |
| 93107_astrocytes_resting | 0.0 | 0.0 |
| 93108_astrocytes_TNFa (4 ng/ml) and | 1.0 | 0.3 |
| IL1b (1 ng/ml) | | |
| 92666_KU-812 (Basophil)_resting | 0.0 | 0.0 |
| 92667_KU-812 (Basophil)_{—} | 0.4 | 0.1 |
| PMA/ionoycin | | |
| 93579_CCD1106 (Keratinocytes)_{—} | 0.4 | 0.0 |
| none | | |
| 93580_CCD1106 (Keratinocytes)_{—} | 0.3 | 0.9 |
| TNFa and IFNg** | | |
| 93791_Liver Cirrhosis | 2.3 | 1.5 |
| 93792_Lupus Kidney | 1.8 | 0.8 |
| 93577_NCI-H292 | 0.0 | 0.4 |
| 93358_NCI-H292_IL-4 | 0.3 | 0.2 |
| 93360_NCI-H292_IL-9 | 0.0 | 0.0 |
| 93359_NCI-H292_IL-13 | 0.0 | 0.2 |
| 93357_NCI-H292_IFN gamma | 0.0 | 0.0 |
| 93777_HPAEC_- | 0.3 | 0.0 |
| 93778_HPAEC_IL-1 beta/TNA alpha | 0.0 | 0.0 |
| 93254_Normal Human Lung Fibro- | 0.3 | 0.0 |
| blast_none | | |
| 93253_Normal Human Lung Fibro- | 0.3 | 0.0 |
| blast_TNFa (4 ng/ml) and IL- | | |
| 1b (1 ng/ml) | | |
| 93257_Normal Human Lung Fibro- | 0.4 | 0.4 |
| blast_IL-4 | | |
| 93256_Normal Human Lung Fibro- | 0.0 | 0.0 |
| blast_IL-9 | | |
| 93255_Normal Human Lung Fibro- | 0.0 | 0.0 |
| blast_IL-13 | | |
| 93258_Normal Human Lung Fibro- | 0.0 | 0.3 |
| blast_IFN gamma | | |
| 93106_Dermal Fibroblasts CCD1070_{—} | 0.7 | 0.2 |
| resting | | |
| 93361_Dermal Fibroblasts CCD1070_{—} | 0.4 | 0.0 |
| TNF alpha 4 ng/ml | | |
| 93105_Dermal Fibroblasts CCD1070_{—} | 0.0 | 0.1 |
| IL-1 beta 1 ng/ml | | |
| 93772_dermal fibroblast_IFN gamma | 0.0 | 0.0 |
| 93771_dermal fibroblast_IL-4 | 0.4 | 0.2 |
| 93259_IBD Colitis 1** | 1.5 | 1.6 |
| 93260_IBD Colitis 2 | 0.0 | 0.2 |
| 93261_IBD Crohns | 0.6 | 0.7 |
| 735010_Colon_normal | 4.1 | 4.1 |
| 735019_Lung_none | 7.5 | 8.0 |
| 64028-1_Thymus_none | 0.9 | 0.1 |
| 64030-1_Kidney_none | 4.8 | 3.0 |

Panel 2D Summary:

The expression of NOV6 in panel 2D is widespread, as it appears to be expressed in most samples in this panel. Of particular interest is the differential expression of this gene in renal cell carcinoma samples when compared to their normal adjacent tissues. These data indicate that this gene may be of utility as a target for therapeutic intervention in kidney cancers.

Panel 3D Summary:

Expression of this gene is highest in the Ramos cell line stimulated with PMA and ionomycin for 16 hrs, followed by lower expression in cell lines derived from gastric carcinoma and pancreatic ductal carcinoma, and lower still in small cell lung carcinoma, medulloblastoma and myelogenous leukemia.

Panel 4D Summary:

The expression of NOV6 is limited to LPS activated monocytes and cell types related (macrophages) or derived from monocytes (dendritic cells). The putative sialoadhesin encoded for by this transcript could be utilized as an adhesion molecule for directing monocyte extravasation into tissues, and as a cell:cell interaction molecule. Protein therapeutics designed from the protein encoded for by this molecule could block monocyte extravasation. Antibody therapeutics could also block extravasation. These therapies could reduce or inhibit inflammation associated with asthma, psoriasis, emphysema, arthritis, and other inflammatory diseases. [Reference: van den Berg et al., J Immunol Mar. 15, 2001;166(6):3637-40 Cutting edge: CD43 functions as a T cell counterreceptor for the macrophage adhesion receptor sialoadhesin (Siglec-1). Sialoadhesin (Siglec-1) is a macrophage-restricted sialic acid-binding receptor that mediates interactions with hemopoietic cells, including lymphocytes. In this study, we identify sialoadhesin counterreceptors on T lymphocytes. Several major glycoproteins (85, 130, 240 kDa) were precipitated by sialoadhesin-Fc fusion proteins from a murine T cell line (TK-1). Binding of sialoadhesin to these glycoproteins was sialic acid dependent and was abolished by mutation of a critical residue (R97A) of the sialic acid binding site in the membrane distal Ig-like domain of sialoadhesin. The 130- and 240-kDa sialoadhesin-binding glycoproteins were identified as the sialomucins CD43 and P-selectin glycoprotein ligand 1 (CD162), respectively. CD43 expressed in COS cells supported increased binding to immobilized sialoadhesin. Finally, sialoadhesin bound different glycoforms of CD43 expressed in Chinese hamster ovary cells, including unbranched (core 1) and branched (core 2) O:-linked glycans, that are normally found on CD43 in resting and activated T cells, respectively. These results identify CD43 as a T cell counterreceptor for sialoadhesin and suggest that in addition to its anti-adhesive role CD43 may promote cell-cell interactions.]

NOV7

Expression of NOV7 was assessed using the primer-probe set Ag582, described in Table S. Results of the RTQ-PCR runs are shown in Table T, U and V.

TABLE S
Probe Name: Ag582

| | | | | | |
|---|---|---|---|---|---|
| Forward | 5′-AGGCTGTAGATAAAGGGGTTCA-3′ | (SEQ ID NO:88) | 59.2 | 22 | 76 |
| Probe | TET-5′-TGAGAGCCACAATGACATCCTTGTCA-3′-TAMRA | (SEQ ID NO:89) | 69.1 | 26 | 122 |
| Reverse | 5′-TTCCCGACTGTAAGCAGTTCTA-3′ | (SEQ ID NO:90) | 59 | 22 | 149 |
| Forward | 5′-AGGCTGTAGATAAAGGGGTTCA-3′ | (SEQ ID NO:91) | 59.2 | 22 | 76 |

TABLE T
Panel 1.1

| | Rel. Expr., % | Rel. Expr., % |
|---|---|---|
| Tissue Name | 1.1tm754f_ag582 | 1.1tm855f_ag582 |
| Adipose | 0.0 | 0.0 |
| Adrenal gland | 0.0 | 0.0 |
| Bladder | 37.6 | 1.7 |
| Brain (amygdala) | 0.0 | 0.0 |
| Brain (cerebellum) | 15.7 | 17.7 |
| Brain (hippocampus) | 0.0 | 0.0 |
| Brain (substantia nigra) | 4.4 | 4.4 |
| Brain (thalamus) | 0.0 | 0.0 |
| Cerebral Cortex | 0.4 | 0.2 |
| Brain (fetal) | 9.2 | 15.3 |
| Brain (whole) | 0.4 | 3.3 |
| CNS ca. (glio/astro) U-118-MG | 6.7 | 8.7 |
| CNS ca. (astro) SF-539 | 0.0 | 2.6 |
| CNS ca. (astro) SNB-75 | 3.3 | 5.3 |
| CNS ca. (astro) SW1783 | 9.5 | 5.6 |
| CNS ca. (glio) U251 | 7.2 | 3.8 |
| CNS ca. (glio) SF-295 | 33.2 | 33.9 |
| CNS ca. (glio) SNB-19 | 21.3 | 15.3 |
| CNS ca. (glio/astro) U87-MG | 11.0 | 11.3 |
| CNS ca.* (neuro; met) SK-N-AS | 3.1 | 2.7 |
| Mammary gland | 0.0 | 0.0 |
| Breast ca. BT-549 | 7.3 | 4.2 |
| Breast ca. MDA-N | 3.1 | 2.9 |
| Breast ca.* (pl. effusion) T47D | 3.7 | 4.0 |
| Breast ca.* (pl. effusion) MCF-7 | 6.3 | 3.6 |
| Breast ca.* (pl. ef) MDA-MB-231 | 10.7 | 14.2 |
| Small intestine | 0.1 | 0.0 |
| Colorectal | 0.0 | 0.0 |
| Colon ca. HT29 | 5.3 | 2.8 |
| Colon ca. CaCo-2 | 2.5 | 0.2 |
| Colon ca. HCT-15 | 0.4 | 0.0 |
| Colon ca. HCT-116 | 1.4 | 1.3 |
| Colon ca. HCC-2998 | 14.3 | 15.3 |
| Colon ca. SW480 | 0.3 | 0.0 |
| Colon ca.* (SW480 met) SW620 | 17.3 | 9.5 |
| Stomach | 0.0 | 0.0 |
| Gastric ca.* (liver met) NCI-N87 | 18.8 | 10.2 |
| Heart | 0.2 | 2.2 |
| Fetal Skeletal | 7.6 | 6.3 |
| Skeletal muscle | 0.0 | 0.0 |
| Endothelial cells | 5.6 | 0.0 |
| Endothelial cells (treated) | 0.0 | 0.0 |
| Kidney | 0.0 | 0.0 |
| Kidney (fetal) | 0.0 | 0.4 |
| Renal ca. 786-0 | 26.2 | 16.7 |
| Renal ca. A498 | 29.7 | 0.0 |
| Renal ca. ACHN | 0.7 | 20.2 |
| Renal ca. TK-10 | 22.4 | 17.0 |
| Renal ca. UO-31 | 59.0 | 20.3 |
| Renal ca. RXF 393 | 12.3 | 5.8 |
| Liver | 0.0 | 0.0 |
| Liver (fetal) | 0.0 | 0.0 |
| Liver ca. (hepatoblast) HepG2 | 1.6 | 0.3 |
| Lung | 0.0 | 0.0 |
| Lung (fetal) | 0.0 | 0.0 |
| Lung ca. (non-s. cell) HOP-62 | 100.0 | 100.0 |
| Lung ca. (large cell) NCI-H460 | 24.7 | 16.8 |
| Lung ca. (non-s. cell) NCI-H23 | 5.0 | 2.0 |
| Lung ca. (non-s. cl) NCI-H522 | 20.2 | 16.7 |
| Lung ca. (non-sm. cell) A549 | 14.9 | 8.7 |
| Lung ca. (s. cell var.) SHP-77 | 2.6 | 0.6 |
| Lung ca. (small cell) LX-1 | 13.8 | 10.2 |
| Lung ca. (small cell) NCI-H69 | 1.1 | 3.4 |
| Lung ca. (squam.) SW 900 | 53.6 | 45.7 |
| Lung ca. (squam.) NCI-H596 | 18.3 | 9.0 |
| Lymph node | 0.0 | 0.0 |
| Spleen | 0.0 | 0.0 |
| Thymus | 0.0 | 0.0 |
| Ovary | 0.0 | 0.0 |
| Ovarian ca. IGROV-1 | 28.9 | 6.1 |
| Ovarian ca. OVCAR-3 | 12.6 | 0.5 |
| Ovarian ca. OVCAR-4 | 1.4 | 0.9 |
| Ovarian ca. OVCAR-5 | 36.3 | 28.7 |
| Ovarian ca. OVCAR-8 | 17.6 | 17.1 |
| Ovarian ca.* (ascites) SK-OV-3 | 23.3 | 19.5 |
| Pancreas | 0.0 | 0.0 |
| Pancreatic ca. CAPAN 2 | 0.6 | 2.9 |
| Pituitary gland | 2.1 | 1.5 |
| Plancenta | 2.3 | 0.0 |
| Prostate | 0.0 | 1.7 |
| Prostate ca.* (bone met) PC-3 | 8.0 | 6.7 |
| Salavary gland | 0.0 | 0.0 |
| Trachea | 0.0 | 0.0 |
| Spinal cord | 1.4 | 0.0 |
| Testis | 0.0 | 0.0 |
| Thyroid | 0.0 | 0.0 |
| Uterus | 0.6 | 1.0 |
| Melanoma M14 | 0.0 | 1.2 |
| Melanoma LOX IMVI | 12.3 | 7.5 |
| Melanoma UACC-62 | 0.4 | 1.4 |
| Melanoma SK-MEL-28 | 27.7 | 26.2 |
| Melanoma* (met) SK-MEL-5 | 13.1 | 17.3 |
| Melanoma Hs688(A).T | 8.1 | 9.2 |
| Melanoma* (met) Hs688(B).T | 2.2 | 5.4 |

TABLE U
Panel 2D

| | Rel. Expr., % | | Rel. Expr., % |
|---|---|---|---|
| Tissue Name | 2dtm2899f_ag582 | Tissue Name | 2dtm2899f_ag582 |
| Normal Colon GENPAK | 68.3 | Kidney NAT Clontech 8120608 | 1.2 |
| 061003 | | | |
| 83219 CC Well to Mod Diff | 24.1 | Kidney Cancer Clontech | 3.3 |
| (ODO3866) | | 8120613 | |
| 83220 CC NAT (ODO3866) | 8.0 | Kidney NAT Clontech 8120614 | 1.1 |
| 83221 CC Gr.2 rectosigmoid | 23.0 | Kidney Cancer Clontech | 32.5 |
| (ODO3868) | | 9010320 | |
| 83222 CC NAT (ODO3868) | 6.5 | Kidney NAT Clontech 9010321 | 16.3 |
| 83235 CC Mod Diff | 62.0 | Normal Uterus GENPAK | 11.0 |
| (ODO3920) | | 061018 | |
| 83236 CC NAT (ODO3920) | 15.7 | Uterus Cancer GENPAK | 13.1 |
| | | 064011 | |
| 83237 CC Gr.2 ascend colon | 23.2 | Normal Thyroid Clontech A + | 1.6 |
| (ODO3921) | | 6570-1 | |
| 83238 CC NAT (ODO3921) | 2.7 | Thyroid Cancer GENPAK | 8.7 |
| | | 064010 | |
| 83241 CC from Partial | 11.2 | Thyroid Cancer INVITROGEN | 2.5 |
| Hepatectomy (ODO4309) | | A302152 | |
| 83242 Liver NAT (ODO4309) | 9.3 | Thyroid NAT INVITROGEN | 1.1 |
| | | A302153 | |
| 87472 Colon mets to lung | 8.2 | Normal Breast GENPAK | 12.1 |
| (OD04451-01) | | 061019 | |
| 87473 Lung NAT (OD04451- | 9.9 | 84877 Breast Cancer | 4.2 |
| 02) | | (OD04566) | |
| Normal Prostate Clontech A + | 21.8 | 85975 Breast Cancer | 49.0 |
| 6546-1 | | (OD04590-01) | |
| 84140 Prostate Cancer | 40.9 | 85976 Breast Cancer Mets | 32.5 |
| (OD04410) | | (OD04590-03) | |
| 84141 Prostate NAT | 31.4 | 87070 Breast Cancer Metastasis | 28.3 |
| (OD04410) | | (OD04655-05) | |
| 87073 Prostate Cancer | 21.6 | GENPAK Breast Cancer | 24.8 |
| (OD04720-01) | | 064006 | |
| 87074 Prostate NAT | 24.0 | Breast Cancer Res. Gen. 1024 | 40.1 |
| (OD04720-02) | | | |
| Normal Lung GENPAK 061010 | 28.5 | Breast Cancer Clontech | 28.5 |
| | | 9100266 | |
| 83239 Lung Met to Muscle | 8.8 | Breast NAT Clontech 9100265 | 10.5 |
| (ODO4286) | | | |
| 83240 Muscle NAT | 11.7 | Breast Cancer INVITROGEN | 13.5 |
| (ODO4286) | | A209073 | |
| 84136 Lung Malignant Cancer | 6.8 | Breast NAT INVITROGEN | 0.9 |
| (OD03126) | | A2090734 | |
| 84137 Lung NAT (OD03126) | 10.5 | Normal Liver GENPAK | 3.2 |
| | | 061009 | |
| 84871 Lung Cancer | 6.1 | Liver Cancer GENPAK 064003 | 13.0 |
| (OD04404) | | | |
| 84872 Lung NAT (OD04404) | 18.4 | Liver Cancer Research Genetics | 3.2 |
| | | RNA 1025 | |
| 84875 Lung Cancer (OD04565) | 55.9 | Liver Cancer Research Genetics | 2.6 |
| | | RNA 1026 | |
| 84876 Lung NAT (OD04565) | 10.7 | Paired Liver Cancer Tissue | 7.2 |
| | | Research Genetics RNA 6004- | |
| | | T | |
| 85950 Lung Cancer (OD04237- | 91.4 | Paired Liver Tissue Research | 15.8 |
| 01) | | Genetics RNA 6004-N | |
| 85970 Lung NAT (OD04237- | 19.8 | Paired Liver Cancer Tissue | 0.5 |
| 02) | | Research Genetics RNA 6005- | |
| | | T | |
| 83255 Ocular Mel Met to Liver | 2.9 | Paired Liver Tissue Research | 0.0 |
| (ODO4310) | | Genetics RNA 6005-N | |
| 83256 Liver NAT (ODO4310) | 7.6 | Normal Bladder GENPAK | 21.8 |
| | | 061001 | |
| 84139 Melanoma Mets to Lung | 6.2 | Bladder Cancer Research | 13.2 |
| (OD04321) | | Genetics RNA 1023 | |
| 84138 Lung NAT (OD04321) | 15.0 | Bladder Cancer INVITROGEN | 5.5 |
| | | A302173 | |
| Normal Kidney GENPAK | 11.8 | 87071 Bladder Cancer | 46.0 |
| 061008 | | (OD04718-01) | |
| 83786 Kidney Ca, Nuclear | 17.4 | 87072 Bladder Normal | 16.3 |
| grade 2 (OD04338) | | Adjacent (OD04718-03) | |
| 83787 Kidney NAT | 5.5 | Normal Ovary Res. Gen. | 2.5 |
| (OD04338) | | | |
| 83788 Kidney Ca Nuclear grade | 54.0 | Ovarian Cancer GENPAK | 7.7 |
| 1/2 (OD04339) | | 064008 | |
| 83789 Kidney NAT (OD04339) | 11.1 | 87492 Ovary Cancer | 19.8 |
| | | (OD04768-07) | |
| 83790 Kidney Ca, Clear cell | 100.0 | 87493 Ovary NAT (OD04768- | 7.2 |
| type (OD04340) | | 08) | |
| 83791 Kidney NAT (OD04340) | 12.2 | Normal Stomach GENPAK | 29.7 |
| | | 061017 | |
| 83792 Kidney Ca, Nuclear | 35.6 | Gastric Cancer Clontech | 1.8 |
| grade 3 (OD04348) | | 9060358 | |
| 83793 Kidney NAT (OD04348) | 14.9 | NAT Stomach Clontech | 2.0 |
| | | 9060359 | |
| 87474 Kidney Cancer | 6.3 | Gastric Cancer Clontech | 1.9 |
| (OD04622-01) | | 9060395 | |
| 87475 Kidney NAT | 2.3 | NAT Stomach Clontech | 3.5 |
| (OD04622-03) | | 9060394 | |
| 85973 Kidney Cancer | 24.0 | Gastric Cancer Clontech | 5.5 |
| (OD04450-01) | | 9060397 | |
| 85974 Kidney NAT | 8.2 | NAT Stomach Clontech | 1.5 |
| (OD04450-03) | | 9060396 | |
| Kidney Cancer Clontech | 0.0 | Gastric Cancer GENPAK | 9.7 |
| 8120607 | | 064005 | |

TABLE V
Panel 4D

| | Rel. Expr., % | Rel. Expr., % |
|---|---|---|
| | 4dtm2938f_{—} | 4dx4tm5034f_{—} |
| Tissue Name | ag582 | ag582_a1 |
| 93768_Secondary Th1_anti-CD28/ | 13.7 | 3.2 |
| anti-CD3 | | |
| 93769_Secondary Th2_anti-CD28/ | 7.1 | 1.0 |
| anti-CD3 | | |
| 93770_Secondary Tr1_anti-CD28/ | 12.9 | 3.8 |
| anti-CD3 | | |
| 93573_Secondary Th1_resting day 4- | 1.7 | 0.0 |
| 6 in IL-2 | | |
| 93572_Secondary Th2_resting day 4- | 3.2 | 4.3 |
| 6 in IL-2 | | |
| 93571_Secondary Tr1_resting day 4- | 2.2 | 2.6 |
| 6 in IL-2 | | |
| 93568_primary Th1_anti-CD28/anti- | 4.9 | 2.8 |
| CD3 | | |
| 93569_primary Th2_anti-CD28/anti- | 1.1 | 4.0 |
| CD3 | | |
| 93570_primary Tr1_anti-CD28/anti- | 6.6 | 4.4 |
| CD3 | | |
| 93565_primary Th1_resting dy 4- | 0.0 | 6.2 |
| 6 in IL-2 | | |
| 93566_primary Th2_resting dy 4- | 0.0 | 7.2 |
| 6 in IL-2 | | |
| 93567_primary Tr1_resting dy 4- | 1.2 | 2.3 |
| 6 in IL-2 | | |
| 93351_CD45RA CD4 lymphocyte_{—} | 55.9 | 12.5 |
| anti-CD28/anti-CD3 | | |
| 93352_CD45RO CD4 lymphocyte_{—} | 15.5 | 1.3 |
| anti-CD28/anti-CD3 | | |
| 93251_CD8 Lymphocytes_anti-CD28/ | 0.0 | 0.7 |
| anti-CD3 | | |
| 93353_chronic CD8 Lymphocytes | 0.0 | 0.0 |
| 2ry_resting dy 4-6 in IL-2 | | |
| 93574_chronic CD8 Lymphocytes | 4.7 | 0.0 |
| 2ry_activated CD3/CD28 | | |
| 93354_CD4_none | 0.6 | 0.0 |
| 93252_Secondary Th1/Th2/Tr1_anti- | 4.2 | 3.7 |
| CD95 CH11 | | |
| 93103_LAK cells_resting | 7.7 | 2.3 |
| 93788_LAK cells_IL-2 | 3.0 | 7.6 |
| 93787_LAK cells_IL-2 + IL-12 | 1.7 | 1.7 |
| 93789_LAK cells_IL-2 + IFN gamma | 0.0 | 4.0 |
| 93790_LAK cells_IL-2 + IL-18 | 0.0 | 6.8 |
| 93104_LAK cells_PMA/ | 20.4 | 16.6 |
| ionomycin and IL-18 | | |
| 93578_NK Cells IL-2_resting | 0.9 | 4.1 |
| 93109_Mixed Lymphocyte Reaction_{—} | 9.2 | 5.7 |
| Two Way MLR | | |
| 93110_Mixed Lymphocyte Reaction_{—} | 2.1 | 3.9 |
| Two Way MLR | | |
| 93111_Mixed Lymphocyte Reaction_{—} | 2.5 | 0.0 |
| Two Way MLR | | |
| 93112_Mononuclear Cells (PBMCs)_{—} | 4.0 | 5.6 |
| resting | | |
| 93113_Mononuclear Cells (PBMCs)_{—} | 2.4 | 5.8 |
| PWM | | |
| 93114_Mononuclear Cells (PBMCs)_{—} | 3.4 | 10.1 |
| PHA-L | | |
| 93249_Ramos (B cell)_none | 0.0 | 0.0 |
| 93250_Ramos (B cell)_ionomycin | 0.8 | 2.0 |
| 93349_B lymphocytes_PWM | 2.4 | 15.1 |
| 93350_B lymphoytes_CD40L and | 6.4 | 17.4 |
| IL-4 | | |
| 92665_EOL-1 (Eosinophil)_dbcAMP | 20.0 | 5.8 |
| differentiated | | |
| 93248_EOL-1 (Eosinophil)_dbcAMP/ | 5.7 | 3.1 |
| PMAionomycin | | |
| 93356_Dendritic Cells_none | 8.8 | 8.0 |
| 93355_Dendritic Cells_LPS 100 ng/ml | 6.8 | 8.4 |
| 93775_Dendritic Cells_anti-CD40 | 6.5 | 10.6 |
| 93774_Monocytes_resting | 20.6 | 21.7 |
| 93776_Monocytes_LPS 50 ng/ml | 12.2 | 11.4 |
| 93581_Macrophages_resting | 7.5 | 19.2 |
| 93582_Macrophages_LPS 100 ng/ml | 2.2 | 6.4 |
| 93098_HUVEC (Endothelial)_none | 5.3 | 11.2 |
| 93099_HUVEC (Endothelial)_starved | 7.4 | 41.9 |
| 93100_HUVEC (Endothelial)_IL-1b | 8.2 | 10.0 |
| 93779_HUVEC (Endothelial)_IFN | 100.0 | 7.2 |
| gamma | | |
| 93102_HUVEC (Endothelial)_TNF | 60.3 | 38.1 |
| alpha + IFN gamma | | |
| 93101_HUVEC (Endothelial)_TNF | 33.9 | 32.6 |
| alpha + IL4 | | |
| 93781_HUVEC (Endothelial)_IL-11 | 15.4 | 8.0 |
| 93583_Lung Microvascular Endothelial | 21.2 | 26.4 |
| Cells_none | | |
| 93584_Lung Microvascular Endothelial | 37.1 | 65.7 |
| Cells_TNFa (4 ng/ml) and IL1b (1 ng/ | | |
| ml) | | |
| 92662_Microvascular Dermal | 26.6 | 34.5 |
| endothelium_none | | |
| 92663_Microsvasular Dermal | 19.6 | 42.8 |
| endothelium_TNFa (4 ng/ml) and | | |
| IL1b (1 ng/ml) | | |
| 93773_Bronchial epithelium_TNFa | 8.6 | 22.5 |
| (4 ng/ml) and IL1b (1 ng/ml)** | | |
| 93347_Small Airway Epithelium_none | 6.9 | 10.6 |
| 93348_Small Airway Epithelium_{—} | 12.9 | 44.3 |
| TNFa (4 ng/ml) and IL1b (1 ng/ml) | | |
| 92668_Coronery Artery SMC_resting | 8.8 | 8.9 |
| 92669_Coronery Artery SMC_TNFa | 21.0 | 4.1 |
| (4 ng/ml) and IL1b (1 ng/ml) | | |
| 93107_astrocytes_resting | 59.9 | 32.8 |
| 93108_astrocytes_TNFa (4 ng/ml) and | 38.4 | 19.2 |
| IL1b (1 ng/ml) | | |
| 92666_KU-812 (Basophil)_resting | 3.3 | 0.0 |
| 92667_KU-812 (Basophil)_PMA/ | 3.3 | 8.9 |
| ionoycin | | |
| 93579_CCD1106 (Keratinocytes)_{—} | 8.4 | 12.5 |
| none | | |
| 93580_CCD1106 (Keratinocytes)_{—} | 39.0 | 6.1 |
| TNFa and IFNg** | | |
| 93791_Liver Cirrhosis | 7.7 | 11.9 |
| 93792_Lupus Kidney | 2.0 | 11.6 |
| 93577_NCI-H292 | 15.6 | 15.5 |
| 93358_NCI-H292_IL-4 | 18.2 | 31.0 |
| 93360_NCI-H292_IL-9 | 5.5 | 30.7 |
| 93359_NCI-H292_IL-13 | 32.8 | 10.2 |
| 93357_NCI-H292_IFN gamma | 28.1 | 14.6 |
| 93777_HPAEC_- | 31.6 | 15.4 |
| 93778_HPAEC_IL-1 beta/TNA alpha | 20.2 | 29.9 |
| 93254_Normal Human Lung Fibro- | 21.5 | 20.4 |
| blast_none | | |
| 93253_Normal Human Lung Fibro- | 4.4 | 8.8 |
| blast_TNFa (4 ng/ml) and IL-1b (1 ng/ | | |
| ml) | | |
| 93257_Normal Human Lung Fibro- | 33.2 | 35.1 |
| blast_IL-4 | | |
| 93256_Normal Human Lung Fibro- | 26.6 | 54.1 |
| blast_IL-9 | | |
| 93255_Normal Human Lung Fibro- | 25.9 | 55.3 |
| blast_IL-13 | | |
| 93258_Normal Human Lung Fibro- | 17.6 | 38.4 |
| blast_IFN gamma | | |
| 93106_Dermal Fibroblasts CCD1070_{—} | 28.1 | 77.0 |
| resting | | |
| 93361_Dermal Fibroblasts CCD1070_{—} | 30.1 | 100.0 |
| TNF alpha 4 ng/ml | | |
| 93105_Dermal Fibroblasts CCD1070_{—} | 10.2 | 38.1 |
| IL-1 beta 1 ng/ml | | |
| 93772_dermal fibroblast_IFN gamma | 25.0 | 8.8 |
| 93771_dermal fibroblast_IL-4 | 39.5 | 16.0 |
| 93260_IBD Colitis 2 | 2.0 | 1.2 |
| 93261_IBD Crohns | 0.0 | 0.0 |
| 735010_Colon_normal | 6.6 | 2.1 |
| 735019_Lung_none | 5.4 | 7.2 |
| 64028-1_Thymus_none | 8.3 | 9.9 |
| 64030-1_Kidney_none | 11.3 | 21.8 |

Panel 1.1 Summary:

NOV7 is expressed at moderately high levels in the brain, bladder, heart, pituitary and uterus. Notably, its expression is seen to be high in fetal skeletal muscle and absent in adult tissue. Therefore this gene may be used as therapy for tissue regeneration. Moreover, this gene is overexpressed in lung cancer, renal cancer, ovarian cancer, CNS cancer, melanoma and breast cancer, with levels in normal tissue being low/undetectable. Therefore, therapies targeted towards this protein may be effective therapeutics in these kinds of cancer.

Panel 2D Summary:

Overall, the expression of this gene shows varied expression across panel 2D. However, there appears to be cancer-associated expression in the samples derived from lung, colon and kidney cancers, when compared to their respective normal adjacent tissues. This is consistent with expression in panel 1.1. Thus, targeting of this gene may provide therapeutic utility in these diseases.

Panel 4D Summary:

Expression of this gene in the two panels does not replicate very well and therefore no firm conclusions can be drawn.

NOV8a

Expression of NOV8a was assessed using the primer-probe set Ag850, described in Table W. Results of the RTQ-PCR runs are shown in Table X.

TABLE W
Probe Name: Ag850

| | | | | | Start |
|---|---|---|---|---|---|
| Primers | Sequences | | TM | Length | Position |
| Forward | 5′-CCTTTCTTCTCTTCCTCCTCAA-3′ | (SEQ ID NO:92) | 591 | 22 | 25 |
| Probe | FAM-5′-CACCTGGCGAGTGCTCCTCTCTG-3′-TAMRA | (SEQ ID NO:93) | 70 | 23 | 71 |
| Reverse | 5′-GGTGGATGGCGTTGTAGAG-3′ | (SEQ ID NO:94) | 59.1 | 19 | 96 |

TABLE X
Panel 4.1D

| | Rel. Expr., % | | Rel. Expr., % |
|---|---|---|---|
| Tissue Name | 4.1dx4tm6089f_ag850_b2 | Tissue Name | 4.1dx4tm6089f_ag850_b2 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.8 |
| CD28/anti-CD3 | | (Endothelial)_IFN gamma | |
| 93770_Secondary Tr1_anti- | 0.0 | 93102_HUVEC | 1.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha + IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.7 | 93583_Lung Microvascular | 0.0 |
| day 4-6 in IL-2 | | Endothelial Cells_none | |
| 93568_primary Th1_anti- | 0.2 | 93584_Lung Microvascular | 0.0 |
| CD28/anti-CD3 | | Endothelial Cells_TNFa | |
| | | (4 ng/ml) and IL1b (1 ng/ml) | |
| 93569_primary Th2_anti- | 0.0 | 92662_Microvascular Dermal | 0.3 |
| CD28/anti-CD3 | | endothelium_none | |
| 93570_primary Tr1_anti- | 0.6 | 92663_Microsvasular Dermal | 0.0 |
| CD28/anti-CD3 | | endothelium_TNFa (4 ng/ | |
| | | ml) and IL1b (1 ng/ml) | |
| 93565_primary Th1_resting | 0.0 | 93773_Bronchial | 0.9 |
| dy 4-6 in IL-2 | | epithelium_TNFa (4 ng/ml) and | |
| | | IL1b (1 ng/ml)** | |
| 93566_primary Th2_resting | 0.0 | 93347_Small Airway | 1.8 |
| dy 4-6 in IL-2 | | Epithelium_none | |
| 93567_primary Tr1_resting | 0.0 | 93348_Small Airway | 4.0 |
| dy 4-6 in IL-2 | | Epithelium_TNFa (4 ng/ml) and | |
| | | IL1b (1 ng/ml) | |
| 93351_CD45RA CD4 | 7.4 | 92668_Coronery Artery | 1.8 |
| lymphocyte anti-CD28/anti- | | SMC_resting | |
| CD3 | | | |
| 93352_CD45RO CD4 | 0.2 | 92669_Coronery Artery | 1.3 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 31.9 |
| CD28/anti-CD3 | | | |
| 93353_chronic CD8 | 0.2 | 93108_astrocytes_TNFa | 33.4 |
| Lymphocytes 2ry_resting dy | | (4 ng/ml) and IL1b (1 ng/ml) | |
| 4-6 in IL-2 | | | |
| 93574_chronic CD8 | 0.3 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.1 | 93579_CCD1106 | 0.0 |
| Th1/Th2/Tr1_anti-CD95 CH11 | | (Keratinocytes)_none | |
| 93103_LAK cells_resting | 0.0 | 93580_CCD1106 | 0.0 |
| | | (Keratinocytes)_TNFa and | |
| | | IFNg** | |
| 93788_LAK cells_IL-2 | 0.0 | 93791_Liver Cirrhosis | 1.0 |
| 93787_LAK cells_IL-2 + IL-12 | 0.0 | 93577_NCI-H292 | 2.2 |
| 93789_LAK cells_IL-2 + IFN | 0.0 | 93358_NCI-H292_IL-4 | 1.1 |
| gamma | | | |
| 93790_LAK cells IL-2 + IL-18 | 0.0 | 93360_NCI-H292_IL-9 | 1.5 |
| 93104_LAK cells_PMA/ | 0.6 | 93359_NCI-H292_IL-13 | 0.9 |
| ionomycin and IL-18 | | | |
| 93578_NK Cells IL-2_resting | 0.0 | 93357_NCI-H292_IFN gamma | 0.3 |
| 93109_Mixed Lymphocyte | 0.0 | 93777_HPAEC_- | 0.0 |
| Reaction_Two Way MLR | | | |
| 93110_Mixed Lymphocyte | 0.0 | 93778_HPAEC_IL-1 beta/TNA | 0.0 |
| Reaction_Two Way MLR | | alpha | |
| 93111_Mixed Lymphocyte | 0.2 | 93254_Normal Human Lung | 0.0 |
| Reaction_Two Way MLR | | Fibroblast_none | |
| 93112_Mononuclear Cells | 0.0 | 93253_Normal Human Lung | 0.0 |
| (PBMCs)_resting | | Fibroblast_TNFa (4 ng/ml) and | |
| | | IL-1b (1 ng/ml) | |
| 93113_Mononuclear Cells | 0.0 | 93257_Normal Human Lung | 0.1 |
| (PBMCs)_PWM | | Fibroblast_IL-4 | |
| 93114_Mononuclear Cells | 0.0 | 93256_Normal Human Lung | 0.0 |
| (PBMCs)_PHA-L | | Fibroblast_IL-9 | |
| 93249_Ramos (B cell)_none | 0.0 | 93255_Normal Human Lung | 0.1 |
| | | Fibroblast_IL-13 | |
| 93250_Ramos (B | 0.0 | 93258_Normal Human Lung | 0.8 |
| cell)_ionomycin | | Fibroblast_IFN gamma | |
| 93349_B lymphocytes_PWM | 0.0 | 93106_Dermal Fibroblasts | 14.5 |
| | | CCD1070_resting | |
| 93350_B lymphoytes_CD40L | 0.1 | 93361_Dermal Fibroblasts | 15.2 |
| and IL-4 | | CCD1070_TNF alpha 4 ng/ml | |
| 92665_EOL-1 | 0.0 | 93105_Dermal Fibroblasts | 10.1 |
| (Eosinophil)_dbcAMP | | CCD1070_IL-1 beta 1 ng/ml | |
| differentiated | | | |
| 93248_EOL-1 | 0.0 | 93772_dermal fibroblast_IFN | 0.0 |
| (Eosinophil)_dbcAMP/ | | gamma | |
| PMAionomycin | | | |
| 93356_Dendritic Cells_none | 0.0 | 93771_dermal fibroblast_IL-4 | 0.0 |
| 93355_Dendritic Cells_LPS | 0.3 | 93892_Dermal fibroblasts_none | 0.0 |
| 100 ng/ml | | | |
| 93775_Dendritic Cells_anti- | 0.0 | 99202_Neutrophils_TNFa + LPS | 0.0 |
| CD40 | | | |
| 93774_Monocytes_resting | 0.0 | 99203_Neutrophils_none | 0.0 |
| 93776_Monocytes_LPS | 0.0 | 735010_Colon_normal | 0.6 |
| 50 ng/ml | | | |
| 93581_Macrophages_resting | 0.0 | 735019_Lung_none | 0.9 |
| 93582_Macrophages_LPS | 0.8 | 64028-1_Thymus_none | 6.0 |
| 100 ng/ml | | | |
| 93098_HUVEC | 0.0 | 64030-1_Kidney_none | 100.0 |
| (Endothelial)_none | | | |
| 93099_HUVEC | 0.0 | | |
| (Endothelial)_starved | | | |

Panel 4.1D Summary:

The NOV8a transcript is highly expressed in normal kidney and astrocytes but not in most other tissues. The expression of this transcript or of the protein it encodes may function as a marker for normal kidney or for astrocytes.

NOV8c

Expression of NOV8c was assessed using the primer-probe sets Ag217, described in Table Y. Results of the RTQ-PCR runs are shown in Tables Z and AA.

TABLE Y
Probe Name: Ag217

| | | | | | Start |
|---|---|---|---|---|---|
| Primers | Sequences | | TM | Length | Position |
| Forward | 5′-ATCTGTGCTGAGGCATGTTCCT-3′ | (SEQ ID NO:95) | | 22 | 163 |
| Probe | FAM-5′-ATCCTCCTCCCTCCCCGGCTCTC-3′-TAMRA | (SEQ ID NO:96) | | 23 | 192 |
| Reverse | 5′-CTGCATGGCTGGTGTGATG-3′ | (SEQ ID NO:97) | | 19 | 222 |

TABLE Z
Panel 1

| | Rel. Expr., % | | Rel. Expr., % |
|---|---|---|---|
| | tm303f | | tm303f |
| Tissue Name | (ag217) | Tissue Name | (ag217) |
| Endothelial cells | 0.0 | Kidney (fetal) | 0.0 |
| Endothelial cells (treated) | 0.0 | Renal ca. 786-0 | 0.0 |
| Pancreas | 0.0 | Renal ca. A498 | 0.0 |
| Pancreatic ca. CAPAN 2 | 5.4 | Renal ca. RXF 393 | 0.0 |
| Adipose | 0.0 | Renal ca. ACHN | 0.0 |
| Adrenal gland | 0.0 | Renal ca. UO-31 | 0.0 |
| Thyroid | 0.0 | Renal ca. TK-10 | 0.0 |
| Salavary gland | 0.0 | Liver | 0.0 |
| Pituitary gland | 0.0 | Liver (fetal) | 0.0 |
| Brain (fetal) | 0.0 | Liver ca. (hepatoblast) HepG2 | 0.0 |
| Brain (whole) | 0.0 | Lung | 0.0 |
| Brain (amygdala) | 0.0 | Lung (fetal) | 0.0 |
| Brain (cerebellum) | 0.0 | Lung ca. (small cell) LX-1 | 3.1 |
| Brain (hippocampus) | 0.0 | Lung ca. (small cell) NCI-H69 | 0.2 |
| Brain (substantia nigra) | 0.0 | Lung ca. (s. cell var.) SHP-77 | 0.0 |
| Brain (thalamus) | 0.0 | Lung ca. (large cell) NCI-H460 | 0.0 |
| Brain (hypothalamus) | 0.0 | Lung ca. (non-sm. cell) A549 | 2.2 |
| Spinal cord | 0.0 | Lung ca. (non-s. cell) NCI-H23 | 0.0 |
| CNS ca. (glio/astro) U87- | 0.0 | Lung ca. (non-s. cell) HOP-62 | 0.0 |
| MG | | | |
| CNS ca. (glio/astro) U-118- | 0.0 | Lung ca. (non-s. cl) NCI-H522 | 0.0 |
| MG | | | |
| CNS ca. (astro) SW1783 | 0.3 | Lung ca. (squam.) SW 900 | 0.6 |
| CNS ca.* (neuro; met) SK-N- | 6.7 | Lung ca. (squam.) NCI-H596 | 0.2 |
| AS | | | |
| CNS ca. (astro) SF-539 | 0.0 | Mammary gland | 0.0 |
| CNS ca. (astro) SNB- | 0.0 | Breast ca.* (pl. effusion) MCF- | 0.0 |
| 75 | | 7 | |
| CNS ca. (glio) SNB- | 0.0 | Breast ca.* (pl. ef) MDA-MB- | 0.0 |
| 19 | | 231 | |
| CNS ca. (glio) | 0.6 | Breast ca.* (pl. effusion) | 0.0 |
| U251 | | T47D | |
| CNS ca. (glio) SF-295 | 15.3 | Breast ca. BT-549 | 0.0 |
| Heart | 0.0 | Breast ca. MDA- | 0.0 |
| | | N | |
| Skeletal muscle | 0.0 | Ovary | 0.0 |
| Bone marrow | 0.0 | Ovarian ca. OVCAR- | 0.0 |
| | | 3 | |
| Thymus | 0.0 | Ovarian ca. OVCAR- | 0.0 |
| | | 4 | |
| Spleen | 0.0 | Ovarian ca. OVCAR- | 0.1 |
| | | 5 | |
| Lymph node | 0.0 | Ovarian ca. OVCAR-8 | 0.0 |
| Colon (ascending) | 0.0 | Ovarian ca. IGROV-1 | 0.0 |
| Stomach | 0.0 | Ovarian ca.* (ascites) SK-OV-3 | 0.0 |
| Small intestine | 0.0 | Uterus | 7.4 |
| Colon ca. SW480 | 2.4 | Plancenta | 100.0 |
| Colon ca.* (SW480 | 4.1 | Prostate | 0.0 |
| met) SW620 | | | |
| Colon ca. HT29 | 0.0 | Prostate ca.* (bone met) PC-3 | 0.0 |
| Colon ca. HCT-116 | 0.0 | Testis | 4.2 |
| Colon ca. CaCo-2 | 0.0 | Melanoma Hs688(A).T | 0.0 |
| Colon ca. HCT-15 | 0.0 | Melanoma* (met) Hs688(B).T | 0.0 |
| Colon ca. HCC-2998 | 0.0 | Melanoma UACC-62 | 0.0 |
| Gastric ca.* (liver met) NCI- | 45.4 | Melanoma M14 | 0.0 |
| N87 | | | |
| Bladder | 0.0 | Melanoma LOX IMVI | 0.0 |
| Trachea | 0.0 | Melanoma* (met) SK-MEL-5 | 0.0 |
| Kidney | 4.1 | Melanoma SK-MEL-28 | 0.0 |

TABLE AA
Panel 4D

| | Rel. Expr., % | Rel. Expr., % |
|---|---|---|
| | 4dx4tm5043f_{—} | 4dx4tm5056f_{—} |
| Tissue Name | ag217_b1 | ag217_b1 |
| 93768_Secondary Th1_anti-CD28/ | 0.0 | 1.2 |
| anti-CD3 | | |
| 93769_Secondary Th2_anti-CD28/ | 0.0 | 0.0 |
| anti-CD3 | | |
| 93770_Secondary Tr1_anti-CD28/ | 2.6 | 0.0 |
| anti-CD3 | | |
| 93573_Secondary Th1_resting day 4- | 1.8 | 0.0 |
| 6 in IL-2 | | |
| 93572_Secondary Th2_resting day 4- | 0.0 | 0.0 |
| 6 in IL-2 | | |
| 93571_Secondary Tr1_resting day 4- | 7.0 | 1.6 |
| 6 in IL-2 | | |
| 93568_primary Th1_anti-CD28/anti- | 0.0 | 0.0 |
| CD3 | | |
| 93569_primary Th2_anti-CD28/anti- | 0.0 | 0.0 |
| CD3 | | |
| 93570_primary Tr1_anti-CD28/anti- | 0.8 | 0.0 |
| CD3 | | |
| 93565_primary Th1_resting dy 4- | 0.0 | 0.0 |
| 6 in IL-2 | | |
| 93566_primary Th2_resting dy 4- | 0.0 | 0.0 |
| 6 in IL-2 | | |
| 93567_primary Tr1_resting dy 4- | 0.0 | 1.3 |
| 6 in IL-2 | | |
| 93351_CD45RA CD4 lymphocyte_{—} | 8.1 | 9.2 |
| anti-CD28/anti-CD3 | | |
| 93352_CD45RO CD4 lymphocyte_{—} | 0.0 | 0.0 |
| anti-CD28/anti-CD3 | | |
| 93251_CD8 Lymphocytes_anti-CD28/ | 0.0 | 0.0 |
| anti-CD3 | | |
| 93353_chronic CD8 Lymphocytes | 0.0 | 0.0 |
| 2ry_resting dy 4-6 in IL-2 | | |
| 93574_chronic CD8 Lymphocytes | 2.1 | 2.5 |
| 2ry_activated CD3/CD28 | | |
| 93354_CD4_none | 0.0 | 0.0 |
| 93252_Secondary Th1/Th2/Tr1_anti- | 0.0 | 0.0 |
| CD95 CH11 | | |
| 93103_LAK cells_resting | 0.0 | 0.0 |
| 93788_LAK cells_IL-2 | 0.0 | 0.0 |
| 93787_LAK cells_IL-2 + IL-12 | 0.0 | 0.0 |
| 93789_LAK cells_IL-2 + IFN gamma | 0.0 | 0.0 |
| 93790_LAK cells_IL-2 + IL-18 | 2.2 | 0.0 |
| 93104_LAK cells_PMA/ | 0.0 | 0.0 |
| ionomycin and IL-18 | | |
| 93578_NK Cells IL-2_resting | 0.0 | 0.0 |
| 93109_Mixed Lymphocyte Reaction_{—} | 0.0 | 0.0 |
| Two Way MLR | | |
| 93110_Mixed Lymphocyte Reaction_{—} | 0.0 | 0.0 |
| Two Way MLR | | |
| 93111_Mixed Lymphocyte Reaction_{—} | 0.0 | 1.5 |
| Two Way MLR | | |
| 93112_Mononuclear Cells (PBMCs)_{—} | 0.0 | 0.0 |
| resting | | |
| 93113_Mononuclear Cells (PBMCs)_{—} | 0.0 | 0.0 |
| PWM | | |
| 93114_Mononuclear Cells (PBMCs)_{—} | 0.0 | 0.0 |
| PHA-L | | |
| 93249_Ramos (B cell)_none | 0.0 | 0.0 |
| 93250_Ramos (B cell)_ionomycin | 0.0 | 1.5 |
| 93349_B lymphocytes_PWM | 0.0 | 3.4 |
| 93350_B lymphoytes_CD40L and | 0.0 | 1.7 |
| IL-4 | | |
| 92665_EOL-1 (Eosinophil)_dbcAMP | 0.0 | 0.0 |
| differentiated | | |
| 93248_EOL-1 (Eosinophil)_dbcAMP/ | 0.0 | 0.0 |
| PMAionomycin | | |
| 93356_Dendritic Cells_none | 0.0 | 0.0 |
| 93355_Dendritic Cells_LPS 100 ng/ml | 1.9 | 1.4 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 1.5 |
| 93582_Macrophages_LPS 100 ng/ml | 6.4 | 0.0 |
| 93098_HUVEC (Endothelial)_none | 0.0 | 0.0 |
| 93099_HUVEC (Endothelial)_starved | 0.0 | 0.0 |
| 93100_HUVEC (Endothelial)_IL-1b | 0.0 | 0.0 |
| 93779_HUVEC (Endothelial)_IFN | 0.0 | 0.0 |
| gamma | | |
| 93102_HUVEC (Endothelial)_TNF | 0.0 | 0.0 |
| alpha + IFN gamma | | |
| 93101_HUVEC (Endothelial)_TNF | 0.0 | 0.0 |
| alpha + IL4 | | |
| 93781_HUVEC (Endothelial)_IL-11 | 0.0 | 2.8 |
| 93583_Lung Microvascular Endothelial | 0.0 | 1.1 |
| Cells_none | | |
| 93584_Lung Microvascular Endothelial | 0.0 | 0.0 |
| Cells_TNFa (4 ng/ ml) and IL1b (1 ng/ | | |
| ml) | | |
| 92662_Microvascular Dermal | 3.7 | 0.0 |
| endothelium_none | | |
| 92663_Microsvasular Dermal | 0.0 | 0.0 |
| endothelium_TNFa (4 ng/ml) and | | |
| IL1b (1 ng/ml) | | |
| 93773_Bronchial epithelium_TNFa | 3.9 | 3.2 |
| (4 ng/ml) and IL1b (1 ng/ml)** | | |
| 93347_Small Airway Epithelium_none | 6.2 | 4.5 |
| 93348_Small Airway Epithelium_{—} | 11.2 | 9.7 |
| TNFa (4 ng/ml) and IL1b (1 ng/ml) | | |
| 92668_Coronery Artery SMC_resting | 5.8 | 5.9 |
| 92669_Coronery Artery SMC_TNFa | 4.2 | 1.9 |
| (4 ng/ml) and IL1b (1 ng/ml) | | |
| 93107_astrocytes_resting | 61.1 | 41.5 |
| 93108_astrocytes_TNFa (4 ng/ml) and | 78.6 | 69.9 |
| IL1b (1 ng/ml) | | |
| 92666_KU-812 (Basophil)_resting | 0.0 | 0.0 |
| 92667_KU-812 (Basophil)_PMA/ | 0.0 | 0.0 |
| ionoycin | | |
| 93579_CCD1106 (Keratinocytes)_{—} | 0.0 | 0.0 |
| none | | |
| 93580_CCD1106 (Keratinocytes)_{—} | 0.0 | 0.0 |
| TNFa and IFNg** | | |
| 93791_Liver Cirrhosis | 10.9 | 5.4 |
| 93792_Lupus Kidney | 0.0 | 2.3 |
| 93577_NCI-H292 | 11.6 | 2.8 |
| 93358_NCI-H292_IL-4 | 2.9 | 3.3 |
| 93360_NCI-H292_IL-9 | 2.3 | 1.8 |
| 93359_NCI-H292_IL-13 | 2.8 | 0.0 |
| 93357_NCI-H292_IFN gamma | 3.8 | 0.0 |
| 93777_HPAEC_- | 0.0 | 0.0 |
| 93778_HPAEC_IL-1 beta/TNA alpha | 0.0 | 0.0 |
| 93254_Normal Human Lung Fibro- | 0.0 | 0.0 |
| blast_none | | |
| 93253_Normal Human Lung Fibro- | 0.0 | 0.0 |
| blast_TNFa (4 ng/ml) and IL-1b (1 ng/ | | |
| ml) | | |
| 93257_Normal Human Lung Fibro- | 0.0 | 0.0 |
| blast_IL-4 | | |
| 93256_Normal Human Lung Fibro- | 0.0 | 0.0 |
| blast_IL-9 | | |
| 93255_Normal Human Lung Fibro- | 0.0 | 0.0 |
| blast_IL-13 | | |
| 93258_Normal Human Lung Fibro- | 0.0 | 1.3 |
| blast_IFN gamma | | |
| 93106_Dermal Fibroblasts CCD1070_{—} | 40.7 | 30.0 |
| resting | | |
| 93361_Dermal Fibroblasts CCD1070_{—} | 13.1 | 17.9 |
| TNF alpha 4 ng/ml | | |
| 93105_Dermal Fibroblasts CCD1070_{—} | 27.0 | 19.2 |
| IL-1 beta 1 ng/ml | | |
| 93772_dermal fibroblast_IFN gamma | 0.0 | 0.0 |
| 93771_dermal fibroblast_IL-4 | 2.3 | 2.6 |
| 93259_IBD Colitis 1** | 0.0 | 0.0 |
| 93260_IBD Colitis 2 | 2.3 | 0.0 |
| 93261_IBD Crohns | 0.0 | 1.1 |
| 735010_Colon_normal | 18.6 | 5.9 |
| 735019_Lung_none | 13.2 | 8.1 |
| 64028-1_Thymus_none | 100.0 | 100.0 |
| 64030-1_Kidney_none | 3.9 | 1.6 |

Panel 1 Summary:

Expression of NOV8c is highest in the placenta, followed by the uterus, testis and kidney. Interestingly, expression in adult kidney is considerably higher than in fetal kidney. Among disease tissues, strong expression is seen in gastric cancer, with lower levels in lung, colon, CNS, ovarian and pancreatic cancers. This pattern indicates that this gene may be a therapeutic target in these conditions.

Panel 4D Summary:

This transcript is highly expressed in the thymus and astrocytes, with lower levels of this transcript are seen in colon and lung, as well as in dermal fibroblasts (either resting or IL-1-treated or TNF-treated). Thymus expression of the transcript suggests that it may be important in T cell development. Retinoic acid has been shown to have effects on T cell devlopment. Agonistic or antagonistic therapies directed against the protein encoded for by this transcript could be used for immune regulation during organ engraftment or as treatment for T cell cancers. [Reference: Yagi J. et al., Cell Immunol Nov. 1, 1997;181(2):153-62. Influence of retinoic acid on the differentiation pathway of T cells in the thymus. This study investigated the ability of retinoic acid (RA) to influence T cell differentiation. All-trans-RA had marked effects on T cell differentiation in murine fetal thymic organ cultures (FTOCs). The time course of the effect of all-trans-RA in FTOC of day 14 C57BL/6 embryos revealed a twofold increase in the frequency of CD4 single-positive (SP) cells and a high level of CD3-bearing cells (CD3high cells) at a later stage of T cell development. At an earlier stage, all-trans-RA induced a twofold increase in the frequency of CD4 SP cells, but significantly suppressed the upregulation of CD3 and TCR. Reverse transcription-PCR using RA receptor (RAR) subtype-specific primers showed that RAR alpha but not beta and gamma is expressed during T cell development in the thymus and that its expression was associated with the generation of CD4/CD8 double-positive (DP) cells. In FTOC of day 16 BALB/c embryos, the level of V beta 3high cells was greatly reduced (1.4% of the CD3high cells) in response to the mouse mammary tumor virus-6-encoded superantigen, but V beta 3-bearing cells were rescued from the deletion in the presence of all-trans-RA (5.6% of the CD3high cells). Further, the inhibitory effect of all-trans-RA on thymocyte deletion was observed when the deletion was induced by a low concentration of staphylococcal enterotoxin B in FTOC. Taken together, these data suggest that RA increases the frequency of mature and self-reactive T cells in the thymus, possibly by inhibiting the process of negative selection at the DP stage of T cell differentiation.]

NOV9

Expression of gene NOV9 was assessed using the primer-probe Ag 1249, described in Table BB. Results of the RTQ-PCR runs are shown in Table CC and DD.

TABLE BB
Probe Name: Ag1249

| | | | | | Start |
|---|---|---|---|---|---|
| Primers | Sequences | | TM | Length | Position |
| Forward | 5′-CAAATGAAGGAGCATGAGAAAG-3′ | (SEQ ID NO:98) | 59 | 22 | 76 |
| Probe | FAM-5′-CCCTGAAATGCTAACTGATCTCCAATG-3′-TAMRA | (SEQ ID NO:99) | 66.8 | 27 | 99 |
| Reverse | 5′-TGGGATACTTGCATAGGACTTG-3′ | (SEQ ID NO:100) | 59.1 | 22 | 135 |

TABLE CC
Panel 1.2

| | Rel. Expr., % | | Rel. Expr., % |
|---|---|---|---|
| Tissue Name | 1.2tm1420f_ag1249 | Tissue Name | 1.2tm1420f_ag1249 |
| Endothelial cells | 0.0 | Kidney (fetal) | 0.0 |
| Endothelial cells (treated) | 0.0 | Renal ca. 786-0 | 0.0 |
| Pancreas | 0.0 | Renal ca. A498 | 0.0 |
| Pancreatic ca. CAPAN 2 | 0.0 | Renal ca. RXF 393 | 0.0 |
| Adrenal Gland (new lot*) | 0.0 | Renal ca. ACHN | 0.0 |
| Thyroid | 0.0 | Renal ca. UO-31 | 0.0 |
| Salavary gland | 0.0 | Renal ca. TK-10 | 0.0 |
| Pituitary gland | 0.0 | Liver | 0.0 |
| Brain (fetal) | 0.0 | Liver (fetal) | 0.0 |
| Brain (whole) | 0.0 | Liver ca. (hepatoblast) HepG2 | 0.0 |
| Brain (amygdala) | 0.0 | Lung | 0.0 |
| Brain (cerebellum) | 0.0 | Lung (fetal) | 0.0 |
| Brain (hippocampus) | 0.0 | Lung ca. (small cell) LX-1 | 0.0 |
| Brain (thalamus) | 0.0 | Lung ca. (small cell) NCI-H69 | 0.0 |
| Cerebral Cortex | 0.0 | Lung ca. (s. cell var.) SHP-77 | 0.0 |
| Spinal cord | 0.0 | Lung ca. (large cell) NCI-H460 | 0.0 |
| CNS ca. (glio/astro) U87- | 0.0 | Lung ca. (non-sm. cell) A549 | 0.0 |
| MG | | | |
| CNS ca. (glio/astro) U-118- | 0.0 | Lung ca. (non-s. cell) NCI-H23 | 0.0 |
| MG | | | |
| CNS ca. (astro) SW1783 | 0.0 | Lung ca (non-s. cell) HOP-62 | 0.0 |
| CNS ca.* (neuro; met) SK-N- | 0.0 | Lung ca. (non-s. cl) NCI-H522 | 0.0 |
| AS | | | |
| CNS ca. (astro) SF-539 | 0.0 | Lung ca. (squam.) SW 900 | 0.0 |
| CNS ca. (astro) SNB-75 | 0.0 | Lung ca. (squam.) NCI-H596 | 0.0 |
| CNS ca. (glio) SNB- | 0.0 | Mammary gland | 0.0 |
| 19 | | | |
| CNS ca. (glio) | 0.0 | Breast ca.* (pl. effusion) MCF- | 0.0 |
| U251 | | 7 | |
| CNS ca. (glio) SF-295 | 0.0 | Breast ca.* (pl. ef) MDA-MB- | 0.0 |
| | | 231 | |
| Heart | 0.0 | Breast ca.* (pl. effusion) T47D | 0.0 |
| Skeletal Muscle (new lot*) | 0.0 | Breast ca. BT-549 | 0.0 |
| Bone marrow | 0.0 | Breast ca. MDA- | 0.0 |
| | | N | |
| Thymus | 0.0 | Ovary | 0.0 |
| Spleen | 0.0 | Ovarian ca. OVCAR-3 | 0.0 |
| Lymph node | 0.0 | Ovarian ca. OVCAR-4 | 0.0 |
| Colorectal | 0.0 | Ovarian ca. OVCAR-5 | 0.0 |
| Stomach | 0.0 | Ovarian ca. OVCAR-8 | 0.0 |
| Small intestine | 0.0 | Ovarian ca. IGROV-1 | 0.0 |
| Colon ca. SW480 | 0.0 | Ovarian ca.* (ascites) SK-OV-3 | 11.7 |
| Colon ca.* (SW480 met) SW620 | 0.0 | Uterus | 0.0 |
| Colon ca. HT29 | 0.0 | Plancenta | 0.0 |
| Colon ca. HCT-116 | 0.0 | Prostate | 0.0 |
| Colon ca. CaCo-2 | 0.0 | Prostate ca.* (bone met) PC-3 | 0.0 |
| 83219 CC Well to Mod Diff | 0.0 | Testis | 0.0 |
| (ODO3866) | | | |
| Colon ca. HCC-2998 | 0.0 | Melanoma Hs688(A).T | 0.0 |
| Gastric ca.* (liver met) NCI- | 0.0 | Melanoma* (met) Hs688(B).T | 0.0 |
| N87 | | | |
| Bladder | 0.0 | Melanoma UACC-62 | 0.0 |
| Trachea | 0.0 | Melanoma M14 | 0.0 |
| Kidney | 0.0 | Melanoma LOX IMVI | 0.0 |

TABLE DD
Panel 4D

| | Rel. Expr., % | Rel. Expr., % |
|---|---|---|
| | 4Dtm2108f_{—} | 4Dtm2161f_{—} |
| Tissue Name | ag1249 | ag1249 |
| 93768_Secondary Th1_anti-CD28/ | 0.0 | 0.0 |
| anti-CD3 | | |
| 93769_Secondary Th2_anti-CD28/ | 0.0 | 0.0 |
| anti-CD3 | | |
| 93770_Secondary Tr1_anti-CD28/ | 0.0 | 0.0 |
| anti-CD3 | | |
| 93573_Secondary Th1_resting day 4- | 0.1 | 0.0 |
| 6 in IL-2 | | |
| 93572_Secondary Th2_resting day 4- | 0.0 | 0.0 |
| 6 in IL-2 | | |
| 93571_Secondary Tr1_resting day 4- | 0.2 | 0.0 |
| 6 in IL-2 | | |
| 93568_primary Th1_anti-CD28/anti- | 0.0 | 3.6 |
| CD3 | | |
| 93569_primary Th2_anti-CD28/anti- | 0.0 | 0.0 |
| CD3 | | |
| 93570_primary Tr1_anti-CD28/anti- | 0.0 | 0.0 |
| CD3 | | |
| 93565_primary Th1_resting dy 4- | 0.0 | 0.0 |
| 6 in IL-2 | | |
| 93566_primary Th2_resting dy 4- | 0.0 | 0.0 |
| 6 in IL-2 | | |
| 93567_primary Tr1_resting dy 4- | 100.0 | 0.0 |
| 6 in IL-2 | | |
| 93351_CD45RA CD4 lymphocyte_{—} | 0.0 | 0.0 |
| anti-CD28/anti-CD3 | | |
| 93352_CD45RO CD4 lymphocyte_{—} | 0.0 | 0.0 |
| anti-CD28/anti-CD3 | | |
| 93251_CD8 Lymphocytes_anti-CD28/ | 0.0 | 0.0 |
| anti-CD3 | | |
| 93353_chronic CD8 Lymphocytes | 0.0 | 0.0 |
| 2ry_resting dy 4-6 in IL-2 | | |
| 93574_chronic CD8 Lymphocytes | 0.0 | 0.0 |
| 2ry_activated CD3/CD28 | | |
| 93354_CD4_none | 0.0 | 0.0 |
| 93252_Secondary Th1/Th2/Tr1_anti- | 0.0 | 0.0 |
| CD95 CH11 | | |
| 93103_LAK cells_resting | 0.0 | 0.0 |
| 93788_LAK cells_IL-2 | 0.0 | 0.0 |
| 93787_LAK cells_IL-2 + IL-12 | 0.0 | 0.0 |
| 93789_LAK cells_IL-2 + IFN gamma | 0.0 | 0.0 |
| 93790_LAK cells_IL-2 + IL-18 | 0.0 | 0.0 |
| 93104_LAK cells_PMA/ | 0.0 | 0.0 |
| ionomycin and IL-18 | | |
| 93578_NK Cells IL-2_resting | 0.0 | 0.0 |
| 93109_Mixed Lymphocyte Reaction_{—} | 0.0 | 0.0 |
| Two Way MLR | | |
| 93110_Mixed Lymphocyte Reaction_{—} | 0.8 | 0.0 |
| Two Way MLR | | |
| 93111_Mixed Lymphocyte Reaction_{—} | 0.0 | 0.0 |
| Two Way MLR | | |
| 93112_Mononuclear Cells (PBMCs)_{—} | 0.0 | 0.0 |
| resting | | |
| 93113_Mononuclear Cells (PBMCs)_{—} | 0.0 | 0.0 |
| PWM | | |
| 93114_Mononuclear Cells (PBMCs)_{—} | 0.0 | 0.0 |
| PHA-L | | |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_CD40L and | 0.0 | 0.0 |
| IL-4 | | |
| 92665_EOL-1 (Eosinophil)_dbcAMP | 0.0 | 0.0 |
| differentiated | | |
| 93248_EOL-1 (Eosinophil)_{—} | 0.0 | 0.0 |
| dbcAMP/PMAionomycin | | |
| 93356_Dendritic Cells_none | 0.0 | 0.0 |
| 93355_Dendritic Cells_LPS 100 ng/ml | 0.0 | 0.0 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/ml | 0.0 | 0.0 |
| 93098_HUVEC (Endothelial)_none | 0.0 | 0.0 |
| 93099_HUVEC (Endothelial)_starved | 0.0 | 0.0 |
| 93100_HUVEC (Endothelial)_IL-1b | 0.0 | 0.0 |
| 93779_HUVEC (Endothelial)_IFN | 0.0 | 0.0 |
| gamma | | |
| 93102_HUVEC (Endothelial)_TNF | 0.0 | 0.0 |
| alpha + IFN gamma | | |
| 93101_HUVEC (Endothelial)_TNF | 0.0 | 0.0 |
| alpha + IL4 | | |
| 93781_HUVEC (Endothelial)_IL-11 | 0.0 | 0.0 |
| 93583_Lung Microvascular Endothelial | 0.0 | 9.5 |
| Cells_none | | |
| 93584_Lung Microvascular Endothelial | 0.0 | 0.0 |
| Cells_TNFa (4 ng/ml) and IL1b (1 ng/ | | |
| ml) | | |
| 92662_Microvascular Dermal | 0.0 | 0.0 |
| endothelium_none | | |
| 92663_Microsvasular Dermal | 0.0 | 0.0 |
| endothelium_TNFa (4 ng/ml) and IL1b | | |
| (1 ng/ml) | | |
| 93773_Bronchial epithelium_TNFa | 0.0 | 6.6 |
| (4 ng/ml) and IL1b (1 ng/ml)** | | |
| 93347_Small Airway Epithelium_none | 0.0 | 0.0 |
| 93348_Small Airway Epithelium_{—} | 0.6 | 0.0 |
| TNFa (4 ng/ml) and IL1b (1 ng/ml) | | |
| 92668_Coronery Artery SMC_resting | 0.0 | 0.0 |
| 92669_Coronery Artery SMC_TNFa | 0.0 | 0.0 |
| (4 ng/ml) and IL1b (1 ng/ml) | | |
| 93107_astrocytes_resting | 0.0 | 0.0 |
| 93108_astrocytes_TNFa (4 ng/ml) and | 0.0 | 0.0 |
| IL1b (1 ng/ml) | | |
| 92666_KU-812 (Basophil)_resting | 0.0 | 0.0 |
| 92667_KU-812 (Basophil)_PMA/ | 0.0 | 0.0 |
| ionoycin | | |
| 93579_CCD1106 (Keratinocytes)_{—} | 1.3 | 0.0 |
| none | | |
| 93580_CCD1106 (Keratinocytes)_{—} | 0.6 | 18.0 |
| TNFa and IFNg** | | |
| 93791_Liver Cirrhosis | 4.4 | 20.0 |
| 93792_Lupus Kidney | 0.6 | 0.0 |
| 93577_NCI-H292 | 0.0 | 0.0 |
| 93358_NCI-H292_IL-4 | 0.0 | 0.0 |
| 93360_NCI-H292_IL-9 | 0.0 | 0.0 |
| 93359_NCI-H292_IL-13 | 0.0 | 8.0 |
| 93357_NCI-H292_IFN gamma | 0.0 | 0.0 |
| 93777_HPAEC_- | 0.0 | 0.0 |
| 93778_HPAEC_IL-1 beta/TNA alpha | 0.0 | 0.0 |
| 93254_Normal Human Lung Fibro- | 0.0 | 0.0 |
| blast_none | | |
| 93253_Normal Human Lung Fibro- | 0.0 | 0.0 |
| blast_TNFa (4 ng/ml) and IL-1b (1 ng/ | | |
| ml) | | |
| 93257_Normal Human Lung Fibro- | 0.0 | 0.0 |
| blast_IL-4 | | |
| 93256_Normal Human Lung Fibro- | 0.0 | 0.0 |
| blast_IL-9 | | |
| 93255_Normal Human Lung Fibro- | 0.0 | 0.0 |
| blast_IL-13 | | |
| 93258_Normal Human Lung Fibro- | 0.0 | 0.0 |
| blast_IFN gamma | | |
| 93106_Dermal Fibroblasts CCD1070_{—} | 0.0 | 0.0 |
| resting | | |
| 93361_Dermal Fibroblasts CCD1070_{—} | 0.0 | 0.0 |
| TNF alpha 4 ng/ml | | |
| 93105_Dermal Fibroblasts CCD1070_{—} | 1.0 | 0.0 |
| IL-1 beta 1 ng/ml | | |
| 93772_dermal fibroblast_IFN gamma | 0.0 | 0.0 |
| 93771_dermal fibroblast_IL-4 | 0.0 | 0.0 |
| 93259_IBD Colitis 1** | 10.6 | 100.0 |
| 93260_IBD Colitis 2 | 2.2 | 5.9 |
| 93261_IBD Crohns | 0.7 | 0.0 |
| 735010_Colon_normal | 0.7 | 0.0 |
| 735019_Lung_none | 0.0 | 0.0 |
| 64028-1_Thymus_none | 1.6 | 0.0 |
| 64030-1_Kidney_none | 0.2 | 0.0 |

Panel 1.2 Summary:

Highest expression of NOV9 is in a sample of adipose tissue, which is known to be contaminated with genomic DNA. The only other sample that shows evidence of expression of this gene was derived from a metastatic ovarian cancer cells line that grew as an ascites. This type of tumor growth, as a liquid cell suspension in a body cavity, is quite unique. The expression of this gene may portent to this type of growth and thus, therapeutic targeting of this gene may have therapeutic benefit for acities growth.

Panel 4D Summary:

Run 2108 does not show a good amplification plot and is therefore not being considered for this analysis. Run 2161 shows highest expression in the colitis 1 sample, probably due to genomic DNA contamination. Levels in other samples are below the detectable level.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, and 34;
(b) a variant of a mature form of an amino acid sequence selected from the group consisting of SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS:2, 4, 6, 8, 10, 12, 14, 16, 18, 20, 22, 24, 26, 28, 30, 32, and 34; and
(d) a variant of an amino acid sequence selected from the group consisting of SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting SEQ ID NOS:2, 4, 6, 8, 10, 12, 14, 16, 18, 20, 22, 24, 26, 28, 30, 32, and 34.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7, 9, 11, 13, 15, 17, 19, 21, 23, 25, 27, 29, 31, and 33.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 26, 28, 30, 32, and 34;
(b) a variant of a mature form of an amino acid sequence selected from the group consisting of SEQ ID NOS:2, 4, 6, 8, 10, 12, 14, 16, 18, 20, 22, 24, 26, 28, 30, 32, and 35, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 26, 28, 30, 32, and 34;
(d) a variant of an amino acid sequence selected from the group consisting SEQ ID NOS:2, 4, 6, 8, 10, 12, 14, 16, 18, 20, 22, 24, 26, 28, 30, 32, and 34, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 26, 28, 30, 32, and 34, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 25, 27, 29, 31, and 33.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, 25, 27, 29, 31, and 33;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, 25, 27, 29, 31, and 33, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 5, 7, 9, 11, 13, 15, 17, 19, 21, 23, 25, 27, 29, 31, and 33, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide,
thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule,
thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 26, 28, 30, 32, and 34, or a biologically active fragment thereof.

49. A method of treating a pathological state in a mammal, the method comprising administering to the mammal the antibody of claim 15 in an amount sufficient to alleviate the pathological state.
